SEQUENCE LISTING (100 sequences)

SEQ ID NO: 1 - DNA, 908 bp, Artificial
  /note = Positive strand of the 907 bp GAL1 promoter
gacaggttatcagcaacaacacagtcatatccattctcaattagctctaccacagtgtgtgaaccaatgtatccagcaccacctgtaaccaaaacaattttagaagtactttcactttgtaactgagctgtcatttatattgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaactataatgactaaatctcattcagaagaagtgattgtacctgagttcaattctagcgcaaaggaattaccaagaccattggccgaaaagtgcaaagtgc

SEQ ID NO: 2 - DNA, 675 bp, Artificial
  /note = Positive strand of the 675 bp GAL1 promoter
cttgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaacccc

SEQ ID NO: 3 - DNA, 650 bp, Artificial
  /note = Positive strand of the 650 bp GAL1 promoter
acatggcattaccaccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggcgacagccctccgacggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacctctatactttaacgtcaaggagaaaaaactataatgactaaatctcattcagaagaagtgattgtacctgagttcaattctag

SEQ ID NO: 4 - DNA, 461 bp, Artificial
  /note = Positive strand of the 461bp GAL1 promoter
attgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaacccc

SEQ ID NO: 5 - DNA, 40 bp, Artificial
  /note = 5′ PCR primer for amplifying a NgoMIV-BamHI 907 bp GAL1 promoter fragment
atgccggcgacaggttatcagcaacaacacagtcatatcc

SEQ ID NO: 6 - DNA, 46 bp, Artificial
  /note = 3′ PCR primer for amplifying a NgoMIV-BamHI 907 bp GAL1 promoter fragment
atggatccgcactttgcacttttcggccaatggtcttggtaattcc

SEQ ID NO: 7 - DNA, 42 bp, Artificial
  /note = 5′ PCR primer for amplifying a NgoMIV-BamHI 675 bp GAL1 promoter fragment
atgccggccttgaattttcaaaaattcttactttttttttgg

SEQ ID NO: 8 - DNA, 42 bp, Artificial
  /note = 3′ PCR primer for amplifying a NgoMIV-BamHI 675 bp GAL1 promoter fragment
atggatccggggttttttctccttgacgttaaagtatagagg

SEQ ID NO: 9 - DNA, 41 bp, Artificial
  /note = 5′ PCR primer for amplifying a NgoMIV-BamHI 650 bp GAL1 promoter fragment
atgccggcacatggcattaccaccatatacatatccatatc

SEQ ID NO: 10 - DNA, 42 bp, Artificial
  /note = 3′ PCR primer for amplifying a NgoMIV-BamHI 650 bp GAL1 promoter fragment
atggatccctagaattgaactcaggtacaatcacttcttctg

SEQ ID NO: 11 - DNA, 38 bp, Artificial
  /note = 5′ PCR primer for amplifying a NgoMIV-BamHI 461 bp GAL1 promoter fragment
atgccggcattgaagtacggattagaagccgccgagcg

SEQ ID NO: 12 - DNA, 42 bp, Artificial
  /note = 3′ PCR primer for amplifying a NgoMIV-BamHI 461 bp GAL1 promoter fragment
atggatcccctctatactttaacgtcaaggagaaaaaacccc

SEQ ID NO: 13 - DNA, 269 bp, Artificial
  /note = Positive strand of the 269 bp CYC1 core promoter, a HindIII-BamHI fragment
gcaagcttcagatccgccaggcgtgtatatagcgtggatggccaggcaactttagtgctgacacatacaggcatatatatatgtgtgcgacgacacatgatcatatggcatgcatgtgctctgtatgtatataaaactcttgttttcttcttttctctaaatattctttccttatacattaggtcctttgtagcataaattactatacttctatagacacgcaaacacaaatacacacactaaattaataatgactgaattggatccat

SEQ ID NO: 14 - DNA, 34 bp, Artificial
  /note = 5′ PCR primer for amplifying a HindIII-BamHI 269 bp CYC1core promoter fragment
gcaagcttcagatccgccaggcgtgtatatagcg

SEQ ID NO: 15 - DNA, 34 bp, Artificial
  3′ PCR primer for amplifying a HindIII-BamHI 269 bp CYC1 core promoter fragment
atggatccaattcagtcattattaatttagtgtg

SEQ ID NO: 16 - DNA, 385 bp, Artificial
  /note = Positive strand of the 385 bp GAL1UAS promoter, a NgoMIV-HindIII fragment
gcggccggctcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatcaagcttgc

SEQ ID NO: 17 - DNA, 32 bp, Artificial
  5′ PCR primer for amplifying a NgoMIV-HindIII 385 bp GAL1UASpromoter fragment
gcggccggctcttagcctaaaaaaaccttctc

SEQ ID NO: 18 - DNA, 31 bp, Artificial
  /note = 3′ PCR primer for amplifying a NgoMIV-HindIII 385 bpGAL1UAS promoter fragment
gcaagcttgatcaaaaatcatcgcttcgctg

SEQ ID NO: 19 - DNA, 385 bp, Artificial
  /note = Positive strand of the 385 bp GAL10UAS promoter, a NgoMIV-HindIII fragment
gcggccggcgatcaaaaatcatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccttcggagggctgtcacccgctcggcggcttctaatccgtacttcaatatagcaatgagcagttaagcgtattactgaaagttccaaagagaaggtttttttaggctaagaaagcttgc

SEQ ID NO: 20 - DNA, 32 bp, Artificial
  /note = 5′ PCR primer for amplifying a NgoMIV-HindIII 385 bpGAL10UAS promoter fragment
gcggccggcgatcaaaaatcatcgcttcgctg

SEQ ID NO: 21 - DNA, 31 bp, Artificial
  /note = 3′ PCR primer for amplifying a NgoMIV-HindIII 385 bpGAL10UAS promoter fragment
gcaagctttcttagcctaaaaaaaccttctc

SEQ ID NO: 22 - DNA, 1623 bp, Artificial
  /note = Positive strand of the 1623 bp coding sequence of the modified human CYP1B1 gene that lacks the sequence encoding amino acids 2-4 (GTS) of the wild type protein
atgctcagcccgaacgacccttggccgctaaacccgctgtccatccagcagaccacgctcctgctactcctgtcggtgctggccactgtgcatgtgggccagcggctgctgaggcaacggaggcggcagctccggtccgcgcccccgggcccgtttgcgtggccactgatcggaaacgcggcggcggtgggccaggcggctcacctctcgttcgctcgcctggcgcggcgctacggcgacgttttccagatccgcctgggcagctgccccatagtggtgctgaatggcgagcgcgccatccaccaggccctggtgcagcagggctcggccttcgccgaccggccggccttcgcctccttccgtgtggtgtccggcggccgcagcatggctttcggccactactcggagcactggaaggtgcagcggcgcgcagcccacagcatgatgcgcaacttcttcacgcgccagccgcgcagccgccaagtcctcgagggccacgtgctgagcgaggcgcgcgagctggtggcgctgctggtgcgcggcagcgcggacggcgccttcctcgacccgaggccgctgaccgtcgtggccgtggccaacgtcatgagtgccgtgtgtttcggctgccgctacagccacgacgaccccgagttccgtgagctgctcagccacaacgaagagttcgggcgcacggtgggcgcgggcagcctggtggacgtgatgccctggctgcagtacttccccaacccggtgcgcaccgttttccgcgaattcgagcagctcaaccgcaacttcagcaacttcatcctggacaagttcttgaggcactgcgaaagccttcggcccggggccgccccccgcgacatgatggacgcctttatcctctctgcggaaaagaaggcggccggggactcgcacggtggtggcgcgcggctggatttggagaacgtaccggccactatcactgacatcttcggcgccagccaggacaccctgtccaccgcgctgcagtggctgctcctcctcttcaccaggtatcctgatgtgcagactcgagtgcaggcagaattggatcaggtcgtggggagggaccgtctgccttgtatgggtgaccagcccaacctgccctatgtcctggccttcctttatgaagccatgcgcttctccagctttgtgcctgtcactattcctcatgccaccactgccaacacctctgtcttgggctaccacattcccaaggacactgtggtttttgtcaaccagtggtctgtgaatcatgacccagtgaagtggcctaacccggagaactttgatccagctcgattcttggacaaggatggcctcatcaacaaggacctgaccagcagagtgatgattttttcagtgggcaaaaggcggtgcattggcgaagaactttctaagatgcagctttttctcttcatctccatcctggctcaccagtgcgatttcagggccaacccaaatgagcctgcgaaaatgaatttcagttatggtctaaccattaaacccaagtcatttaaagtcaatgtcactctcagagagtccatggagctccttgatagtgctgtccaaaatttacaagccaaggaaacttgccaataa

SEQ ID NO: 23 - DNA, 44 bp, Artificial
  /note = 5′ PCR primer for amplifying a BamHI-Xba 1623 bp modified human CYP1B1 gene
atggatccaacagatcatgctcagcccgaacgacccttggccgc

SEQ ID NO: 24 - DNA, 32 bp, Artificial
  /note = 3′ PCR primer for amplifying a BamHI-Xba 1623 bp modified human CYP1B1 gene
gctctagattattggcaagtttccttggcttg

SEQ ID NO: 25 - protein, 677 aa, Homo sapiens
  /note = Wild type human P450 reductase
MGDSHVDTSSTVSEAVAEEVSLFSMTDMILFSLIVGLLTYWFLFRKKKEEVPEFTKIQTLTSSVRESSFVEKMKKTGRNIIVFYGSQTGTAEEFANRLSKDAHRYGMRGMSADPEEYDLADLSSLPEIDNALVVFCMATYGEGDPTDNAQDFYDWLQETDVDLSGVKFAVFGLGNKTYEHFNAMGKYVDKRLEQLGAQRIFELGLGDDDGNLEEDFITWREQFWPAVCEHFGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDVVMSLNNLDEESNKKHPFPCPTSYRTALTYYLDITNPPRTNVLYELAQYASEPSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPVGENGGRALVPMFVRKSQLRLPFKATTPVIMVGPGTGWHPFIGFIQERAWLRQQGKEVGETLLYYGCRRSDEDYLYREELAQFHRDGALTQLNVAFSREQSHKVYVQHLLKQDREHLWKLIEGGAHIYVCGDARNMARDVQNTFYDIVAELGAMEHAQAVDYIKKLMTKGRYSLDVWS

SEQ ID NO: 26 - protein, 653 aa, Artificial
  /note = delta-N24hRD human P450 reductase
MTDMILFSLIVGLLTYWFLFRKKKEEVPEFTKIQTLTSSVRESSFVEKMKKTGRNIIVFYGSQTGTAEEFANRLSKDAHRYGMRGMSADPEEYDLADLSSLPEIDNALVVFCMATYGEGDPTDNAQDFYDWLQETDVDLSGVKFAVFGLGNKTYEHFNAMGKYVDKRLEQLGAQRIFELGLGDDDGNLEEDFITWREQFWPAVCEHFGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDVVMSLNNLDEESNKKHPFPCPTSYRTALTYYLDITNPPRTNVLYELAQYASEPSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPVGENGGRALVPMFVRKSQLRLPFKATTPVIMVGPGTGWHPFIGFIQERAWLRQQGKEVGETLLYYGCRRSDEDYLYREELAQFHRDGALTQLNVAFSREQSHKVYVQHLLKQDREHLWKLIEGGAHIYVCGDARNMARDVQNTFYDIVAELGAMEHAQAVDYIKKLMTKGRYSLDVWS

SEQ ID NO: 27 - protein, 670 aa, Artificial
  /note = delta-N24hRD-cmyc tag human P450 reductase
MTDMILFSLIVGLLTYWFLFRKKKEEVPEFTKIQTLTSSVRESSFVEKMKKTGRNIIVFYGSQTGTAEEFANRLSKDAHRYGMRGMSADPEEYDLADLSSLPEIDNALVVFCMATYGEGDPTDNAQDFYDWLQETDVDLSGVKFAVFGLGNKTYEHFNAMGKYVDKRLEQLGAQRIFELGLGDDDGNLEEDFITWREQFWPAVCEHFGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDVVMSLNNLDEESNKKHPFPCPTSYRTALTYYLDITNPPRTNVLYELAQYASEPSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPVGENGGRALVPMFVRKSQLRLPFKATTPVIMVGPGTGWHPFIGFIQERAWLRQQGKEVGETLLYYGCRRSDEDYLYREELAQFHRDGALTQLNVAFSREQSHKVYVQHLLKQDREHLWKLIEGGAHIYVCGDARNMARDVQNTFYDIVAELGAMEHAQAVDYIKKLMTKGRYSLDVWSSSEQKLISEEDLNGSRL

SEQ ID NO: 28 - DNA, 291 bp, Artificial
  /note = Positive strand of the SUC2 terminator
aggttataaaacttattgtcttttttatttttttcaaaagccattctaaagggctttagcaacgagtgacgaatgtaaaactttatgatttcaaagaataacctccaaaccattgaaaatgtatttttatttttattttctcccgaccccagttacctggaatttgttctttatgtactttatataagtataattctcttaaaaatttttactactttgcaatagacatcattttttcacgtaataaacccacaatcgtaatgtagttgccttacactactaggatggacc

SEQ ID NO: 29 - DNA, 30 bp, Artificial
  /note = 5′ PCR primer for amplifying the XbaI-SacI SUC2 terminator fragment (303 bp)
attctagaaggttataaaacttattgtctt

SEQ ID NO: 30 - DNA, 31 bp, Artificial
  /note = 3′ PCR primer for amplifying the X baI -SacI SUC2 terminator fragment (303 bp)
atgagctcggtccatcctagtagtgtaaggc

SEQ ID NO: 31 - DNA, 42 bp, Artificial
  /note = The coding strand of the c-myc Tag (encodes the peptideEQKLISEEDLNG, SEQ ID No. 103), with yeast-biased codons.  5′-end has an XbaI overhang, whereas 3′-end a SpeI overhand.
ctagagaacaaaagttgatctctgaagaagacttgaacggta

SEQ ID NO: 32 - DNA, 42 bp, Artificial
  /note = The non-coding strand of the c-myc Tag (encodes the peptide EQKLISEEDLNG, SEQ ID No. 103) , with yeast-biased codons.  5′-end has an XbaI overhang, whereas 3′-end an SpeI overhand.
ctagtaccgttcaagtcttcttcagagatcaacttttgttct

SEQ ID NO: 33 - DNA, 42 bp, Artificial
  /note = 5′ PCR primer for amplifying the XhoI-BamHI 675 bp GAL1 promoter fragment
tcctcgagcttgaattttcaaaaattcttactttttttttgg

SEQ ID NO: 34 - DNA, 1974 bp, Artificial
  /note = The coding strand of the delta-N24hRD human P450 reductase gene (1974 bp) with a 3′-end Stop codon, a BamHI-XbaI fragment
ggatccatgacggacatgattctgttttcgctcatcgtgggtctcctaacctactggttcctcttcagaaagaaaaaagaagaagtccccgagttcaccaaaattcagacattgacctcctctgtcagagagagcagctttgtggaaaagatgaagaaaacggggaggaacatcatcgtgttctacggctcccagacggggactgcagaggagtttgccaaccgcctgtccaaggacgcccaccgctacgggatgcgaggcatgtcagcggaccctgaggagtatgacctggccgacctgagcagcctgccagagatcgacaacgccctggtggttttctgcatggccacctacggtgagggagaccccaccgacaatgcccaggacttctacgactggctgcaggagacagacgtggatctctctggggtcaagttcgcggtgtttggtcttgggaacaagacctacgagcacttcaatgccatgggcaagtacgtggacaagcggctggagcagctcggcgcccagcgcatctttgagctggggttgggcgacgacgatgggaacttggaggaggacttcatcacctggcgagagcagttctggccggccgtgtgtgaacactttggggtggaagccactggcgaggagtccagcattcgccagtacgagcttgtggtccacaccgacatagatgcggccaaggtgtacatgggggagatgggccggctgaagagctacgagaaccagaagcccccctttgatgccaagaatccgttcctggctgcagtcaccaccaaccggaagctgaaccagggaaccgagcgccacctcatgcacctggaattggacatctcggactccaaaatcaggtatgaatctggggaccacgtggctgtgtacccagccaacgactctgctctcgtcaaccagctgggcaaaatcctgggtgccgacctggacgtcgtcatgtccctgaacaacctggatgaggagtccaacaagaagcacccattcccgtgccctacgtcctaccgcacggccctcacctactacctggacatcaccaacccgccgcgtaccaacgtgctgtacgagctggcgcagtacgcctcggagccctcggagcaggagctgctgcgcaagatggcctcctcctccggcgagggcaaggagctgtacctgagctgggtggtggaggcccggaggcacatcctggccatcctgcaggactgcccgtccctgcggccccccatcgaccacctgtgtgagctgctgccgcgcctgcaggcccgctactactccatcgcctcatcctccaaggtccaccccaactctgtgcacatctgtgcggtggttgtggagtacgagaccaaggccggccgcatcaacaagggcgtggccaccaactggctgcgggccaaggagcctgtcggggagaacggcggccgtgcgctggtgcccatgttcgtgcgcaagtcccagttacgcctgcccttcaaggccaccacgcctgtcatcatggtgggccccggcaccgggtggcaccctttcataggcttcatccaggagcgggcctggctgcgacagcagggcaaggaggtgggggagacgctgctgtactacggctgccgccgctcggatgaggactacctgtaccgggaggagctggcgcagttccacagggacggtgcgctcacccagctcaacgtggccttctcccgggagcagtcccacaaggtctacgtccagcacctgctaaagcaagaccgagagcacctgtggaagttgatcgaaggcggtgcccacatctacgtctgtggggatgcacggaacatggccagggatgtgcagaacaccttctacgacatcgtggctgagctcggggccatggagcacgcgcaggcggtggactacatcaagaaactgatgaccaagggccgctactccctggacgtgtggagctagtctaga

SEQ ID NO: 35 - DNA, 35 bp, Artificial
  /note = 5′ PCR primer for amplifying the BamHI-XbaI delta-N24hRD human P450 reductase gene (1974 bp) with or without a 3′-end Stop codon
atggatccatgacggacatgattctgttttcgctc

SEQ ID NO: 36 - DNA, 37 bp, Artificial
  /note = 3′ PCR primer for amplifying the BamHI-XbaI delta-N24hRD human P450 reductase gene (1974 bp) with a 3′-end Stop codon
attctagactagctccacacgtccagggagtagcggc

SEQ ID NO: 37 - DNA, 1971 bp, Artificial
  /note = The coding strand of the delta-N24hRD human P450 reductase gene (1971 bp) with no 3′-end Stop codon, a BamHI-XbaI fragment
ggatccatgacggacatgattctgttttcgctcatcgtgggtctcctaacctactggttcctcttcagaaagaaaaaagaagaagtccccgagttcaccaaaattcagacattgacctcctctgtcagagagagcagctttgtggaaaagatgaagaaaacggggaggaacatcatcgtgttctacggctcccagacggggactgcagaggagtttgccaaccgcctgtccaaggacgcccaccgctacgggatgcgaggcatgtcagcggaccctgaggagtatgacctggccgacctgagcagcctgccagagatcgacaacgccctggtggttttctgcatggccacctacggtgagggagaccccaccgacaatgcccaggacttctacgactggctgcaggagacagacgtggatctctctggggtcaagttcgcggtgtttggtcttgggaacaagacctacgagcacttcaatgccatgggcaagtacgtggacaagcggctggagcagctcggcgcccagcgcatctttgagctggggttgggcgacgacgatgggaacttggaggaggacttcatcacctggcgagagcagttctggccggccgtgtgtgaacactttggggtggaagccactggcgaggagtccagcattcgccagtacgagcttgtggtccacaccgacatagatgcggccaaggtgtacatgggggagatgggccggctgaagagctacgagaaccagaagcccccctttgatgccaagaatccgttcctggctgcagtcaccaccaaccggaagctgaaccagggaaccgagcgccacctcatgcacctggaattggacatctcggactccaaaatcaggtatgaatctggggaccacgtggctgtgtacccagccaacgactctgctctcgtcaaccagctgggcaaaatcctgggtgccgacctggacgtcgtcatgtccctgaacaacctggatgaggagtccaacaagaagcacccattcccgtgccctacgtcctaccgcacggccctcacctactacctggacatcaccaacccgccgcgtaccaacgtgctgtacgagctggcgcagtacgcctcggagccctcggagcaggagctgctgcgcaagatggcctcctcctccggcgagggcaaggagctgtacctgagctgggtggtggaggcccggaggcacatcctggccatcctgcaggactgcccgtccctgcggccccccatcgaccacctgtgtgagctgctgccgcgcctgcaggcccgctactactccatcgcctcatcctccaaggtccaccccaactctgtgcacatctgtgcggtggttgtggagtacgagaccaaggccggccgcatcaacaagggcgtggccaccaactggctgcgggccaaggagcctgtcggggagaacggcggccgtgcgctggtgcccatgttcgtgcgcaagtcccagttacgcctgcccttcaaggccaccacgcctgtcatcatggtgggccccggcaccgggtggcaccctttcataggcttcatccaggagcgggcctggctgcgacagcagggcaaggaggtgggggagacgctgctgtactacggctgccgccgctcggatgaggactacctgtaccgggaggagctggcgcagttccacagggacggtgcgctcacccagctcaacgtggccttctcccgggagcagtcccacaaggtctacgtccagcacctgctaaagcaagaccgagagcacctgtggaagttgatcgaaggcggtgcccacatctacgtctgtggggatgcacggaacatggccagggatgtgcagaacaccttctacgacatcgtggctgagctcggggccatggagcacgcgcaggcggtggactacatcaagaaactgatgaccaagggccgctactccctggacgtgtggagctctaga

SEQ ID NO: 38 - DNA, 34 bp, Artificial
  /note = 3′ PCR primer for amplifying the BamHI-XbaI delta-N24hRD human P450 reductase gene (1974 bp) with no 3′-end Stop codon
attctagagctccacacgtccagggagtagcggc

SEQ ID NO: 39 - DNA, 2046 bp, Artificial
  /note = The coding strand of full length 2046 bp human P450reductase gene (hRD_fl) with a 3′-end Stop codon, a BamHI-XbaI fragment
ggatccatgggagactcccacgtggacaccagctccaccgtgtccgaggcggtggccgaagaagtatctcttttcagcatgacggacatgattctgttttcgctcatcgtgggtctcctaacctactggttcctcttcagaaagaaaaaagaagaagtccccgagttcaccaaaattcagacattgacctcctctgtcagagagagcagctttgtggaaaagatgaagaaaacggggaggaacatcatcgtgttctacggctcccagacggggactgcagaggagtttgccaaccgcctgtccaaggacgcccaccgctacgggatgcgaggcatgtcagcggaccctgaggagtatgacctggccgacctgagcagcctgccagagatcgacaacgccctggtggttttctgcatggccacctacggtgagggagaccccaccgacaatgcccaggacttctacgactggctgcaggagacagacgtggatctctctggggtcaagttcgcggtgtttggtcttgggaacaagacctacgagcacttcaatgccatgggcaagtacgtggacaagcggctggagcagctcggcgcccagcgcatctttgagctggggttgggcgacgacgatgggaacttggaggaggacttcatcacctggcgagagcagttctggccggccgtgtgtgaacactttggggtggaagccactggcgaggagtccagcattcgccagtacgagcttgtggtccacaccgacatagatgcggccaaggtgtacatgggggagatgggccggctgaagagctacgagaaccagaagcccccctttgatgccaagaatccgttcctggctgcagtcaccaccaaccggaagctgaaccagggaaccgagcgccacctcatgcacctggaattggacatctcggactccaaaatcaggtatgaatctggggaccacgtggctgtgtacccagccaacgactctgctctcgtcaaccagctgggcaaaatcctgggtgccgacctggacgtcgtcatgtccctgaacaacctggatgaggagtccaacaagaagcacccattcccgtgccctacgtcctaccgcacggccctcacctactacctggacatcaccaacccgccgcgtaccaacgtgctgtacgagctggcgcagtacgcctcggagccctcggagcaggagctgctgcgcaagctggcctcctcctccggcgagggcaaggagctgtacctgagctgggtggtggaggcccggaggcacatcctggccatcctgcaggactgcccgtccctgcggccccccatcgaccacctgtgtgagctgctgccgcgcctgcaggcccgctactactccatcgcctcatcctccaaggtccaccccaactctgtgcacatctgtgcggtggttgtggagtacgagaccaaggccggccgcatcaacaagggcgtggccaccaactggctgcgggccaaggagcctgccggggagaacggcggccgtgcgctggtgcccatgttcgtgcgcaagtcccagttacgcctgcccttcaaggccaccacgcctgtcatcatggtgggccccggcaccggggtggcacccttcataggcttcatccaggagcgggcctggctgcgacagcagggcaaggaggtgggggagacgctgctgtactacggctgccgccgctcagatgaggactacctgtaccgggaggagctggcgcagttccacagggacggtgcgctcacccagctcaacgtggccttctcccgggagcagtcccacaaggtctacgtccagcacctgctaaagcaagaccgagagcacctgtggaagttgatcgaaggcggtgcccacatctacgtctgtggggatgcacggaacatggccagggatgtgcagaacaccttctacgacatcgtggctgagctcggggccatggagcacgcgcaggcggtggactacatcaagaaactgatgaccaagggccgctactccctggacgtgtggagctagtctaga

SEQ ID NO: 40 - DNA, 42 bp, Artificial
  /note = 5′ PCR primer for amplifying the BamHI-XbaI hRD_fl human P450 reductase gene (2046 bp) with a 3′-end Stop codon
atggatccatgggagactcccacgtggacaccagctccaccg

SEQ ID NO: 41 - DNA, 48 bp, Artificial
  /note = 3′ PCR primer for amplifying the BamHI-XbaI hRD_fl human P450 reductase gene (2046 bp) with a 3′-end Stop codon
attctagactagctccacacgtccagggagtagcggcccttggtcatc

SEQ ID NO: 42 - DNA, 2013 bp, Artificial
  /note = The coding strand of the delta-N24hRD-cmyc tag human P450 reductase gene (2013 bp) delta-N24hRD with a 3′-end c-myc tag sequence
atgacggacatgattctgttttcgctcatcgtgggtctcctaacctactggttcctcttcagaaagaaaaaagaagaagtccccgagttcaccaaaattcagacattgacctcctctgtcagagagagcagctttgtggaaaagatgaagaaaacggggaggaacatcatcgtgttctacggctcccagacggggactgcagaggagtttgccaaccgcctgtccaaggacgcccaccgctacgggatgcgaggcatgtcagcggaccctgaggagtatgacctggccgacctgagcagcctgccagagatcgacaacgccctggtggttttctgcatggccacctacggtgagggagaccccaccgacaatgcccaggacttctacgactggctgcaggagacagacgtggatctctctggggtcaagttcgcggtgtttggtcttgggaacaagacctacgagcacttcaatgccatgggcaagtacgtggacaagcggctggagcagctcggcgcccagcgcatctttgagctggggttgggcgacgacgatgggaacttggaggaggacttcatcacctggcgagagcagttctggccggccgtgtgtgaacactttggggtggaagccactggcgaggagtccagcattcgccagtacgagcttgtggtccacaccgacatagatgcggccaaggtgtacatgggggagatgggccggctgaagagctacgagaaccagaagcccccctttgatgccaagaatccgttcctggctgcagtcaccaccaaccggaagctgaaccagggaaccgagcgccacctcatgcacctggaattggacatctcggactccaaaatcaggtatgaatctggggaccacgtggctgtgtacccagccaacgactctgctctcgtcaaccagctgggcaaaatcctgggtgccgacctggacgtcgtcatgtccctgaacaacctggatgaggagtccaacaagaagcacccattcccgtgccctacgtcctaccgcacggccctcacctactacctggacatcaccaacccgccgcgtaccaacgtgctgtacgagctggcgcagtacgcctcggagccctcggagcaggagctgctgcgcaagatggcctcctcctccggcgagggcaaggagctgtacctgagctgggtggtggaggcccggaggcacatcctggccatcctgcaggactgcccgtccctgcggccccccatcgaccacctgtgtgagctgctgccgcgcctgcaggcccgctactactccatcgcctcatcctccaaggtccaccccaactctgtgcacatctgtgcggtggttgtggagtacgagaccaaggccggccgcatcaacaagggcgtggccaccaactggctgcgggccaaggagcctgtcggggagaacggcggccgtgcgctggtgcccatgttcgtgcgcaagtcccagttacgcctgcccttcaaggccaccacgcctgtcatcatggtgggccccggcaccgggtggcaccctttcataggcttcatccaggagcgggcctggctgcgacagcagggcaaggaggtgggggagacgctgctgtactacggctgccgccgctcggatgaggactacctgtaccgggaggagctggcgcagttccacagggacggtgcgctcacccagctcaacgtggccttctcccgggagcagtcccacaaggtctacgtccagcacctgctaaagcaagaccgagagcacctgtggaagttgatcgaaggcggtgcccacatctacgtctgtggggatgcacggaacatggccagggatgtgcagaacaccttctacgacatcgtggctgagctcggggccatggagcacgcgcaggcggtggactacatcaagaaactgatgaccaagggccgctactccctggacgtgtggagctctagtgaacaaaagttgatctctgaagaagacttgaacggttctaggttataa

SEQ ID NO: 43 - DNA, 34 bp, Artificial
  /note = 5′ PCR primer for amplifying the human P450 reductase gene variants integrated into yeast chromosomal loci (confirmation of integration) (a part of the beginning of the human reductase gene)
cgcggatccatgacggacatgattctgttttcgc

SEQ ID NO: 44 - DNA, 22 bp, Artificial
  /note = 3′ PCR primer for amplifying the human P450 reductase gene variants integrated into yeast chromosomal loci (confirmation of integration) (a sequence from the middle of thehuman reductase gene)
ccggcacgccatcctgcatccc

SEQ ID NO: 45 - DNA, 30 bp, Artificial
  /note = 5′ PCR primer for confirming the integration of two copies of the hRD variant genes, integrated at different yeast chromosomal loci (bp 351-bp 380 of GAL1-675 promoter sequence; SEQ ID No 2)
gattctacaatactagcttttatggttatg

SEQ ID NO: 46 - DNA, 25 bp, Artificial
  /note = 3′ PCR primer for confirming the integration of two copies of the hRD variant genes, integrated at different yeast chromosomal loci (bp 51-bp 75 of the SUC2 terminator sequence; SEQ ID No 28)
gggctttagcaacgagtgacgaatg

SEQ ID NO: 47 - DNA, 840 bp, Saccharomyces cerevisiae
  /note = Positive strand of the full-length S. cerevisiae TRP1 gene, a BamHI-XhoI fragment
cgggatccaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgccctcgagcgg

SEQ ID NO: 48 - DNA, 46 bp, Artificial
  5′ PCR primer for amplifying the BamHI-XhoI full length yeastTRP1 gene (840 bp)
cgggatccaattcggtcgaaaaaagaaaaggagagggccaagaggg

SEQ ID NO: 49 - DNA, 46 bp, Artificial
  /note = 3′ PCR primer for amplifying the BamHI-XhoI full length yeast TRP1 gene (840 bp)
ccgctcgagggcaagtgcacaaacaatacttaaataaatactactc

SEQ ID NO: 50 - DNA, 1218 bp, Saccharomyces cerevisiae
  /note = Positive strand of the S cerevisiae PRA1 gene coding sequence
atgttcagcttgaaagcattattgccattggccttgttgttggtcagcgccaaccaagttgctgcaaaagtccacaaggctaaaatttataaacacgagttgtccgatgagatgaaagaagtcactttcgagcaacatttagctcatttaggccaaaagtacttgactcaatttgagaaagctaaccccgaagttgttttttctagggagcatcctttcttcactgaaggtggtcacgatgttccattgacaaattacttgaacgcacaatattacactgacattactttgggtactccacctcaaaacttcaaggttattttggatactggttcttcaaacctttgggttccaagtaacgaatgtggttccttggcttgtttcctacattctaaatacgatcatgaagcttcatcaagctacaaagctaatggtactgaatttgccattcaatatggtactggttctttggaaggttacatttctcaagacactttgtccatcggggatttgaccattccaaaacaagacttcgctgaggctaccagcgagccgggcttaacatttgcatttggcaagttcgatggtattttgggtttgggttacgataccatttctgttgataaggtggtccctccattttacaacgccattcaacaagatttgttggacgaaaagagatttgccttttatttgggagacacttcaaaggatactgaaaatggcggtgaagccacctttggtggtattgacgagtctaagttcaagggcgatatcacttggttacctgttcgtcgtaaggcttactgggaagtcaagtttgaaggtatcggtttaggcgacgagtacgccgaattggagagccatggtgccgccatcgatactggtacttctttgattaccttgccatcaggattagctgaaatgattaatgctgaaattggggccaagaagggttggaccggtcaatatactctagactgtaacaccagagacaatctacctgatctaattttcaacttcaatggctacaacttcactattgggccatacgattacacgcttgaagtttcaggctcctgtatctctgcaattacaccaatggatttcccagaacctgttggcccactggccatcgttggtgatgccttcttgcgtaaatactattctatttacgatttgggcaacaatgcggttggtttggccaaagcaatttga

SEQ ID NO: 51 - DNA, 455 bp, Artificial
  /note = Positive strand of the 5′end of the PRA1 gene (455bp)isloated as a SacI-BamHI fragment
gcgagctcatgttcagcttgaaagcattattgccattggccttgttgttggtcagcgccaaccaagttgctgcaaaagtccacaaggctaaaatttataaacacgagttgtccgatgagatgaaagaagtcactttcgagcaacatttagctcatttaggccaaaagtacttgactcaatttgagaaagctaaccccgaagttgttttttctagggagcatcctttcttcactgaaggtggtcacgatgttccattgacaaattacttgaacgcacaatattacactgacattactttgggtactccacctcaaaacttcaaggttattttggatactggttcttcaaacctttgggttccaagtaacgaatgtggttccttggcttgtttcctacattctaaatacgatcatgaagcttcatcaagctacaaagctaatggtactgggatcccg

SEQ ID NO: 52 - DNA, 44 bp, Artificial
  /note = 5′ PCR primer for amplifying the SacI-BamHI 5′end of the PRA1 gene (455 bp)
gcgagctcatgttcagcttgaaagcattattgccattggccttg

SEQ ID NO: 53 - DNA, 35 bp, Artificial
  /note = 3′ PCR primer for amplifying the SacI-BamHI 5′end of the PRA1 gene (455 bp)
cgggatcccagtaccattagctttgtagcttgatg

SEQ ID NO: 54 - DNA, 446 bp, Artificial
  /note = The positive strand of the 3′end of the PRA1 gene (446 bp) isolated as a XhoI-KpnI fragment
ccgctcgaggttcgtcgtaaggcttactgggaagtcaagtttgaaggtatcggtttaggcgacgagtacgccgaattggagagccatggtgccgccatcgatactggtacttctttgattaccttgccatcaggattagctgaaatgattaatgctgaaattggggccaagaagggttggaccggtcaatatactctagactgtaacaccagagacaatctacctgatctaattttcaacttcaatggctacaacttcactattgggccatacgattacacgcttgaagtttcaggctcctgtatctctgcaattacaccaatggatttcccagaacctgttggcccactggccatcgttggtgatgccttcttgcgtaaatactattctatttacgatttgggcaacaatgcggttggtttggccaaagcaatttgaggtacccc

SEQ ID NO: 55 - DNA, 43 bp, Artificial
  /note = 5′ PCR primer for amplifying the XhoI-KpnI 3′end of the PRA1 gene (446 bp)
ccgctcgaggttcgtcgtaaggcttactgggaagtcaagtttg

SEQ ID NO: 56 - DNA, 45 bp, Artificial
  3′ PCR primer for amplifying the XhoI-KpnI 3′end of the PRA1 gene (446 bp)
ggggtacctcaaattgctttggccaaaccaaccgcattgttgccc

SEQ ID NO: 57 - DNA, 1656 bp, Saccharomyces cerevisiae
  /note = Positive stand of the S. cerevisiae HRD1 gene coding sequence
atggtgccagaaaatagaaggaaacagttggcaatttttgtagttgtcacatatttgctcacattttattgcgtgtattcagccaccaagacaagcgtttcctttttgcaagtaacactgaagctaaatgaaggcttcaatctaatggttttgtcgatattcatcttattaaattctaccttactatggcaactcctaacgaaactattatttggtgaactgaggcttattgagcatgagcacatttttgaaaggttaccatttaccattataaacaccttgtttatgtcctcactgttccacgaacggtattttttcacagtggcattttttggactattactactctatctgaaagttttccattggattttaaaggataggctggaggccttattacagtcaataaatgattccaccacaatgaaaacccttatctttagtagattctcatttaacctcgtactattggcggttgtagactaccagataataacacgatgcatctcctccatatatacaaaccaaaagagtgatattgaatccacatccctttacctgatacaagtaatggagtttaccatgcttttgattgatttgctaaatttattcctacagacttgtttgaatttctgggaattttatcgctcacaacaaagtctgtctaatgagaacaaccatattgtccatggcgatcctacagatgaaaacacggttgagtctgatcaatctcagccagtgctgaatgacgacgacgatgacgacgatgatgatagacaatttaccggcctggagggtaaattcatgtatgaaaaagcaattgacgtattcacaagattcttaaaaacggcacttcatttgtctatgctaataccatttaggatgcctatgatgcttttgaaagatgtggtgtgggatatcttggcactatatcaaagtggcacaagtttgtggaaaatctggagaaataacaaacagctcgacgacactcttgtcactgtcaccgtagaacagctacaaaattctgcaaatgatgacaatatttgtatcatttgtatggatgagttaatacattctccaaaccagcagacgtggaagaataaaaacaagaaacccaaaaggttaccttgtggccacatacttcatttgtcgtgtttaaagaattggatggaacgttctcagacttgtcctatttgtagattgcctgtctttgatgaaaaaggtaatgttgtgcaaacgactttcacttccaatagtgatatcacgacacagaccaccgtaacagatagcactgggatagcgacagatcaacaaggtttcgcaaacgaagtagatctacttcccacaagaacaacttcccctgatataaggatagtgcctactcaaaatatagacacattagcaatgagaacaaggtcaacctctacaccatctcctacgtggtatacgttcccattacataaaactggtgataattctgttgggtcaagccgatcagcctacgaatttttgatcacaaattcagatgagaaagaaaatggtattcctgtcaaattaacaatagaaaatcacgaagtaaattctctgcatggagacgggggcgagcaaattgccaagaaaattgtcataccagataaatttatccagcatatctag

SEQ ID NO: 58 - DNA, 520 bp, Artificial
  /note = The positive strand of the 5′end of the HRD1 gene (520 bp) isolated as a SacI-BamHI fragment
gcgagctcatggtgccagaaaatagaaggaaacagttggcaatttttgtagttgtcacatatttgctcacattttattgcgtgtattcagccaccaagacaagcgtttcctttttgcaagtaacactgaagctaaatgaaggcttcaatctaatggttttgtcgatattcatcttattaaattctaccttactatggcaactcctaacgaaactattatttggtgaactgaggcttattgagcatgagcacatttttgaaaggttaccatttaccattataaacaccttgtttatgtcctcactgttccacgaacggtattttttcacagtggcattttttggactattactactctatctgaaagttttccattggattttaaaggataggctggaggccttattacagtcaataaatgattccaccacaatgaaaacccttatctttagtagattctcatttaacctcgtactattggcggttgtagactaccagataataacacgatgcggatcccg

SEQ ID NO: 59 - DNA, 40 bp, Artificial
  /note = 5′ PCR primer for amplifying the SacI-BamHI 5′end of the HRD1 gene (520 bp)
gcgagctcatggygccagaaaatagaaggaaacagttggc

SEQ ID NO: 60 - DNA, 42 bp, Artificial
  /note = 3′ PCR primer for amplifying the SacI-BamHI 5′end of the HRD1 gene (520 bp)
cgggatccgcatcgtgttattatctggtagtctacaaccgcc

SEQ ID NO: 61 - DNA, 553 bp, Artificial
  /note = The positive strand of the 3′end of the HRD1 gene (553 bp) isolated as a XhoI-KpnI fragment
ccgctcgagggttaccttgtggccacatacttcatttgtcgtgtttaaagaattggatggaacgttctcagacttgtcctatttgtagattgcctgtctttgatgaaaaaggtaatgttgtgcaaacgactttcacttccaatagtgatatcacgacacagaccaccgtaacagatagcactgggatagcgacagatcaacaaggtttcgcaaacgaagtagatctacttcccacaagaacaacttcccctgatataaggatagtgcctactcaaaatatagacacattagcaatgagaacaaggtcaacctctacaccatctcctacgtggtatacgttcccattacataaaactggtgataattctgttgggtcaagccgatcagcctacgaatttttgatcacaaattcagatgagaaagaaaatggtattcctgtcaaattaacaatagaaaatcacgaagtaaattctctgcatggagacgggggcgagcaaattgccaagaaaattgtcataccagataaatttatccagcatatctagggtacccc

SEQ ID NO: 62 - DNA, 41 bp, Artificial
  /note = 5′ PCR primer for amplifying the XhoI-KpnI 3′end of the HRD1 gene (553 bp)
ccgctcgagggttaccttgtggccacatacttcatttgtcg

SEQ ID NO: 63 - DNA, 41 bp, artificial
  /note = 3′ PCR primer for amplifying the XhoI-KpnI 3′end of the HRD1 gene (553 bp)
ggggtaccctagatatgctggataaatttatctggtatgac

SEQ ID NO: 64 - DNA, 2982 bp, Saccharomyces cerevisiae
  /note = Positive strand of the S. cerevisiae HRD2 gene coding sequence
atggtagacgaaagtgataagaaacaacagactattgatgaacaatcgcagataagtcctgaaaagcagacaccaaataagaaggataaaaaaaaggaggaagaggagcagctatccgaagaagatgcaaagcttaaaacagatttagagctattggttgaaaggctgaaagaagacgattcatctttatatgaagcgtctttgaatgccttaaaggaatcgatcaaaaattctacgagttctatgaccgccgttcccaaacctttgaaattccttcgtccgacatatccagacctatgttctatctacgacaaatggacagatccaaacttgaaatcctctttagcggatgtcttgtccattttagccatgacatactctgaaaatggtaaacacgattcattgagatatagattactctccgacgtttctgattttgaagggtggggacatgagtacatccgtcatttagctttggagatcggtgaagtatataatgatcaagtggaaaaagatgctgaagatgaaacatcatctgatggatctaaaagcgatggatctgcggctacttccgggttcgaattttccaaggaagatactcttcgtctatgtttagacattgtcccatactttttgaaacacaacggtgaagaagacgcggtagatttgcttttggaaattgaatctattgacaagctacctcaatttgtcgatgagaataccttccaaagagtttgtcaatatatggttgcatgtgttccacttttgccacctcctgaagacgttgcctttttaaaaaccgcatattctatttatctatctcagaacgaacttacagatgcaattgctttggcagttagactaggggaagaggatatgatcagatccgttttcgatgctactagcgatccagtgatgcataaacaattagcatatattttagccgcacaaaagacatcgtttgagtatgaaggtgttcaggatataattggaaatggtaaattgtctgaacactttttatatcttgctaaagaattgaatctaactgggccaaaagtcccagaagacatttataagagccatttggataactctaaatctgtgttctcaagcgccggcctagattctgctcaacaaaatctagcttcgtcatttgtcaacgggttcttaaacttaggttattgtaacgataaattaattgtggataatgataattgggtatataaaaccaaaggtgacggtatgacttccgcggtagctagtattggatctatataccagtggaatctcgatggtttgcaacaactggataagtatctctacgttgatgagccagaagttaaagctggtgcattattaggtattggtatttccgcttccggtgtgcatgatggtgaagttgaaccagctttgttacttttgcaagattatgttactaacccagacactaaaattagttcagcagctattcttggcctgggtattgcatttgctggtagcaagaatgacgaagtgttgggtttgttattaccaattgccgcatccactgacttgcctattgaaactgcagctatggcttccttagctttggctcatgttttcgttggtacttgtaatggtgacattacaacttctatcatggacaactttttagagcgtacagccatcgagttgaagaccgactgggtaaggtttttggcacttgctttaggtattctatacatgggccaaggtgaacaggtggatgatgttttagaaactattagtgctattgaacatccaatgacatcagctattgaagtattagttggttcatgtgcttatactggtacgggtgatgttttgttaattcaagatttgttacatcgtttgacccctaaaaacgtaaaaggtgaagaagatgcagatgaagaagaaactgccgaaggacagactaatagcataagtgactttttgggtgagcaagtgaatgaacctaccaagaacgaggaagctgagatcgaagtggacgaaatggaggttgatgccgaaggagaggaagttgaagttaaagcagaaataacagagaagaagaatggtgagagtttagaaggtgaagagataaaatccgaagaaaagaagggaaaatcttccgataaggatgctactacggacgggaaaaatgatgatgaggaggaagaaaaagaagcaggcatagtcgatgaacttgcttatgcagttttgggtattgctttgattgcccttggtgaagatattggcaaggaaatgtctttacgtcattttggtcatttaatgcattatggtaatgaacacatccgtcgtatggttcctttagcgatgggtattgtttccgtttctgatccacaaatgaaggtgttcgataccttaactcgtttttcacatgacgccgatttagaagtttcaatgaattcaattttcgctatgggtctatgtggtgctggtactaacaatgcaaggttagctcaactattaagacagttggcaagttattattcacgtgaacaagacgctttgtttatcaccagattagcacaagggttattgcatcttggtaaaggtactatgacaatggatgtgttcaatgatgcacatgttttgaataaggtgacattagcatcaatattaactaccgcagttggtttagtatcgccaagtttcatgttgaagcatcatcaattgttctacatgttgaatgccggtataaggccaaagtttattctagcattaaacgatgagggagagccaataaaggttaatgtgcgcgttggtcaagcagtggaaactgtcggccaagcaggcagaccaaaaaagattactggctggattacgcaatccactcctgttttactaaatcatggcgaaagagcagaactagaaactgatgaatatattagttacacaagccatattgagggcgtagtaattttaaagaagaaccctgactatcgtgaagaggagtaa

SEQ ID NO: 65 - DNA, 443 bp, Artificial
  /note = The positive strand of the 5′end of the HRD2 gene (443 bp) isolated as a SacI-BamHI fragment
gcgagctcatggtagacgaaagtgataagaaacaacagactattgatgaacaatcgcagataagtcctgaaaagcagacaccaaataagaaggataaaaaaaaggaggaagaggagcagctatccgaagaagatgcaaagcttaaaacagatttagagctattggttgaaaggctgaaagaagacgattcatctttatatgaagcgtctttgaatgccttaaaggaatcgatcaaaaattctacgagttctatgaccgccgttcccaaacctttgaaattccttcgtccgacatatccagacctatgttctatctacgacaaatggacagatccaaacttgaaatcctctttagcggatgtcttgtccattttagccatgacatactctgaaaatggtaaacacgattcattgagatatagattactctccgacgggatcccg

SEQ ID NO: 66 - DNA, 40 bp, Artificial
  /note = 5′ PCR primer for amplifying the SacI-BamHI 5′end of the HRD2 gene (443 bp)
gcgagctcatggtagacgaaagtgataagaaacaacagac

SEQ ID NO: 67 - DNA, 42 bp, Artificial
  /note = 3′ PCR primer for amplifying the SacI-BamHI 5′end of the HRD2 gene (443 bp)
cgggatcccgtcggagagtaatctatatctcaatgaatcgtg

SEQ ID NO: 68 - DNA, 526 bp, Artificial
  /note = The positive strand of the 3′end of the HRD2 gene (526 bp) isolated as a XhoI-KpnI fragment
ccgctcgaggcaaggttagctcaactattaagacagttggcaagttattattcacgtgaacaagacgctttgtttatcaccagattagcacaagggttattgcatcttggtaaaggtactatgacaatggatgtgttcaatgatgcacatgttttgaataaggtgacattagcatcaatattaactaccgcagttggtttagtatcgccaagtttcatgttgaagcatcatcaattgttctacatgttgaatgccggtataaggccaaagtttattctagcattaaacgatgagggagagccaataaaggttaatgtgcgcgttggtcaagcagtggaaactgtcggccaagcaggcagaccaaaaaagattactggctggattacgcaatccactcctgttttactaaatcatggcgaaagagcagaactagaaactgatgaatatattagttacacaagccatattgagggcgtagtaattttaaagaagaaccctgactatcgtgaagaggagtaaggtaccc

SEQ ID NO: 69 - DNA, 41 bp, Artificial
  /note = 5′ PCR primer for amplifying the XhoI-KpnI 3′end of the HRD2 gene (526 bp)
ccgctcgaggcaaggttagctcaactattaagacagttggc

SEQ ID NO: 70 - DNA, 39 bp, Artificial
  /note = 3′ pcr primer for amplifying the xhoi-kpni 5′end of the hrd2 gene (526 bp)
ggggtaccttactcctcttcacgatagtcagggttcttc

SEQ ID NO: 71 - DNA, 1139 bp, Saccharomyces cerevisiae
  /note = Positive strand of the complete S. cerevisiae UBC7 gene
ctgcagcagtcaccttcaatttgtgcaccattttcgtattctgtactttgcgatgtagagtctactaaaatagcgtctctgatagccatgggtgaaggtcttcctctagttctcaccttaattagcattcggttgagaatgcctgcatgttgaagagcgatgccctctgatgcacgatgcacacgcatatttgttcccattaaatattatcatctctgatagagcttgaagaacttaccagactgtttcaagtttaaaacaaggcgcctcatcgcatctacctcacgaagatgcagcagcttattgtctcgagatccttcttataactcctttttcgccattacccgaaaacgagattacagcctctaaaccaagagctcgaaaagcgccaaagtaaactctggcgtttagcgtacgaaggagattatcctaaaaggaacttccctagtaatagtgtaatttggaagggcatagcatgtcgaaaaccgctcagaaacgtctcctcaaggagcttcaacagttaattaaagattctccacctggtatagtggctggtcccaaatcggagaataacatattcatttgggactgcctaattcaagggcctccagatacgccatacgctgatggtgtttttaatgctaagctagagtttcctaaagactatccgttatctccacctaaacttactttcacacccagcatactacatccaaatatttatccaaatggggaagtgtgcatatccattctacactcccctggtgatgatcctaacatgtacgaattagcggaagaaagatggtcgccagtgcaaagtgtagaaaaaattctattaagtgttatgagcatgttgagtgagcccaatatcgaaagtggtgccaacattgatgcttgcatcttgtggagagataatagacctgaatttgagagacaggtaaagttatccattttgaaatcattaggattctgaaagcaggtagcaggttaatgatcatttggtcttccttttaactgttctctatatacataaataagcatatctaccatctaaaagaaagcaaatgtcacataataaatatgttaaaatctcaatgtcttcctggcttttcaggaattcaaaccatttccatttcgtt

SEQ ID NO: 72 - DNA, 513 bp, Artificial
  /note = The positive strand of the 5′end of the UBC7 gene (513 bp) isolated as a SacI-BamHI fragment
gcgagctcccttcaatttgtgcaccattttcgtattctgtactttgcgatgtagagtctactaaaatagcgtctctgatagccatgggtgaaggtcttcctctagttctcaccttaattagcattcggttgagaatgcctgcatgttgaagagcgatgccctctgatgcacgatgcacacgcatatttgttcccattaaatattatcatctctgatagagcttgaagaacttaccagactgtttcaagtttaaaacaaggcgcctcatcgcatctacctcacgaagatgcagcagcttattgtctcgagatccttcttataactcctttttcgccattacccgaaaacgagattacagcctctaaaccaagagctcgaaaagcgccaaagtaaactctggcgtttagcgtacgaaggagattatcctaaaaggaacttccctagtaatagtgtaatttggaagggcatagcatgtcgaaaaccgctcagaaacgtctcctcaaggggatcccg

SEQ ID NO: 73 - DNA, 39 bp, Artificial
  /note = 5′ PCR primer for amplifying the SacI-BamHI 5′end of the UBC7 gene (513 bp)
gcgagctcccttcaatttgtgcaccattttcgtattctg

SEQ ID NO: 74 - DNA, 42 bp, Artificial
  /note = 3′ PCR primer for amplifying the SacI-BamHI 5′end of the UBC7 gene (513 bp)
cgggatccccttgaggagacgtttctgagcggttttcgacat

SEQ ID NO: 75 - DNA, 443 bp, Artificial
  /note = The positive strand of the 3′end of the UBC7 gene (443bp)isolated as a XhoI-KpnI fragment
ccgctcgaggtggctggtcccaaatcggagaataacatattcatttgggactgcctaattcaagggcctccagatacgccatacgctgatggtgtttttaatgctaagctagagtttcctaaagactatccgttatctccacctaaacttactttcacacccagcatactacatccaaatatttatccaaatggggaagtgtgcatatccattctacactcccctggtgatgatcctaacatgtacgaattagcggaagaaagatggtcgccagtgcaaagtgtagaaaaaattctattaagtgttatgagcatgttgagtgagcccaatatcgaaagtggtgccaacattgatgcttgcatcttgtggagagataatagacctgaatttgagagacaggtaaagttatccattttgaaatcattaggattctgaggtacccc

SEQ ID NO: 76 - DNA, 42 bp, Artificial
  /note = 5′ PCR primer for amplifying the XhoI-KpnI 3′end of the UBC7 gene (443 bp)
ccgctcgaggtggctggtcccaaatcggagaataacatattc

SEQ ID NO: 77 - DNA, 53 bp, Artificial
  /note = 3′ PCR primer for amplifying the XhoI-KpnI 5′end of the UBC7 gene (443 bp)
ggggtacctcagaatcctaatgatttcaaaatggataactttacctgtctctc

SEQ ID NO: 78 - DNA, 2090 bp, Saccharomyces cerevisiae
  /note = Positive strand of the complete S. cerevisiae P450 reductase gene (2090 bp), yRD includes its promoter & terminator
ttcagatctatgccgtttggaatagacaacaccgacttcactgtcctggcggggctagtgcttgccgtgctactgtacgtaaagagaaactccatcaaggaactgctgatgtccgatgacggagatatcacagctgtcagctcgggcaacagagacattgctcaggtggtgaccgaaaacaacaagaactacttggtgttgtatgcgtcgcagactgggactgccgaggattacgccaaaaagttttccaaggagctggtggccaagttcaacctaaacgtgatgtgcgcagatgttgagaactacgactttgagtcgctaaacgatgtgcccgtcatagtctcgatttttatctctacatatggtgaaggagacttccccgacggggcggtcaactttgaagactttatttgtaatgcggaagcgggtgcactatcgaacctgaggtataatatgtttggtctgggaaattctacttatgaattctttaatggtgccgccaagaaggccgagaagcatctctccgccgcgggcgctatcagactaggcaagctcggtgaagctgatgatggtgcaggaactacagacgaagattacatggcctggaaggactccatcctggaggttttgaaagacgaactgcatttggacgaacaggaagccaagttcacctctcaattccagtacactgtgttgaacgaaatcactgactccatgtcgcttggtgaaccctctgctcactatttgccctcgcgtcagttgaaccgcaacgcagacggcatccaattgggtcccttcgatttgtctcaaccgtatattgcacccatcgtgaaatctcgcgaactgttctcttccaatgaccgtaattgcatccactctgaatttgacttgtccggctctaacatcaagtactccactggtgaccatcttgctgtttggccttccaacccattggaaaaggtcgaacagttcttatccatattcaacctggaccctgaaaccatttttgacttgaagcccctggatcccaccgtcaaagtgcccttcccaacgccaactactattggcgctgctattaaacactatttggaaattacaggacctgtctccagacaattgttttcatctttgattcagttcgcccccaacgctgacgtcaaggaaaaattgactctgctttcgaaagacaaggaccaattcgccgtcgagataacctccaaatatttcaacatcgcagatgctctgaaatatttgtctgatggcgccaaatgggacaacgtacccatgcaattcttggtcgaatcagttccccaaatgactcctcgttactactctatctcttcctcttctctgtctgaaaagcaaaccgtccatgtcacctccattgtggaaaactttcctaacccagaattgcctgatgctcctccaggtgttggtgttacgactaacttgttaagaaacattcaattggctcaaaacaatgttaacattgccgaaactaacctacctgttcactacgatttaaatggcccgcgtaaacttttcgccaattacaaattgcccgtccacgttcgtcgttctaacttcagattgccttccaacccttccaccccagttatcatgatcggtccaggtaccggtgttgccccattccgtgggtttatcagagagcgtgtcgcgttcctcgaatcacaaaagaagggcggtaacaacgtttcgctaggtaagcatatactgttttatggatcccgtaacactgatgatttcttgtaccaggacgaatggccagaatacgccaaaaaattggatggttcgttcgaaatggtcgtggcccattccaggttgccaaacaccaaaaaagtttatgttcaagataaattaaaggattacgaagaccaagtatttgaaatgattaacaacggtgcatttatctacgtctgtggtgatgcaaagggtatggccaagggtgtgtcaaccgcattggttggcatcttatcccgtggtaaatccattaccactgatgaagcaacagagctaatcaagatgctcaagacttcaggtagataccaagaagatgtctggtctagaat

SEQ ID NO: 79 - DNA, 33 bp, Artificial
  /note = 5′ PCR primer for amplifying the BglII-XbaI yeast P450reductase complete gene fragment (2090 bp)
atagatctatgccgtttggaatagacaacaccg

SEQ ID NO: 80 - DNA, 40 bp, Artificial
  /note = 3′ PCR primer for amplifying the BglII-XbaI yeast P450reductase complete gene fragment (2090 bp)
attctagattaccagacatcttcttggtatctacctgaag

SEQ ID NO: 81 - DNA, 1279 bp, Unknown
  /note = Delta-yRD gene
atcacagctgtcagctcgggcaacagagacattgctcaggtggtgaccgaaaacaacaagaactacttggtgttgtatgcgtcgcagactgggactgccgaggattacgccaaaaagttttccaaggagctggtggccaagttcaacctaaacgtgatgtgcgcagatgttgagaactacgactttgagtcgctaaacgatgtgcccgtcatagtctcgatttttatctctacatatggtgaaggagacttccccgacggggcggtcaactttgaagactttatttgtaatgcggaagcgggtgcactatcgaacctgaggtataatatgtttggtctgggaaattctacttatgaattctttaatggtgccgccaagaaggccgagaagcatctctccgccgcgggcgctatcagactaggcaagctcggtgaagctgatgatggtgcaggaactacagacgaagattacatggcctggaaggactccatcctggaggttttgaaagacgaactgcatttggacgaacaggaagccaagttcacctctcaattccagtacactgtgttgaacgaaatcactgactccatgtcgcttggtgaaccctctgctcactatttgccctcgcgtcagttgaaccgcaacgcagacggcatccaattaattggctcaaaacaatgttaacattgccgaaactaacctacctgttcactacgatttaaatggcccgcgtaaacttttcgccaattacaaattgcccgtccacgttcgtcgttctaacttcagattgccttccaacccttccaccccagttatcatgatcggtccaggtaccggtgttgccccattccgtgggtttatcagagagcgtgtcgcgttcctcgaatcacaaaagaagggcggtaacaacgtttcgctaggtaagcatatactgttttatggatcccgtaacactgatgatttcttgtaccaggacgaatggccagaatacgccaaaaaattggatggttcgttcgaaatggtcgtggcccattccaggttgccaaacaccaaaaaagtttatgttcaagataaattaaaggattacgaagaccaagtatttgaaatgattaacaacggtgcatttatctacgtctgtggtgatgcaaagggtatggccaagggtgtgtcaaccgcattggttggcatcttatcccgtggtaaatccattaccactgatgaagcaacagagctaatcaagatgctcaagacttcaggtagataccaagaagatgtctgg

SEQ ID NO: 82 - DNA, 22 bp, Artificial
  /note = Forward primer for amplification of delta-yRD to confirmyRD gene disruption
gacattgctcaggtggtgaccg

SEQ ID NO: 83 - DNA, 33 bp, Artificial
  /note = Reverse primer for amplification of delta-yRD to confirmyRD gene disruption
ctagtctagattaccagacatcttcttggtatc

SEQ ID NO: 84 - DNA, 559 bp, Artificial
  /note = The 3′ end fragment of delta-yRD after digestion with SwaI (559bps)
aaatggcccgcgtaaacttttcgccaattacaaattgcccgtccacgttcgtcgttctaacttcagattgccttccaacccttccaccccagttatcatgatcggtccaggtaccggtgttgccccattccgtgggtttatcagagagcgtgtcgcgttcctcgaatcacaaaagaagggcggtaacaacgtttcgctaggtaagcatatactgttttatggatcccgtaacactgatgatttcttgtaccaggacgaatggccagaatacgccaaaaaattggatggttcgttcgaaatggtcgtggcccattccaggttgccaaacaccaaaaaagtttatgttcaagataaattaaaggattacgaagaccaagtatttgaaatgattaacaacggtgcatttatctacgtctgtggtgatgcaaagggtatggccaagggtgtgtcaaccgcattggttggcatcttatcccgtggtaaatccattaccactgatgaagcaacagagctaatcaagatgctcaagacttcaggtagataccaagaagatgtctgg

SEQ ID NO: 85 - DNA, 720 bp, Artificial
  /note = The 5′ end fragment of delta-yRD after digestion with SwaI (720bps)
atcacagctgtcagctcgggcaacagagacattgctcaggtggtgaccgaaaacaacaagaactacttggtgttgtatgcgtcgcagactgggactgccgaggattacgccaaaaagttttccaaggagctggtggccaagttcaacctaaacgtgatgtgcgcagatgttgagaactacgactttgagtcgctaaacgatgtgcccgtcatagtctcgatttttatctctacatatggtgaaggagacttccccgacggggcggtcaactttgaagactttatttgtaatgcggaagcgggtgcactatcgaacctgaggtataatatgtttggtctgggaaattctacttatgaattctttaatggtgccgccaagaaggccgagaagcatctctccgccgcgggcgctatcagactaggcaagctcggtgaagctgatgatggtgcaggaactacagacgaagattacatggcctggaaggactccatcctggaggttttgaaagacgaactgcatttggacgaacaggaagccaagttcacctctcaattccagtacactgtgttgaacgaaatcactgactccatgtcgcttggtgaaccctctgctcactatttgccctcgcgtcagttgaaccgcaacgcagacggcatccaattaattggctcaaaacaatgttaacattgccgaaactaacctacctgttcactacgattt

SEQ ID NO: 86 - DNA, 421 bp, Artificial
  /note = A BglII-SpeI fragment of human cytochrome b5 (421 bp)
gcagatctatggcagagcagtcggacgaggccgtgaagtactacaccctagaggagattcagaagcacaaccacagcaagagcacctggctgatcctgcaccacaaggtgtacgatttgaccaaatttctggaagagcatcctggtggggaagaagttttaagggaacaagctggaggtgacgctactgagaactttgaggatgtcgggcactctacagatgccagggaaatgtccaaaacattcatcattggggagctccatccagatgacagaccaaagttaaacaagcctccggaaactcttatcactactattgattctagttccagttggtggaccaactgggtgatccctgccatctctgcagtggccgtcgccttgatgtatcgcctatacatggcagaggactgaactagtcg

SEQ ID NO: 87 - DNA, 35 bp, Artificial
  /note = 5′ PCR primer for amplifying the BglII-SpeI human cytochrome b5 gene fragment (421 bp)
gcagatctatggcagagcagtcggacgaggccgtg

SEQ ID NO: 88 - DNA, 39 bp, Artificial
  /note = 3′ PCR primer for amplifying the BglII-SpeI human cytochrome b5 gene fragment (421 bp)
cgactagttcagtcctctgccatgtataggcgatacatc

SEQ ID NO: 89 - DNA, 602 bp, Unknown
  /note = The ADH2 promoter
ccggtcgacgccggcggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaaaaaaaaaaggcagaggagagcatagaaatggggttcactttttggtaaagctatagcatgcctatcacatataaatagagtgccagtagcgacttttttcacactcgaaatactcttactactgctctcttgttgtttttatcacttcttgtttcttcttggtaaatagaatatcaagctacaaaaagcatacaatcaactatcaactattaactatatcgtaatacacaaagcttggatcccg

SEQ ID NO: 90 - DNA, 41 bp, Artificial
  /note = 5′ PCR primer for amplifying the SalI(NgoMIV)-(HindIII) BamHI yeast ADH2 promoter fragment (602 bp)
ccggtcgacgccggcggcaaaacgtaggggcaaacaaacgg

SEQ ID NO: 91 - DNA, 38 bp, Artificial
  /note = 3′ PCR primer for amplifying the SalI(NgoMIV)-(HindIII) BamHI yeast ADH2 promoter fragment (602 bp)
cgggatccaagctttgtgtattacgatatagttaatag

SEQ ID NO: 92 - DNA, 1512 bp, Homo sapiens
  /note = Human CYP2D6 gene
ggatccaaaaaaatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctacaccactgccgtgattcatgaggtgcagcgctttggggacatcgtccccctgggtatgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagtctaga

SEQ ID NO: 93 - DNA, 1573 bp, Homo sapiens
  /note = Human CYP1A2 gene
atggatccaaaaaaatggcattgtcccagtctgttcccttctcggccacagagcttctcctggcctctgccatcttctgcctggtattctgggtgctcaagggtttgaggcctcgggtccccaaaggcctgaaaagtccaccagagccatggggctggcccttgctcgggcatgtgctgaccctggggaagaacccgcacctggcactgtcaaggatgagccagcgctacggggacgtcctgcagatccgcattggctccacgcccgtgctggtgctgagccgcctggacaccatccggcaggccctggtgcggcagggcgacgatttcaagggccggcctgacctctacacctccaccctcatcactgatggccagagcttgaccttcagcacagactctggaccggtgtgggctgcccgccggcgcctggcccagaatgccctcaacaccttctccatcgcctctgacccagcttcctcatcctcctgctacctggaggagcatgtgagcaaggaggctaaggccctgatcagcaggttgcaggagctgatggcagggcctgggcacttcgacccttacaatcaggtggtggtgtcagtggccaacgtcattggtgccatgtgcttcggacagcacttccctgagagtagcgatgagatgctcagcctcgtgaagaacactcatgagttcgtggagactgcctcctccgggaaccccctggacttcttccccatccttcgctacctgcctaaccctgccctgcagaggttcaaggccttcaaccagaggttcctgtggttcctgcagaaaacagtccaggagcactatcaggactttgacaagaacagtgtccgggacatcacgggtgccctgttcaagcacagcaagaaggggcctagagccagcggcaacctcatcccacaggagaagattgtcaaccttgtcaatgacatctttggagcaggatttgacacagtcaccacagccatctcctggagcctcatgtaccttgtgaccaagcctgagatacagaggaagatccagaaggagctggacactgtgattggcagggagcggcggccccggctctctgacagaccccagctgccctacttggaggccttcatcctggagaccttccgacactcctccttcttgcccttcaccatcccccacagcacaacaagggacacaacgctgaatggcttctacatccccaagaaatgctgtgtcttcgtaaaccagtggcaggtcaaccatgacccagagctgtgggaggacccctctgagttccggcctgagcggttcctcaccgccgatggcactgccattaacaagcccttgagtgagaagatgatgctgtttggcatgggcaagcgccggtgtatcggggaagtcctggccaagtgggagatcttcctcttcctggccatcctgctacagcaactggagttcagcgtgccgccgggcgtgaaagtcgacctgacccccatctacgggctgaccatgaagcacgcccgctgtgaacatgtccaggcgcggctgcgcttctccatcaactgactcgagat

SEQ ID NO: 94 - DNA, 672 bp, Unknown
  /note = The PGK1 promoter (672 bp)
atgtcgacgccggccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaggatccta

SEQ ID NO: 95 - DNA, 43 bp, Artificial
  /note = 5′ PCR primer for amplifying the SalI(NgoMIV)-BamHI yeast PGK1 promoter fragment (672 bp)
atgtcgacgccggccgatttgggcgcgaatcctttattttggc

SEQ ID NO: 96 - DNA, 42 bp, Artificial
  /note = 3′ PCR primer for amplifying the SalI(NgoMIV)-BamHI yeast PGK1 promoter fragment (672 bp)
taggatcctgttttatatttgttgtaaaaagtagataattac

SEQ ID NO: 97 - DNA, 696 bp, Unknown
  /note = The pBR-GAPDH promoter (696 bp)
atgtcgacgccggcgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgatccccagcttagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaatctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaaggatccta

SEQ ID NO: 98 - DNA, 43 bp, Artificial
  /note = 5′ PCR primer for amplifying the SalI(NgoMIV)-BamHI the pBR-GAPDH promoter fragment (696 bp)
atgtcgacgccggcgctctcccttatgcgactcctgcattagg

SEQ ID NO: 99 - DNA, 45 bp, Artificial
  /note = 3′ PCR primer for amplifying the SalI(NgoMIV)-BamHI the pBR-GAPDH promoter fragment (696 bp)
taggatcctttgtttatgtgtgtttattcgaaactaagttcttgg

SEQ ID NO: 100 - protein, 12 aa, Artificial
  /note = C-terminal epitope tag
EQKLISEEDLNG

(19) United States
(12) Patent Application Publication
(10) Pub. No.: US 2009/0186415 A1
(43) Pub. Date: Jul. 23, 2009

(54) METHOD FOR PRODUCTION OF CYTOCHROME P450 WITH N-TERMINAL TRUNCATED P450 REDUCTASE

(76) Inventor: Bhabatosh Chaudhuri, (US)

Correspondence Address: ELMORE PATENT LAW GROUP, PC, 515 Groton Road, Unit 1R, Westford, MA, 01886, US

(21) Appl. No.: 12/264,365

(22) Filed: Nov. 4, 2008

Related U.S. Application Data
(63) Continuation of application No. PCT/GB2007/001617, filed on May 2, 2007.

(30) Foreign Application Priority Data
May 5, 2006 (GB) 0608941.1

(51) Int. Cl. C12N 15/81; C07H 21/04; C12N 9/02; C12N 15/63; C12N 1/19

(52) U.S. Cl. 435/471; 536/23.2; 435/189; 435/320.1; 435/254.2

(57) ABSTRACT

The present invention provides a P450 reductase lacking N-terminal amino acids, as well as a nucleic acid encoding the P450 reductase. When co-expressed with a cytochrome P450, the P450 reductase increases the activity and/or expression of the cytochrome P450 compared to the activity and/or expression of the cytochrome P450 when co-expressed with a wild type P450 reductase. The invention also provides the use of certain promoters to increase expression of cytochrome P450, P450 reductase and/or b5, as well as the use of protease-deficient strains of yeast in which to express the proteins of the present invention.

### RELATED APPLICATION

This application is a continuation of PCT/GB2007/001617 filed on May 2, 2007, which claims priority under 35 U.S.C. § 119 or 365 to United Kingdom, Application No. 0608941.1, filed on May 5, 2006. The entire teachings of the above applications are incorporated herein by reference.

### SUMMARY OF THE INVENTION

The present invention relates to methods of expressing proteins. In particular, the present invention relates to cytochrome P450 expression systems and to the 5 production of cytochrome P450.

### BRIEF DESCRIPTION OF THE DRAWINGS

FIG. 1 illustrates the clone pBluSK(+)/Ngo-Bam/Gal1p-907;

FIG. 2 illustrates the clone pSYEGal1p-907;

FIG. 3 illustrates the clone pBluSK/Ngo-Bam/Gal1-p675;

FIG. 4 illustrates the clone pSYE224;

FIG. 5 illustrates the clone pBluSK/Ngo-Bam/Gal1p-650;

FIG. 6 illustrates the clone pSYEGal1p-650;

FIG. 7 illustrates the clone pBluSK/Ngo-Bam/Gal1p-461;

FIG. 8 illustrates the clone pSYEGal1p-461;

FIG. 9 illustrates the clone pSYECYC1p-core;

FIG. 10 illustrates the clone pSYECYC1-GAL1UAS;

FIG. 11 illustrates the clone pSYECYC1-GAL10UAS;

FIG. 12 illustrates the clone pBluSK/Bam-Xba/mCYP1B1;

FIG. 13 illustrates the clone pSYEGal1p907/m1B1;

FIG. 14 illustrates the clone pSYE225;

FIG. 15 illustrates the clone pSYEGal1p650/m1B1;

FIG. 16 illustrates the clone pSYEGal1p461/m1B1;

FIG. 17 illustrates the clone pSYECYC1-GAL1UAS/m1B1;

FIG. 18 illustrates the clone pSYECYC1-GAL10UAS;

FIG. 19 illustrates the comparative analysis of expression of human CYP1B1 from the different GAL promoters;

FIG. 20 illustrates the clone pBluSK(+)/Xba-Sac/SUC2t;

FIG. 21 illustrates the clone pBluKS(+)/Xba-Sac/mSUC2t;

FIG. 22 illustrates the clone pBluKS(+)/Gal1mS;

FIG. 23 illustrates the clone YILeuGAL1MS;

FIG. 24 illustrates the yeast integrating plasmid YIpAdGAL1MS;

FIG. 25 illustrates the yeast integrating plasmid YIHisGAL1MS;

FIG. 26 illustrates the yeast integrating plasmid YITrpGAL1MS;

FIG. 27 illustrates the plasmid pBluKS(+)/DelN24hRDStop;

FIG. 28 illustrates the plasmid pBluKS(+)/DelN24hRDw/oStop;

FIG. 29 illustrates the plasmid pBluKS(+)/hRD_fl;

FIG. 30 illustrates the plasmid pSYI210;

FIG. 31 illustrates the plasmid pSYI201;

FIG. 32 illustrates the plasmid pSYI205;

FIG. 33 illustrates the P450 reductase activities of yeast strains YI001, YI002, YI003 and YI004;

FIG. 34 illustrates the P450 amounts obtained from the yeast strains YI005, YI006, YI007 and YI008;

FIG. 35 illustrates the relative activities of CYP1B1 in microsomes obtained from yeast strains YI005, YI006, YI007 and YI008;

FIG. 36 illustrates the plasmid pSYI217;

FIG. 37 illustrates the plasmid pSYI224;

FIG. 38 illustrates the plasmid pSYI222;

FIG. 39 illustrates the P450 reductase activities of yeast strains YI001, YI009, YI010, YI011 and the control strain YI004;

FIG. 40 illustrates the P450 reductase activities of yeast strains YI005, YI012, YI013, YI014 and the control strain YI008;

FIG. 41 illustrates the CYP1B1P450 activities in microsomes obtained from yeast strains YI005, YI012, YI013, YI014 and the control strain YI008;

FIG. 42 illustrates the plasmid pSYI215;

FIG. 43 illustrates the plasmid pSP73/Gal1mS;

FIG. 44 illustrates the plasmid pSP73/Gal1hRDStopmS;

FIG. 45 illustrates the plasmid pSP73/Gal1hRDw/oStopmS;

FIG. 46 illustrates the plasmid pSYI211;

FIG. 47 illustrates the plasmid pSYI202;

FIG. 48 illustrates the plasmid pSYI218;

FIG. 49 illustrates the plasmid pSYI240;

FIG. 50 illustrates the P450 reductase activities of yeast strains YI001, YI002, YI009, YI015, YI016, YI017, YI018 and YI019;

FIG. 51 illustrates the P450 amounts obtained from the yeast strains YI005, YI006, YI012, YI020, YI021, YI022, YI023 and YI024;

FIG. 52 illustrates the CYP1B1 P450 activities in microsomes obtained from yeast strains YI005, YI006, YI012, YI020, YI021, YI022, YI023 and YI024;

FIG. 53 illustrates the plasmid pBluKS+/TRP1;

FIG. 54 illustrates the plasmid pBlu/5′PRA1-TRP1;

FIG. 55 illustrates the gene disruption plasmid PSL001;

FIG. 56 illustrates the plasmid pBlu/5′HRD1-TRP1;

FIG. 57 illustrates the gene disruption plasmid pSL002;

FIG. 58 illustrates the plasmid pBlu/5′HRD2-TRP1;

FIG. 59 illustrates the gene disruption plasmid pSL003;

FIG. 60 illustrates the plasmid pBlu/5′UBC7-TRP1;

FIG. 61 illustrates the gene disruption plasmid pSL004;

FIG. 62 illustrates the general strategy used for gene disruption, using the disruption of the PEP4 gene as an example;

FIG. 63 illustrates the P450 amounts in microsomes obtained from yeast strains YI005, YI021, YI033, YI034, YI034, YI035, YI036, YI037, YI038, YI039 and YI040;

FIG. 64 illustrates the plasmid pSP73/BglII-XbaI/yRD;

FIG. 65 illustrates the plasmid pSP73/delta-yRD;

FIG. 66 illustrates the plasmid pAUR101/delta-yRD;

FIG. 67 illustrates the unique restriction sites of the plasmid pAUR101/delta-yRD;

FIG. 68 illustrates the plasmid pSYI220;

FIG. 69 illustrates the plasmid pSYI209;

FIG. 70 illustrates the plasmid pSYI225;

FIG. 71 illustrates the plasmid pSYI223;

FIG. 72 illustrates the reductase activities in yeast strains containing GAL1p-675 promoter yRD;

FIG. 73 illustrates the plasmid pYESLEU;

FIG. 74 illustrates the plasmid pSYE257;

FIG. 75 illustrates the plasmid pBGal1b5mS;

FIG. 76 illustrates the plasmid YITrpGal1b5mS;

FIG. 77 illustrates the plasmid pSYE209;

FIG. 78 illustrates the plasmid pAUR111/Gal1pb5S;

FIG. 79 illustrates the plasmid pAUR135/Gal1pb5S;

FIG. 80 illustrates a representative example of the increase of cytochrome P450 activity from Example 10;

FIG. 81 illustrates the plasmid pBluKS(+)/ADH2p-573;

FIG. 82 illustrates the plasmid pSYE263;

FIG. 83 illustrates the plasmid pSYE264;

FIG. 84 illustrates the plasmid pSYE265;

FIG. 85 illustrates the plasmid YILEUADH2MS;

FIG. 86 illustrates the plasmid YILEUADH2MS/delN24hRD;

FIG. 87 illustrates the plasmid pBluKS(+)/Sal-Bam/PGK1p-650;

FIG. 88 illustrates the plasmid pSYE239;

FIG. 89 illustrates the plasmid pSYE278;

FIG. 90 illustrates the plasmid pSYE279;

FIG. 91 illustrates the plasmid YILEUPGK1MS;

FIG. 92 illustrates the plasmid YILEUPGK1MS/delN24hRD;

FIG. 93 illustrates the plasmid pBluKS(+)/Sal-Bam/pBR-GAPDHp;

FIG. 94 illustrates the plasmid pSYE280;

FIG. 95 illustrates the plasmid pSYE281;

FIG. 96 illustrates the plasmid pSYE282;

FIG. 97 illustrates the plasmid YILEUpBRGAPDHMS;

FIG. 98 illustrates the plasmid YILEUpBRGAPDHMS/delN24hRD;

FIG. 99 illustrates the plasmid pSYE224/hCYP2D6;

FIG. 100 illustrates the plasmid pSYE224/hCYP1A2;

FIG. 101 is a graph showing the amount of CYP2D6 produced for each of the GAL1, GAPDH, PGK1 and ADH2 promoters (expressed as absorbance units versus wavelength of light); and

FIG. 102 is a graph showing the amount of CYP 1A1 produced for each of the GAL1, GAPDH, PGK1 and ADH2 promoters (expressed as absorbance units versus wavelength of light).

### DETAILED DESCRIPTION OF THE INVENTION

Cytochrome P450 (CYP) belongs to a large family of detoxifying enzymes (present in different parts of the human body especially in the liver, kidneys, lung, the central nervous system) that are involved in the break-up (i.e. metabolism) of diverse xenobiotics, which include most pharmaceuticals, many dietary substances and a wide variety of environmental chemicals. Xenobiotics are defined as alien chemical substances that are introduced into the human organism either accidentally or deliberately. CYPs are involved in 90% of the metabolism of xenbiotics that occurs in the human body. CYPs metabolise xenobiotics by the action of oxygen, which makes them more soluble and easier to excrete.

During the drug development process, it is imperative that the rate of metabolism, and the nature and toxicity of the products is determined before a compound is introduced in human clinical trials.

The present main commercial use of CYPs is for the investigation of the metabolism of drug compounds that are already in development. The CYPs are mostly used sparingly (because of high costs) in secondary assays to confirm a metabolic pathway.

However, the use of CYPs to screen a vast number of potential drug candidates in pre-clinical research could greatly reduce the cost to pharmaceutical companies of late stage drug development failures.

There therefore exists a need to develop an improved system for expressing CYPs with high activities and/or at high expression levels to enable screening of drug compounds and in particular drug candidates for toxicity.

P450 reductase (RD) acts as a co-factor which is essential for the activity of the cytochrome P450 isozymes. Unusually, as a co-factor, P450 reductase possesses enzymatic activity. It abstracts electrons from NADPH and transfers to the active site of the P450 isozymes. Inherently, the P450 reductase enzyme generates reactive oxygen species (ROS). Therefore, expression of P450 reductase enzyme is harmful to a living cell. Indeed, in human and insect cells, co-expression of P450 reductase has been reported to be deleterious for P450 expression. However, as noted above, P450 reductase must be co-expressed with cytochrome P450 to result in an active cytochrome P450.

A first aspect of the present invention provides an isolated or recombinant nucleic acid molecule comprising a nucleotide sequence encoding a P450 reductase lacking N-terminal amino acids, wherein the P450 reductase, when co-expressed with a cytochrome P450, increases the activity and/or expression of the cytochrome P450 compared to the activity and/or expression of the cytochrome P450 when co-expressed with a wild type P450 reductase.

An increase in activity and/or expression occurs when the activity and/or expression of cytochrome P450 when co-expressed with the P450 reductase encoded by the nucleic acid molecule is greater than the activity and/or expression of the cytochrome P450 when co-expressed with wild type reductase. The increase may be a 1-50 fold increase and may be a 1-40 fold, 1-30 fold, 1-20 fold or 1-10 fold increase. The increase may be 1, 2, 3, 4, 5, 6, 7, 8, 9 or 10 fold.

The expression of cytochrome P450 can be measured by measuring the concentration of cytochrome P450 produced using CO-difference spectra. The use of CO-difference spectra is well known to those skilled in the art (Omura T & Sato R. The carbon monoxide binding pigment of liver microsomes. I—evidence for its hemoprotein nature. Journal of Biological Chemistry 1964 239 2370-2378). The difference spectrum may be measured at 400-500 nm to calculate the concentration of cytochrome P450.

The activity of cytochrome P450 may be measured by the measurement of the activity of 7-ethoxyresorufin O-deethylase (Klotz A. V., S. J. J. a. W. C. An alternative 7-ethoxyresorufin O-deethylase activity assay: a continuous visible spectrophotometric method for measurement of cytochrome P450 monooxygenase activity. Analytical Biochemistry 140: 138-145, 1984). This enzyme converts 7-ethoxyresorufin to resorufin which can be flourimetrically detected in presence of NADPH and oxygen and the assay is known as the EROD assay.

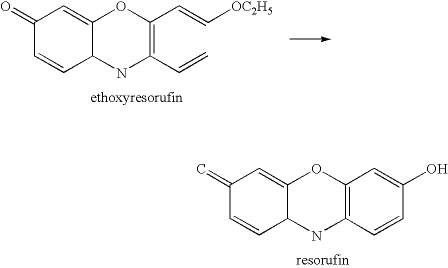

The amount of resorufin produced may be measured in a spectrophotometer after the addition of NADPH and the samples to be tested.

The EROD assay may be used to determine activity of most cytochrome P450s but preferably activities of the CYP1 family of enzymes. Alternatively, other established methods can be used. The methods that are used depend upon which cytochrome P450 activity is being measured, and hence which cytochrome P450 substrate should be utilised in an assay. A variety of these substrates (mostly fluorescent but also colorometric) may be used. The substrates are well documented in the art. Indeed, the skilled person would use one or more of the known substrates depending on which cytochrome P450 activity is being measured.

The invention also provides polypeptides, such as a P450 reductase, encoded by the nucleic acid molecules of the present invention. The P450 reductase encoded by the nucleic acid molecule may be mammalian P450 reductase and may be a human P450 reductase. Alternatively, the P450 reductase may be a yeast P450 reductase. The P450 reductase may lack at least the 24 N-terminal amino acids. The P450 reductase may lack at least the 32 N-terminal amino acids, at least the 41 N-terminal amino acids, at least the 44 N-terminal amino acids, or at least the 56 N-terminal amino acids or at least the 60 N-terminal amino acids. The P450 reductase may lack the 1-24, 1-32, 1-41, 1-44, 1-56 or 1-60 N-terminal amino acids. The human P450 reductase may comprise or consist of the amino acid sequence of SEQ ID NO: 26. The present invention is described generally herein with reference to human P450 reductase, although it is not to be considered as being limited to human P450 reductase. It will be appreciated that those skilled in the art can engineer other mammalian P450 reductases in a similar fashion to the human P450 reductase, for example by comparing the respective sequences of the P450 reductases.

In a second aspect of the present invention there is provided an isolated or recombinant nucleic acid molecule comprising or consisting of:

a) a nucleotide sequence encoding the P450 reductase of the first aspect;
b) a nucleotide sequence of SEQ ID NO: 34 or 37;
c) a nucleotide sequence having at least 80% identity to the sequence of a) or b) and encoding a P450 reductase which, when co-expressed with a cytochrome P450, increases the activity and/or expression of the cytochrome P450 compared to the activity and/or expression of the cytochrome P450 when co-expressed with a wild type P450 reductase;
d) a nucleotide sequence which is complementary to the sequence of a), b) or c); or
e) a nucleotide sequence which codes for the same polypeptide as the sequence of a), b), c) or d).

A variant of the human P450 reductase (hRD) has been expressed to obtain hRD activity that is not deleterious for cytochrome P450 expression and/or activity. The variant enables the provision of an optimal system that allows high expression levels and/or high activity of cytochrome P450 isozymes and in particular recombinant heterologous cytochrome P450 isozymes in yeast, but also in insect and mammalian cells.

The P450 reductase variant encoded by the sequence of SEQ ID NO: 34 or 37 lacks the charged N-terminal 24 amino acids of wild type hRD. In the wild type hRD, the hydrophobic membrane anchor is constituted by amino acids 20-39 (http://www.enzim.hu/hmmtop/html/submit.html) or 25-44 (http://www.ch.embnet.org/software/TMPRED_form.html).

The P450 reductase encoded by the nucleic acid molecule of the present invention may further comprise an amino acid sequence at the C-terminal end comprising an epitope tag. The amino acid sequence may be provided at the C-terminal end of the P450 reductase. The epitope tag may be c-myc which may comprise the amino acid sequence EQKLISEEDLNG. The c-myc tag may be linked to the P450 reductase with the linker SS. The amino acid sequence may additionally comprise the amino acids SRL at the C-terminal end thereof. One P450 reductase in accordance with the present invention may be encoded by the nucleotide sequence of SEQ ID NO: 42. The P450 reductase may comprise or consist of the amino acid sequence of SEQ ID NO: 27. This P450 reductase lacks the charged N-terminal 24 amino acids and the COOH-terminal Stop codon of human P450 reductase, but contains the c-myc epitope tag EQKLISEEDLNG at the C-terminal end. The 12 amino acid c-myc tag is a negatively charged peptide and is linked to the C-terminus through the linker, SS (coded for by TCTAGT formed through the ligation of the restriction sites SpeI and XbaI). The nucleotide sequence encoding the c-myc tag may be chemically synthesised using yeast-biased codons.

The terms “nucleic acid molecule” and “nucleotide sequence” include double and single stranded DNA and RNA molecules and backbone modifications thereof. A given RNA molecule has a sequence which is complementary to that of a given DNA molecule, allowing for the fact that in RNA ‘U’ replaces ‘T’ in the genetic code. The nucleic acid molecule of the present invention may be in isolated, recombinant or chemically synthetic form.

As used herein with respect to nucleic acid molecules, “isolated or “recombinant” means any of a) amplified in vitro by, for example, polymerase chain reaction (PCR), b) recombinantly produced by cloning, c) purified by, for example, gel separation, or d) synthesised, such as by chemical synthesis.

The nucleic acid molecules of the present invention, including DNA and RNA, may be synthesised using methods known in the art, such as using conventional chemical approaches or polymerase chain reaction (PCR) amplification. The nucleic acid molecules of the present invention also permit the identification and cloning of the identified genes, for instance by screening cDNA libraries, genomic libraries or expression libraries.

The present invention includes nucleic acid molecules comprising a sequence complementary to the sequence as defined above. Thus, for example, both strands of a double stranded nucleic acid molecule are included within the scope of the present invention (whether or not they are associated with one another). Also included are mRNA molecules and complementary DNA molecules (e.g. cDNA molecules).

The nucleotide sequence of c) above may have at least 85%, 90% or 95% identity to the sequence of a) or b). The nucleotide sequence may have at least 96%, 97%, 98% or 99% identity to the sequence of a) or b).

The “percent identity” of two amino acid sequences or of two nucleic acid (nucleotide) sequences is generally determined by aligning the sequences for optimal comparison purposes (e.g., gaps can be introduced in either sequences for best alignment with the other sequence) and comparing the amino acid residues or nucleotides at corresponding positions. The “best alignment” is an alignment of two sequences that results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search.

The nucleic acid molecule of the present invention may further comprise a promoter or other regulatory sequence which controls expression of the nucleotide sequence. The promoter may be an inducible promoter, which may be a GAL promoter. The promoter may comprise a truncated GAL promoter. Alternatively, the inducible promoter may be a ADH2 promoter.

In a third aspect there is provided an isolated or recombinant nucleic acid molecule comprising a truncated GAL promoter for controlling the expression of a nucleotide sequence.

The truncated GAL promoter may be a truncated GAL1 promoter. The truncated GAL1 promoter may be a GAL1 promoter truncated at nucleotide 202. The truncated GAL1 promoter may comprise or consist of the sequence of SEQ ID NO: 2.

The nucleic acid molecule of the invention may further comprise a transcription termination sequence, which may be downstream of the promoter. The nucleic acid molecule may comprise unique restriction sites between the GAL promoter and termination sequence that allow insertion of a nucleotide sequence under the control of the promoter. The transcription termination sequence may be immediately downstream of the inserted nucleotide sequence or separated by a minimal distance. The transcription termination sequence may be separated from the inserted nucleotide sequence by 5-25 nucleotides. It may be separated by 5-20, 5-15, 15-20, 5-10, 6-9 or 6-8 nucleotides. It may be separated by 5, 6, 7, 8, 9, 10, 11, 12, 13, 14, 15, 16, 17, 18, 19 or 20 nucleotides.

The termination sequence may be a termination sequence from any expressed gene and may be selected from SUC2 (SUC2t), PHO5, ADH1, ADH2 or CYC1. The termination sequence may be a SUC2 (SUC2t) termination sequence, and may comprise or consist of the sequence of SEQ ID NO: 28.

The promoter may control the expression of a nucleotide sequence encoding a P450 reductase lacking N-terminal amino acids. The nucleotide sequence may be a nucleic acid molecule of the first and/or second aspect. Alternatively, the promoter may control the expression of a nucleotide sequence encoding cytochrome P450. The nucleotide sequence encoding cytochrome P450 may be selected from sequences known in the art, including human cytochrome P450 and cytochrome P450s from other organisms (http://drnelson.utmem.edu/CytochromeP450.html). The nucleotide sequence may encode human cytochrome P450 and may comprise or consist of a nucleotide sequence selected from:

a) the nucleotide sequence of SEQ ID NO: 22;

b) a nucleotide sequence having at least 80% identity to the sequence of a) and encoding a cytochrome P450;

c) a nucleotide sequence which is complementary to the sequence of a) or b); or

d) a nucleotide sequence which codes for the same polypeptide as the sequence of a), b) or c).

The nucleotide sequence may be inserted into the nucleic acid molecule between the promoter and termination sequence as a BamH1-XbaI, BamH1H-XhoI, BglII-XbaI, BglII-XhoI, BamH1-SpeI or BglII-SpeI fragment.

The nucleotide sequence of b) above may have at least 85%, 90% or 95% identity to the sequence of a). The nucleotide sequence may have at least 96%, 97%, 98% or 99% identity to the sequence of a).

The nucleic acid molecule of the present invention may be provided in the form of a vector.

Therefore, in a fourth aspect there is provided a vector comprising a nucleic acid molecule of the first, second and/or third aspect.

The term “vector” refers to a nucleic acid molecule having a nucleotide sequence that can assimilate new nucleic acid molecules, and propagate those new sequences in an appropriate host.

The vector may cause expression of the nucleic acid molecule in a target cell. The target cell may be a eukaryotic cell and may be a yeast, mammalian or insect cell. The vector may be an integrating vector. The vector may be capable of integration into the genome of the target cell. The vector may be selected from plasmid vectors, cosmid vectors, phage vectors, episomally replicating vectors, retroviral vectors, lentiviral vectors, adenovirus-associated virus (AAV) vectors, adenoviral vectors or baculovirus vectors. Such vectors are known in the art and any of these may be employed in the present invention. The vector may be a yeast integrating vector.

The vector may comprise one or more expressed markers such as selective markers and/or reporter genes which enable selection of cells transfected (or transformed: the terms are used interchangeably in this text) with them and preferably, to enable a selection of cells containing vectors incorporating heterologous DNA. A suitable start and stop signal will generally be present and if the vector is intended for expression, sufficient regulatory sequences to drive expression will be present.

Examples of reporter genes that may be used include alkaline phosphatase (AP), beta galactosidase (LacZ), beta glucoronidase (GUS), chloramphenicol acetyltransferase (CAT), green fluorescent protein (GFP), horseradish peroxidase (HRP), and luciferase (Luc). Possible antibiotic selectable markers include those that confer resistance to ampicillin, aureobasidine, bleomycin, chloramphenicol, gentamycin, hygromycin, kanamycin, lincomycin, methotrexate, phosphinothricin, puromycin, and tetracycline. The selective marker may be an auxotrophic selective marker gene. The auxotrophic selective marker gene enables growth of transformed cells in minimal media, and may be selected from LEU2, TRP1, ADE2, URA3, HIS3, LYS2, HIS4 or MET15 or AUR1-C.

The vector may allow integration of the nucleic acid molecule at the locus of any specific gene in the genome of the target cell.

The selectable marker gene may enable integration into the genome of the target cell at a particular chromosomal locus. The integration may be by homologous recombination using techniques known in the art. The chromosomal locus may be selected from LEU2, TRP1, ADE2, URA3, HIS3, LYS2, HIS4, MET15 or AUR1-C or several other marker genes that confer resistance against antibiotics or other toxic compounds.

The vector may be capable of integration into a yeast cell and may be capable if integration into the yeast genome.

The vectors and nucleic acid molecules of the invention may be integrated into the host cell genome by random integration or by homologous recombination. Alternatively, they may be targeted to a specific location in the host cell by methods known in the art such as a site specific recombinase or integrase for integration into a specific site. This may allow the vector and/or nucleic acid molecule to be targeted into a known region with particular characteristics such as being permissive for expression or to avoid integration in a gene of the host cell.

The host may be a eukaryote, and may be selected from yeast, mammals or insects.

The nucleic acid molecules and/or vectors of the present invention may be introduced into cells using a variety of methods known in the art. Where the nucleic acid molecules and/or vectors are introduced into a cell in vitro, conventional techniques such as transfection, liposomes, viruses or lipid reagents may be employed. Electroporation may be used to introduce the nucleic acid molecules and/or vectors into cells, and in particular into mammalian cells.

After transformation (or transfection: the terms are used interchangeably in this text) of the target cell by a vector and/or nucleic acid molecule of the present invention, various selection and/or screening techniques may be employed to identify clones in which the vector and/or nucleic acid molecule has integrated and to further characterise them. By employing a selectable marker or reporter gene this may allow selection of the clones in which the vector and/or nucleic acid molecule has integrated such as by looking for expression of a reporter gene, antibiotic selection or by growth on minimal media.

Typically, after transformation the cells will be grown for a sufficient period of time such that transient expression will not be the reason for drug resistance, reporter gene expression or growth on minimal media. For example, the cells may be grown for 3 to 5 days. The cells may be grown for more than a week, preferably for ten days and more preferably for two weeks before selection and characterisation. For example, yeast cells may be grown for 3 to 4 days.

Clones which have integrated a vector and/or nucleic acid molecule of the invention may be further characterised. For example, Southern blotting or PCR may be carried out to check the vector and/or nucleic acid molecule integration, determine the site of integration and copy number of the integrated vector and/or nucleic acid molecule. The site of integration may be characterised to ensure that it is not an endogenous gene or other important element that has been disturbed. Northern blotting or other such techniques may be carried out to determine whether the nucleotide sequence is being expressed.

The vector may comprise two copies of a nucleic acid molecule of the first, second and/or third aspect of the present invention, each copy under the control of a respective promoter. The promoters may be inducible promoters and may be GAL promoters. The promoters may be truncated GAL promoters. These vectors enable higher expression of a nucleic acid molecule of the invention after transformation of a cell and integration into the host cell genome. Therefore, such vectors may provide for further high expression levels and improved production of cytochrome P450 isozymes with high activity in the cell.

A fifth aspect of the invention provides a cell transformed with the nucleic acid molecule of the first, second or third aspect, and/or vector of the fourth aspect. The nucleic acid molecule of the first, second or third aspect and/or vector of the fourth aspect may be integrated into the genome of the cell. The transformed (or transfected: the terms are used interchangeably in this text) cell may be a eukaryotic cell and may be a yeast, mammalian or insect cell.

A sixth aspect of the invention provides a method of expressing a nucleic acid molecule of the first, second or third aspect in a cell, comprising transforming the cell with a nucleic acid molecule of the first, second or third aspect and/or vector of the fourth aspect which directs the expression of the nucleic acid molecule.

A seventh aspect provides a method of expressing a nucleotide sequence encoding cytochrome P450 in a cell, comprising transforming the cell with a vector of the fourth aspect which directs the expression of the nucleotide sequence encoding cytochrome P450.

The nucleotide sequence may encode heterologous cytochrome P450 and may be selected from sequences known in the art, including human cytochrome P450 sequences and sequences from other organisms (see for example http://drnelson.utmem.edu/CytochromeP450.html). The nucleotide sequence may comprise or consist of a sequence selected from:

a) the nucleotide sequence of SEQ ID NO: 22;

b) a nucleotide sequence having at least 80% identity to the sequence of a) and encoding a cytochrome P450;

c) a nucleotide sequence which is complementary to the sequence of a) or b); or

d) a nucleotide sequence which codes for the same polypeptide as the sequence of a), b) or c).

The nucleotide sequence of b) above may have at least 85%, 90% or 95% identity to the sequence of a). The nucleotide sequence may have at least 96%, 97%, 98% or 99% identity to the sequence of a).

The cell may be a eukaryotic cell. The cell may be a yeast cell, insect cell or mammalian cell.

In an eighth aspect of the invention there is provided a method of producing a yeast strain expressing a nucleic acid molecule of the first, second or third aspect, comprising transforming a yeast strain with a nucleic acid molecule of the first, second or third aspects and/or with a vector of the fourth aspect.

The present invention further provides in a ninth aspect a yeast strain produced by the method of the eighth aspect or transformed with a vector of the fourth aspect.

The yeast strain may be Saccharomyces cerevisiae. The yeast strain may be selected from those known in the art. The yeast strain may be selected from the yeast strains JL20 (Daum, G et al. Yeast Functional Analysis Report. Yeast Volume 15, Issue 7, Pages 601-614) and W303B (Furuchi, T et al. Functions of yeast helicase Ssl2p that are essential for viability are also involved in protection from the toxicity of adriamycin. Nucleic Acids Res. 2004; 32(8): 2578-2585).

A yeast strain expressing a nucleic acid molecule of the first, second or third aspect enables the provision of a system for providing increased activity and/or expression of cytochrome P450.

An eleventh aspect of the present invention provides a protein expression system comprising:

i) a cell of the fifth aspect or yeast strain of the ninth aspect; and

ii) a vector comprising a nucleotide sequence encoding a target protein, said sequence under the control of a promoter which causes expression of the nucleotide sequence.

The nucleotide sequence may encode a cytochrome P450, which may be a heterologous cytochrome P450 and may be human cytochrome P450.

The nucleotide sequence may alternatively encode a cytochrome b5 protein, which may be a heterologous cytochrome b5 protein. The cytochrome b5 protein may be a human b5 protein. The nucleotide sequence may comprise or consist of:

a) the nucleotide sequence of SEQ ID NO: 86;

b) a nucleotide sequence having at least 80% identity to the sequence of a) and encoding a cytochrome b5 protein;

c) a nucleotide sequence which is complementary to the sequence of a) or b); or

d) a nucleotide sequence which codes for the same polypeptide as the sequence of a), b) or c).

The nucleotide sequence of b) above may have at least 85%, 90% or 95% identity to the sequence of a). The nucleotide sequence may have at least 96%, 97%, 98% or 99% identity to the sequence of a).

Cytochrome b5 protein is a co-factor that contributes to cytochrome P450 activity.

The vector may comprise a nucleotide sequence encoding a cytochrome P450 and a nucleotide sequence encoding a cytochrome b5 protein, the sequences under the control of diverse promoters.

The vector may cause expression of the, or each, nucleotide sequence on integration into the cell or yeast genome.

The vector may be as defined in the fourth aspect.

The present invention also provides, in a twelfth aspect, a method of producing a cytochrome P450 with increased activity and/or increased expression levels, comprising transforming a cell of the fifth aspect or yeast strain of the ninth aspect with a vector of the fourth aspect capable of directing the expression of cytochrome P450.

The cytochrome P450 may be heterologous cytochrome P450 and may be human cytochrome P450.

The present invention also provides, in a thirteenth aspect, a method of producing cytochrome P450 with increased activity and/or increased expression levels, comprising transforming a cell with a nucleic acid molecule of the first, second or third aspects and/or with a vector of the fourth aspect.

The method may further comprise transforming the cell with a nucleic acid molecule comprising a nucleotide sequence encoding a cytochrome P450 and/or a vector of the fourth aspect which directs expression of the nucleotide sequence encoding the cytochrome P450.

The nucleotide sequence may encode heterologous cytochrome P450 and may be human cytochrome P450.

The method of the twelfth and/or thirteenth aspects may further comprise transforming the cell with a nucleic acid molecule comprising a nucleotide sequence encoding a cytochrome b5 protein and/or a vector which directs the expression of a nucleotide sequence encoding a cytochrome b5 protein.

The activity of cytochrome P450 may be measured by the EROD assay or by other assays known in the art. The expression of cytochrome P450 may be measured using CO-difference spectra as is known in the art. An increase in activity and/or expression occurs when the expression and/or activity measured is greater than the expression and/or activity of cytochrome P450 in a cell that has not been transformed with a nucleic acid molecule of the first, second or third aspect and/or with a vector of the fourth aspect.

The cell may be a eukaryotic cell and may be selected from a yeast cell, insect cell or mammalian cell.

The present invention in a fourteenth aspect provides a P450 reductase produced by the method of the sixth aspect.

A fifteenth aspect provides an isolated or recombinant polypeptide comprising or consisting of:

a) a P450 reductase lacking N-terminal amino acids, wherein the P450 reductase, when co-expressed with a cytochrome P450, increases the activity and/or expression of the cytochrome P450 compared to the activity and/or expression of the cytochrome P450 when co-expressed with a wild type P450 reductase;
b) a homologue of the polypeptide of a), the homologue when co-expressed with a cytochrome P450, increases the activity and/or expression of the cytochrome P450 compared to the activity and/or expression of the cytochrome P450 when co-expressed with a wild type P450 reductase; or
c) a fragment of the polypeptide of a) or homologue of b), the fragment when co-expressed with a cytochrome P450, increases the activity and/or expression of the cytochrome P450 compared to the activity and/or expression of the cytochrome P450 when co-expressed with a wild type P450 reductase.

The polypeptide may be a P450 reductase which may be a human P450 reductase. The P450 reductase may lack the 24 N-terminal amino acids. The human P450 reductase may comprise or consist of the amino acid sequence of SEQ ID NO: 26.

The polypeptide may further comprise an amino acid sequence at the C-terminal end comprising an epitope tag. The epitope tag may be c-myc which may comprise the amino acid sequence EQKLISEEDLNG. The c-myc tag may be linked to the P450 reductase with the linker SS. The amino acid sequence may additionally comprise the amino acids SRL at the C-terminal end thereof. One polypeptide in accordance with the present invention may comprise or consist of the sequence of SEQ ID NO: 27. This polypeptide is a P450 reductase which lacks the charged N-terminal 24 amino acids and the COOH-terminal Stop codon of human P450 reductase, but contains the c-myc epitope tag EQKLISEEDLNG at the C-terminal end. The 12 amino acid c-myc tag is a negatively charged peptide and is linked to the C-terminus through the linker, SS (coded for by TCTAGT formed through the ligation of the restriction sites SpeI and XbaI).

Polypeptides which include one or more additions, deletions, substitutions or the like are encompassed by the present invention. In addition, it may be possible to replace one amino acid with another of similar “type”. For instance, a hydrophobic amino acid may be replaced with another.

The polypeptide of the present invention may be modified either by natural processes, such as processing or other post-translational modifications, or by chemical modification techniques which are well known in the art. Among the numerous known modifications which may be present include, but are not limited to, acetylation, acylation, amidation, ADP-ribosylation, glycosylation, GPI anchor formation, covalent attachment of a lipid or lipid derivative, methylation, myristylation, pegylation, prenylation, phosphorylation, ubiquitination, or any similar process.

In the case of homologues, the degree of identity with a polypeptide as described herein is less important than that the homologue should retain the function of the polypeptide. However, suitably, homologues having at least 60% identity with the polypeptides described herein are provided. Preferably, homologues having at least 70% identity, more preferably at least 80% identity are provided. Most preferably, homologues having at least 85%, 90%, 95%, 96%, 97%, 98% or even 99% or greater identity are provided.

The polypeptides of the present invention can be coded for by a large variety of nucleic acid molecules, taking into account the well known degeneracy of the genetic code. All of these molecules are within the scope of the present invention. They can be inserted into vectors and cloned to provide large amounts of DNA or RNA for further study. Suitable vectors may be introduced into host cells to enable the expression of polypeptides used in the present invention using techniques known to the person skilled in the art.

The polypeptides, homologues or fragments thereof of the present invention may be provided in isolated or recombinant form, and may be fused to other moieties. The polypeptides, homologues or fragments thereof may be provided in substantially pure form, that is to say free, to a substantial extent, from other proteins. Thus, a polypeptide may be provided in a composition in which it is the predominant component present (i.e. it is present at a level of at least 50%; preferably at least 75%, at least 90%, or at least 95%; when determined on a weight/weight basis excluding solvents or carriers).

It is often advantageous to reduce the length of a polypeptide, provided that the resultant reduced length polypeptide still has a desired activity or can give rise to useful antibodies. Feature c) of this aspect of the present invention therefore covers fragments of the polypeptide of a) or homologue of b).

“Fragment” refers to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a). The fragment possesses the functional activity of the polypeptide defined in a).

As used herein with respect to polypeptides, “isolated” means separated from its native environment and present in sufficient quantity to permit its identification or use. Isolated, when referring to a protein or polypeptide, means, for example: (i) selectively produced by expression cloning or (ii) purified by chromatography or electrophoresis. Isolated polypeptides may, but need not be, substantially pure. The term “substantially pure” means that the polypeptides are essentially free of other substances with which they may be found in nature or in vivo systems to an extent practical and appropriate for their intended use.

A “recombinant polypeptide” is a polypeptide isolated, purified, or identified by virtue of expression in a heterologous cell, said cell having been transformed or transfected, either transiently or stably, with a recombinant vector engineered to drive expression of the polypeptide in the host cell.

To provide for increased activity and/or expression of cytochrome P450 in a target cell, the inventors have identified that degradation of cytochrome P450 and/or cytochrome P450 reductase can be prevented by disrupting the respective protease gene in the genome of the target cell.

The yeast PEP4/PRA-1 gene encodes a vacuolar proteinase A (yscA), a critical enzyme in the post-translational processing and functional maturation of vacuolar protease. Some publications indicate that certain integral ER proteins, including CYP2B1, CYP2E1, and NADPH P450 reductase, may incur lysosomal rather than proteasomal degradation (Masaki, R. et al. 1987. Cytochrome P-450 and NADPH-cytochrome P-450 reductase are degraded in the autolysosomes in rat liver. J Cell Biol, 104: 1207-1215; Ronis, M. et al. 1991. Acetone-regulated synthesis and degradation of cytochrome P450E1 and cytochrome P4502B1 in rat liver. Eur J Biochem, 198(2): 383-389; Murray, B. et al. 2002. Native CYP2C11: heterologous expression in Saccharomyces cerevisiae reveals a role for vacuolar proteases rather than the proteasome system in the degradation of this endoplasmic reticulum protein. Mol Pharmacol, 61: 1146-1153). The yeast vacuole is analogous to the mammalian lysosomes as a degradation site of various proteins, including cytosolic, vacuolar, and integral membrane proteins (Wolf, D. H.2004. From lysosome to proteasome: the power of yeast in the dissection of proteinase function in cellular regulation and waste disposal. Cell Mol Life Sci, 61(13): 1601-1614).

UBC (Ubiqutin, Ub, conjugation), HRD (3-hydroxy-3-methylgutaryl-CoA reductase degradation) and DER (degradation in ER) genes have been identified as UBC/HRD/DER machinery critical for ER-associated degradation (ERAD) of Hmg2p and CPY* (a misfolded carboxypeptidase mutant that is retained in the ER lumen) (Hampton, R. Y. 2000. ER-associated degradation in protein quality control and cellular regulation. Curr Opin Cell Biol, 14(4): 476-482; Kostova, Z., and Wolf, D. 2003. For whom the bell tolls: protein quality control of the endoplasmic reticulum and the ubiquitin-proteasome connection. EMBO J, 22(10): 2309-2317).

Studies on the ER-associated Ub-conjugating enzymes (Ubc1p, Ubc6p, and Ubc7p) have led to the characterization of the role of ubiquitination in the degradation of various proteins and to the identification of the specific Ubc-s that are involved (Biederer, T., Volkwein, C., and Sommer, T. 1997. Role of cuelp in ubiquitination and degradation at the ER surface. Science, 278(5344): 1806-1809; Hampton, R. Y., and Bhakta, H.1997. Ubiquitin-mediated regulation of 3-hydroxy-3-methylglutaryl-CoA reductase. Proc Natl Acad Sci USA, 94(24): 12944-12948). In particular, two Ubcs (Ubc6p and Ubc7p) have been identified as key enzymes in the degradation of ER lumenal and membrane-bound proteins in yeast. Ubc6p is an integral, C-terminal anchored ER-membrane protein with its catalytic domain facing the cytosol. Ubc7p is a cytosolic protein that in yeast requires assembly with its partner, Cue1p, an integral membrane-anchored ER protein, for the degradation of ER-membrane bound proteins (such as Sec61p), ER-lumenal proteins (such as CPY*) and even soluble proteins (Biederer, T., Volkwein, C., and Sommer, T. 1997. Role of cuelp in ubiquitination and degradation at the ER surface. Science, 278(5344): 1806-1809; Sommer, T., and Wolf, D. H.1997. Endoplasmic reticulum degradation: reverse protein flow of no return. FASEB J, 11(14): 1227-1233).

Hrd2p is a 19S subunit that is essential for 26S proteasome function. Hrd1p/Hrd3p complex is an ER-associated Ub ligase. The HRD1 gene product is identical to Der3p, an integral ER-membrane protein with two distinct domains: an N-terminal, hydrophobic region with multiple predicted transmembrane spans and a cytosolic C-terminal hydrophilic RING-H motif required for the degradation of ER-lumenal proteins (Sommer, T., and Wolf, D. H.1997. Endoplasmic reticulum degradation: reverse protein flow of no return. FASEB J, 11(14): 1227-1233; Plemper, R. K. et al. 1999a. Genetic interactions of Hrd3p and Der3p/Hrd1p with Sec61p suggest a retro-translocation complex mediating protein transport for ER degradation. J Cell Sci, 112(22): 4123-4134). Der3p is identical to the integral protein Hmg2p (Gardner, R. G. et al. 2000. Endoplasmic reticulum degradation requires lumen to cytosol signaling: transmembrane control of Hrd1p by Hrd3p. J Cell Biol, 151(1): 69-82). The function of the HRD3 gene product, also an ER resident glycoprotein with single C-terminal membrane-anchor and a large N-terminal domain in the ER-lumen, has been recently elucidated (Gardner, R. et al. 2000. Endoplasmic reticulum degradation requires lumen to cytosol signaling: transmembrane control of Hrd1p by Hrd3p. J Cell Biol, 151(1): 69-82). Both Hrd1p and Hrd3p have been shown to form an ER-associated Ub-ligase complex that facilitates the Ubc7p-dependent ubiquitination and subsequent delivery of the polytopic HMGR to the 26S proteasome (Gardner, R. G. et al. 2000. Endoplasmic reticulum degradation requires lumen to cytosol signaling: transmembrane control of Hrd1p by Hrd3p. J Cell Biol, 151(1): 69-82 Wilhovsky, S. et al. 2000. HRD Gene dependence of endoplasmic reticulum associated degradation. Mol Biol Cell, 11(5): 1697-1708; Bays, N. M. et al. 2001. Hrd1p/Der3p is a membrane-anchored ubiquitin ligase required for ER-associated degradation. Nat Cell Biol, 3(1): 24-29). In addition, the Hrd/Der proteins not only have been proposed to function together with Sec61p in protein transport for ER degradation (Plemper, R. K. et al. 1999a. Genetic interactions of Hrd3p and Der3p/Hrd1p with Sec61p suggest a retro-translocation complex mediating protein transport for ER degradation. J Cell Sci, 112(22): 4123-4134; Plemper, R. K., and Wolf, D. H. 1999b. Endoplasmic reticulum degradation. Reverse protein transport and its end in the proteasome. Mol Biol Rep, 26(1-2): 125-130), but also are believed to play a central mechanistic role in the ER-associated degradation of several lumenal and integral ER proteins as well as in ER quality control in the removal of misfolded or otherwise aberrant proteins (Plemper, R. K. et al. 1999a. Genetic interactions of Hrd3p and Der3p/Hrd1p with Sec61p suggest a retro-translocation complex mediating protein transport for ER degradation. J Cell Sci, 112(22): 4123-4134; Wilhovsky, S. et al. 2000. HRD Gene dependence of endoplasmic reticulum associated degradation. Mol Biol Cell, 11(5): 1697-1708).

Cdc48p-Ufd1p-Hrd4p is a complex responsible for the recognition of polyubiquitinated ER proteins, their ER dislocation, and subsequent delivery to the 26S proteasome.

Mammalian homologues of the yeast HRD/DER machinery attest to the high evolutionary conservation of the ERAD process (Kostova, Z., and Wolf, D. 2003. For whom the bell tolls: protein quality control of the endoplasmic reticulum and the ubiquitin-proteasome connection. EMBO J, 22(10): 2309-2317).

The disruption of a gene encoding a protease in a target cell allows decreased degradation of cytochrome P450 and/or P450 reductase, and hence production of cytochrome P450s and/or P450 reductases with increased activity and/or increased expression. Indeed, the provision of protease deficient cells is useful in increasing production of cytochrome P450s and/or P450 reductases with high activity in target cells.

A sixteenth aspect of the present invention provides an isolated or recombinant nucleic acid molecule that enables disruption of a target gene encoding a protease degradation enzyme in a cell.

The cell may be a eukaryotic cell and may be a yeast, mammalian or insect cell. The cell may be a yeast cell. The nucleic acid molecule may enable deletion of the target gene in the cell.

The target gene may encode a protease degradation enzyme which may be a vacuolar or proteosomal protease. The vacuolar protease may be selected from PRA1(PEP4), protease B (PRB1) and carboxypeptidases Y & S(CPY & CPS), aminopeptidases, yscl and yscCo which participate in protein degradation. Proteasomal proteases may be selected from HRD (including HRD 1, HRD2 and HRD3), UBC (including UBC6 and UBC7), CUE1; and a Cdc48p-Ufd1p-Hrd4p complex. The nucleic acid molecule may be capable of integration into the genome of the cell. The nucleic acid molecule may be integrated into the genome through homologous recombination. The nucleic acid may enable deletion of a protease by targeted homologous recombination. The nucleic acid molecule may enable disruption of a vacuolar protease.

The nucleic acid molecule may comprise a nucleotide sequence that enables integration into the genome of the cell, flanked on either side by nucleotide sequences substantially identical to the target gene.

The nucleic acid molecule may comprise a selectable marker gene flanked on either side by nucleotide sequences substantially identical to the target gene. The flanking nucleotide sequences may be substantially identical to coding regions or regulatory regions of the target gene. The flanking nucleotide sequences may be substantially identical to 5′ and 3′ fragments of the target gene.

The 5′ fragment of the gene to be disrupted may comprise or consist of a nucleotide sequence selected from:

a) a nucleotide sequence of SEQ ID NO: 51, 58, 65 or 72;
b) a nucleotide sequence having at least 80% identity to the nucleotide sequence of a), wherein the nucleotide sequence causes disruption of the target gene when provided as part of a nucleic acid molecule of this aspect;
c) a nucleotide sequence that is complementary to the sequence of a) or b);
d) a fragment of a), b) or c), wherein the fragment causes disruption of the target gene when provided as part of a nucleic acid molecule of this aspect.

The nucleotide sequence of b) above may have at least 85%, 90% or 95% identity to the sequence of a). The nucleotide sequence may have at least 96%, 97%, 98% or 99% identity to the sequence of a).

The 3′ fragment of the gene to be disrupted may comprise or consist of a nucleotide sequence selected from:

a) a nucleotide sequence of SEQ ID NO: 54, 61, 68 or 75;
b) a nucleotide sequence having at least 80% identity to the nucleotide sequence of a), wherein the nucleotide sequence causes disruption of the target gene when provided as part of a nucleic acid molecule of this aspect;
c) a nucleotide sequence that is complementary to the sequence of a) or b);
d) a fragment of a), b) or c), wherein the fragment causes disruption of the target gene when provided as part of a nucleic acid molecule of this aspect.

The nucleotide sequence of b) above may have at least 85%, 90% or 95% identity to the sequence of a). The nucleotide sequence may have at least 96%, 97%, 98% or 99% identity to the sequence of a).

The fragments of d) may be of a suitable length to enable the gene to be disrupted via homologous recombination. The fragments may comprise at least 15 nucleotides, at least 20, at least 25, at least 30, at least 35, at least 40, at least 45, at least 50, at least 55, or at least 60 nucleotides.

When the protease gene to be disrupted is PRA1(PEP4) the 5′ fragment of PRA1 flanking the selectable marker may comprise or consist of the nucleotide sequence of SEQ ID NO: 51. The 3′ fragment of PRA1 may comprise or consist of the nucleotide sequence of SEQ ID NO: 54. When the protease gene to be disrupted is HRD1 the 5′ fragment of HRD1 flanking the selectable marker may comprise or consist of the nucleotide sequence of SEQ ID NO: 58. The 3′ fragment of HRD1 may comprise or consist of the nucleotide sequence of SEQ ID NO: 61. When the protease gene to be disrupted is HRD2 the 5′ fragment of HRD2 flanking the selectable marker may comprise or consist of the nucleotide sequence of SEQ ID NO: 65. The 3′ fragment of HRD2 may comprise or consist of the nucleotide sequence of SEQ ID NO: 68. When the protease gene to be disrupted is UBC7, the 5′ fragment of UBC7 flanking the selectable marker may comprise or consist of the nucleotide sequence of SEQ ID NO: 72. The 3′ fragment of UBC7 may comprise or consist of the nucleotide sequence of SEQ ID NO: 75.

The selectable marker gene may be selected from TRP1, LEU2, ADE2, URA3, HIS3, LYS2, HIS4 or MET15 or AUR1-C. The selectable marker gene may be TRP1 and may comprise or consist of the sequence of SEQ ID NO: 47.

On transformation of a yeast target cell with the nucleic acid molecule, the target gene is disrupted by the nucleic acid molecule comprising the selectable marker gene and the flanking 5′ and 3′ fragments of the target gene. Homologous recombination between the ends of the nucleic acid molecule replaces the target gene with the disrupted gene sequence. The gene disruption may be verified by PCR amplification using primers.

The nucleic acid molecule of the sixteenth aspect may be in the form of a vector. Therefore, in a seventeenth aspect there is provided a vector that enables disruption of a gene encoding a protease degradation enzyme in a target cell after transformation of the cell, the vector comprising a nucleic acid molecule of the sixteenth aspect.

An eighteenth aspect of the invention provides a cell transformed with a nucleic acid molecule of the sixteenth aspect and/or a vector of the seventeenth aspect. The nucleic acid molecule of the sixteenth aspect and/or vector of the seventeenth aspect may be integrated in the genome of the cell. The cell may be a eukaryotic cell and may be a yeast, mammalian or insect cell.

A nineteenth aspect of the invention provides a method of disrupting a gene encoding a protease degradation enzyme in a cell, comprising transforming the cell with a nucleic acid molecule of the sixteenth aspect and/or vector of the seventeenth aspect.

A twentieth aspect provides a method of producing a protease deficient yeast strain, comprising transforming a yeast strain with a nucleic acid molecule of the sixteenth aspect and/or vector of the seventeenth aspect.

The yeast strain may be Saccharomyces cerevisiae. The yeast strain may be selected from those known in the art. The yeast strain may be selected from the yeast strains JL20 (Daum, G et al. Yeast Functional Analysis Report. Yeast Volume 15, Issue 7, Pages 601-614) and W303B (Furuchi, T et al. Functions of yeast helicase Ssl2p that are essential for viability are also involved in protection from the toxicity of adriamycin. Nucleic Acids Res. 2004; 32(8): 2578-2585).

A twenty-first aspect provides a yeast strain produced by the method of the twentieth aspect.

The provision of protease deficient yeast strains provides for increased levels of activity and/or expression of cytochrome P450, which may be heterologous cytochrome P450, in yeast.

Therefore, in a twenty-second aspect, the present invention provides a method of producing a cytochrome P450 with increased activity and/or increased expression levels, comprising transforming a cell of the eighteenth aspect or yeast strain of the twenty first aspect with a vector of the fourth aspect capable of directing the expression of cytochrome P450.

The cytochrome P450 may be heterologous cytochrome P450. The cytochrome P450 may be human cytochrome P450.

To provide further increased activity and/or expression of cytochrome P450, the method may further comprise transforming the resultant cell or yeast strain with a nucleic acid molecule of the first, second or third aspects or with a vector of the fourth aspect capable of directing the expression of a nucleic acid molecule of the first, second or third aspects.

Alternatively, the method may further comprise transforming the cell or yeast strain with a nucleic acid molecule of the first, second or third aspects or with a vector of the fourth aspect capable of directing the expression of a nucleic acid molecule of the first, second or third aspects prior to transforming the yeast strain with the vector of the fourth aspect capable of directing the expression of cytochrome P450.

In addition, the method may further comprise transforming the cell or yeast strain with a vector capable of directing expression of a nucleotide sequence encoding a cytochrome b5 protein.

Alternatively, the method may comprise transforming the cell or yeast strain with a vector capable of directing expression of a nucleotide sequence encoding a cytochrome P450 and a nucleotide sequence encoding a cytochrome b5 protein, the sequences under the control of diverse promoters.

The cytochrome b5 protein may be a heterologous cytochrome b5 protein. The cytochrome b5 protein may be a human b5 protein.

Therefore, it is possible to generate cells and/or yeast strains expressing cytochrome P450 with increased activity and/or expression levels in view of the cell or yeast strain being deficient in a protease and also, if desired, expressing a P450 reductase, such as encoded by a nucleic acid molecule of the first, second or third aspects, and/or expressing a cytochrome b5 protein.

There is therefore provided in a twenty-third aspect a cell of the eighteenth aspect or yeast strain of the twenty first aspect transformed with a vector of the fourth aspect capable of driving expression of cytochrome P450 and/or a vector of the fourth aspect capable of driving expression of a nucleic acid molecule of the first, second or third aspects and/or a vector capable of directing expression of a nucleotide sequence encoding a cytochrome b5 protein.

The invention also provides in a twenty-fourth aspect, a protein expression system comprising:

i) a cell of the eighteenth aspect or yeast strain of the twenty first aspect; and

ii) a vector comprising a nucleotide sequence encoding a target protein, said sequence under the control of a promoter which causes expression of the target protein.

The nucleotide sequence may encode a cytochrome P450, which may be a heterologous cytochrome P450. The cytochrome P450 may be a human cytochrome P450. The vector may cause expression of the nucleotide sequence on integration into the cell or yeast strain. The vector may be as defined in the fourth aspect.

The protein expression system may further comprise a vector of the fourth aspect capable of expressing a nucleic acid molecule of the first, second or third aspect.

The protein expression system may further comprise a vector capable of expressing a nucleotide sequence encoding a cytochrome b5 protein.

The inventors have also found that to increase activity and expression of cytochrome P450 in yeast expression systems, yeast strains which are not contaminated with endogenous cytochrome P450 reductase (yRD) can be provided. Further it has been found that it is possible to provide yeast strains with different yRD activities by expressing yRD at different chromosomal loci in yeast strains not contaminated with endogenous yRD.

A twenty-fifth aspect provides an isolated or recombinant nucleic acid molecule that enables disruption of a target gene encoding endogenous P450 reductase in a cell.

The cell may be a eukaryotic cell and may be a yeast, mammalian or insect cell.

The nucleic acid molecule may comprise a nucleotide sequence that enables integration into the genome of the cell, flanked on either side by nucleotide sequences substantially identical to the target gene. The nucleic acid molecule may comprise a selectable marker gene flanked by nucleotide sequences substantially identical to the target gene. The nucleotide sequences substantially identical to the target gene may be selected such that on homologous recombination the target gene is disrupted.

The flanking nucleotide sequences may comprise or consist of:

a) i) a nucleotide sequence of SEQ ID NO: 84;

ii) a nucleotide sequence having at least 80% identity to the nucleotide sequence of ii), wherein the nucleotide sequence causes disruption of the target gene when provided as part of a nucleic acid molecule of this aspect;

iii) a nucleotide sequence that is complementary to the sequence of i) or ii); or

iv) a fragment of i), ii) or iii), wherein the fragment causes disruption of the target gene when provided as part of a nucleic acid molecule of this aspect; and

b) i) a nucleotide sequence of SEQ ID NO: 85;

ii) a nucleotide sequence having at least 80% identity to the nucleotide sequence of i), wherein the nucleotide sequence causes disruption of the target gene when provided as part of a nucleic acid molecule of this aspect;

iii) a nucleotide sequence that is complementary to the sequence of i) or ii); or

iv) a fragment of i), ii) or iii), wherein the fragment causes disruption of the target gene when provided as part of a nucleic acid molecule of this aspect.

The nucleotide sequence of a) ii) and b) ii) above may have at least 85%, 90% or 95% identity to the sequence of a) i) and b) i), respectively. The nucleotide sequences may have at least 96%, 97%, 98% or 99% identity to the sequence of a) i) or b) i).

The fragments of a) iii) and b) iii) may be of a suitable length to enable the gene to be disrupted via homologous recombination. The fragments may comprise at least 15 nucleotides, at least 20, at least 25, at least 30, at least 35, at least 40, at least 45, at least 50, at least 55, or at least 60 nucleotides.

The selectable marker gene may be selected from LEU2, TRP1, ADE2, URA3, HIS3, LYS2, HIS4, MET15 or AUR1-C or other marker genes.

On transformation of a yeast target cell (Klebe, L. K. et al. 1983. A general method for polyethylenglycol-induced genetic transformation of bacteria and yeast. Gene, 25(2-3): 333-341; Schiestl, R. H., and Gietz, R. D. 1989. High efficiency transformation of intact yeast cells by using single stranded nucleic acid as carrier. Curr Genet, 16(5-6): 339-346) with the nucleic acid molecule, the target gene is disrupted by the nucleic acid molecule comprising the selectable marker gene and the flanking nucleotide sequence. Homologous recombination between the ends of the nucleic acid molecule replaces the target gene with the disrupted gene sequence. The gene disruption may be verified by PCR amplification using primers.

The nucleic acid molecule may be in the form of a vector. Therefore, in a twenty-sixth aspect, there is provided a vector that enables disruption of an endogenous gene encoding a P450 reductase in a target cell after transformation of the cell, the vector comprising a nucleic acid molecule of the twenty fifth aspect.

Alternatively, a twenty-seventh aspect provides a vector that enables disruption of an endogenous gene encoding a P450 reductase in a target cell after transformation of the cell, the vector comprising a nucleotide sequence substantially identical to the target gene and a nucleotide sequence that enables integration into the genome of the cell, the nucleotide sequence substantially identical to the target gene comprising one or more unique restriction sites to enable the vector to be linearised to enable integration into the genome of the target cell.

The nucleotide sequence substantially identical to the target gene may have at least 80% identity, at least 85%, 90%, 95%, 96%, 97%, 98% or even 99% or greater identity to the target gene. The nucleotide sequence may comprise or consist of a sequence selected from:

a) the nucleotide sequence of SEQ ID No: 81;

b) a nucleotide sequence having at least 80% identity to the sequence of a);

c) a nucleotide sequence which is complementary to the sequence of a) or b); or

d) a nucleotide sequence which codes for the same polypeptide as the sequence of a), b) or c).

A twenty-eighth aspect provides a cell transformed with a nucleic acid molecule of the twenty fifth aspect and/or a vector of the twenty-sixth or twenty-seventh aspects.

The cell may be a eukaryotic cell and may be a yeast, mammalian or insect cell.

A twenty-ninth aspect provides a method of disrupting an endogenous gene encoding a P450 reductase, comprising transforming a cell with a nucleic acid molecule of the twenty-fifth aspect and/or a vector of the twenty-sixth or twenty-seventh aspects.

A thirtieth aspect provides a method of producing a cell or yeast strain deficient in a cytochrome P450 reductase, comprising transforming a yeast strain with a nucleic acid molecule of the twenty-fifth aspect and/or a vector of the twenty-sixth or twenty-seventh aspects.

The yeast strain may be Saccharomyces cerevisiae. The yeast strain may be selected from those known in the art. The yeast strain may be selected from the yeast strains JL20 (Daum, G et al. Yeast Functional Analysis Report. Yeast Volume 15, Issue 7, Pages 601-614) and W303B (Furuchi, T et al. Functions of yeast helicase Ssl2p that are essential for viability are also involved in protection from the toxicity of adriamycin. Nucleic Acids Res. 2004; 32(8): 2578-2585).

A thirty-first aspect provides a yeast strain produced by the method of the thirtieth aspect or transformed with a vector of the twenty-sixth or twenty-seventh aspects.

A thirty-second aspect provides a method of producing cytochrome P450 with increased activity and/or expression in yeast, comprising transforming a cell of the twenty eighth aspect or a yeast strain of the thirty-first aspect with a vector of the fourth aspect capable of directing the expression of a cytochrome P450.

The cytochrome P450 may be heterologous cytochrome P450. The cytochrome P450 may be human cytochrome P450.

The method may further comprise transforming the cell or yeast strain with a vector of the fourth aspect capable of directing the expression of a nucleic acid molecule of the first, second or third aspect.

Alternatively, the method may further comprise transforming the cell or yeast strain with a vector of the fourth aspect capable of directing the expression of a nucleic acid molecule of the first, second or third aspect prior to transforming the cell or yeast strain with a vector of the fourth aspect capable of directing the expression of cytochrome P450.

In addition, the method may further comprise transforming the cell or yeast strain with a vector capable of directing expression of a nucleotide sequence encoding a cytochrome b5 protein.

Alternatively, the method may comprise transforming the cell or yeast strain with a vector capable of directing expression of a nucleotide sequence encoding a cytochrome P450 and a nucleotide sequence encoding a cytochrome b5 protein, the sequences under the control of diverse promoters.

The cytochrome b5 protein may be a heterologous cytochrome b5 protein. The cytochrome b5 protein may be a human b5 protein.

There is further provided, in a thirty-third aspect, a yeast strain of the thirty-first aspect, transformed with a vector of the fourth aspect capable of driving expression of the cytochrome P450 and/or a vector of the fourth aspect capable of directing expression of a nucleic acid molecule of the first, second or third aspects and/or a vector capable of directing expression of a nucleotide sequence encoding a cytochrome b5 protein.

The present invention also provides in a thirty-fourth aspect a protein expression system comprising:

i) a cell of the twenty-eighth aspect or yeast strain of the thirty first aspect; and

ii) a vector comprising a nucleotide sequence encoding a target protein, said sequence under the control of a promoter which causes expression of the target protein.

The nucleotide sequence may encode a cytochrome P450, which may be a heterologous cytochrome P450. The cytochrome P450 may be a human cytochrome P450. The vector may cause expression of the nucleotide sequence on integration into the cell or yeast strain. The vector may be as defined in the fourth aspect.

The protein expression system may further comprise a vector of the fourth aspect capable of expressing a nucleic acid molecule of the first, second or third aspect.

The protein expression system may further comprise a vector capable of expressing a nucleotide sequence encoding a cytochrome b5 protein.

The protein expression system may further comprise a vector comprising a nucleotide sequence encoding endogenous yeast cytochrome P450 reductase under the control of a promoter which causes expression of the yeast cytochrome P450 reductase.

The present invention in a thirty-fifth aspect provides an isolated or recombinant nucleic acid molecule comprising a nucleotide sequence encoding endogenous yeast cytochrome P450 reductase under the control of a promoter which causes expression of the yeast cytochrome P450 reductase. The nucleotide sequence may comprise or consist of:

a) the nucleotide sequence of SEQ ID No: 78;

b) a nucleotide sequence having at least 80% identity to the sequence of a);

c) a nucleotide sequence which is complementary to the sequence of a) or b); or

d) a nucleotide sequence which codes for the same polypeptide as the sequence of a), b) or c).

The nucleotide sequence of b) above may have at least 85%, 90% or 95% identity to the sequence of a). The nucleotide sequence may have at least 96%, 97%, 98% or 99% identity to the sequence of a).

The present invention in a thirty-sixth aspect provides a vector comprising a nucleic acid molecule of the thirty-fifth aspect.

The promoter may be an inducible promoter, which may be a GAL promoter. The promoter may comprise a truncated GAL promoter. The truncated GAL promoter may be a truncated GAL1 promoter. The truncated GAL1 promoter may be a GAL1 promoter truncated at nucleotide 202. The truncated GAL1 promoter may comprise or consist of the sequence of SEQ ID NO: 2. Alternatively, the inducible promoter may be a ADH2 promoter.

The nucleic acid molecule may further comprise a transcription termination sequence which may be downstream of the promoter. The nucleic acid molecule may comprise unique restriction sites between the GAL promoter and termination sequence that allow insertion of a nucleotide sequence under the control of the promoter. The transcription termination sequence may be immediately downstream of the inserted nucleotide sequence or separated by a minimal distance. The transcription termination sequence may be separated from the inserted nucleotide sequence by 5-25 nucleotides. It may be separated by 5-20, 5-15, 15-20, 5-10, 6-9 or 6-8 nucleotides. It may be separated by 5, 6, 7, 8, 9, 10, 11, 12, 13, 14, 15, 16, 17, 18, 19 or 20 nucleotides.

The termination sequence may be a termination sequence from any expressed gene and may be selected from SUC2 (SUC2t), PHO5, ADH1, ADH2 or CYC1. The termination sequence may be a SUC2 (SUC2t) termination sequence, and may comprise or consist of the sequence of SEQ ID NO: 28.

The vector may be adapted to cause integration of the nucleotide sequence encoding yeast cytochrome P450 reductase at a particular chromosomal locus in a target cell, which may be a yeast cell. The vector may comprise a selectable marker gene that enables integration of the vector into a particular chromosomal locus. The selectable marker gene may be selected from LEU2, ADE2, HIS3, TRP1, URA3, LYS2, HIS4, MET15 or AUR1-C.

A thirty-seventh aspect provides a cell transformed with a nucleic acid molecule of the thirty-fifth aspect or with a vector of the thirty-sixth aspect.

A thirty-eighth aspect provides a method of producing a cell or yeast strain expressing endogenous cytochrome P450 reductase, comprising transforming a yeast strain with a nucleic acid molecule of the thirty-fifth aspect or with a vector of the thirty-sixth aspect.

The cell may be a eukaryotic cell which may be a yeast, mammalian or insect cell.

The yeast strain may be Saccharomyces cerevisiae. The yeast strain may be selected from those known in the art. The yeast strain may be selected from the yeast strains JL20 (Daum, G et al. Yeast Functional Analysis Report. Yeast Volume 15, Issue 7, Pages 601-614) and W303B (Furuchi, T et al. Functions of yeast helicase Ssl2p that are essential for viability are also involved in protection from the toxicity of adriamycin. Nucleic Acids Res. 2004; 32(8): 2578-2585.)

A thirty-ninth aspect provides a yeast strain produced by the method of the thirty-eighth aspect or transformed with a vector of the thirty-sixth aspect.

A fortieth aspect provides a method of producing cytochrome P450 with increased activity and/or expression in yeast, comprising transforming a cell of the thirty-seventh aspect or a yeast strain of the thirty-ninth aspect with a vector of the fourth aspect capable of directing the expression of a cytochrome P450.

The cytochrome P450 may be heterologous cytochrome P450. The cytochrome P450 may be human cytochrome P450.

The method may further comprise transforming the cell or yeast strain with a vector of the fourth aspect capable of directing the expression of a nucleic acid molecule of the first, second or third aspect.

Alternatively, the method may further comprise transforming the cell or yeast strain with a vector of the fourth aspect capable of directing the expression of a nucleic acid molecule of the first, second or third aspect prior to transforming the cell or yeast strain with a vector of the fourth aspect capable of directing the expression of cytochrome P450.

In addition, the method may further comprise transforming the cell or yeast strain with a vector capable of directing expression of a nucleotide sequence encoding a cytochrome b5 protein.

Alternatively, the method may comprise transforming the cell or yeast strain with a vector capable of directing expression of a nucleotide sequence encoding a cytochrome P450 and a nucleotide sequence encoding a cytochrome b5 protein, the sequences under the control of diverse promoters.

The cytochrome b5 protein may be a heterologous cytochrome b5 protein. The cytochrome b5 protein may be a human b5 protein. The cytochrome b5 protein may be encoded by a nucleotide sequence comprising or consisting of:

a) the nucleotide sequence of SEQ ID NO: 86;

b) a nucleotide sequence having at least 80% identity to the sequence of a) and encoding a cytochrome b5 protein;

c) a nucleotide sequence which is complementary to the sequence of a) or b); or

d) a nucleotide sequence which codes for the same polypeptide as the sequence of a), b) or c).

The nucleotide sequence of b) above may have at least 85%, 90% or 95% identity to the sequence of a). The nucleotide sequence may have at least 96%, 97%, 98% or 99% identity to the sequence of a).

Alternatively, cytochrome b5 protein may be selected from those known in the art. (For example: Yoo, M et al. The complete nucleotide sequence of human liver cytochrome b-5 mRNA. Biochem. Biophys. Res. Commun. 156, 576-580 (1988).)

There is further provided, in a forty-first aspect, a yeast strain of the thirty-ninth aspect, transformed with a vector of the fourth aspect capable of driving expression of the cytochrome P450 and/or a vector of the fourth aspect capable of directing expression of a nucleic acid molecule of the first, second or third aspects and/or a vector capable of directing expression of a nucleotide sequence encoding a cytochrome b5 protein.

The present invention also provides in a forty-second aspect a protein expression system comprising:

i) a cell of the thirty-seventh aspect or yeast strain of the thirty-ninth aspect; and

ii) a vector comprising a nucleotide sequence encoding a target protein, said sequence under the control of a promoter which causes expression of the target protein.

The nucleotide sequence may encode a cytochrome P450, which may be a heterologous cytochrome P450. The cytochrome P450 may be a human cytochrome P450. The vector may cause expression of the nucleotide sequence on integration into the cell or yeast strain. The vector may be as defined in the fourth aspect.

The protein expression system may further comprise a vector of the fourth aspect capable of expressing a nucleic acid molecule of the first, second or third aspect.

The protein expression system may further comprise a vector capable of expressing a nucleotide sequence encoding a cytochrome b5 protein.

Like P450 reductase, the cytochrome b5 protein is a co-factor that contributes to cytochrome P450 activity. The inventors have found that to improve expression of cytochrome P450 with high activities, cytochrome b5 protein can be co-expressed in the target cell.

There is therefore provided in a forty third aspect an isolated or recombinant nucleic acid molecule comprising a nucleotide sequence encoding a cytochrome b5 protein under the control of a promoter.

The promoter may be a constitutive or inducible promoter. The promoter may be a GAL promoter which may be a truncated GAL promoter. Alternatively it may be the ADH2 promoter. The constitutive promoter may be selected from promoters from the ACT1, ADH1, GAPDH, PGK1, PMA1, TEF, TPI genes. The inducible promoter may be selected from the ADH2, PHO5, MET25, CYC1 genes.

The nucleic acid molecule may further comprise a transcription termination sequence which may be downstream of the promoter. The nucleic acid molecule may comprise unique restriction sites between the GAL promoter and termination sequence that allow insertion of a nucleotide sequence under the control of the promoter. The transcription termination sequence may be immediately downstream of the inserted nucleotide sequence or separated by a minimal distance. The transcription termination sequence may be separated from the inserted nucleotide sequence by 5-25 nucleotides. It may be separated by 5-20, 5-15, 15-20, 5-10, 6-9 or 6-8 nucleotides. It may be separated by 5, 6, 7, 8, 9, 10, 11, 12, 13, 14, 15, 16, 17, 18, 19 or 20 nucleotides.

The termination sequence may be a termination sequence from any expressed gene and may be selected from SUC2 (SUC2t), PHO5, ADH1, ADH2 or CYC1 genes. The termination sequence may be a SUC2 (SUC2t) termination sequence, and may comprise or consist of the sequence of SEQ ID NO: 28.

The nucleotide sequence encoding the cytochrome b5 protein may encode a mammalian cytochrome b5 protein which may be a human cytochrome b5 protein. The nucleotide sequence may comprise or consist of a sequence selected from:

a) the nucleotide sequence of SEQ ID NO: 86;

b) a nucleotide sequence having at least 80% identity to the sequence of a) and encoding a cytochrome b5 protein;

c) a nucleotide sequence which is complementary to the sequence of a) or b); or

d) a nucleotide sequence which codes for the same polypeptide as the sequence of a), b) or c).

The nucleotide sequence of b) above may have at least 85%, 90% or 95% identity to the sequence of a). The nucleotide sequence may have at least 96%, 97%, 98% or 99% identity to the sequence of a).

The nucleotide sequence may alternatively comprise a sequence selected from sequences known in the art.

The nucleic acid molecule may be in the form of a vector. Therefore in a forty-fourth aspect there is provided a vector comprising a nucleic acid molecule of the forty-second aspect.

The vector may be adapted to cause integration of the nucleotide sequence encoding cytochrome b5 protein at a particular chromosomal locus in a target cell, which may be a yeast cell. The vector may comprise a selectable marker gene that enables integration of the vector into a particular chromosomal locus. The selectable marker gene may be selected from LEU2, ADE2, HIS3, TRP1, URA3, LYS2, HIS4 or MET15 or AUR1-C.

A forty-fifth aspect provides a cell transformed with a nucleic acid molecule of the forty third aspect or with a vector of the forty fourth aspect.

A forty-sixth aspect provides a method of producing a cell or yeast strain expressing cytochrome b5 protein, comprising transforming a yeast strain with a nucleic acid molecule of the forty third aspect or with a vector of the forty fourth aspect.

A forty-seventh aspect provides a yeast strain produced by the method of the forty sixth aspect or transformed with a vector of the forty fourth aspect.

A forty-eighth aspect provides a method of producing cytochrome P450 with increased activity and/or expression in yeast, comprising transforming a cell of the forty-fifth aspect or a yeast strain of the forty seventh aspect with a vector of the fourth aspect capable of directing the expression of a cytochrome P450.

The cytochrome P450 may be heterologous cytochrome P450. The cytochrome P450 may be human cytochrome P450.

The method may further comprise transforming the cell or yeast strain with a vector of the fourth aspect capable of directing the expression of a nucleic acid molecule of the first, second or third aspect.

Alternatively, the method may further comprise transforming the cell or yeast strain with a vector of the fourth aspect capable of directing the expression of a nucleic acid molecule of the first, second or third aspect prior to transforming the cell or yeast strain with a vector of the fourth aspect capable of directing the expression of cytochrome P450.

In addition, the method may further comprise transforming the cell or yeast strain with a vector of the thirty sixth aspect capable of directing expression of endogenous cytochrome P450 reductase.

There is further provided, in a forty-ninth aspect, a yeast strain of the forty seventh aspect, transformed with a vector of the fourth aspect capable of directing expression of a cytochrome P450 and/or a vector of the fourth aspect capable of directing expression of a nucleic acid molecule of the first, second or third aspects and/or a vector of the thirty-sixth aspect capable of directing expression of endogenous cytochrome P450 reductase.

The present invention also provides in a fiftieth aspect a protein expression system comprising:

i) a cell of the forty-fifth aspect or yeast strain of the forty-seventh aspect; and

ii) a vector comprising a nucleotide sequence encoding a target protein, said sequence under the control of a promoter which causes expression of the target protein.

The nucleotide sequence may encode a cytochrome P450, which may be a heterologous cytochrome P450. The cytochrome P450 may be a human cytochrome P450. The vector may cause expression of the nucleotide sequence on integration into the cell or yeast strain. The vector may be as defined in the fourth aspect.

The protein expression system may further comprise a vector of the fourth aspect capable of expressing a nucleic acid molecule of the first, second or third aspect.

The protein expression system may further comprise a vector of the thirty-sixth aspect capable of directing expression of endogenous cytochrome P450 reductase.

The inventors have also found that cytochrome P450 with increased activity can be produced by co-expressing cytochrome P450 with one or more of a P450 reductase of the present invention, a cytochrome b5 protein or a yeast cytochrome P450 reductase in separate cells and then blending the cells to provide a cell mixture. Microsomes can then be prepared from the cell mixture to produce increased levels of cytochrome P450 activity.

Therefore, in fifty-first aspect there is provided a method of producing cytochrome P450 with increased activity, comprising blending cells transformed with a vector of the fourth aspect capable of directing expression of a cytochrome P450 with one or more of:

a) one or more cells of the fifth aspect;
b) one or more cells of the eighteenth aspect;
c) one or more cells of the twenty-eighth aspect;
d) one or more cells of the thirty-seventh aspect; and/or
e) one or more cells of the forty-fifth aspect.

The method may comprise a further step of isolating the cells from culture before blending the cells. The cells may be resuspended before blending. The blending of cells may comprise mixing the cells.

The cells may be blended to produce a homogenous cell mixture. The cell mixture may contain different ratios of cells containing a cytochrome P450 and also cells from the fifth, eighteenth, twenty-eighth, thirty-seventh and/or forty-fifth aspects. In this way, it is possible for the cell mixture to be used to make microsomes to obtain a batch of homogeneous microsomes that contain a defined amount of cytochrome P450 with a defined P450 reductase activity which would provide for a defined cytochrome P450 specific activity. The method therefore may further comprise preparing microsomes from the cell mixture. The microsomes may be prepared by methods well known in the art (Renaud J P et al., Recombinant yeast in drug metabolism. Toxicology. 1993 Oct. 5; 82(1-3):39-52; Simula A P et al., Heterologous expression of drug-metabolizing enzymes in cellular and whole animal models. Toxicology. 1993 Oct. 5; 82(1-3):3-20; Guengerich F P et al., Expression of human cytochrome P450 enzymes in yeast and bacteria and relevance to studies on catalytic specificity. Toxicology. 1993 Oct. 5; 82(1-3):21-37; Hayashi K et al., Coexpression of genetically engineered fused enzyme between yeast NADPH-P450 reductase and human cytochrome P450 3A4 and human cytochrome b5 in yeast. Arch Biochem Biophvs. 2000 Sep. 1; 381(1):164-70; Pompon D, et al. Yeast expression of animal and plant P450s in optimized redox environments. Methods Enzymol. 1996; 272:51-64; Guengerich F P et al. Expression of mammalian cytochrome P450 enzymes using yeast-based vectors. Methods Enzmmol. 1991; 206:130-45).

The activity of cytochrome P450 can be measured by the measurement of the activity either using fluorescent or luminescence assays. For example, measurement of CYP1B1, CYP1A1, CYP1A2 utilises the 7-ethoxyresorufin O-deethylase assay (Klotz A. V., S. J. J. a. W. C. An alternative 7-ethoxyresorufin O-deethylase activity assay: a continuous visible spectrophotometric method for measurement of cytochrome P450 monooxygenase activity. Analytical Biochemistry 140: 138-145, 1984). These enzyme convert 7-ethoxyresorufin to resorufin which can be flourimetrically detected in presence of NADPH and oxygen and the assay is known as the EROD assay. Other assays may be used for other cytochrome P450 isozymes. Such assays are known in the art.

An increase in cytochrome P450 activity is observed when the activity of cytochrome P450 after blending of cells is greater than cytochrome P450 activity before blending of cells.

A fifty-second aspect provides the use of a cell or yeast strain of the invention to evaluate the toxicity of a candidate drug compound. An assay may be used to evaluate the toxicity. The assay may be a high throughput assay.

A fifty-third aspect provides a method of evaluating the toxicity of a candidate drug compound, comprising:

a) reacting the compound with a cell or yeast strain of the present invention which expresses cytochrome P450; and
b) determining the IC₅₀ concentrations for inhibition.

The IC₅₀ concentrations may be determined using assays known in the art, such as fluorescent or luminescent assays. The toxicity of a compound may be assessed by the observed inhibition of cytochrome P450. The determination of the IC₅₀ at different concentrations of a compound enables the concentration of the compound at which the cytochrome P450 enzyme is inhibited by 50%. This would in turn enable the toxicity of the compound to be evaluated.

A fifty-fourth aspect provides a method of evaluating compounds for inhibition of cytochrome P450, the method comprising:

a) reacting a test compound with a cell or yeast strain of the present invention which expresses cytochrome P450; and
b) determining whether the compound reduces cytochrome P450 activity.

The cell or yeast strain may be transformed with a vector of the present invention which directs the expression of a nucleotide sequence encoding a cytochrome P450. The cytochrome P450 may be a heterologous cytochrome P450 and may be human cytochrome P450.

The determination step b) may comprise:

a) growing the cells;

b) adding a substrate for cytochrome P450 to the cells; and

c) measuring the activity of the cytochrome P450.

The activity may be measured by measuring the metabolism of the substrate. The substrate may be a fluorescent or luminescent substrate. The substrate may be selected based on the cytochrome P450 expressed by the cells. The skilled person in the art can readily select the appropriate substrate to use in the method.

The activity may be measured at various time points during growth of the cells. The activity may be measured by removing cells after addition of a reagent to induce expression of the cytochrome P450 in the cells. The reagent may be galactose.

The measurements may be performed at a specific excitation/emission wavelength using a spectrophotometer to determine the activity of the cytochrome P450.

The cell or yeast strain may be selected for use in the method that express cytochrome P450 with high activity. The cell or yeast cell may be selected by using an assay comprising the steps of:

a) growing cells;

b) adding a substrate for cytochrome P450 to the cells; and

c) measuring the activity of the cytochrome P450.

In a further aspect there is provided a cytochrome P450 produced by a method of the seventh, twelfth, thirteenth, twenty-second, thirty-second, fortieth, forty-eighth or fifty-first aspects of the present invention.

Preferred features of each aspect of the invention are as for each of the other aspects mutatis mutandis. The prior art documents mentioned herein are incorporated to the fullest extent permitted by law.

### Example 1

### Construction of Yeast GAL1/GAL10 Promoter Variants that are Inducible by Galactose and Comparative Analysis of the Expression of CYP1B1 in the Presence of Endogenous Yeast Reductase

There are five types of GAL1/GAL10 promoters (GAL1/GAL10 is a bi-cistronic promoter inducible by the sugar galactose and repressed by glucose) that have been reported in the literature:

(1) A ˜907 bp full length GAL1/GAL10 promoter (have been used by academic labs),
(2) A ˜650 bp truncated GAL1 promoter (have been used by academic labs),
(3) A ˜675 bp GAL1 promoter fragment has been used by academic labs and also is present in pESC-based plasmids commercially available from Stratagene,
(4) A ˜461 bp truncated GAL1 promoter in the pYES2-based plasmids available commercially from Invitrogen,
(5) Hybrid promoters that contain the upstream activation sequences (UAS-s) of the GAL1/GAL10 promoter that are linked to the core (i.e. basal) CYC1 promoter. One variant of these promoters (CYC1-GAL10UAS) has been used in U.S. Pat. No. 5,635,369. The other promoters mentioned in U.S. Pat. No. 5,635,369 are constitutively active. Strong constitutive expression of P450 isozymes and its co-factor P450 reductase is very likely deleterious to the yeast cell (i.e. strong constitutive promoters invariably produce less P450 protein than inducible promoters).

All five different types of promoters were used to express the CYP1B1 gene (a cytochrome P450 isozyme) to compare the amounts of P450 produced in basic yeast strains that contain only the endogenous yeast P450 reductase. It was found that in the strains JL20 and W303-B (two Mat a strains), that contain the endogenous yeast P450 reductase, the 675 bp GAL1 promoter provides the best expression.

### Example 1.1

### The DNA Sequence Comparison of the 907, 675, 650 & 461 bp GAL1 Promoters (SEQ ID NOS: 1, 2, 3 & 4 respectively) is Illustrated Below (Sequences which are Identical are Highlighted in Yellow)

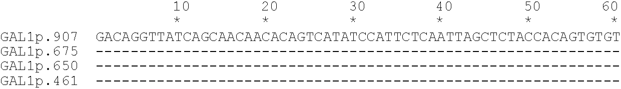

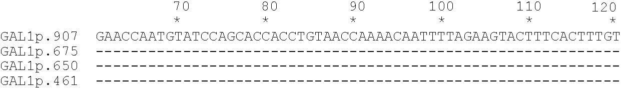

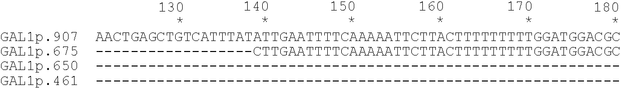

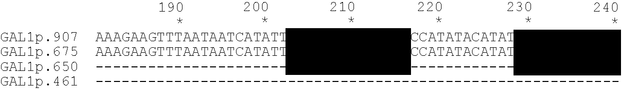

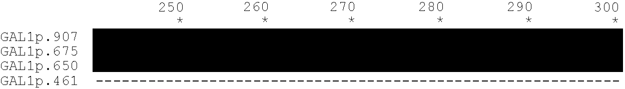

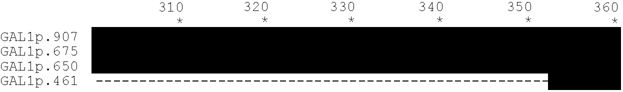

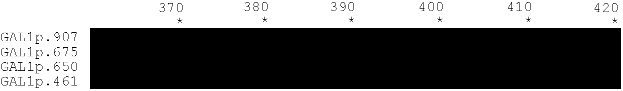

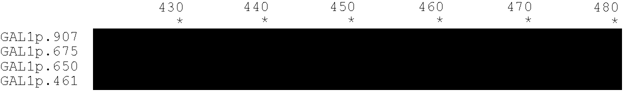

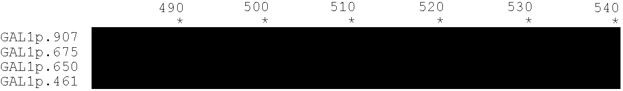

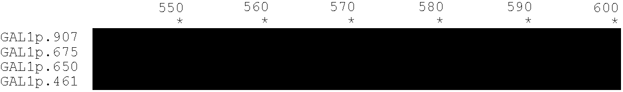

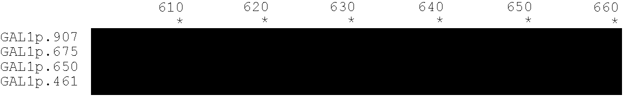

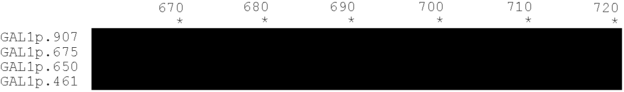

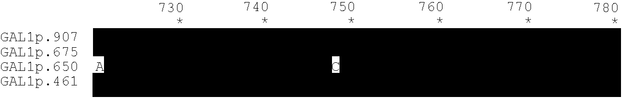

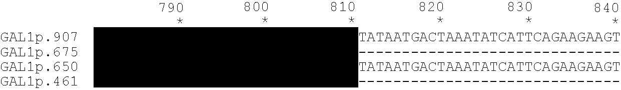

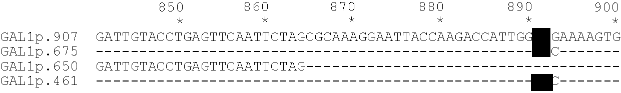

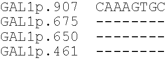

### Example 1.2

### Cloning of a 907 bp GAL1 Promoter as a NgoMIV-BamHI Fragment

The cloning was performed in three steps. The first step involved the polymerase chain reaction (PCR). It was used to amplify the 907 bp GAL1 promoter (SEQ ID No. 1) as an NgoMIV-BamHI fragment. 100 pmoles each of the PCR primers (5′ PCR primer: 5′-ATgccggc GACAGGTTAT CAGCAACAAC ACAGTCATAT CC-3′ (SEQ ID NO: 5-letters in lower casing represent the NgoMIV site) &: 3′ PCR primer: 5′-ATggatcc GCACTTTGCA CTTTTCGGCC AATGGTCTTG GTAATTCC-3′ (SEQ ID NO: 6-letters in lower casing represent the BamHI site)) were used with genomic DNA (50 ng), from the Saccharomyces cerevisiae strain S288C (Invitrogen, Cat No. 40802), as template.

The ˜907 bp PCR product was purified on a QiaQuick column (Qiagen UK, Cat No. 28104).

In the second step, the purified DNA was digested with the restriction enzymes NgoMIV and BamHI (both obtained from New England BioLabs, UK) and ˜100 ng of the restricted DNA (the insert) was ligated to 50 ng of pBlueScriptII SK(+) (Stratagene, UK), the vector, which had been digested with NgoMIV and BamHI. Ligation was performed between the vector and the insert using DNA ligase (Roche Diagnostics, UK) in a 15 μl of reaction mixture, following the supplier's protocol.

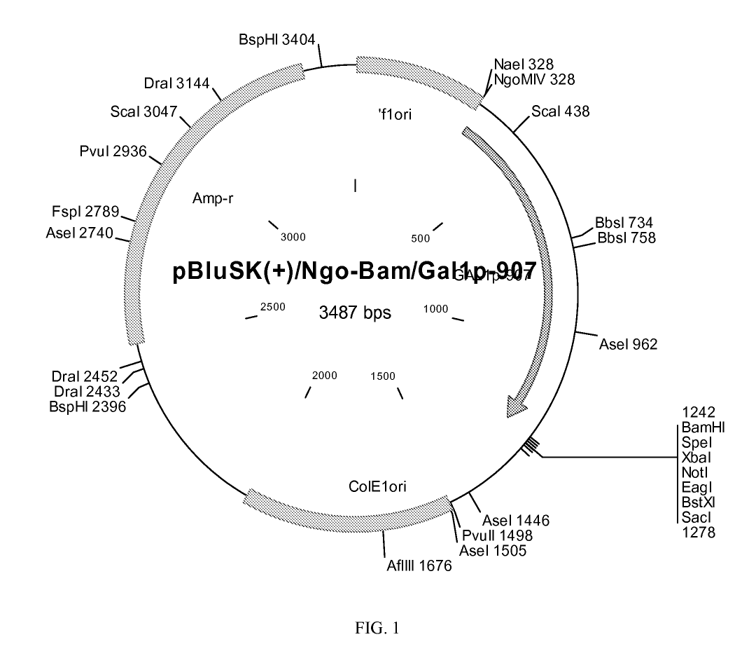

In the final step, 3 μl of the ligation mixture was transformed into Escherichia coli DH5alpha competent cells, made competent with the standard CaCl₂ protocol [1]. DNA from six individual clones was isolated and the veracity of the clones was confirmed by restriction enzyme analysis and further corroborated by DNA sequencing. One correct clone was named pBluSK(+)/Ngo-Bam/Gal1p-907 (FIG. 1) and was used further for cloning in a 2-micron yeast expression vector.

Future cloning of all promoter and gene fragments was performed as in this Example 1.2.

### Example 1.3

### Cloning of the NgoMIV-BamHI 907 bp GAL1 Promoter Fragment in a Yeast 2-Micron Expression Vector

A NgoMIV-BamHI GAL1 907 bp promoter fragment from pBluSK(+)/Ngo-Bam/Gal1p-907 (FIG. 1) was isolated and ligated to a 5190 bp fragment of pYES2 (Invitrogen; a commercial 2-micron vector that bears the URA3 auxotrophic marker) which had already been digested with NgoMIV-BamHI.

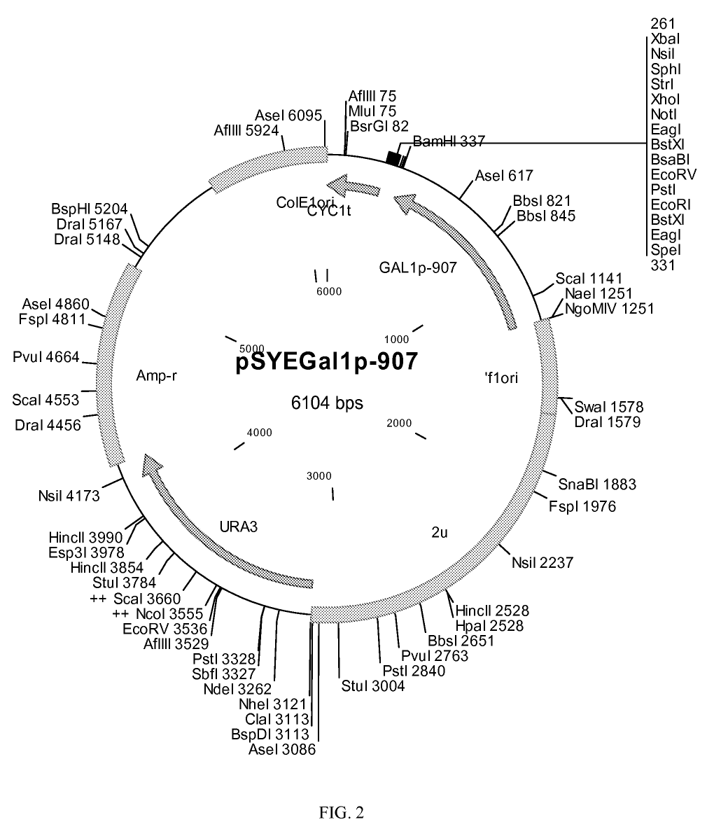

One correct clone obtained after ligation and transformation in DH5alpha bacterial cells was named pSYEGal1p-907 (FIG. 2) and was used for expression of genes encoding cytochrome P450 isozymes (CYP-s). The veracity of the clone was confirmed by restriction enzyme analysis.

### Example 1.4

### Cloning of the 675 bp GAL1 Promoter as a NgoMIV-BamHI Fragment

The cloning of a NgoMIV-BamHI GAL1 promoter fragment (SEQ ID No. 2) in pBlueScriptII SK(+) was performed as in Example 1.2, using GAL1 sequence specific primers ((5′ PCR primer: 5′-ATgccggcCTTGAATTTTCAAAAATTCTTACTTTTTTTTTGG-3′ (letters in lower casing represent the NgoMIV site—SEQ ID NO: 7) & 3′ PCR primer: 5′-ATggatcc GGGGTTTTTTCTCCTTGACGTTAAAGTATAGAGG-3′ (letters in lower casing represent the BamHI site—SEQ ID NO: 8)). The amplified fragment, digested with NgoMIV-BamHI, was cloned in pBlueScriptII SK(+) digested with NgoMIV-BamHI.

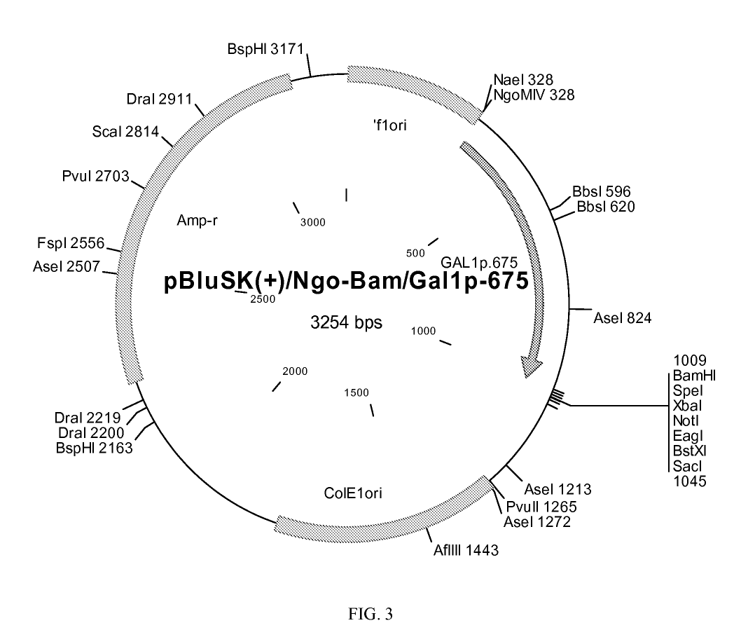

One correct clone obtained after ligation and transformation in DH5alpha bacterial cells was named pBluSK/Ngo-Bam/Gal1p-675 (FIG. 3) and was used for further cloning in a 2-micron yeast expression vector. The veracity of the clone was confirmed by restriction enzyme analysis and corroborated by DNA sequencing.

### Example 1.5

### Cloning of the 675 bp GAL1 Promoter NgoMIV-BamHI Fragment in a Yeast 2-Micron Expression Vector

A NgoMIV-BamHI GAL1 907 bp promoter fragment from pBluSK(+)/Ngo-Bam/Gal1p-675 (FIG. 3) was isolated and ligated to a 5190 bp fragment of pYES2 (Invitrogen; a commercial 2-micron vector that bears the URA3 auxotrophic marker) which had already been digested with NgoMIV-BamHI.

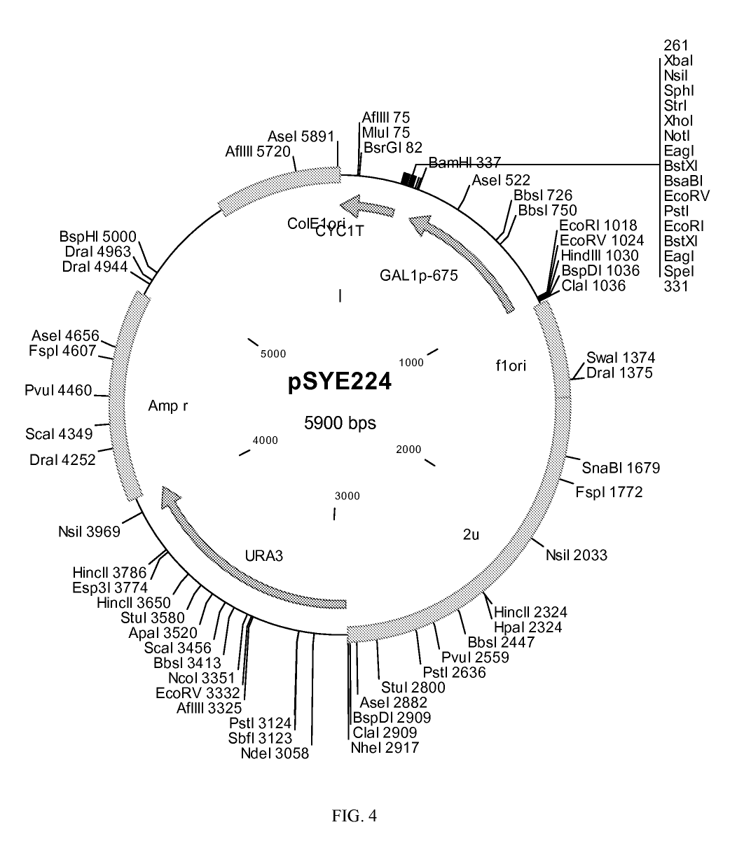

One correct clone obtained after ligation and transformation in DH5alpha bacterial cells was named pSYE224 (FIG. 4) and was used for expression of genes encoding cytochrome P450 isozymes (CYP-s). The veracity of the clone was confirmed by restriction enzyme analysis.

### Example 1.6

### Cloning of the 650 bp GAL1 Promoter as a NgoMIV-BamHI Fragment

The cloning of a NgoMIV-BamHI GAL1 promoter fragment (SEQ ID No. 3) in pBlueScriptII SK(+) was performed as in Example 1.2, using GAL1 sequence specific primers (5′ PCR primer: 5′-ATgccggc ACATGGCATT ACCACCATAT ACATATCCAT ATC-3′ (letters in lower casing represent the NgoMIV site—SEQ ID NO: 9) & 3′ PCR primer: 5′-ATggatcc CTAGAATTGA ACTCAGGTAC AATCACTTCT TCTG-3′ (letters in lower casing represent the BamHI site—SEQ ID NO: 10)). The amplified fragment, digested with NgoMIV-BamHI, was cloned in pBlueScriptII SK(+) digested with NgoMIV-BamHI.

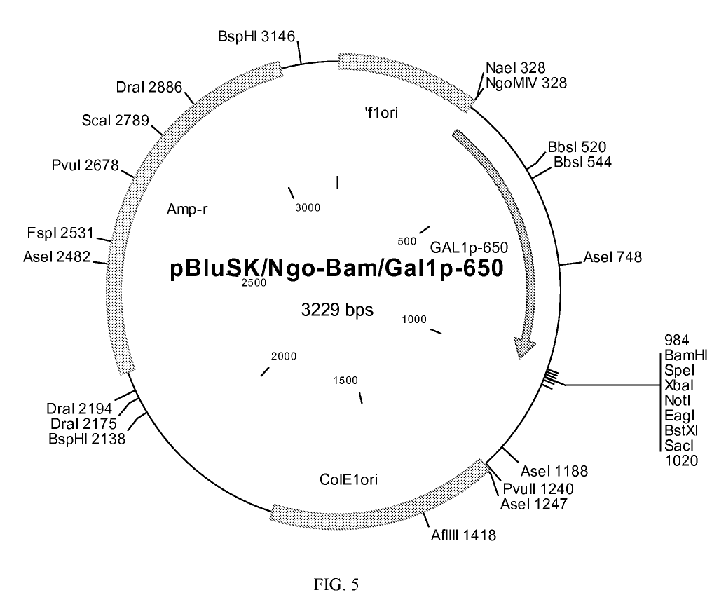

One correct clone obtained after ligation and transformation in DH5alpha bacterial cells was named pBluSK/Ngo-Bam/Gal1p-650 (FIG. 5) and was used for further cloning in a 2-micron yeast expression vector. The veracity of the clone was confirmed by restriction enzyme analysis and corroborated by DNA sequencing.

### Example 1.7

### Cloning of the NgoMIV-BamHI 650 bp GAL1 Promoter Fragment in a Yeast 2-Micron Expression Vector

A NgoMIV-BamHI GAL1 650 bp promoter fragment from pBluSK(+)/Ngo-Bam/Gal1p-650 (FIG. 5) was isolated and ligated to a 5190 bp fragment of pYES2 which had already been digested with NgoMIV-BamHI.

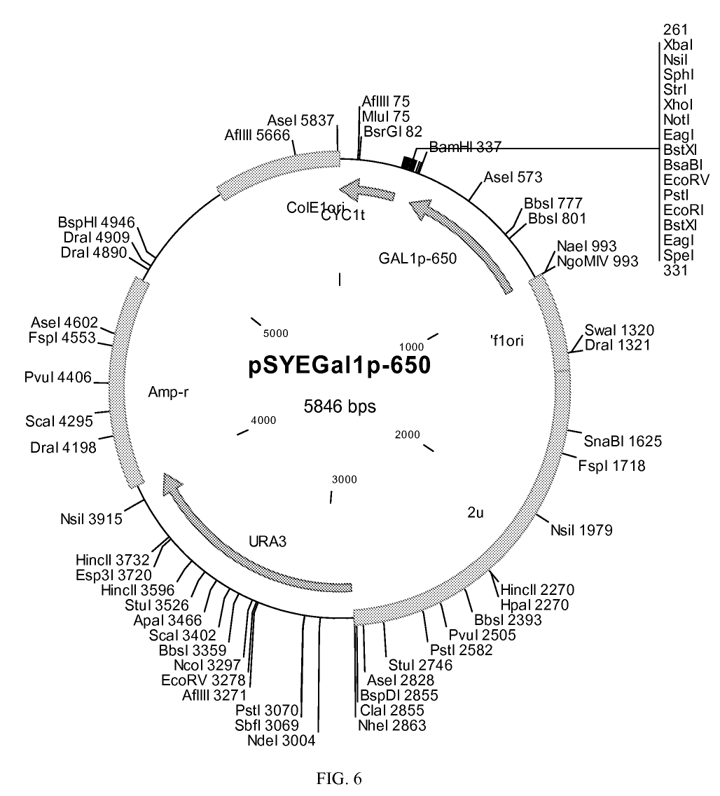

One correct clone obtained after ligation and transformation in DH5alpha bacterial cells was named pSYEGal1p-650 (FIG. 6) and was used for expression of genes encoding cytochrome P450 isozymes (CYP-s). The veracity of the clone was confirmed by restriction enzyme analysis.

### Example 1.8

### Cloning of the 461 bp GAL1 Promoter as a NgoMIV-BamHI Fragment

The cloning of a NgoMIV-BamHI GAL1 promoter fragment (SEQ ID No. 4) in pBlueScriptII SK(+) was performed as in Example 1.2, using GAL1 sequence specific primers (5′ PCR primer: 5′-ATgccggc ATTGAAGTAC GGATTAGAAG CCGCCGAGCG-3′ (letters in lower casing represent the NgoMIV site SEQ ID NO: 11) & 3′ PCR primer 5′-ATggatcc CCTCTATACT TTAACGTCAA GGAGAAAAAA CCCC-3′ (letters in lower casing represent the BamHI site—SEQ ID NO: 12)). The amplified fragment, digested with NgoMIV-BamHI, was cloned in pBlueScriptII SK(+) that was digested with NgoMIV-BamHI.

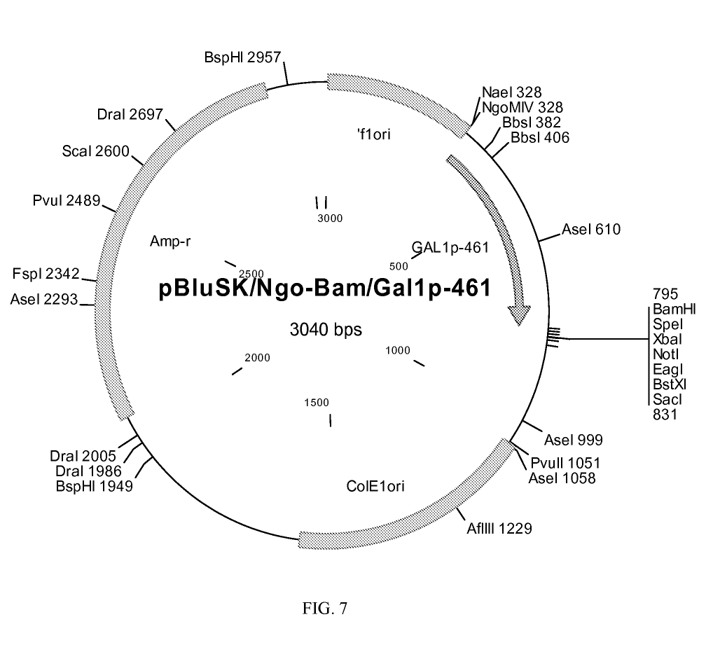

One correct clone obtained after ligation and transformation in DH5alpha bacterial cells was named pBluSK/Ngo-Bam/Gal1p-461 (FIG. 7) and was used for further cloning in a 2-micron yeast expression vector. The veracity of the clone was confirmed by restriction enzyme analysis and corroborated by DNA sequencing.

### Example 1.9

### Cloning of the NgoMIV-BamHI 461 bp GAL1 Promoter Fragment in a Yeast 2-Micron Expression Vector

A NgoMIV-BamHI GAL1 461 bp promoter fragment from pBluSK(+)/Ngo-Bam/Gal1p-461 (FIG. 7) was isolated and ligated to a 5190 bp NgoMIV-BamHI fragment of pYES2.

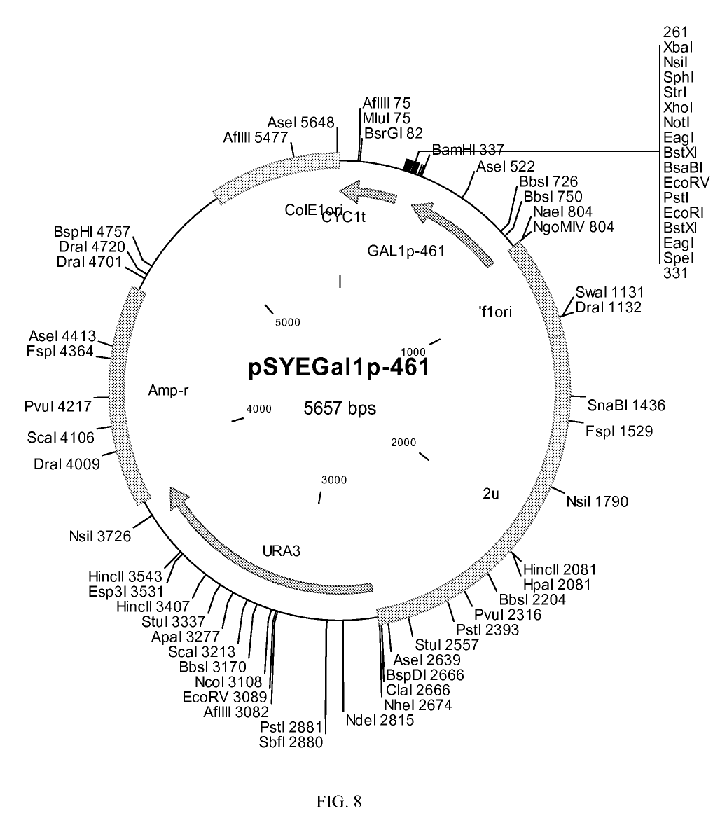

One correct clone obtained after ligation and transformation in DH5alpha bacterial cells was named pSYEGal1p-461 (FIG. 8) and was used for expression of genes encoding cytochrome P450 isozymes (CYP-s). The veracity of the clone was confirmed by restriction enzyme analysis. pSYEGal1p-461 should be the same as the commercial plasmid pYES2.

### Example 1.10

### Cloning of the HindIII-BamHI 269 bp CYCJ Core Promoter Fragment in a Yeast 2-Micron Expression Vector

A 269 bp HindIII-BamHI CYC core promoter (SEQ ID No. 13) PCR fragment was isolated from yeast genomic DNA (as in Example 1.2) using two sequence specific primers (5′ PCR primer: 5′-GCaagcttCA GATCCGCCAG GCGTGTATAT AGCG-3′ (letters in lower casing represent the HindIII site—SEQ ID NO: 14) & 3′ PCR primer: 5′-ATggatccAA TTCAGTCATT ATTAATTTAG TGTG-3′ (letters in lower casing represent the BamHI site—SEQ ID NO: 15)) and the restriction enzyme digested fragment was ligated to a 5839 bp HindIII-BamHI fragment of pYES2.

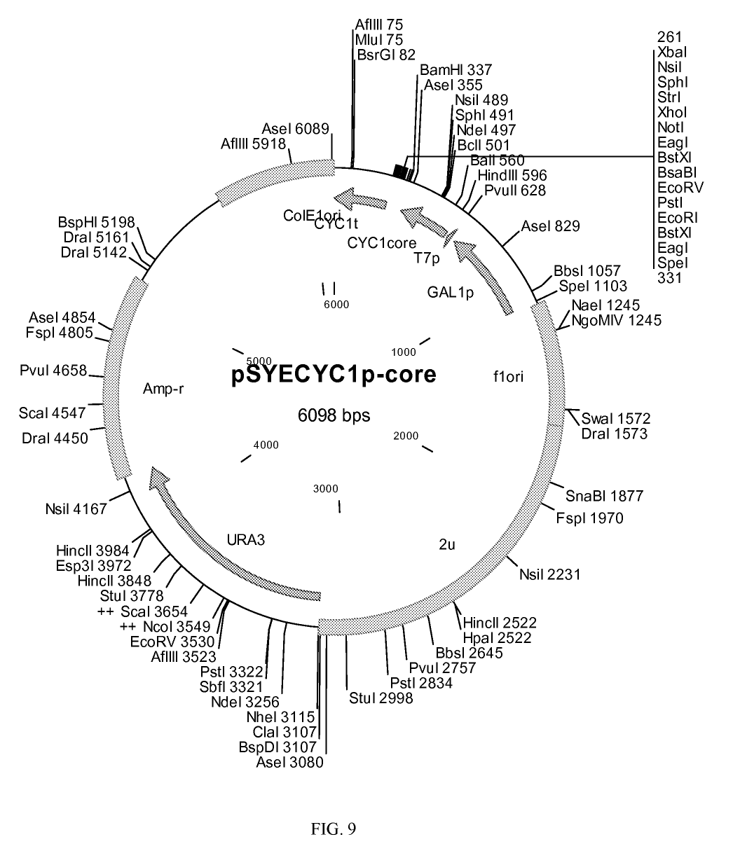

One correct clone obtained after ligation and transformation in DH5alpha bacterial cells was named pSYECYClp-core (FIG. 9). The presence of the insert was confirmed by restriction enzyme analysis and DNA sequencing. The plasmid was used for further introduction of upstream activation sequences (UAS-s), also known as enhancers, from the GAL1 and GAL10 promoters.

### Example 1.11

### Cloning of the NgoMIV-HindIII 385 bp GAL1UAS Promoter Fragment in a Yeast 2-Micron Vector that Already Contains the CYC1 Core Promoter

A NgoMIV-HinIII GAL1UAS 385 bp promoter fragment (SEQ ID No. 16) was isolated by PCR using pBluSK(+)/Ngo-Bam/Gal1p-675 (FIG. 3) as a template and two sequence specific primers (5′ PCR primer: 5′-GCGgccggcT CTTAGCCTAA AAAAACCTTC TC-3′ (letters in lower casing represent the NgoMIV site—SEQ ID NO: 17) & 3′ PCR primer: 5′-GCAAGCTTGA TCAAAAATCA TCGCTTCGCT G-3′ (letters in lower casing represent the HindIII site—SEQ ID NO: 18)) was isolated and ligated to a 5449 bp NgoMIV-HindIII fragment of the plasmid pSYECYC1p-core (FIG. 9).

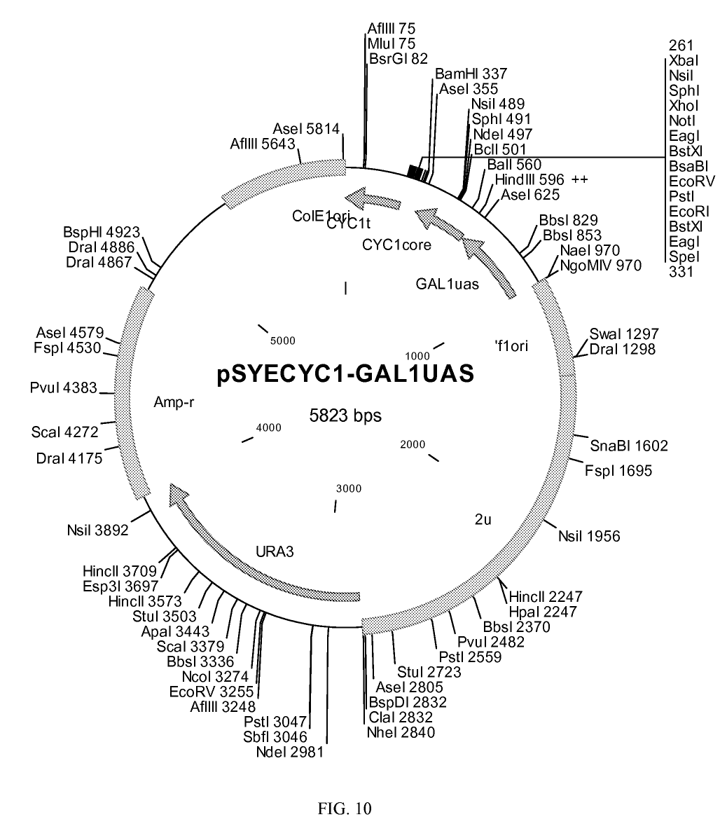

One correct clone obtained after ligation and transformation in DH5alpha bacterial cells was named pSYECYC1-GAL1UAS (FIG. 10) and was used for expression of genes encoding cytochrome P450 isozymes (CYP-s). The veracity of the clone was confirmed by restriction enzyme analysis.

### Example 1.12

### Cloning of the NgoMIV-HindIII 385 bp GAL10UAS Promoter Fragment in a Yeast 2-Micron Vector that Already Contains the CYC1 Core Promoter

A NgoMIV-HinIII GAL10UAS 385 bp promoter fragment (SEQ ID No.) was isolated by PCR using pBluSK(+)/Ngo-Bam/Gal1p-675 (FIG. 3) as a template and two sequence specific primers (5′ PCR primer: 5′-GCGgccggcG ATCAAAAATC ATCGCTTCGC TG-3′ (letters in lower casing represent the NgoMIV site—SEQ ID NO: 20) & 3′ PCR primer: 5′-GCAAGCTTTC TTAGCCTAAA AAAACCTTCT C-3′ (letters in lower casing represent the HindIII site—SEQ ID NO: 21)) was isolated and ligated to a 5449 bp NgoMIV-HindIII fragment of the plasmid pSYECYClp-core (FIG. 9).

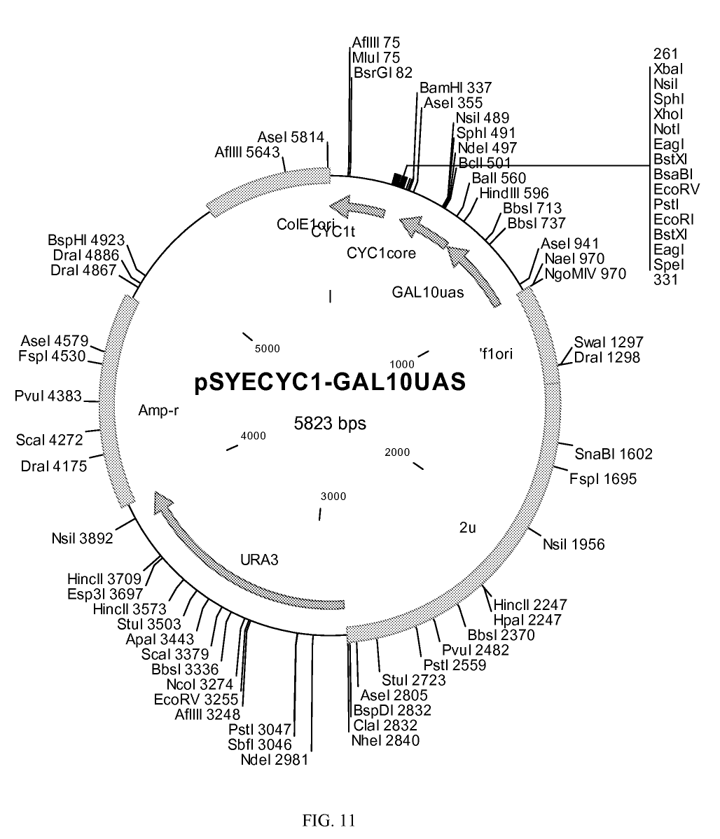

One correct clone obtained after ligation and transformation in DH5alpha bacterial cells was named pSYECYC1-GAL10UAS (FIG. 11) and was used for expression of genes encoding cytochrome P450 isozymes (CYP-s). The veracity of the clone was confirmed by restriction enzyme analysis.

### Example 1.13

### Amplification of the 1623 bp Modified CYP1B1 Gene and Cloning in a pBlueScript Vector

A BamHI-XbaI of the 1623 bp fragment of the modified CYP1B1 gene (SEQ ID No. 22), that lacks the sequence encoding amino acids 2-4 (GTS) of the wild type gene, was amplified by PCR using the sequence specific primers (5′ PCR primer: 5′ ATggatccAACAGATC ATGCTCAGCC CGAACGACCC TTGGCCGC 3′ (letters in lower casing represent the BamHI site whereas the italicised DNA indicates a 5′ consensus sequence for high expression in yeast—SEQ ID NO: 23) & 3′ PCR primer: 5′ GCtctagaTT ATTGGCAAGT TTCCTTGGCT TG 3′ (letters in lower casing represent the XbaI site—SEQ ID NO: 24)) using an adult human liver cDNA library (prepared in-house) as template.

The amplified fragment, digested with BamHI-XbaI, was cloned in pBlueScriptII KS(+) cut with BamHI-XbaI. The veracity of the clone was confirmed by restriction enzyme analysis and corroborated by DNA sequencing.

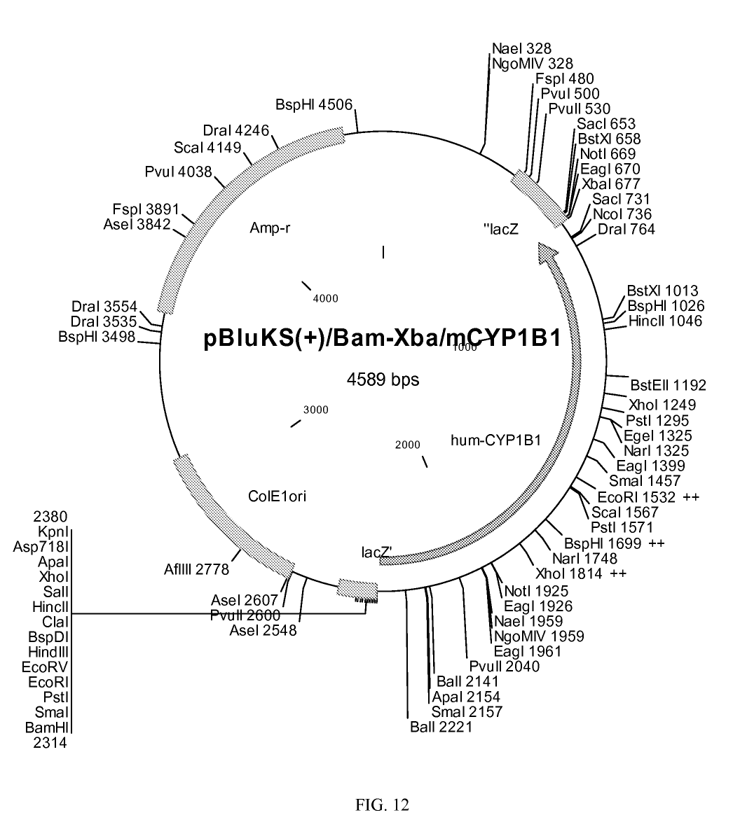

One correct clone obtained after ligation and transformation in DH5alpha bacterial cells was named pBluKS/Bam-Xba/mCYP1B1 (FIG. 12) and was used for further cloning in 2-micron yeast expression vectors that contain variant GAL1 promoters.

### Example 1.14

### Construction of Yeast Expression Plasmids that Contain CYP1B1 Expression Cassettes Driven by the Variant GAL1 Promoters, GAL1p-907, GAL1p-675, GAL1p-650, GAL1p-461

A BamHI-XbaI 1623 bp modified CYP1B1 gene fragment from pBluKS/Bam-Xba/mCYP1B1 (FIG. 12) was isolated and ligated to a

(i) 6028 bp BamHI-XbaI fragment of pSYEGal1p-907,
(ii) 5824 bp BamHI-XbaI fragment of pSYE224,
(iii) 5770 bp BamHI-XbaI fragment of pSYEGal1p-650,
(iv) 5581 bp BamHI-XbaI fragment of pSYEGal1p-461,
(v) 5747 bp BamHI-XbaI fragment of pSYECYC1-GAL1UAS,
(vi) 5747 bp BamHI-XbaI fragment of pSYECYC1-GAL10UAS.

The parent plasmids were all digested with BamHI-XbaI.

A correct clone obtained from each ligation and transformation in DH5 alpha bacterial cells were named

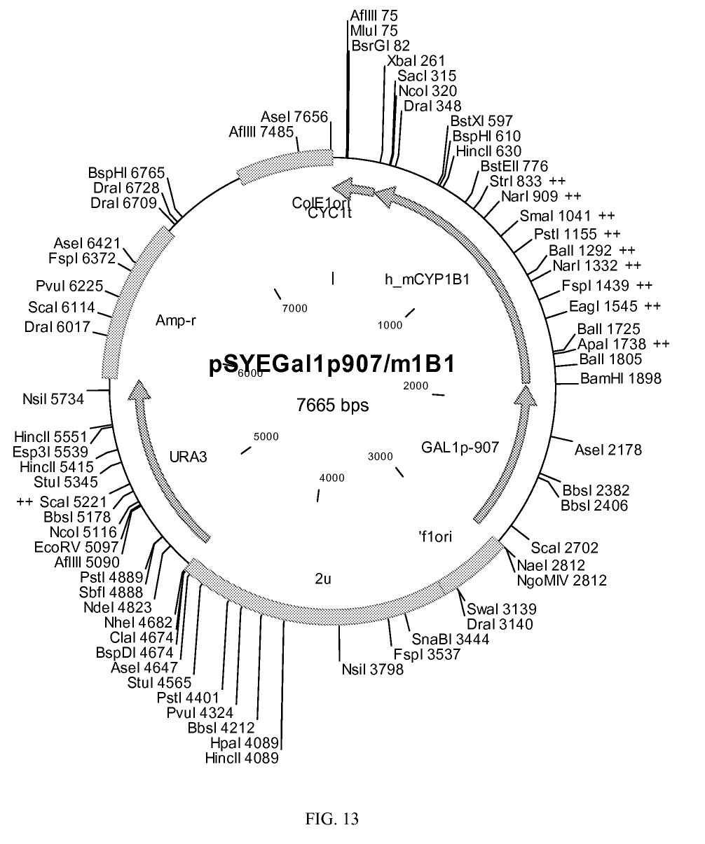

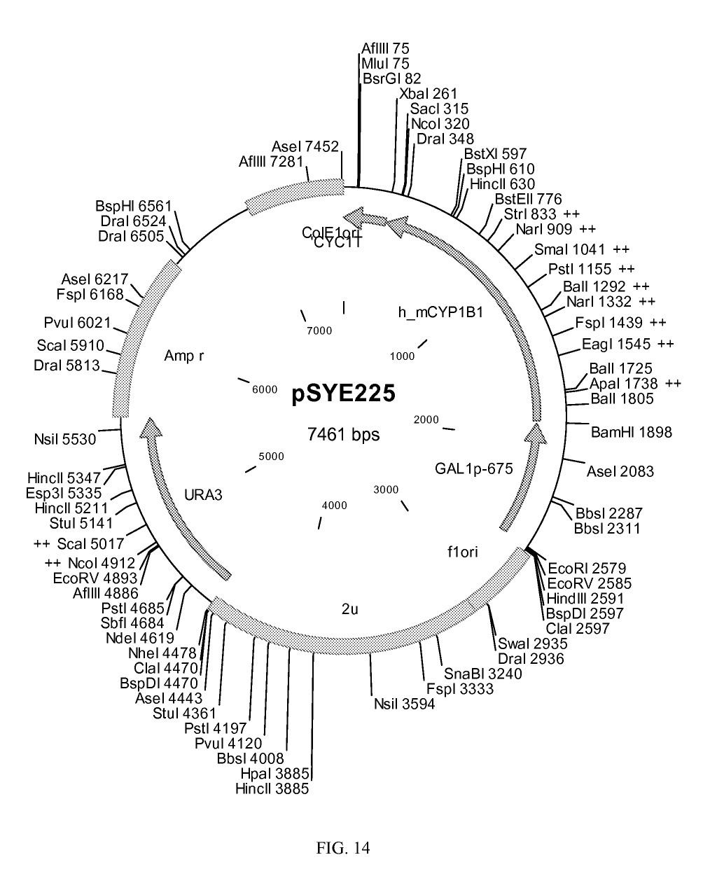

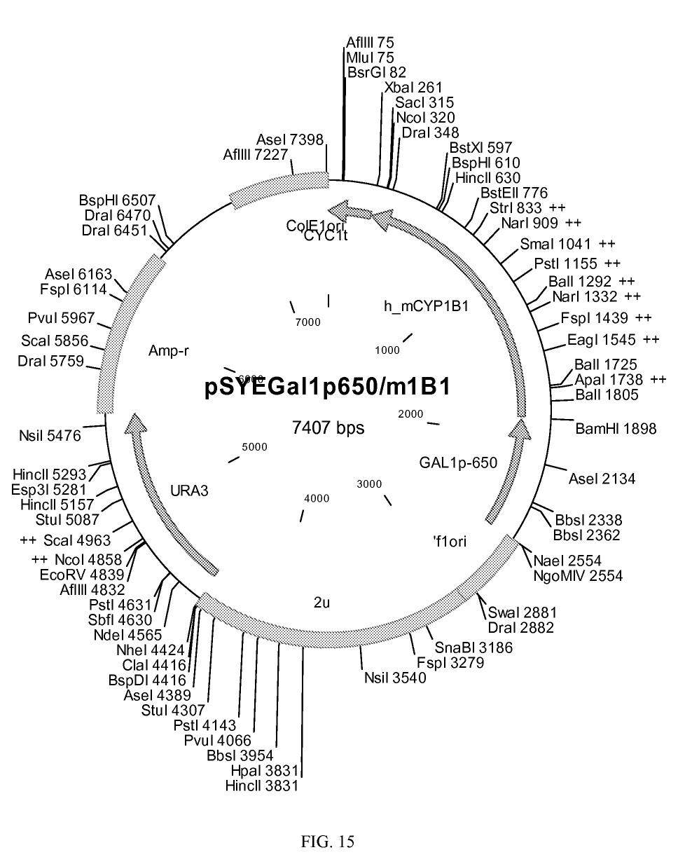

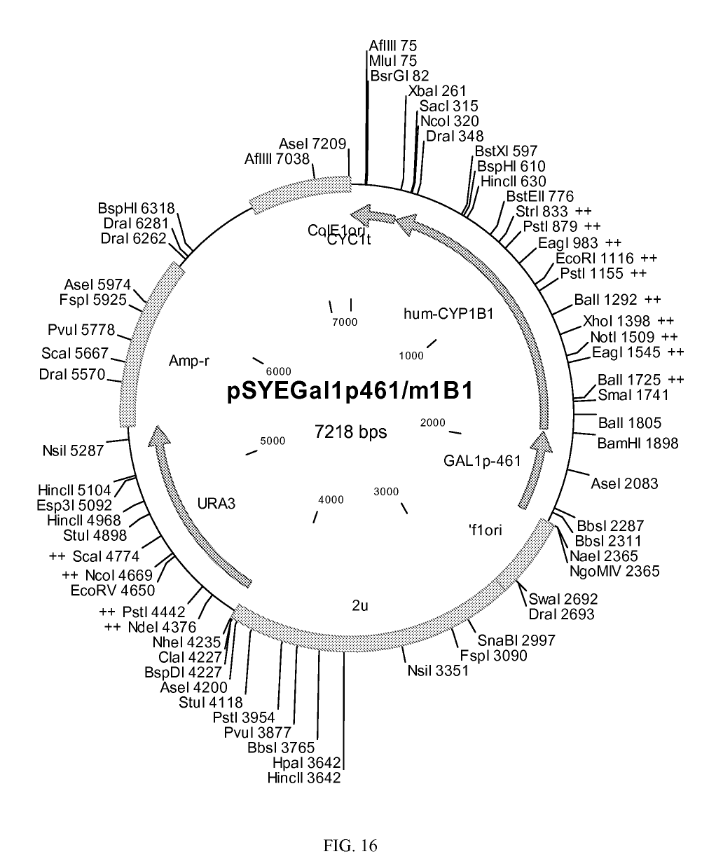

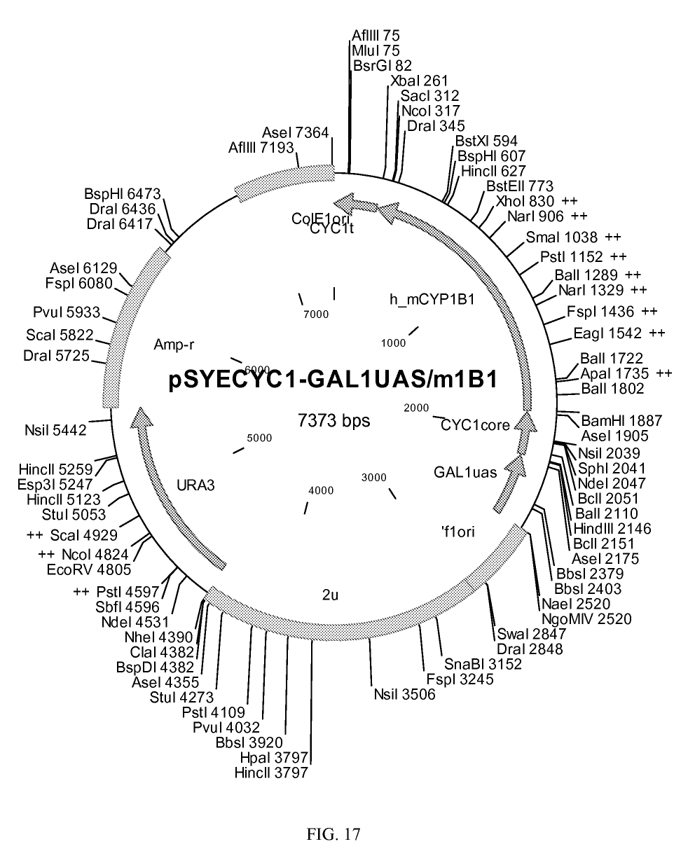

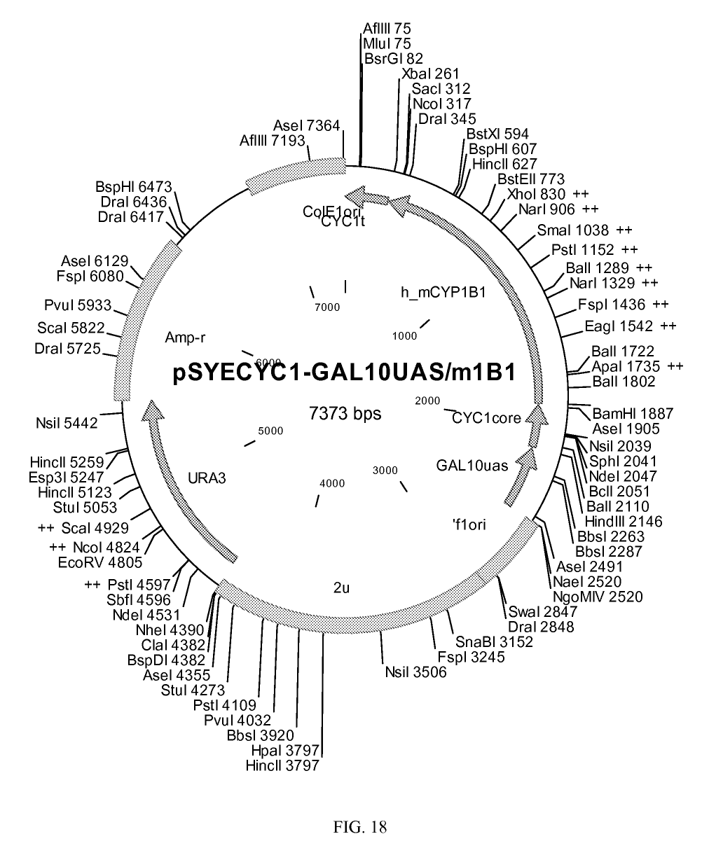

(i) pSYEGal1p907/m1B1 (FIG. 13),
(ii) pSYE225 (FIG. 14),
(iii) pSYEGal1p650/m1B1 (FIG. 15),
(iv) pSYEGal1p461/m1B1 (FIG. 16),
(v) pSYECYC1-GAL1UAS/m1B1 (FIG. 17),
(vi) pSYECYC1-GAL10UAS (FIG. 18).

The veracity of the clones were confirmed by restriction enzyme analyses.

### Example 1.15

### Expression of Human CYP1B1 from the Variant GAL1 Promoters, GAL1p-907, GAL1p-675, GAL1p-650, GAL1p-461 in Baker's Yeast

The Saccharomyces cerevisiae strains

(a) JL20 (MAT a, leu2-3, 2-112, his4-519, ade1-100, ura3-52), and
(b) W303B (MAT a leu2 his3 trp 1 canl-100 ade2 trp1 ura3) were used for transformation of the human CYP1B1 bearing plasmids
(i) pSYEGal1p907/m1B1 (FIG. 13),
(ii) pSYE225 (FIG. 14),
(iii) pSYEGal1p650/m1B1 (FIG. 15),
(iv) pSYEGal1p461/m1B1 (FIG. 16),
(v) pSYECYC1-GAL1UAS/m1B1 (FIG. 17),
(vi) pSYECYC1-GAL1UAS/m1B1 (FIG. 18).

Yeast Transformation: A single colony of the parent strains, JL20 and W303B, was picked up from a minimal medium (SD) plate (supplemented with appropriate nutrients depending on the auxotrophic markers in the yeast strain) and inoculated into 10 ml of YPD medium (2% Bacto Peptone, 1% yeast extract, 2% glucose). The cells were grown overnight at 30° C. with 220 rpm shaking. 1.5 ml of overnight culture was centrifuged at 13,000 rpm for a few seconds to collect the cell pellets. 0.5-2 μg of transforming DNA (i.e. the CYP1B1 bearing expression plasmids as in Example 1.11) and 100 μg of single-stranded salmon sperm DNA were added to pellets and vortexed briefly. 500 μl of PEG solution (40% PEG 3350, 0.1M lithium acetate pH 7.5, 10 mM Tris-HCl pH 7.5, 1 mM EDTA pH7.5) and 5-10% DMSO were added to transformation mixes. All mixes were incubated in a Thermo-mixer for 15 min at 25° C. with shaking at 400 rpm, and then were heat shocked for 15 min at 42° C. After 10 min, 5-10% ethanol was added. The cells were pelleted at 8000 rpm for 1 min and were washed twice in 1×TE buffer and re-suspended in 250 μl-500 μl 1×TE pH7.5. The cells were plated out on SD agar medium and incubated at 30° C. for 2-3 days.

The transformants obtained are depicted in Table 1.

TABLE 1

| Parent | | |
|---|---|---|
| Strain | Plasmid | Transformant |
| JL20 | pSYEGal1p907/m1B1 | JL20: GAL907-m1B1 |
| JL20 | pSYE225 | JL20: GAL675-m1B1 |
| JL20 | pSYEGal1p650/m1B1 | JL20: GAL650-m1B1 |
| JL20 | pSYEGal1p461/m1B1 | JL20: GAL461-m1B1 |
| JL20 | pSYECYC1-GAL1UAS/m1B1 | JL20: GAL1UAS-m1B1 |
| JL20 | pSYECYC1-GAL10UAS/m1B1 | JL20: GAL10UAS-m1B1 |
| W303B | pSYEGal1p907/m1B1 | W303B: GAL907-m1B1 |
| W303B | pSYE225 | W303B: GAL675-m1B1 |
| W303B | pSYEGal1p650/m1B1 | W303B: GAL650-m1B1 |
| W303B | pSYEGal1p461/m1B1 | W303B: GAL461-m1B1 |
| W303B | pSYECYC1-GAL1UAS/m1B1 | W303B: GAL1UAS-m1B1 |
| W303B | pSYECYC1-GAL10UAS/m1B1 | W303B: GAL10UAS-m1B1 |

Yeast cultures for microsome preparation: The growth of yeast cultures for microsome preparation is a five-day experiment.

(1) On day one, a loopful of fresh yeast cells from an SD-agar plate was inoculated in SD media containing required nutrients and Casamino Acid (Sigma, C-7585). The cultures were grown overnight at 30° C. with shaking at 220 rpm.
(2) On day two, once OD₆₀₀ (i.e. OD measured at 600 nm) of the cultures reached 4 to 8 OD-s, the cultures were inoculated into 100 ml YPGE medim (1% Bacto Peptone, 1% yeast extract, 2% glycerol, and 2% ethanol) with appropriate nutrients in 500 ml flasks. The inoculum volumes used from overnight cultures, having specific OD₆₀₀ ranges, are shown on Table 2. The cultures were grown for around 14 hours at 30° C. with shaking at 220 rpm.
(3) On day three, once the OD₆₀₀ reached 3-5, 10 ml of 20% (w/v) filter-sterilised galactose solution was added into each culture together with half the amount of required nutrients. The cultures were grown for 14 hours at 30° C. with shaking at 220 rpm.
(4) On day four, the OD₆₀₀ should reach 170D-s. The final OD₆₀₀ is very important for the whole experiment, since it can be a scale for calculating the amount of lyticase used in microsome preparation. The cultures were transferred into plastic bucket and spun down at 2831 g for 12 min. The supernatants were discarded carefully, and the pellets were washed twice with buffer A (10 mM Tris•Cl pH7.5, 0.65 M Sorbitol, 1 mM EDTA pH8.0). After the second wash, the supernatants were discarded as soon as possible, and the buckets were weighed together with the pellets. The pellet weights are usually between 3.5 and 4.5 g. The pellets can be kept at −80° C. for a length of time before beginning the microsome preparations.

TABLE 2
Inoculum volume for OD₆₀₀ ranges

| OD₆₀₀ ranges | Inoculum volume (ml) |
|---|---|
| 4.5-5.5 | 1.5 |
| 6-6.7 | 1.2 |
| 7 | 0.8 |

Microsome Preparation: The pellets obtained from growth of yeast cultures were re-suspended in 10 ml buffer B (10 mM Tris Cl pH7.5, 2 M Sorbitol, 0.1 mM EDTA pH8.0, 1 mM Pefabloc, 0.1 mM DTT), and transferred into 50 ml conical flasks, then incubated at 30° C. for 15 min with shaking at 220 rpm. Lyticase (ICN cat. No. 152270) was added to cell suspensions at 7.5 U/OD₆₀₀ (amount of lyticase in mg=(final OD₆₀₀×total volume of cells culture×7.5 U)/81.6). The cell suspensions were incubated for 1 hour at 30° C. with 110 rpm shaking. OD₆₀₀ was measured every 20 min. There should be at least a 2.5 fold drop in OD₆₀₀ during the 20-minute time intervals. The following steps were all performed in the cold room or in a refrigerated centrifuge. Cells were centrifuged at 3500 rpm for 10 min at 4° C. The supernatant was discarded, the spheroplast pellets were re-suspended in buffer A with 0.1 mM DTT, 1 mM Pefabloc, and the pellets were dislodged with a homogenizer (Fisher). The homogenized pellets were sonicated 8×10 seconds with 3 min intervals on ice at 30% of total power. Cells were spun at 3500 rpm at 4° C. for 10 min. Supernatants were centrifuged in JA17 Beckman centrifuge at 10,000 rpm twice at 4° C. for 10 min. Afterwards, the supernatants were centrifuged in Ti50 ultracentrifuge (Optima L-100 XP ultracentrifuge, Beckman Coulter) at 45,000 rpm at 4° C. for 90 min. Pellets were washed and re-suspended in buffer C (10 mM Tris•Cl pH7.5, 1 mM EDTA pH8.0, 20% Glycerol, 0.2 mM Pefabloc). Microsome aliquots were snap-frozen in liquid nitrogen and stored—80° C. for obtaining the CO difference spectra, and performing the reductase and/or the EROD assays.

Determination of P450 amounts via CO-difference spectra: Difference spectra of microsomal preparations were measured in a spectrophotometer (Lambda 16, Perkin Elmer) using plastic disposable cuvettes. 850 μl of a solution containing 100 mM potassium phosphate and 20% glycerol (pH7.5) was added to the cuvette, and left for one minute. Then a ‘few grains’ of sodium hydrosulfite was added, mixed gently to prevent any bubble forming in the cuvette and left for another minute. 150 μl of mirosomes were added into the cuvette and the whole suspension was mixed gently. Two cuvettes (one containing sodium hydrosulphite without microsome and the other with microsome) were prepared and a baseline of light absorption of the buffer and microsome mixture was recorded in the dual-beam spectrophotometer from 400 nm to 500 nm. Carbon monoxide was bubbled slowly into one sample cuvette for about one minute, 1 bubble/second. Light absorption was recorded again from 400 nm to 500 nm. The concentration of cytochrome P450 in the cuvette was calculated from the absorption change at 450 nm relative to the absorbance change at 490 nm, using the formula below:

P450 content (nmole/ml)=(A₄₅₀-A₄₉₀)×df×1000/extinction coefficient 450 nm
P450 concentration (nmole/mg protein)=P450 content/total protein
df=dilution factor (total volume in cuvutte/volume microsome)
Extinction Coefficient 420 nm=110 mM-1 cm⁻¹
Extinction Coefficient 450 nm=91 mM-1 cm⁻¹

Extinction coefficient is the fraction of light lost to scattering and absorption per unit distance in a participating medium. It is the sum of absorption coefficient and scattering coefficient.

### Results:

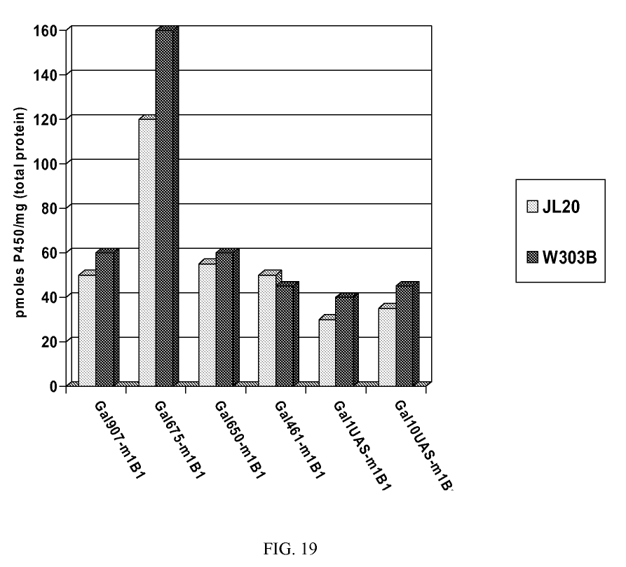

The comparative analysis is depicted in FIG. 19.

Conclusion: Expression of CYP1B1 from the 675 bp GAL1p is the best. The results obtained are an average of 3 observations. This promoter can be used to control the expression of nucleotide sequences, including sequences encoding cytochrome P450 isozymes.

### Example 2

### Construction of Yeast Integrating Plasmids that Bear Variants of the Human P450 Reductase (hRD) Gene Under the Control of the 675 bp GAL1-675 Promoter

Two different variants of the hRD gene have been expressed to obtain hRD activity that may not be deleterious for P450 expression. The aim was to devise an optimal system that allows better production of human P450 isozymes in yeast. The ultimate goal was to find an alternative system for the production of recombinant human P450 isozymes not only in yeast but also in insect and mammalian cells.

(1) The first variant lacks only the charged NH₂(N)-terminal 24 amino acids. In the wild type human reductase, the hydrophobic membrane anchor is constituted by amino acids 25-44.
(2) The second variant lacks the negatively charged (5 negatively charged amino acids+a potential positively charged amino acid) N-terminal 24 amino acids and the COOH-terminal Stop codon but contains the c-myc epitope tag EQKLISEEDLNG at the COOH-terminal end. The 12 amino acid c-myc tag is also a negatively charged peptide (containing 4 negatively charged amino acids and a positively charged amino acid) and is linked to the C-terminus of hRD through the linker, SS (coded for by TCTAGT formed through the ligation of the restriction sites SpeI ligated and XbaI). The c-myc tag also would allow monitoring of P450 reductase protein production inside the cell. The DNA for the c-myc tag was chemically synthesised using yeast-biased codons.
(3) In order to clone the hRD variants and the full-length hRD gene, yeast integrating vectors that would allow expression of hRD under the control of the GAL1-675 promoter from different chromosomal loci of yeast strains was first constructed. The steps involved were:
(1) Cloning of a GAL1-675 promoter with a downstream c-myc-tagged yeast SUC2 terminator in different pBlueScript-based vectors, pRS305, pRS402, pRS403, pRS404 (obtained from ATCC), that already bear the LEU2, ADE2, HIS3 and TRP1 genes, respectively.
(2) Cloning of the hRD variants from a human liver cDNA library in pBlueScript vectors and confirming the inserts via restriction enzyme analysis and DNA sequencing.
(3) Sub-cloning of the hRD variants in yeast integrating vectors, derived from pRS305, pRS402, pRS403 and pRS404, that contain the GALIp-675-cmycSUCt (GAL1-675 promoter+c-myc tagged SUC2t) cassette. The hRD gene variants would be cloned downstream of the GAL1-675 promoter and upstream of the c-myc tagged SUC2 terminator.

### Example 2.1

### A Protein Sequence Comparison of Wild Type P450 Reductase (SEQ ID NO: 25 and the Two Variants, delNhRD (ΔN24hRD) (SEQ ID NO: 26) and delNhRD-cmyc (ΔN24hRD-cmyc) (SEQ ID NO: 27) is Provided Below

Sequences which are identical are highlighted in yellow. The charged N-terminal domain (belonging to hRD) and C-terminal (c-myc) peptide are highlighted in blue. The 24-amino acid N-terminal domain (ΔN24) contains 5 negatively charged amino acids (D or E) and a histidine (H) that has the capability of becoming positively charged at pH<7. In contrast, the c-myc tag contains 4 negatively charged amino acids (D or E) and a positively charged amino acid (K) over a concentrated region of 12 amino acids.

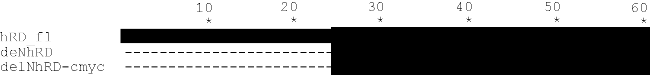

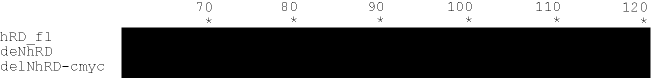

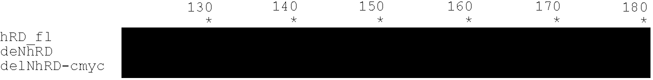

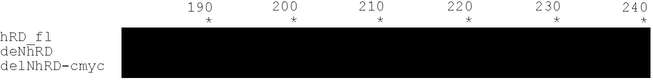

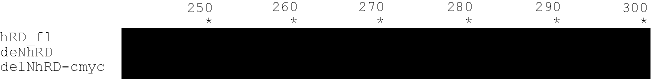

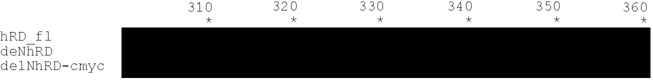

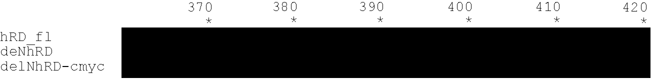

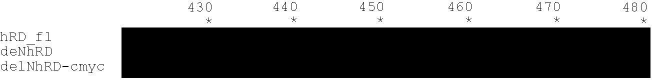

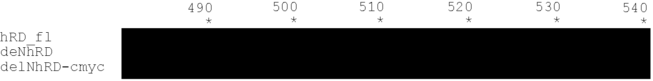

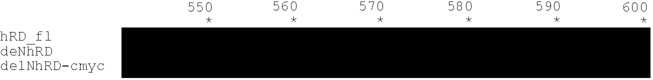

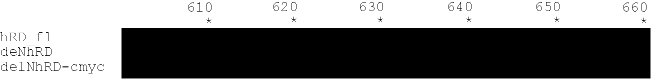

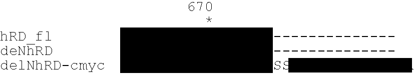

### Example 2.2

### Cloning an XbaI-SacI Fragment of the Terminator from the Yeast SUC2 Gene

The cloning was done in three steps (as in Example 1). The first step involved PCR that was used to amplify the 291 bp terminator from the yeast SUC2 gene (SEQ ID No. 26) as a 303 bp XbaI-SacI fragment. 100 pmoles each of the PCR primers (5′ PCR primer: 5′-ATtctagaAGGTTATAAAACTTATTGTCTT-3′ (letters in lower casing represent the XbaI site—SEQ ID NO: 29) & 3′ PCR primer: 5′-ATgagctcGGTCCATCCTAGTAGTGTAAGGC-3′ (letters in lower casing represent the SacI site—SEQ ID NO: 30)) were used with genomic DNA (50 ng), from the Saccharomyces cerevisiae strain S288C (Invitrogen), as template. The 303 bp PCR product was purified on a QiaQuick column (Qiagen UK).

In the second step, the purified DNA was digested with the restriction enzymes XbaI and SacI (both obtained from New England BioLabs, UK) and ˜100 ng of the restricted DNA (the insert) was ligated with 50 ng of pBlueScriptII KS(+) (Stratagene, UK), the vector, which had been already digested with XbaI and SacI. Ligation was performed between the vector and the insert using DNA ligase (Roche Diagnostics, UK) in 15 μl of reaction mixture, following the supplier's protocol.

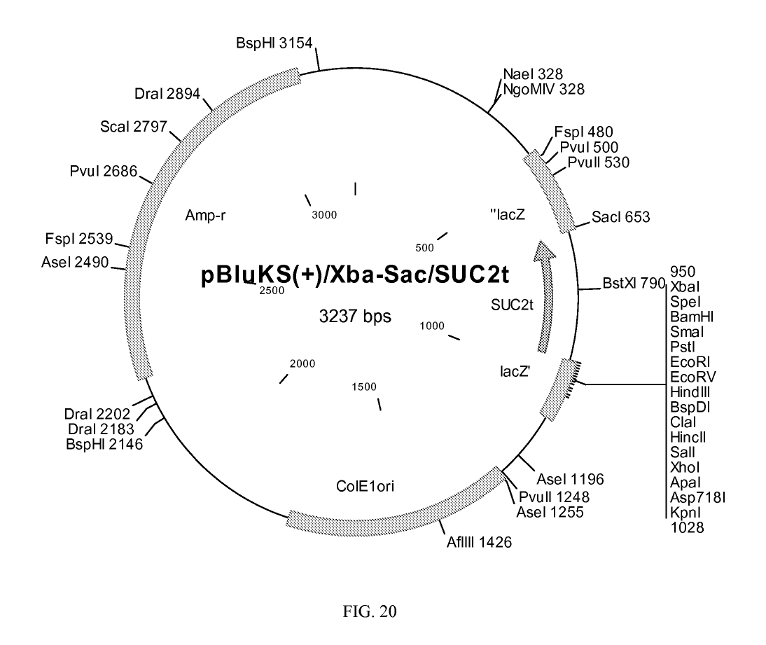

In the final step, 3 μl of the ligation mixture was transformed into DH5alpha competent cells (see Example 1.1). DNA from six individual clones was isolated and the veracity of the clones was confirmed by restriction enzyme analysis and further corroborated by DNA sequencing. One correct clone was named pBluKS(+)/Xba-Sac/SUC2t (FIG. 20) and was used for further experiments.

### Example 2.3

### Unidirectional Cloning of a Fragment that Encodes c-myc Tag

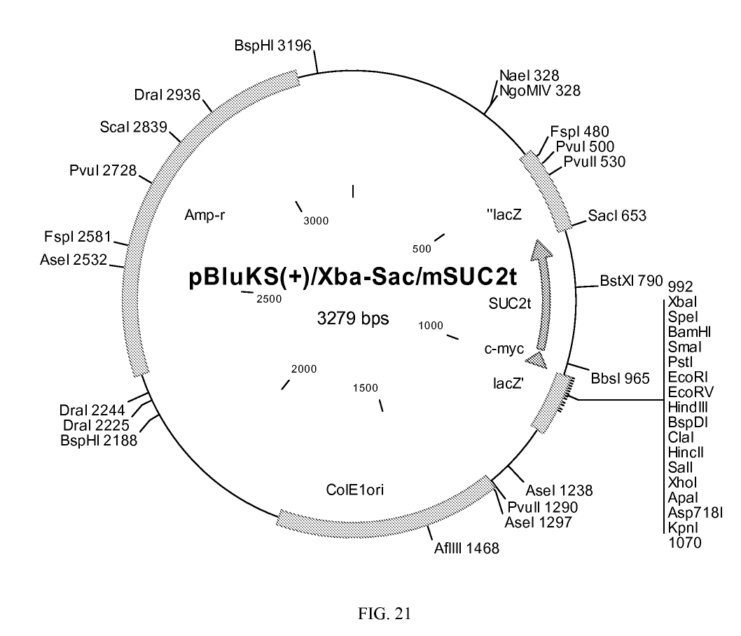

pBluKS(+)/Xba-Sac/SUC2t (see Example 2.2) was cut with XbaI and the linearised DNA was dephosphorylated with bacterial alkaline phosphatase (New England BioLabs). After Proteinase K treatment, the 3237 bp vector fragment was partitioned on an agarose gel and the isolated gel fragment was purified on a QiaQuick column to obtain the vector. The vector was ligated with a pair of deoxyoligonucleotides (SEQ ID Nos 31 & 32) that encode the 12-amino acid c-myc tag and has a XbaI overhang at the 5′-end and a SpeI overhang at the 3′-end. The sequences were chemically synthesised using yeast-biased codons for the c-myc peptide tag (EQKLISEEDLNG), a peptide derived from the c-myc oncogene and often used to tag recombinant proteins. The 5′-end of the sequence re-creates a XbaI site whereas the 3′-end, contiguous to the SUC2 terminator (SUC2t) cannot be re-opened either by XbaI or SpeI and contains a 3′-end STOP codon before the 5-end of SUC2t. One correct clone obtained after ligation and transformation in DH5alpha bacterial cells was named pBluKS(+)/Xba-Sac/mSUC2t (FIG. 21). “m” representing the c-myc tag. The veracity of the clone was confirmed by restriction enzyme analysis and corroborated by DNA sequencing.

### Example 2.4

### Cloning of the GAL1-675 Promoter as a XhoI-BamHI Fragment

The cloning was done in three steps.

The first step involved PCR that was used to amplify the GAL1 promoter (SEQ ID No. 2) (see Example 1) as an XhoI-BamHI fragment. 100 pmoles each of the PCR primers (5′ PCR primer: 5′-TCctcgagCTTGAATTTTCAAAAATTCTTACTTTTTTTTTGG-3′ (letters in lower casing represent the XhoI site—SEQ ID NO: 33) and the primer of SEQ ID NO: 8) were used with BluSK/Eco-Bam/Gal1p (50 ng), as template.

The 675 bp PCR product was purified on a QiaQuick column (Qiagen UK).

In the second step, the purified DNA was digested with the restriction enzymes XhoI and BamHI and ˜100 ng of the restricted DNA (the insert) was ligated to 50 ng of 3228 bp pBluKS(+)/Xba-Sac/mSUC2t (see Example 2.3; FIG. 21), the vector which had been digested with XhoI and BamHI. Ligation was performed between the vector and the insert using DNA ligase.

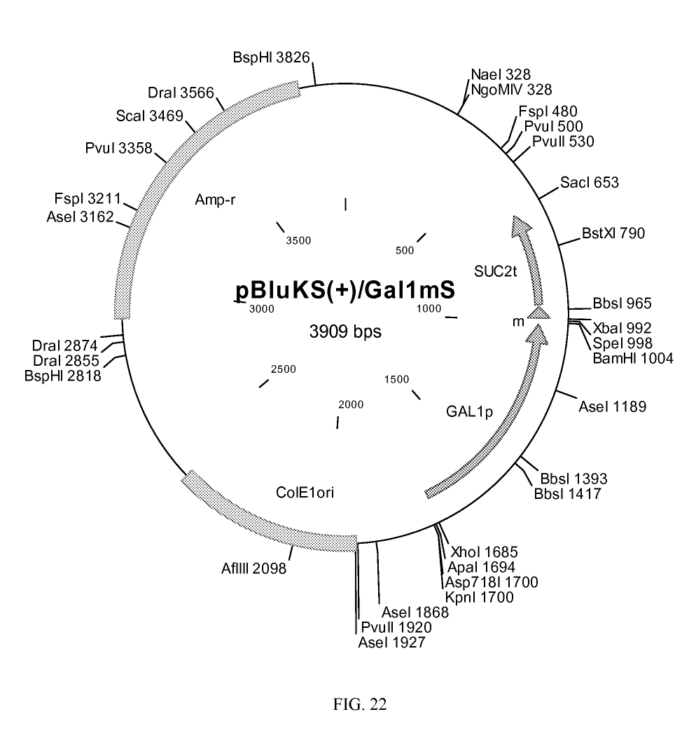

In the final step, 3 μl of the ligation mixture was transformed into E. coli DH5alpha competent cells. DNA from six individual clones was isolated and the veracity of the clones was confirmed by restriction enzyme analysis and further corroborated by DNA sequencing. One correct clone was named pBluKS(+)/Gal1mS (FIG. 22) and was used for further experiments. This pBluescript based plasmid contains a promoter and terminator cassette (i.e. Gal1mS). It also contains suitable restriction sites between the promoter and terminator (i.e. BamHI, SpeI, XbaI) that would allow cloning of any gene of interest (i.e. hRD variants, etc).

### Example 2.5

### Cloning of the Gal1mS Cassette into pRS305, a LEU2 Integrating Plasmid

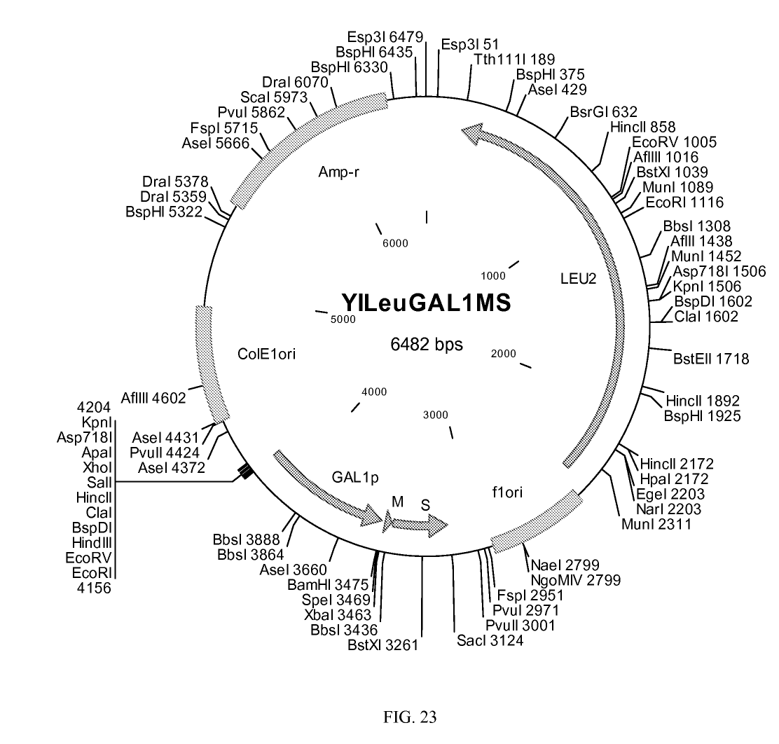

Plasmid pRS305 (ATCC; that bears the Saccharomyces cerevisiae LEU2 gene as selection marker for growth of yeast cells in minimal media that lacks leucine) was digested with XhoI and SacI and the large 5421 bp fragment was isolated. The isolated vector was ligated to the XhoI-SacI promoter-terminator Gal1mS cassette (1028 bp) isolated from pBluKS(+)/Gal1mS (FIG. 22). One correct clone, obtained after ligation and transformation in DH5alpha bacterial cells, was designated as YILeuGAL1MS (FIG. 23). The veracity of the clone was confirmed by restriction enzyme analysis. The plasmid YILeuGAL1MS contains a promoter and terminator cassette (i.e. Gal1mS) on a yeast integrating plasmid. It also contains suitable unique restriction sites between the promoter and terminator (i.e. BamHI, SpeI, XbaI) that would allow cloning of a gene of choice and integration of the plasmid into the yeast genome at the LEU2 locus of any gene of interest.

### Example 2.6

### Cloning of the Gal1mS Cassette into pRS402, an ADE2 Integrating Plasmid

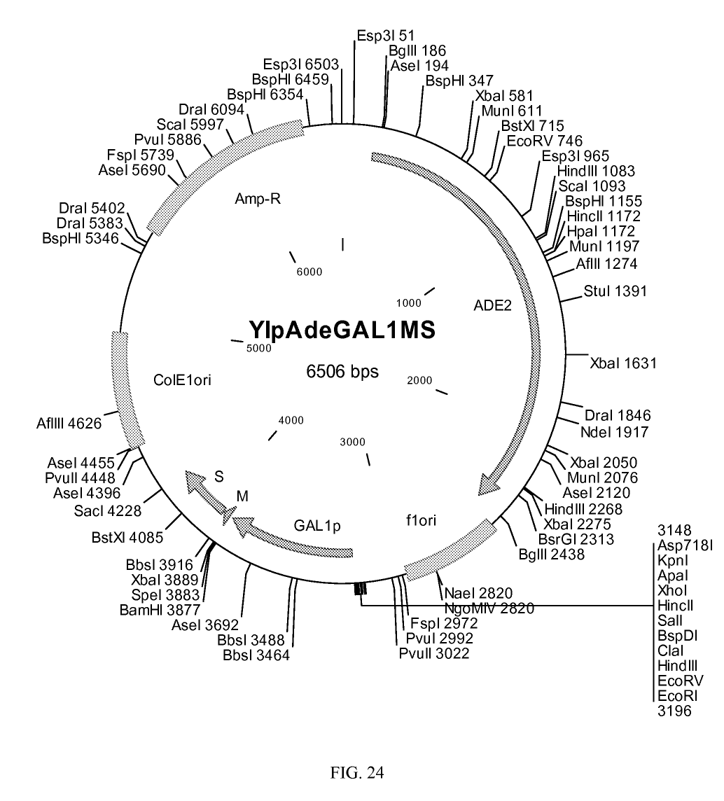

Protocols similar to that in Example 2.5 were followed to obtain the yeast integrating plasmid YIpAdeGAL1MS (FIG. 24) from plasmid pRS402 (ATCC) that bears the Saccharomyces cerevisiae (baker's yeast) ADE2 gene as selection marker for growth of yeast cells in minimal media. The plasmid contains suitable unique restriction sites between the promoter and terminator (i.e. BamHI, SpeI) that would allow cloning and integration into the yeast genome at the ADE2 locus of any gene of interest.

### Example 2.7

### Cloning of the Gal1mS Cassette into pRS403, a HIS3 Integrating Plasmid

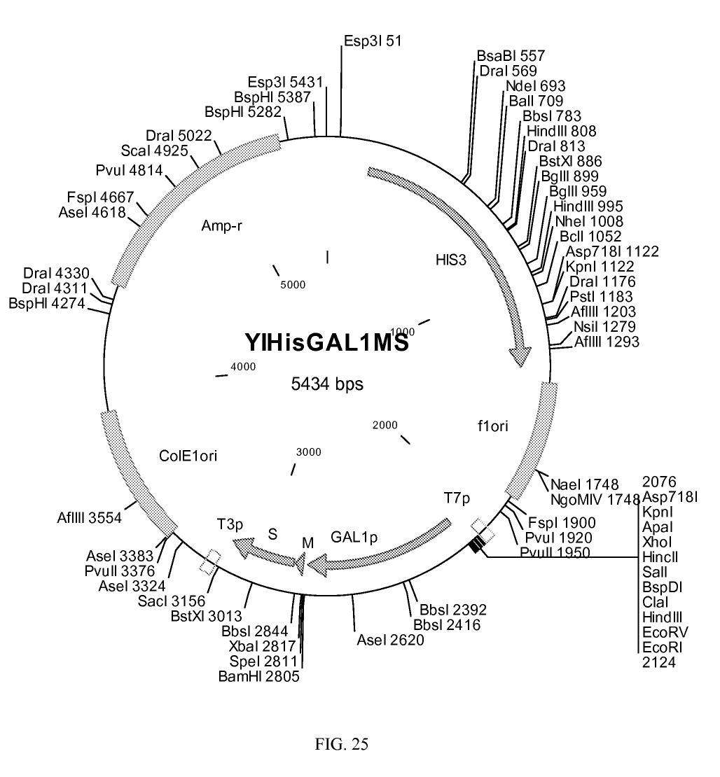

Protocols similar to that in Example 2.5 were followed to obtain the yeast integrating plasmid YIHisGAL1MS (FIG. 25) from plasmid pRS403 (ATCC) that bears the S. cerevisiae HIS3 gene as selection marker for growth of yeast cells in minimal media. The plasmid contains suitable unique restriction sites between the promoter and terminator (i.e. BamHI, SpeI, XbaI) that would allow cloning and integration into the yeast genome at the HIS3 locus of any gene of interest.

### Example 2.8

### Cloning of the Gal1mS Cassette into pRS404, a TRP1 Integrating Plasmid

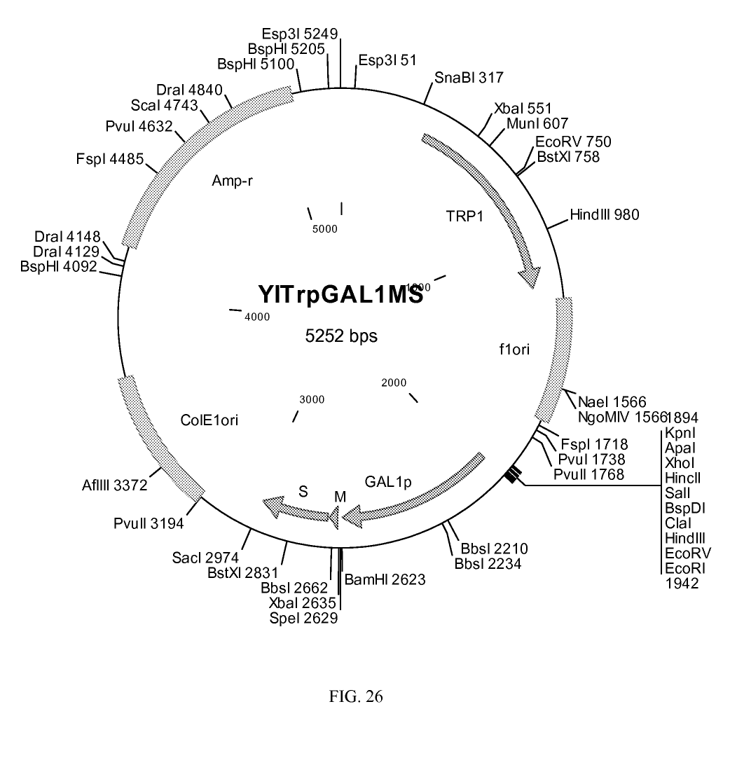

Protocols similar to that in Example 2.5 were followed to obtain the yeast integrating plasmid YITrpGAL1MS (FIG. 26) from plasmid pRS404 (ATCC) that bears the S. cerevisiae TRP1 gene as selection marker for growth of yeast cells in minimal media. The plasmid contains suitable unique restriction sites between the promoter and terminator (i.e. BamHI, SpeI, XbaI) that would allow cloning and integration into the yeast genome at the TRP1 locus of any gene of interest.

### Example 2.9

### Cloning of the ΔN24hRD Gene, with a Stop Codon, from a Liver cDNA Library in the Basic Plasmid pBluescript

The 1974 bp BamHI-XbaI fragment (ΔN24hRD) of the N-terminally truncated human P450 reductase gene with a 3′-end Stop codon, (SEQ ID No. 34), was amplified as a BamHI-XbaI fragment. 100 pmoles each of the PCR primers (5′ PCR primer:

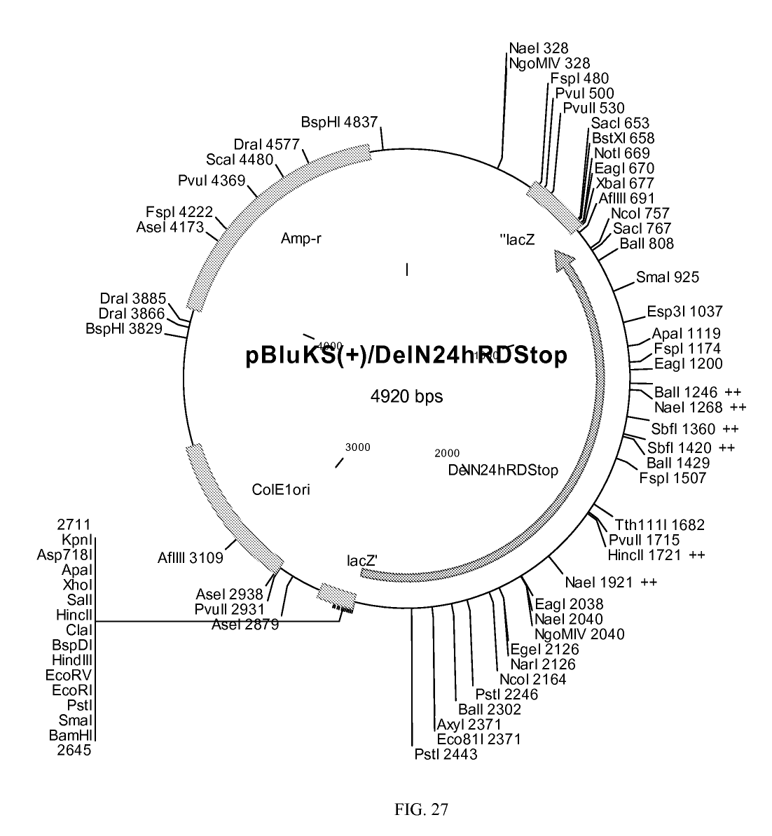

5′-ATggatccATGACGGACATGATTCTGTTTTCGCTC-3′ (letters in lower casing represent the BamHI site—SEQ ID NO: 35) & 3′ PCR primer:
5′-ATtctagaCTAGCTCCACACGTCCAGGGAGTAGCGGC-3′ (letters in lower casing represent the XbaI site—SEQ ID NO: 36)) were used with DNA (500 ng) from a cDNA library, derived from human liver, as template. The ΔN24hRD gene was subcloned, as in Example 1, in pBluescript KS+ to obtain the plasmid pBluKS(+)/DelN24hRD (FIG. 27). The sequence of the insert was confirmed by restriction enzyme analysis and through DNA sequencing.

### Example 2.10

### Cloning of the ΔN24hRD Gene, Without a Stop Codon, from a Liver cDNA Library in the Basic Plasmid pBluescript

The 1974 bp BamHI-XbaI fragment (ΔN24hRD) of the N-terminally truncated human P450 reductase gene without a 3′-end Stop codon, (SEQ ID No. 37), was amplified as a BamHI-XbaI fragment. 100 pmoles each of the PCR primers (5′ primer: SEQ ID No. 35 & 3′ primer:

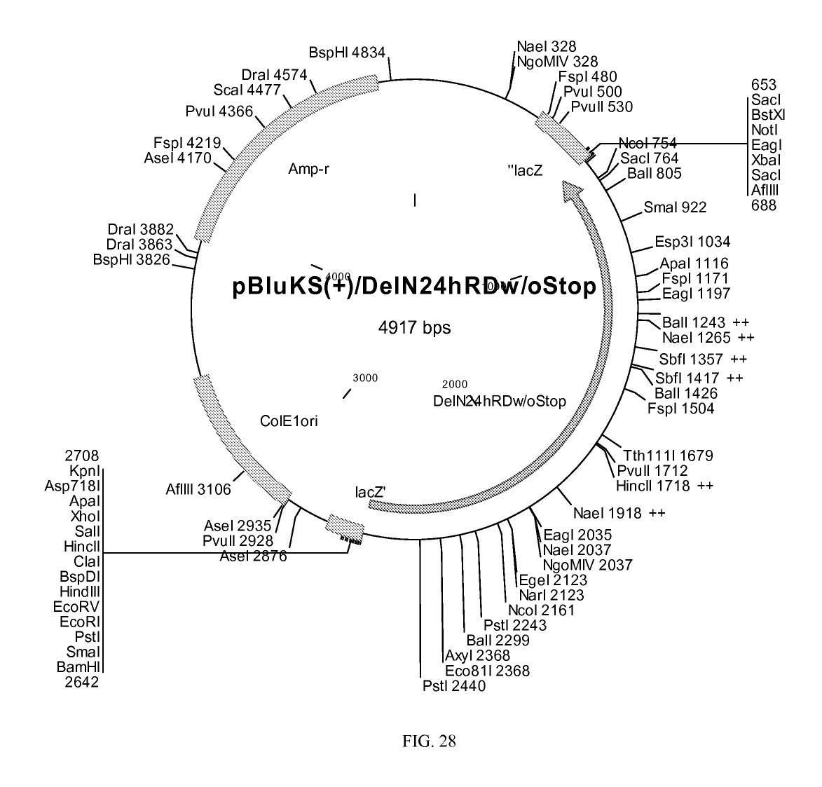

5′-ATtctagaGCTCCACACGTCCAGGGAGTAGCGGC-3′ (letters in lower casing represent the XbaI site—SEQ ID NO: 38)) were used with DNA (500 ng) from a cDNA library, derived from human liver, as template. The ΔN24hRD gene was subcloned, as in Example 1, in pBluescript KS+ to obtain the plasmid pBluKS(+)/DelN24hRDw/oStop (FIG. 28). The sequence of the insert was confirmed by restriction enzyme analysis and through DNA sequencing.

### Example 2.11

### Cloning of the Full Length hRD Gene, with a Stop Codon, from a Liver cDNA Library in the Basic Plasmid pBluescript

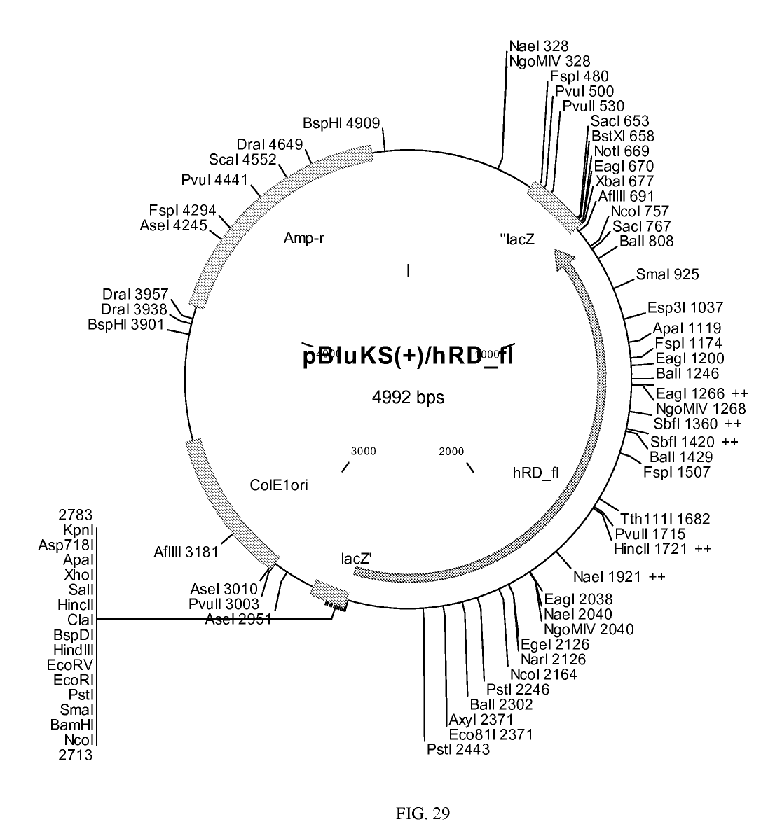

The 2046 bp BamHI-XbaI fragment of the full length human P450 reductase gene (hRD_fl) and containing a 3′-end Stop codon, (SEQ ID No. 39), was amplified as a BamHI-XbaI fragment. 100 pmoles each of the PCR primers (5′ PCR primer: 5′-ATggatccAT GGGAGACTCC CACGTGGACA CCAGCTCCAC CG-3′ (letters in lower casing represent the BamHI site—SEQ ID NO: 40) & 3′ PCR primer: 5′-ATtctagaCT AGCTCCACAC GTCCAGGGAG TAGCGGCCCT TGGTCATC-3′ (letters in lower casing represent the XbaI site—SEQ ID NO: 41)) were used with DNA (500 ng) from a cDNA library, derived from human liver, as template. The hRD_fl gene was subcloned, as in Example 1, in pBluescript KS+ to obtain the plasmid pBluKS(+)/hRD_fl (FIG. 29). The sequence of the insert was confirmed by restriction enzyme analysis and through DNA sequencing.

### Example 2.12

### Cloning of (a) ΔN24hRD Gene, with a Stop Codon, (b) ΔN24hRD Gene, without a Stop Codon, but Contiguous to a 3′-end c-myc Tag (SEQ ID NO: 42), and (c) the Full Length hRD Gene, with a Stop Codon, in the Yeast Integrating Plasmid YILeuGAL1MS

BamHI-XbaI fragments of the

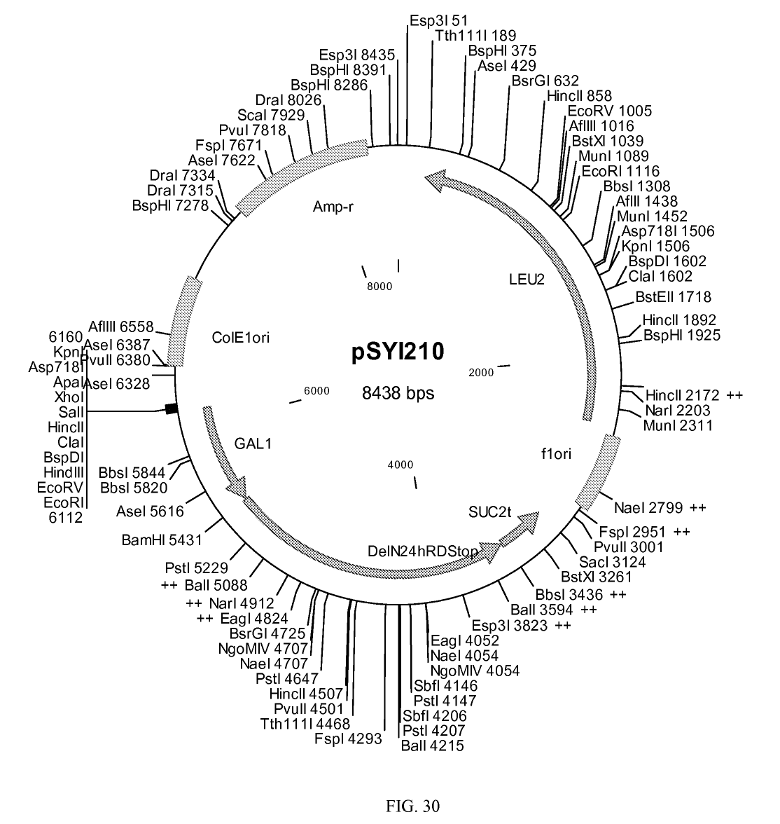

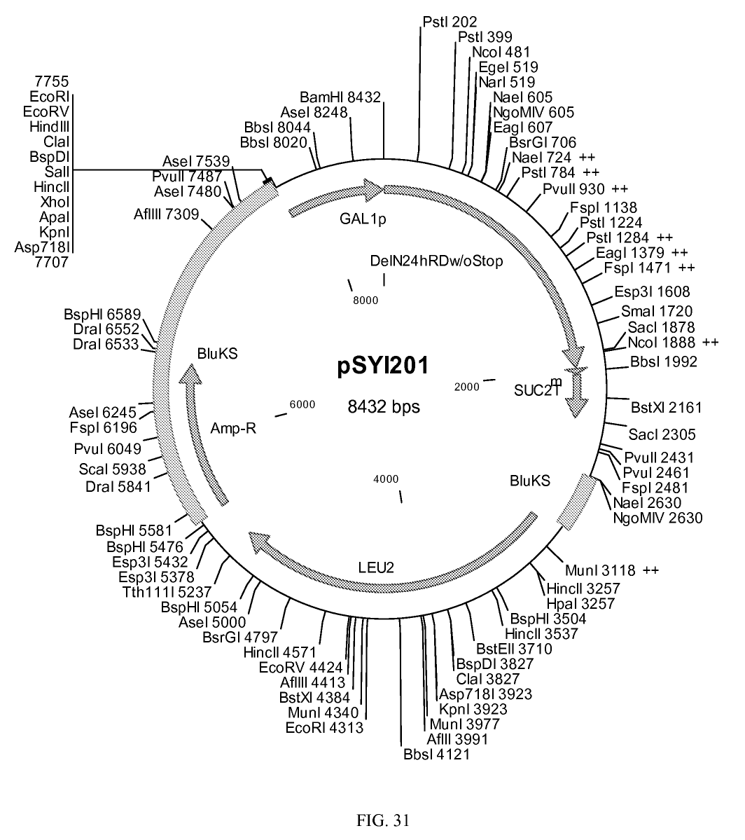

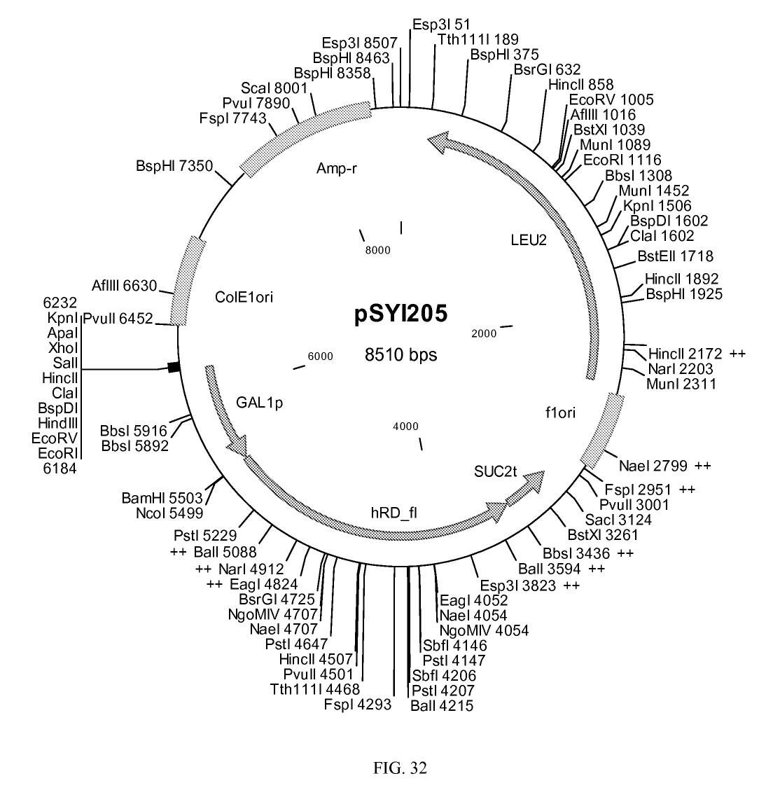

(a) ΔN24hRD gene, with a Stop codon, from plasmid
pBluKS(+)/DelN24hRDStop (FIG. 27),
(b) ΔN24hRD gene, without a Stop codon, from plasmid
pBluKS(+)/DelN24hRDw/oStop (FIG. 28), and
(c) Full-length hRD gene, hRD_fl, with a Stop codon, from plasmid pBluKS(+)/hRD_fl (FIG. 29)
were subcloned in the yeast integrating vector YILeuGAL1MS (FIG. 23) to obtain the following plasmids
(i) pSYI210 (═YILeuG1MS/DelN24hRDStop; FIG. 30),
(ii) pSYI201 (═YILeuG1MS/DelN24hRDw/oStop ═YILeuG1MS/DelN24hRD-cmyc(m)Stop; FIG. 31), and
(iii) pSYI205 (═YILeuG1MS/hRD_flStop; FIG. 32).

### Example 2.13

### Integration of Human NADPHP450 Reductase into Yeast Strains at the LEU2 Locus

In order to integrate at the LEU2 locus of the yeast strain W303B (see Example 1.15), expression cassettes for the three human reductase genes (hRD) variants which are contained in the integrant plasmids pSYI210 (FIG. 30), pSYI201 (FIG. 31), pSYI205 (FIG. 32) were first digested with BstEII. This restriction enzyme cuts uniquely each of the 3 plasmids inside the LEU2 selectable marker gene. The linearised plasmids were then used to transform, using the Bicine method (or using the method described in Example 1.15) of yeast transformation, the yeast strain BC300 and transformants were selected for leucine auxotrophy.

Once integrated, the human reductase genes will be the part of the yeast chromosome and will therefore segregate in mitosis and meiosis with the same high fidelity as any yeast chromosome. Two PCR primers (5′ PCR primer: 5′-CGCGGATCCA TGACGGACAT GATTCTGTTT TCGC-3′ (a part of the beginning of the human reductase gene—SEQ ID NO: 43) & 3′ PCR primer: 5′-CCGGCACGCC ATCCTGCATC CC-3′ (a sequence from the middle of the human reductase gene —SEQ ID NO: 44)) were used for confirmation of integrants and were designed based on the human NADPH P450 reductase gene sequence (SEQ ID No. 39). The expected PCR product using yeast genomic DNA, that bears a human reductase gene cassette, as template is around 1.0 kb when PCR is preformed by using the two primers SEQ ID Nos. 43 & 44. PCR was performed on freshly growing yeast cells and Taq DNA polymerase was used for amplification of DNA.

Strains that show successful integration by PCR were given the names

(1) YI001(W303B:: pSYI210),
(2) YI002 (W303B:: pSYI201),
(3) YI003 (W303B:: pSYI205).
As a control, the yeast strain (4) which had an integrated copy of the plasmid, YILeuG1MS (FIG. 23), with no hRD gene insert, was generated;

### (4) YI004 (W303B:: YILeuG1MS).

The procedure used to generate the strain was exactly as above. The PCR primers that were used to confirm correct integration into yeast had the SEQ ID Nos 7 and 30. The ˜970 bp amplified DNA corresponded to the expected fragment.

Strains (1) to (4) were grown in shake flask cultures (as described in Example 1.15; see above) to assess the microsomal and soluble (i.e. cytosolic) P450 reductase activities present in these strains (as described in Example 2.14; see below).

### Example 2.14

### Evaluation of Human NADPH P450 Reductase Activity in Yeast Strains where the Reductase Gene Variants have been Integrated at the LEU2 Locus of W303B

### Preparation of Microsomes and Cytosolic Fractions from Yeast Cells Bearing hRD Variants for Measurement of P450 Reductase Activity

Microsomes from the different yeast strains were prepared as described in Example 1.15. About 100 μg of microsomes, as measured by the Bio-Rad Bradford assay, were used and the increase in MTT reductase activity was followed over a time period of 400 seconds.

The different yeast strains were grown up in YPGE medium as described in Example 1.15. The cells were harvested and centrifuged to a pellet. 10% of cell pellets were re-suspended in 1 ml of buffer B (10 mM Tris-Cl pH7.5, 2 M Sorbitol, 0.1 mM EDTA pH8.0, 1 mM Pefabloc, 0.1 mM DTT) and re-suspended cells were treated with lyticase (3-5 units/O.D. of cells; 2200 units/mg; Sigma) for about an hour. The yeast cell wall was enzymatically removed using lyticase, the pellets were centrifuged and washed twice with ice cold PBS, finally re-suspended in 1 ml PBS. About 0.5 ml of glass beads were added to the suspension and the cells were vortexed three times with a 5 min interval on ice after each vortexing. The cells lysates were directly used for the MTT-based reductase assay. Equal amount of cells lysates (100 μg, as measured by the Bio-Rad Bradford assay) were used and the increase in MTT reductase activity was followed, as above, over a time period of 400 seconds.

### MTT-Based Cytochrome P450 Reductase Assay

The enzyme NADPH-cytochrome P450 reductase mediates the transfer of electrons from NADPH to cytochrome P450, other microsomal proteins and cytochrome c. It also catalyzes the reduction of many drugs and other compounds such as potassium ferricyanide, 2,6-dichloroindopheonl, 1,1-diphenyl-2-picrylhydrazyl (DPPH), and mitomycin c. Tetrazolium salts are used extensively in cell proliferation and cytotoxicity assays, enzyme assays, histochemical procedures and bacteriological screening. In each of these processes, terazolium salts are metabolically reduced to highly coloured end products called formazans. The compound 3-(4,5-dimethylthiazol-2-yl)-2,5-diphenyltetrazolium bromide (MTT) is a monotetrazolium salt. The reduction of MTT is one of the most frequently used methods for measuring cell proliferation and cytotoxiciy. Reduction of MTT by P450 reductase has been assessed as a method for monitoring yeast produced recombinant P450 reductase activity and the protocol was developed on the procedure published by Yim S-K, et al (Yim S-K., Y. C.-H. Ahn T., Hung H—C and Pan J-G. A continuous Spectrophotometric assay for NADPH-cytochrome P450 reductase activity using 3-(4,5 Dimethylthiazol-2-yl)-2,5-diphenyltetrazolium Bromide. Journal of Biology and Molecular Biology 38: 366-369, 2005). The principal advantage of this substance is that the reduction of MTT can be assayed directly in the reaction medium by a continuous spectrophotometirc method. The electrons released from NADPH by P450 reductase are transferred to MTT, and then the amounts of reduced MTT is assessed spectrophotometrically by measuring the increase in A₆₁₀ values that is due to the formation of blue formazan. The extinction coefficient of MTT is 11.3 mM⁻¹ cm⁻¹. This method offers the advantages of short analysis time with the use of a relatively cheap commercial substrate. The classical assay uses recombinant cytochrome c as a substrate.

### Solutions Used for the MTT Assay

10 mM potassium phosphate buffer: pH7.4: 8 ml of 1M K₂HPO₄ and 2 ml of 1M KH₂PO₄ add ddH₂O to make up to 1 liter.

10 mM MTT: 41.4 mg of MTT (Sigma, Cat No. M2128) into 10 ml of 10 mM potassium phosphate pH7.4 to give 10 mM MTT.

100 mM potassium phosphate buffer: pH7.6: 86.6 ml of 1M K₂HPO₄ and 13.4 ml of 1M KH₂PO₄ add ddH₂O to make up to 1 litre.

Solution A 1 ml stock (stored at −20° C.): 131 μl of 1M Magnesium Chloride solution (Sigma, Cat No.:M1028) in 1 ml ddH₂O to final concentration 66 mM.

NADP⁺ (Sigma, Cat No.: NO₅₀₅, Mr 765.4) 43.5 mg, final concentration 50 mM.

Hydrated salt of disodium D-Glucose-6-phosphate (Sigma, Cat No.: F7250, Mr 304.1) 172 mg, final concentration 500 mM.

Solution B (stored at −20° C.): 17 U Glucose-6-phosphate dehydrogenase (Sigma, Cat No.: G6378, 250 U) in 340 μl of 5 mM sodium citrate (14.7 mg/ml) (trisbasic) (Sigma, cat No.: S46410).

MTT-Based P450 Reductase Assay Modified for Assessing Yeast-Derived Recombinant P450 Reductase

Disposable cuvettes were used for this experiment. 850 μl of potassium phosphate buffer was added to a cuvette. 100 μg of yeast microsomes or 100 μg of cell supernatants containing the cytosolic fraction of yeast was added to the buffer followed by 10 μl of solution B. The contents were mixed gently to prevent any bubble formation in the resulting suspension. 10 μl of solution A was quickly added to the cuvette, and the contents were mixed by inverting a few times. The cuvette was quickly placed into the spectrophotometer together with the blank cuvette and its contents (that contained all components as in the other test cuvette but not the microsomes or cell supernatant) and the increase in the values at 610 nm was measured for a time period of 400 seconds. The electrons released from NADPH by recombinant P450 reductase enzyme were transferred to MTT, and the ability to reduce MTT was assessed spectrophotometrically by measuring the increase in A₆₁₀ values as a result of the formation of blue formazan. The rate of MTT reduction was calculated from the change in A₆₁₀ values using an extinction coefficient of 11.3 mM⁻¹ cm⁻¹ and the formula, ΔA₆₁₀/min/1.3*0.1 mg/ml=μmole reduced MTT/min/mg of protein.

### Results

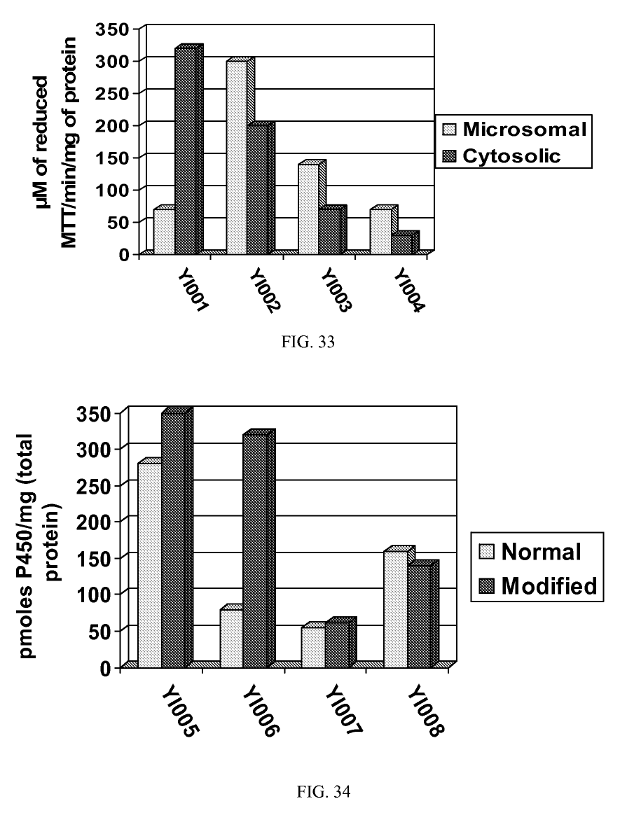

Strains (1) to (4), as elaborated in Example 2.13,
(1) YI001 (W303B:: pSYI210), bearing ΔN24hRDStop,
(2) YI002 (W303B:: pSYI201), bearing ΔN24hRD-cmycStop,
(3) YI003 (W303B:: pSYI205), bearing hRD_flStop,
(4) YI004 (W303B:: YILeuG1MS), the control strain, were grown in shake flask cultures (as described in Example 1.15; see above) to assess the microsomal and soluble (i.e. cytosolic) P450 reductase activities present in these strains. The depicted y-axis values (μM of reduced MTT/min/mg of protein), in FIG. 33, are an average of at least 3 separate determinations.

The results in FIG. 33 show that

(1) Strain 1 bearing the ΔN24hRD mutant (lacking the N-terminal 24-amino acid charged domain) is marginally more active than the Strain 4 which contains only the endogenous yeast reductase driven by the yeast reductase promoter. It should be noted that Strain 1 not only expresses the ΔN24hRD mutant but also endogenous reductase. It appears that most of the activity of ΔN24hRD exists in the soluble cytosolic part of the cell indicating that ΔN24hRD may not be profoundly bound to the endoplasmic reticular (ER) membranes. Since the P450 isozymes are ER membrane bound (i.e. microsome bound), one would assume that, for optimal P450 activity, the reductase also ought to be associated with the ER. Although these experiments indicate that the microsome bound reductase activity of ΔN24hRD is minimal, yet later we will describe (please SEE below) that this activity inexplicably increases when ΔN24hRD is integrated at different yeast chromosomal (genetic) loci or even when strains are cultivated under different growth conditions.
(2) Strain 2 which expresses the ΔN24hRD-cmyc fusion protein is much superior than ΔN24hRD in its ability to bind to the microsomal membranes.
(3) The microsome bound ΔN24hRD-cmyc fusion protein is superior in its reductase activity than the wild type full-length protein. However, its cytosolic activity is also higher.

### CONCLUSIONS

(1) The novel hRD variants ΔN24hRD and ΔN24hRD-cmyc fusion have interesting properties that are helpful in devising improved expression systems for production of cytochrome P450 isozymes (both human and non-human) in yeast.
(2) The interesting properties can be exploited in devising novel production systems that use yeast, insect and/or mammalian cells.

### Example 3

### Expression of Human P450 isozyme CYP1B1 from Yeast Strains that Co-Express the hRD Variants, Protein Expression of Both Proteins Being Driven by the 675 bp GAL1-675 Promoter

As discussed in Example 2, hRD is essential for the activity of the P450 isozymes. Since over-expression of P450 reductase enzyme is harmful to the living cell, it is not unusual that it has been observed that co-expression of P450 reductase adversely affects P450 expression.

In this Example, the possible influences of the novel hRD variants (as described in Example 2) on P450 expression and activity was investigated.

### Example 3.1

### Yeast Transformation

The strains (1) to (4), as elaborated in Example 2.12,

(1) YI001 (W303B:: pSYI210), bearing ΔN24hRDStop,
(2) YI002 (W303B:: pSYI201), bearing ΔN24hRD-cmycStop,
(3) YI003 (W303B:: pSYI205), bearing hRD_flStop,
(4) YI004 (W303B:: YILeuG1MS), the control strain,
were transformed with the plasmid pSYE225 (FIG. 14) that bears the human CYP1B1 gene using the DMSO method of yeast transformation (as described in Example 1.15) to obtain the following strains:
(1) YI005 (YI001::pSYE225),
(2) YI006 (YI002::pSYE225),
(3) YI007 (YI003::pSYE225),
(4) YI008 (YI004::pSYE225).

### Example 3.2

### Growth of yeast cultures for preparation of yeast microsomes

The yeast cells from the strains YI005, YI006, YI007 and YI008 were grown by the method used for “Growing yeast cultures for microsome preparation” as in Example 1.15. In the modified procedure, ethanol and galactose were added together on the 3^{rd} day, instead of ethanol being added on the 2^{nd} day and galactose on the 3^{rd} day (see Example 1.15).

### Example 3.3

### Preparation of Yeast Microsomes

Microsomes from the strains YI005, YI006, YI007 and YI008 were prepared as in Example 1.15.

### Example 3.4

### Measurement of P450 Amounts Via CO-Difference Spectroscopy

P450 amounts obtained from the strains YI005, YI006, YI007 and YI008 were measured using the protocol “Determination of P450 amounts via CO-difference spectroscopy), as described in Example 1.15.

### Results

The values depicted in FIG. 34 are an average of at least 3 individual experiments. It should be noted that all researchers around the world use only the wild type, full-length hRD for all recombinant expression of P450 proteins (human and non-human).

### Conclusions

(1) The strains YI005 and YI006 bearing the ΔN24hRD mutant and the ΔN24hRD-cmyc fusion proteins are the best in their abilities to produce the cytochrome P450 isozyme CYP1B1.
(2) The strain YI007 which bears the wild type, full-length hRD protein (used by all P450 labs around the world) allows comparatively less production of CYP1B1 protein.
(3) The proteins ΔN24hRD and ΔN24hRD-cmyc fusion are helpful in producing higher amounts of any P450 protein than the full-length hRD protein. These two variant hRD proteins can therefore be used for productions of P450s with higher activity.

### Example 3.5

### EROD Assay for Determining P450 Activities in Yeast Microsomes Bearing Human CYP1B1, CYP1A1, CYP1A2 Proteins, etc (Many P450 Enzymes use EROD as a Substrate but with Different Degrees of Efficiency

### Introduction

One of the most well-used and standardized assays for determining P450 enzyme levels is the measurement of the activity of an enzyme called 7-ethoxyresorufin O-deethylase (Klotz A. V., S. J. J. a. W. C. An alternative 7-ethoxyresorufin O-deethylase activity assay: a continuous visible spectrophotometric method for measurement of cytochrome P450 monooxygenase activity. Analytical Biochemistry 140: 138-145, 1984). This enzyme converts 7-ethoxyresorufin to resorufin which can be flourimetrically detected in presence of NADPH and oxygen and the assay is known as the EROD assay.

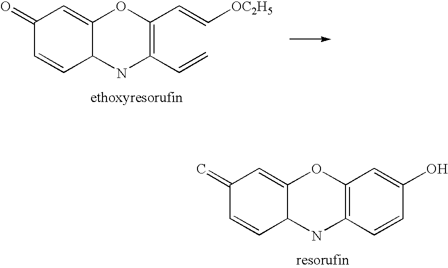

The amount of resorufin produced is measured in a spectrophotometer after the addition of NADPH and the samples to be tested. The samples were microsomal preparations from Saccharomyces cerevisiae.

### Solutions Used for the Assay

Solution A 10 ml stock (Stored at −20° C.):

NADP⁺ (Sigma, Cat No.: NO₅₀₅, Mr 765.4) 200 mg, final concentration 26.13 mM; D-Glucose-6-phosphate disodium salt hydrate (Sigma, Cat No.: F7250, Mr 304.1) 200 mg, final concentration 65.77 mM; MgCl₂ (Sigma, Cat No.:M1028, 1M solution), final concentration 65.42 mM. The solution is made up to 10 ml with deionised water.

Solution B 6.25 ml stock (Stored at −20° C.): 250 U Glucose-6-phosphate Dehydrogenase (Sigma, Cat No.: G6378, 250 U) in 6.25 ml of 5 mM sodium citrate (trisbasic) (Sigma, cat No.: S46410).

Solution C 15 ml freshly prepared: 1.5 ml of 0.5 M KPO₄ pH7.4; 1.5 ml of solution A; 0.3 ml of solution B. The solution is made up to 15 ml with 11.5 ml of deionised water.

0.5 M Potassium Phosphate (KPO₄) pH7.4 (Kpi buffer): 19.8 ml of buffer A (1M; 136.1 g KH₂PO₄/L) and 80.2 ml of buffer B (1 M; 174.2 g K₂HPO₄/L) is used to make up to 200 ml with sterile water and pH was adjusted to 7.4.

0.1 M Potassium Phosphate (KPO₄) pH 7.4: 1.98 ml of buffer A (1M; 136.1 g KH₂PO₄/L) and 8.02 ml of buffer B (1 M; 174.2 g K₂HPO₄/L) is used to make up to 100 ml with sterile water and pH was adjusted to 7.4.

50 mM Sodium Citrate (tribasic): 147 mg of sodium citrate (tribasic)(Sigma, Cat No.: S4641) is dissolved in 10 ml of deionised water.

10% DMSO 100 ml: 10 ml of neat DMSO in 90 ml of deionised water.

1 mM 7-Ethoxyresorufln: 2.1412 mg of 7-ethoxyresorufin (Sigma, Cat. No.: E3763, Fw: 241.2.2) is taken up in 10 ml of neat DMSO. This solution is later diluted to 0.1 mM solution in 10% DMSO on the day of use. This solution is extremely light sensitive so it must be stored in brown bottles and wrapped in tin foil. It is stored at −20° C.

10 mM Resorufln: 23 mg of resorufin (Sigma, Cat. No. R3257, Fw: 235.17) is dissolved in 10 ml of neat DMSO. This solution is also extremely light sensitive so it must be stored in brown bottles and wrapped in tin foil. It is stored at −20° C.

### The Assay Procedure

The reagents used in this assay are light sensitive. All manipulations involved in the assay were carried out in a laboratory with windows stained yellow. The assay used is a modification of the Klotz's procedure (Klotz A. V., S. J. J. a. W. C. An alternative 7-ethoxyresorufin O-deethylase activity assay: a continuous visible spectrophotometric method for measurement of cytochrome P450 monooxygenase activity. Analytical Biochemistry 140: 138-145, 1984). All reactions were performed in a 96-well black transparent flat-bottomed plate (Fisher Cat No.: FB86083) and were used for the detection (with the aid of a Bio-Tek fluorescence spectrophotometer and the software KC4) of produced resorufin (Hahn, M. E., Lamb, T. M., Schultz, M. E., Smolowitz, R. M. and Stegeman, J. J. Cytochrome P4501A induction and inhibition by 3,3′,4,4′-tetrachlorobiphenyl in an Ah receptor-containing fish hepatoma cell line (PLHC-1). Aquatic Toxicology 26: 185-208, 1993).

The black plate and all solutions were pre-warned at 37° C. in the dark before the assay was started. The wavelength for absorption was set up at 530 nm and emission at 590 nm. All reactions were run 20-40 min and the plate was auto-mixed before reading. Endpoint analysis was performed for creating resorufin standard curve.

In the 96-well plate, each reaction contains 1.5 μmol (in approximately 25 μg of total microsomal protein) of yeast microsome associated recombinant P450 protein in 0.1 M KPO₄ buffer. In order to have all reactions contain the same amount of total protein, extra amounts of excess protein derived from microsomes of a basic yeast strain that contain no endogenous yeast reductase gene (i.e. yRD gene disrupted) were used. The protein used for resorufin standard curve was either from boiled microsomes or microsomes prepared from the endogenous yeast reductase disruption strain.

Since in most instances the amount of P450 present in a microsomal preparations are known in advance (via CO-difference spectra), the P450 activity from the EROD assay can be directly expressed as pmol of resorufin produced/min/pmol P450. Alternatively, activities can be expressed as pmol of resorufin produced/min using the standard curve.

### Results

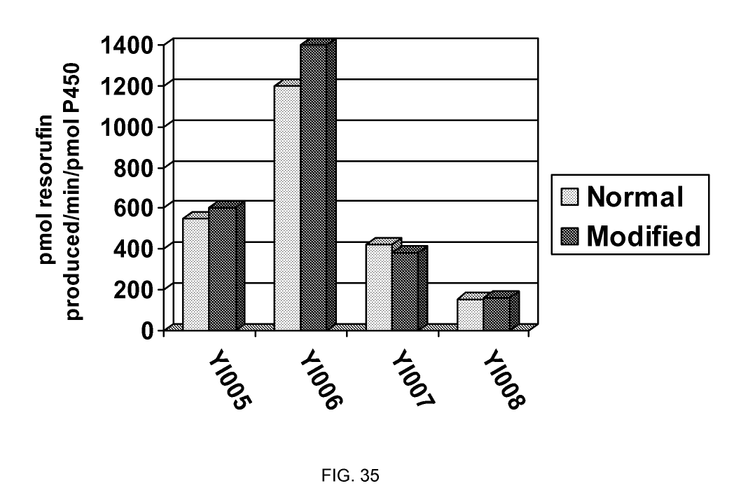

FIG. 35 depicts “relative” activities of CYPIB1 in microsomes obtained from strains YI005, YI006, YI007 and YI008 using the normal (Example 1.15) and modified (Example 3.2) procedures for growing yeast cultures. The results shown are an average of at least 3 individual experiments.

### Conclusions

(1) The strain YI006 that bears the novel ΔN24hRD-cmyc fusion variant of hRD contributes to the best activity of CYP1B1 produced in yeast.
(2) Both the variants of hRD, ΔN24hRD and ΔN24hRD-cmyc, are being used to co-express other P450 isozymes.
(3) It should be noted that YI007 bears the full-length hRD and which is commonly used for co-expression of P450 isozymes in yeast, insect and mammalian cells. The strain YI007 not only produces less P450 protein (see Example 3.3) but also the P450 produced is less active than those produced in YI006 and YI005 (this Example).

### Example 4

### Construction of Yeast Strains that Bear the ΔN24hRD Variant for Integration at Different Chromosomal Loci: Comparative Analysis of hRD Activities and Relationship with CYP Activities

It has been investigated whether there would be a consequence of integrating the ΔN24hRD mutant at different chromosomal loci. This was done mainly to determine if all loci available in the yeast strain W303B were equally efficient in expressing the reductase activity from the ΔN24hRD mutant. Ultimately the goal was to find a reductase locus that would be most efficient in allowing co-expression of P450 isozymes in yeast.

### Example 4.1

### Construction of Plasmids

BamHI-XbaI fragment of the ΔN24hRD gene, with a Stop codon, from plasmid pBluKS(+)/DelN24hRDStop (FIG. 27), was subcloned in the yeast integrating vectors

### (1) YIAdeGAL1MS (FIG. 24),

### (2) YIHisGAL1MS (FIG. 25),

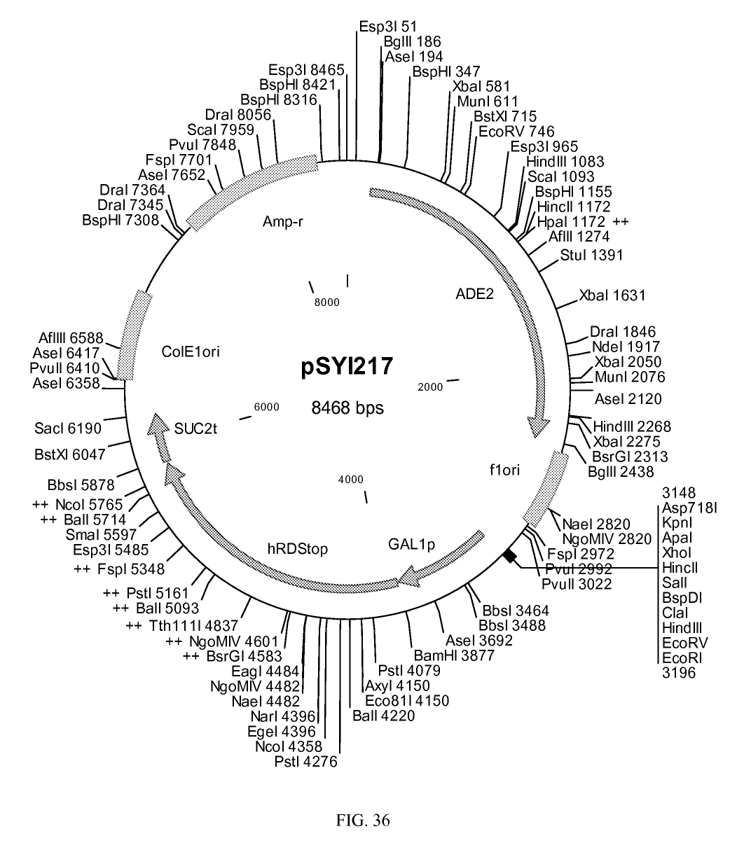

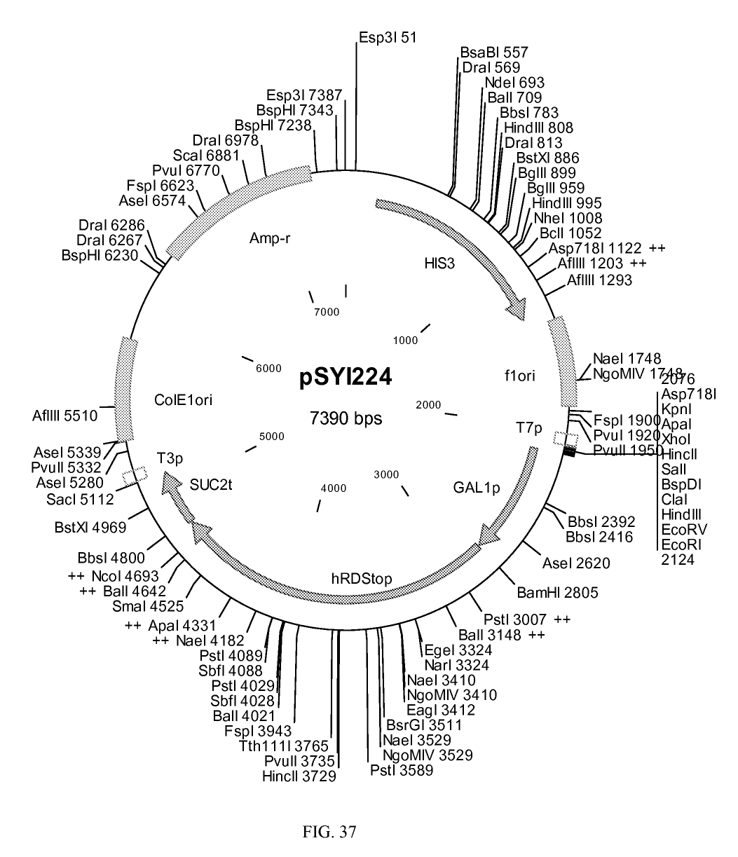

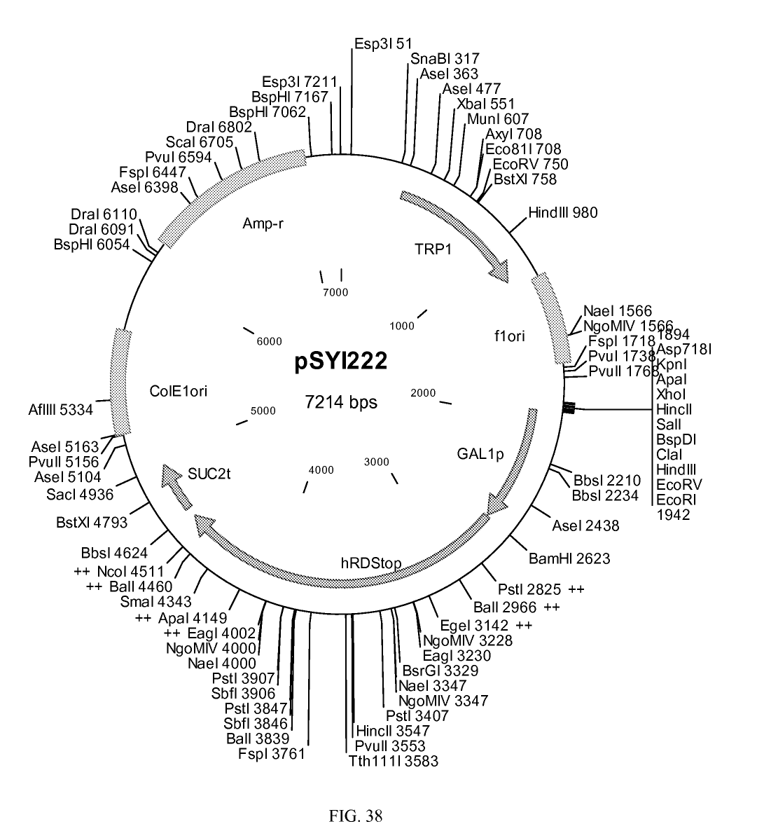

(3) YITrpGAL1MS (FIG. 26), to obtain the following plasmids
(i) pSYI217 (═YIAdeG1MS/DelN24hRDStop; FIG. 36),
(ii) pSYI224 (═YIHisG1MS/DelN24hRDStop; FIG. 37),
(iii) pSYI222 (═YITrpG1MS/DelN24hRDStop; FIG. 38).

### Example 4.2

### Yeast Transformation

### Integration of ΔN24hRD Gene into Different Chromosomal Loci of the Yeast Strain W303B

The following restriction enzymes were used to linearise the plasmids:

(1) StuI for pSYI217,
(2) NdeI for pSYI224,
(3) NarI for pSYI222.

The plasmids pSYI217 and pSYI224 were used for integration at the ADE2 and HIS3 loci of the yeast strain W303B whereas pSYI222 was used to integrate at the yRD gene locus of W303B. The transformation protocol used for yeast transformation was the same as in Example 1.15.

The resultant strains were:

(1) YI009 (W303B:: pSYI217), bearing ΔN24hRDStop at the ADE2 locus,
(2) YI010 (W303B:: pSYI224), bearing ΔN24hRDStop at the HIS3 locus,
(3) YI011 (W303B:: pSYI222), bearing the ΔN24hRDStop gene at the yRD gene locus, implying that the endogenous yRD in W303B was disrupted by introducing a GAL1 promoter driven ΔN24hRDStop gene cassette.

All integant strains were confirmed by PCR using the primers SEQ ID Nos. 43 &44.

### Example 4.3

### Growth of Yeast Cultures for Preparation of Yeast Microsomes

The yeast cells from the strains

(1) YI001 (bearing ΔN24hRDStop at the LEU2 locus),
(2) YI009 (bearing ΔN24hRDStop at the ADE2 locus),
(3) YI010 (bearing ΔN24hRDStop at the HIS3 locus),
(4) YI011 (bearing ΔN24hRDStop at the yRD locus), and
(5) YI004 (W303B:: YILeuG1MS), the control strain were grown by the method used for “Growing yeast cultures for microsome preparation”, as in Example 1.15.

### Example 4.4

### Preparation of Yeast Microsomes

Microsomes from the strains YI001, YI009, YI010, YI011 and the control strain YI004 were prepared according to the protocol described in Example 1.15.

### Example 4.5

### Measurement of Reductase Activities of Strains, Described in Example 4.3, Via the MTT-Based Assay

Reductase activities obtained from the strains YI001, YI009, YI010, YI011 and the control strain YI004 were measured using the protocol described in Example 2.13.

### Results

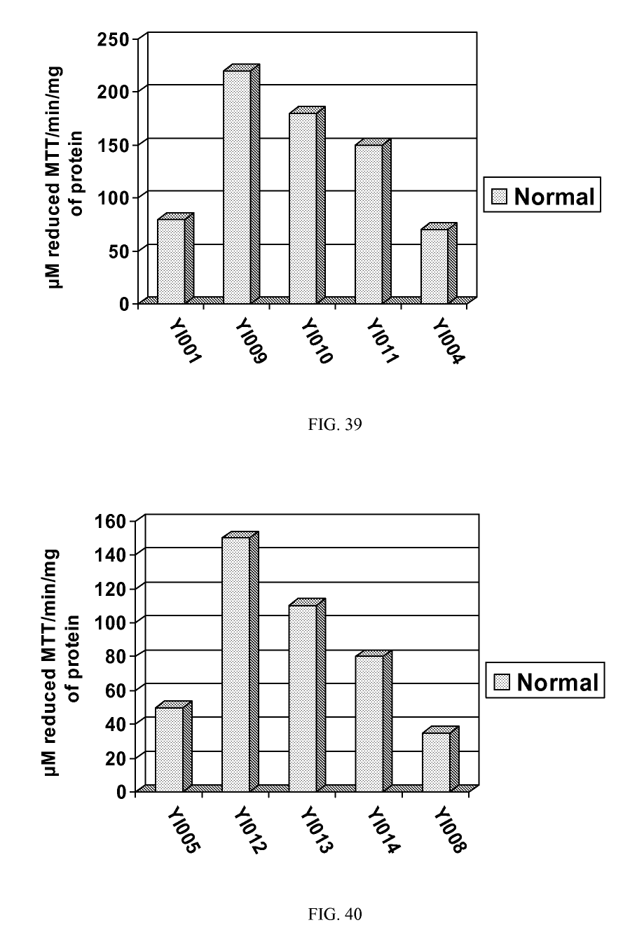

The reductase activity values depicted in FIG. 39 are an average of at least 3 individual experiments.

### Conclusions

(1) Expression of ΔN24hRD from different chromosomal loci results in differential expression of reductase activity.
(2) It is therefore possible to provide a panorama of P450 activities for any P450 recombinant isozyme of choice.

### Example 4.6

### Transformation of pSYE225, the CYPB1 Containing Plasmid, into Yeast Strains that Bear ΔN24hRD at Different Chromosomal Loci

The plasmid pSYE225 was transformed, using the protocol detailed in Example 1.15, into the strains

(1) YI001 (bearing ΔN24hRDStop at the LEU2 locus),
(2) YI009 (bearing ΔN24hRDStop at the ADE2 locus),
(3) YI010 (bearing ΔN24hRDStop at the HIS3 locus),
(4) YI011 (bearing ΔN24hRDStop at the yRD locus), and
(5) YI004 (W303B:: YILeuG1MS), the control strain to obtain the resultant strains (strains (i) and (v) had been obtained earlier in Example 3.1)
(i) YI005 (YI001::pSYE225),
(ii) YI012 (YI009:: pSYE225),
(iii) YI013 (YI010:: pSYE225),
(iv) YI014 (YI011:: pSYE225),
(v) YI008 (YI004::pSYE225).

### Example 4.7

### Measurement of Reductase Activities of Strains, Described in Example 4.3, Via the MTT-Based Assay

Reductase activities obtained from the strains YI005, YI012, YI013, YI014 and the control strain YI008 were measured using the protocol described in Example 2.13.

### Results

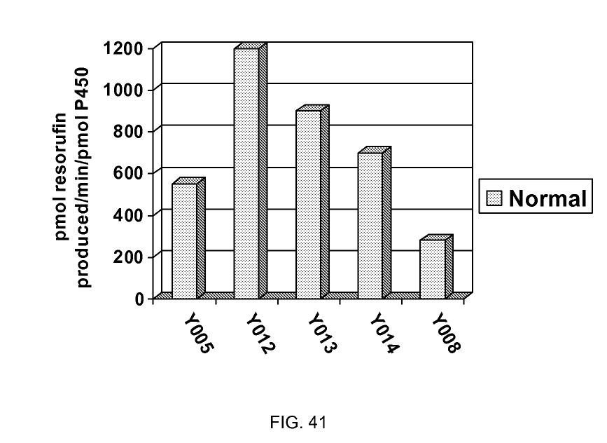

The values depicted in FIG. 40 are an average of at least 3 individual experiments. The cells were grown by the “normal” method used for “Growing yeast cultures for microsome preparation” as in Example 1.15 and the microsomes were also prepared by the same method as in Example 1.15.

### Conclusions

(1) The reductase activities in strains YI005, YI012, YI013, YI014 and the control strain YI008 (all strains bearing the human CYP1B1 gene) parallel the reductase activities in strains YI001, YI009, YI010, YI011 and the control strain YI004 (i.e. strains that do not bear the human CYP1B1 gene).
(2) It appears that the genes in the neighborhood of the region where ΔN24hRD is integrated in the chromosome have an influence on reductase expression and activities (Brem, R. B. et al. Genetic dissection of transcriptional regulation in budding yeast. Science 296(5568): 752-756, 2002).

### Example 4.8

### Measurement of P450 Activities in Strains, Described in Example 4.6, Via the EROD Assay

CYP1B1 P450 activities in the microsomes (prepared under normal growth conditions, as described in Example 1.15) obtained from the strains YI005, YI012, YI013, YI014 and the control strain YI008 were measured using the EROD assay protocol, as described in Example 3.4.

### Results

The values depicted in FIG. 41 are an average of at least 3 individual experiments. The cells were grown by the “normal” method used for “Growing yeast cultures for microsome preparation” as in Example 1.15 and the microsomes were also prepared by the same method as in Example 1.15.

### Conclusions

There appears to be a direct correlation between the reductase activities of mutant ΔN24hRD expressed from different yeast chromosomal loci and CYP1B1 activities. This may be in contrast to the expression of full-length hRD where more reductase activity usually implies drastically reduced levels of P450 protein.

### Example 5

### Construction of Yeast Strains that Bear Two Copies of the 2 hRD Variants at Two Different Chromosomal Loci: Comparative Analysis of hRD Activities and Relationship with CYP Activities

It was totally unexpected that ΔN24hRD and ΔN24hRD-c-myc would behave so differently from full-length hRD in their ability to positively influence the amounts and activity of recombinant P450 proteins produced in yeast. Too much of full-length hRD activity is deleterious for the cell and also hinders P450 expression. The consequence of co-expressing two copies of ΔN24hRD or ΔN24hRD-c-myc together with a P450 isozyme has been investigated.

### Example 5.1

### Construction of Plasmids

Plasmids that will allow integration of a single copy of ΔN24hRD and ΔN24hRD-c-myc into the LEU2 locus of the yeast genome have already been created. The resultant plasmids were named pSYI210 (FIG. 30) and pSYI201 (FIG. 31).

The plasmid that allows integration of a single copy of ΔN24hRD into the ADE2 locus of the yeast genome has already been created. The resultant plasmid was named pSYI217 (FIG. 36).

Construction of a ΔN24hRD-cmyc Containing ADE2 Integrating Plasmid

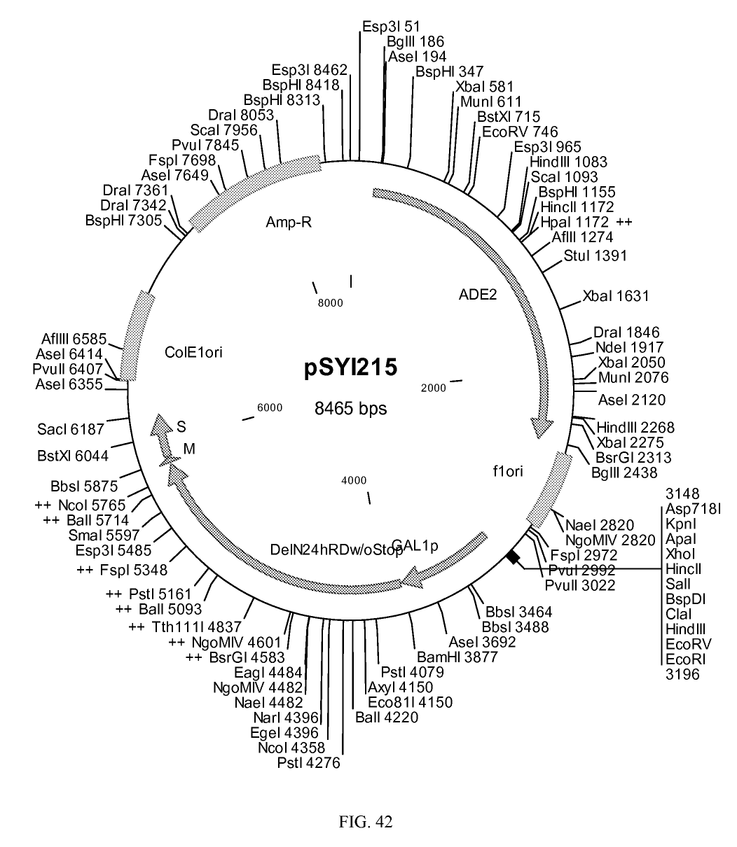

BamHI-XbaI fragment of the ΔN24hRD gene, without a Stop codon, from plasmid pBluKS(+)/DelN24hRDw/oStop (FIG. 25) was subcloned in the yeast integrating vector YIAdeGAL1MS (FIG. 24) to obtain the plasmid pSYI215 (=YIAdeG1MS/DelN24hRDw/oStop; FIG. 42).

Construction of LEU2 and ADE2 Integrating Plasmids that Contain a Second Copy of GAL1-675 Promoter Driven ΔN24hRD and ΔN24hRD-cmyc Expression Cassettes

The making of the DNA constructs involved 3 Steps.

### Step 1

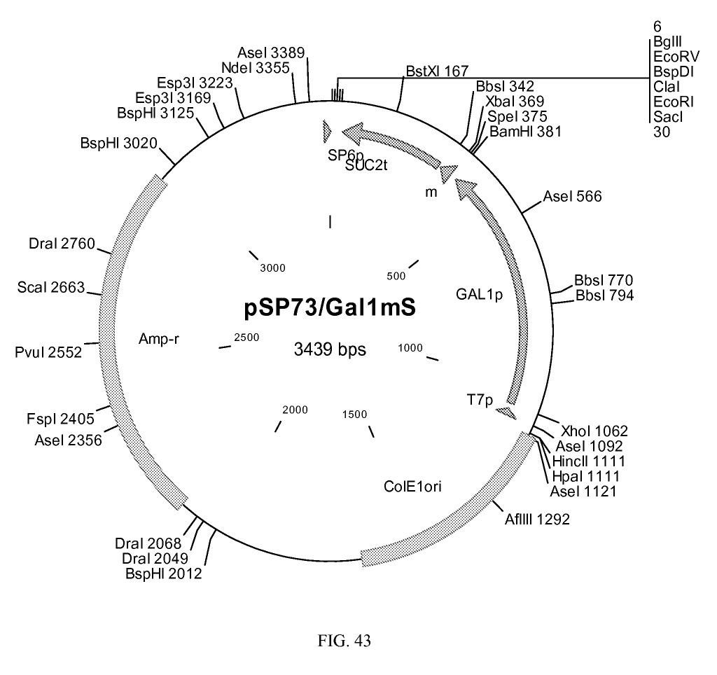

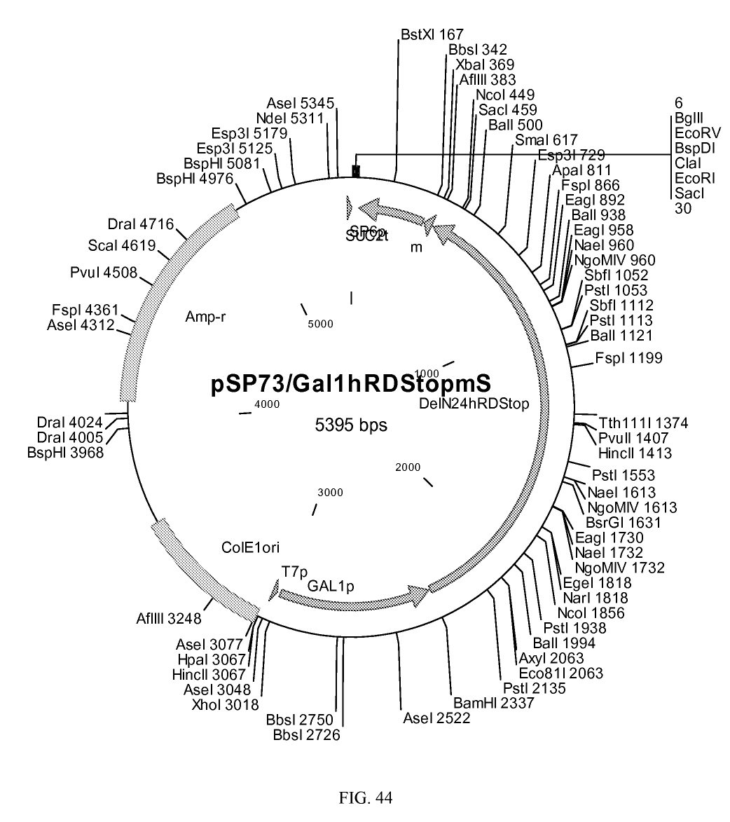

In order to create a second copy of GAL1p675 promoter driven ΔN24hRD (with Stop) and ΔN24hRD (without Stop) expression cassettes, firstly, the XhoI-SacI Gal1mS fragment from pBluKS(+)/Gal1mS (FIG. 22) was subcloned in the basic vector pSP73 (obtained from Promega) to obtain the plasmid pSP73/Gal1mS (FIG. 43).

### Step 2

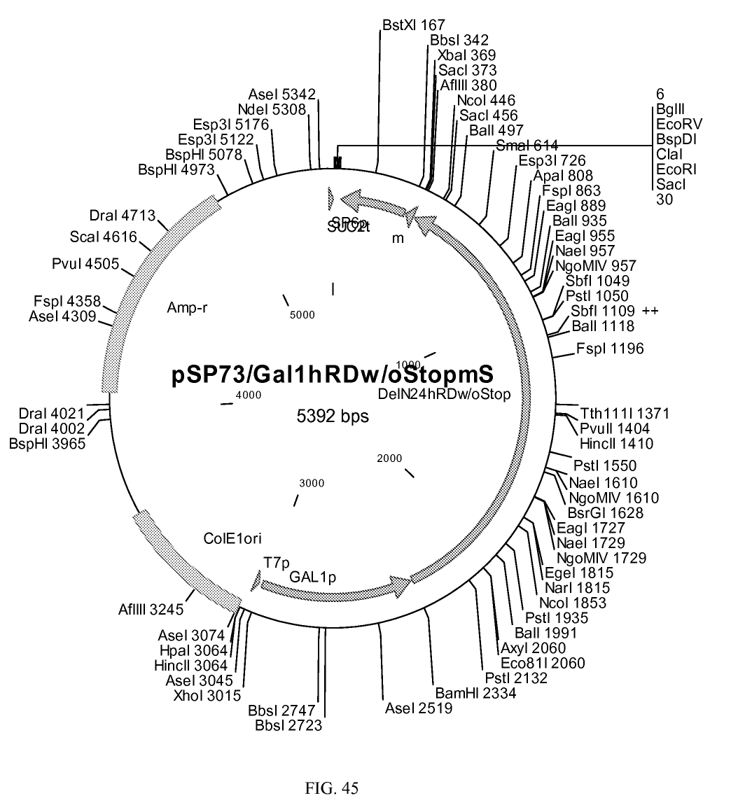

The BamHI-XbaI fragments of ΔN24hRDStop and the ΔN24hRDw/oStop genes were then sublconed in the BamHI, XbaI sites of pSP73/Gal1mS (FIG. 43) to obtain the plasmids pSP73/Gal1hRDStopmS (FIG. 44) and pSP73/Gal1hRDw/oStopmS (FIG. 45).

### Step 3

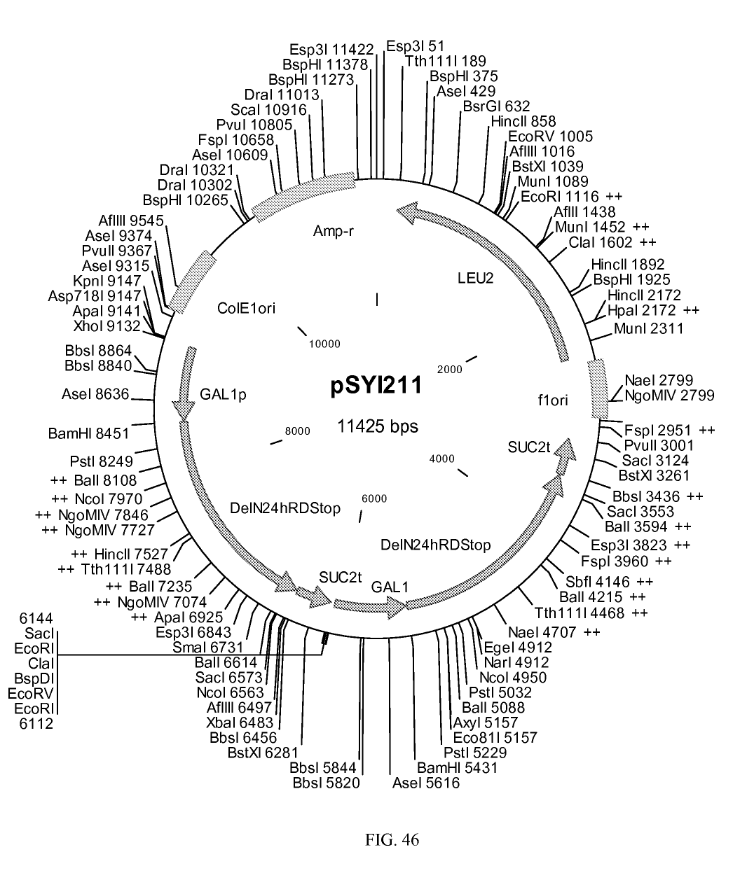

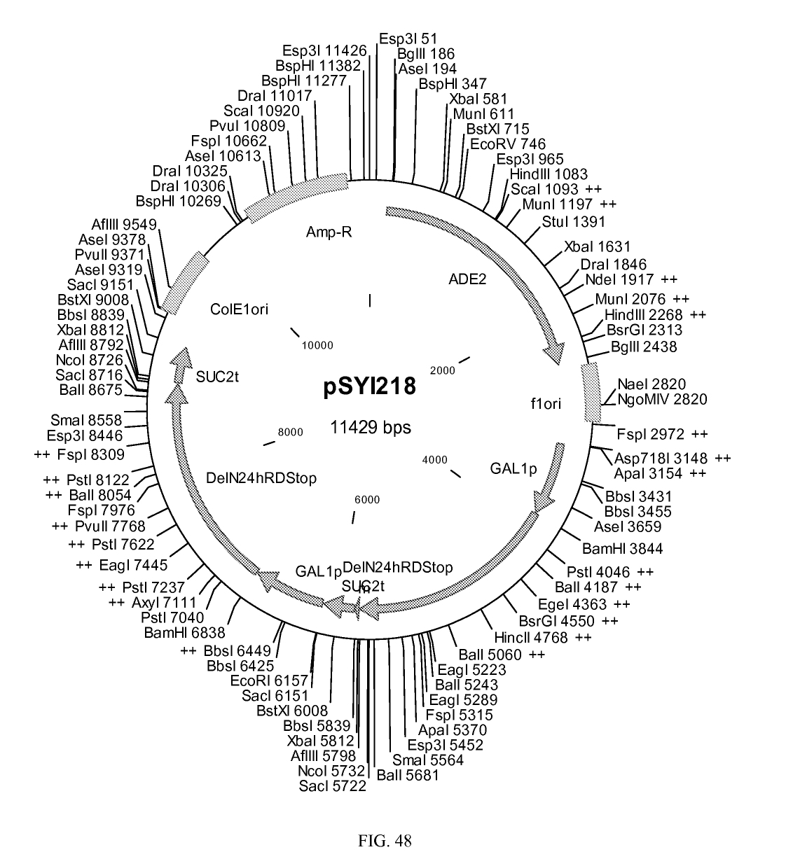

Finally, the XhoI-EcoRV fragment from pSP73/Gal1hRDStopmS (FIG. 44) that contains Gal1hRDStopmS was ligated to HindIII (blunt-ended), XhoI digested pSYT210 (FIG. 30) and pSYT217 (FIG. 36) to obtain the plasmids pSYT211 (FIG. 46) and pSYI218 (FIG. 48). The HindIII site of the two vectors pSYT210 and pSYT217 was blunt-ended by flushing with Klenow polymerase.

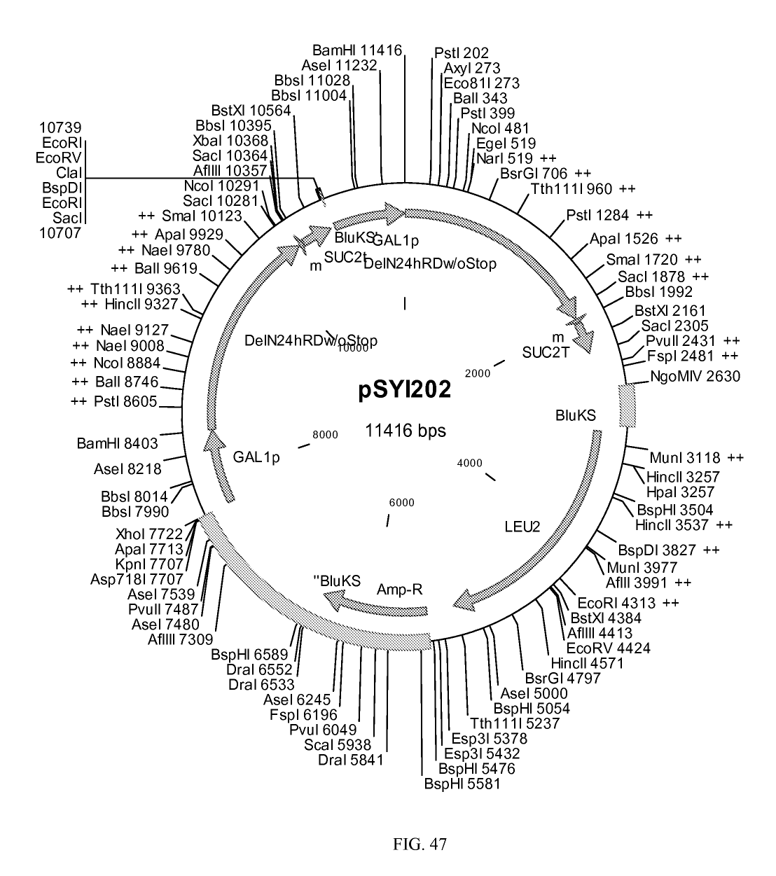

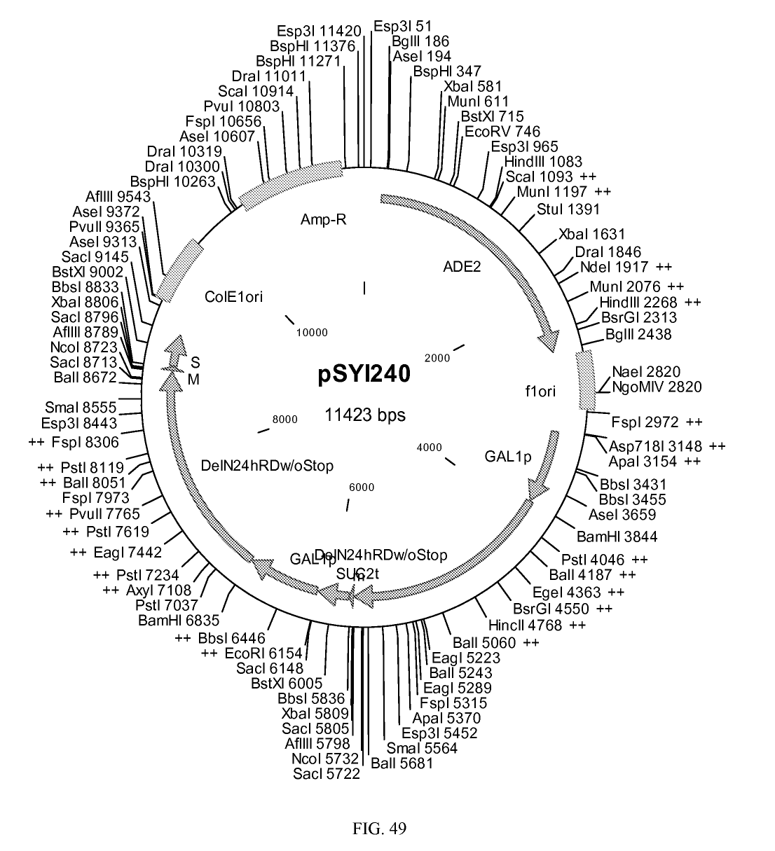

And, the XhoI-EcoRI fragment that contains Gal1hRDw/oStopmS isolated from pSP73/Gal1hRDStopmS (FIG. 44) was ligated to XhoI, EcoRI digested pSYI201 (FIG. 31) and pSYI215 (FIG. 42) to obtain the plasmids pSYI202 (FIG. 47) and pSYI240 (FIG. 49).

HindIII Fragment (HindIII Site Flushed with Klenow Polymerase)

### Example 5.2

### Yeast Transformation

### Integration of 2 Copies of ΔN24hRD and ΔN24hRD-cmyc Genes into the LEU2 and ADE2 Chromosomal Loci of the Yeast Strain W303B

The following restriction enzymes were used to linearise the plasmids:

(1) BstEII for pSYI211 and pSYI202,
(2) StuI for pSYI217, pSYI218 and pSYI240.

The plasmids pSYI211 and pSYI202 were used for integration at the LEU2 locus whereas pSYI218 and pSYI240 were used for integration at the ADE2 locus of the yeast strain W303B. The transformation protocol used for yeast transformation was the same as in Example 1.15.

The resultant strains were:

(1) YI015 (W303B:: pSYI217), bearing a copy of ΔN24hRD-cmyc at the ADE2 locus,
(2) YI016 (W303B:: pSYI211), bearing 2 copies of ΔN24hRDStop at the LEU2 locus,
(3) YI017 (W303B:: pSYI202), bearing 2 copies of ΔN24hRD-cmyc at the LEU2 locus,
(4) YI018 (W303B:: pSYI218), bearing 2 copies of ΔN24hRDStop at the ADE2 locus,
(5) YI019 (W303B:: pSYI240), bearing 2 copies of ΔN24hRD-cmyc at the ADE2 locus.

All integrant strains were confirmed by PCR using the primers SEQ ID Nos. 45 and 46.

### Example 5.3

### Growth of Yeast Cultures for Preparation of Yeast Microsomes

The yeast cells from the strains

(1) YI001 (bearing a copy of ΔN24hRDStop at the LEU2 locus),
(2) YI002 (bearing a copy of ΔN24hRD-cmyc at the LEU2 locus),
(3) YI009 (bearing a copy of ΔN24hRDStop at the ADE2 locus),
(4) YI015 (bearing a copy of ΔN24hRD-cmyc at the ADE2 locus),
(5) YI016 (bearing 2 copies of ΔN24hRDStop at the LEU2 locus),
(6) YI017 (bearing 2 copies of ΔN24hRD-cmyc at the LEU2 locus)
(7) YI018 (bearing 2 copies of ΔN24hRDStop at the ADE2 locus),
(8) YI019 (bearing 2 copies of ΔN24hRD-cmyc at the ADE2 locus) were grown by the method used for “Growing yeast cultures for microsome preparation”, as in Example 1.15.

### Example 5.4

### Preparation of Yeast Microsomes

Microsomes from the strains YI001, YI002, YI009, YI015, YI016, YI017, YI018 and YI019 were prepared using the protocol described in Example 1.15.

### Example 5.5

### Measurement of Reductase Activities of Strains, Described in Example 5.3, Via the MTT-Based Assay

Reductase activities obtained from the strains YI001, YI002, YI009, YI015, YI016, YI017, YI018 and YI019 were measured according to the protocol described in Example 2.13.

### Results

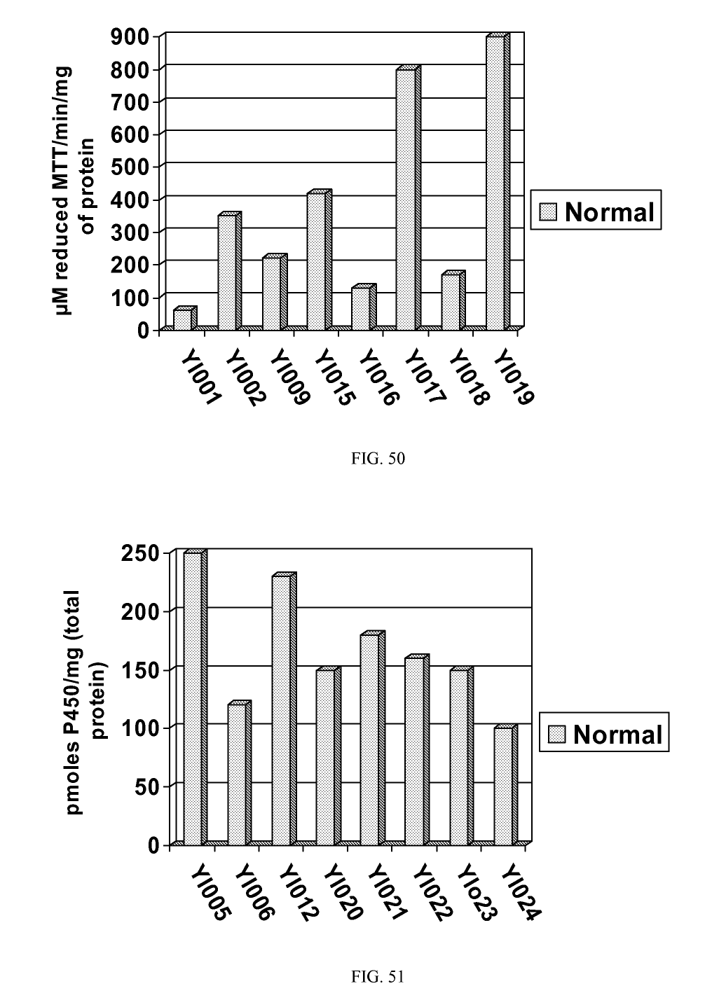

The reductase activity values depicted in FIG. 50 are an average of at least 3 individual experiments.

### Conclusions

(1) It is abundantly clear that the P450 reductase activity depends on the type of reductase mutant that is expressed at a particular chromosomal locus.
(2) One can definitely obtain more reductase activity by introducing two copies, instead of one, of the hRD variant genes at the two different chromosomal loci (i.e. chromosomes at which the LEU2 and ADE2 genes reside in the yeast genome).
(3) With a wider panel of reductase activities, it is possible to provide a broader array of P450 activities for any recombinant P450 isozyme of choice.

### Example 5.6

### Transformation of pSYE225, the CYPB1 Containing Plasmid, into Yeast Strains Described in Example 5.3

The plasmid pSYE225 was transformed, using the protocol detailed in Example 1.15, into the strains

(1) YI001 (bearing a copy of ΔN24hRDStop at the LEU2 locus),
(2) YI002 (bearing a copy of ΔN24hRD-cmyc at the LEU2 locus),
(3) YI009 (bearing a copy of ΔN24hRDStop at the ADE2 locus),
(4) YI015 (bearing a copy of ΔN24hRD-cmyc at the ADE2 locus),
(5) YI016 (bearing 2 copies of ΔN24hRDStop at the LEU2 locus),
(6) YI017 (bearing 2 copies of ΔN24hRD-cmyc at the LEU2 locus),
(7) YI018 (bearing 2 copies of ΔN24hRDStop at the ADE2 locus),
(8) YI019 (bearing 2 copies of ΔN24hRD-cmyc at the ADE2 locus) to obtain the resultant strains (strains (i), (ii) and (iii) had been obtained earlier in Examples 3.1 and 4.6)
(i) YI005 (YI001::pSYE225),
(ii) YI006 (YI002::pSYE225),
(iii) YI012 (YI009:: pSYE225),
(iv) YI020 (YI015:: pSYE225),
(v) YI021 (YI016:: pSYE225),
(vi) YI022 (YI017:: pSYE225),
(vii) YI023 (YI018:: pSYE225),
(viii) YI024 (YI019:: pSYE225).

### Example 5.7

### Measurement of P450 Amounts in Strains, Described in Example 5.6, Via CO-Difference Spectroscopy

P450 amounts obtained from the strains YI005, YI006, YI012, YI020, YI021, YI022, YI023 and YI024 were measured using the protocol “Determination of P450 amounts via CO-difference spectroscopy), as described in Example 1.15.

### Results

The values depicted in FIG. 51 are an average of at least 3 individual experiments. The cells were grown by the “Normal” method of “Growing yeast cultures for microsome preparation” as in Example 1.15 and the microsomes were also prepared by the same method as in Example 1.15.

### Conclusion

There are differences in amounts of CYP1B1 produced in the different strains. However, there may not be a strict correlation between the reductase activities manifested by the reductase mutants and the amounts of P450 produced. The strains bearing ΔN24hRD-cmyc fusion definitely produce less P450 under normal growth conditions. As indicated above (Example 3.2), production levels in the ΔN24hRD-cmyc bearing strains can be elevated using “modified” growth conditions.

### Example 5.8

### Measurement of P450 Activities in Strains, Described in Example 5.6, Via the EROD Assay

CYP1B1 P450 activities in the microsomes (prepared under “normal” growth conditions, as described in Example 1.15) obtained from the strains YI005, YI006, YI012, YI020, YI021, YI022, YI023 and YI024 were measured using the EROD assay protocol, as described in Example 3.4.

### Results

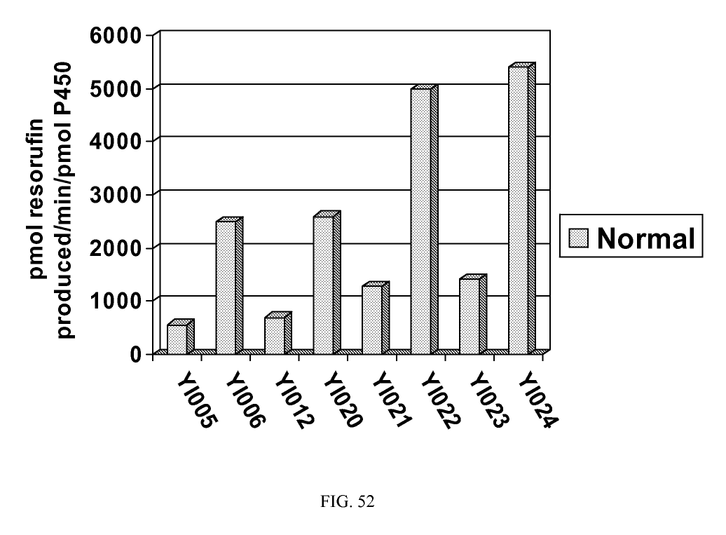

The values depicted in FIG. 52 are an average of at least 3 individual experiments. The cells were grown by the “normal” method used for “Growing yeast cultures for microsome preparation” as in Example 1.15 and the microsomes were also prepared by the same method as in Example 1.15.

### Conclusion

(1) There appears to be a direct correlation between P450 reductase activities and CYP1B1 P450 activities, as measured via the EROD fluorescence-based assay.
(2) The ability to generate different P450 reductase activities in yeast will allow the production of recombinant P450-s with a variety of different catalytic activities.

### Example 6

### Construction of Protease Deficient Yeast Strains for Improved Expression of CYP Isozymes

### Example 6.1

### Construction of TRP1 Bearing Plasmid

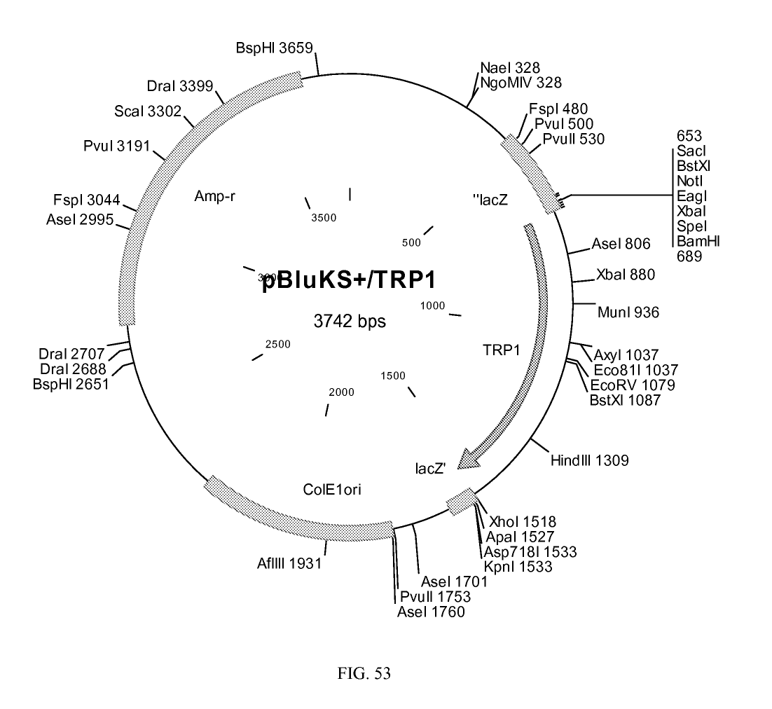

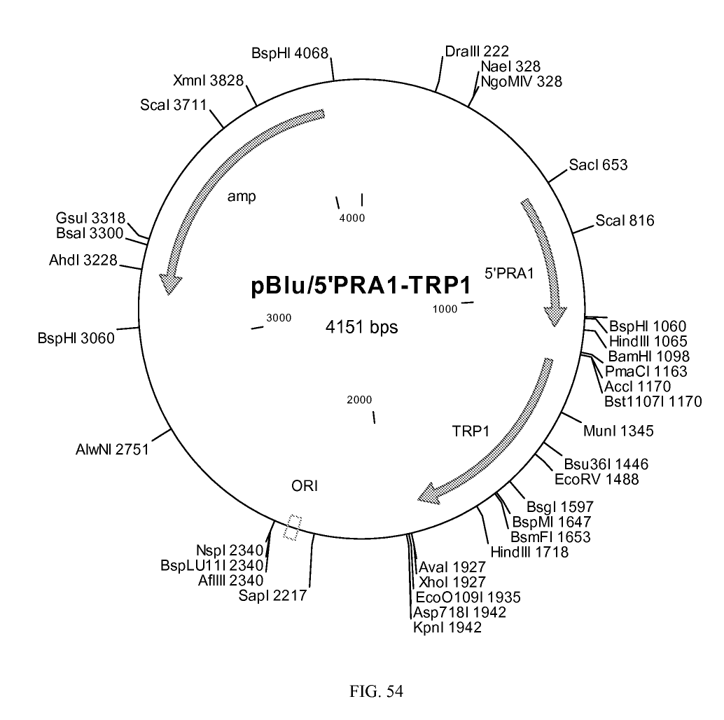

A 829 bp BamHI-XhoI fragment of the S. cerevisiae TRP1 gene (SEQ ID No. 47) was cloned, using two PCR primers (5′ PCR primer: 5′-CGggatccAA TTCGGTCGAA AAAAGAAAAG GAGAGGGCCA AGAGGG-3′ (letters in lower casing represent the BamHI site—SEQ ID NO: 48) & 3′ PCR primer: 5′-CCGctcgagG GCAAGTGCAC AAACAATACT TAAATAAATA CTACTC-3′ (letters in lower casing represent the XhoI site—SEQ ID NO: 48)) in the basic pBlueScriptKS+ to obtain the plasmid pBluKS+/TRP1 (FIG. 53).

### Example 6.2

### Construction of the Plasmid that would Allow PRA1 (Pep4) Deletion in a Yeast Strain Through Homologous Recombination

In order to delete the PRA1 gene (SEQ ID No 50) from its chromosomal locus, a disruption plasmid was constructed in 2 steps.

First, a SacI-BamHI fragment of the 5′ end of the PRA1 gene (520 bp, SEQ ID No 51) was isolated by PCR (using 5′ PCR primer: 5′-GC GAGCTC ATG TTC AGC TTG AAA GCA TTA TTG CCA TTG GCC TTG 3′ (the letters in italics signify the SacI site—SEQ ID NO: 52) & 3′ PCR primer: 5′-CG GGATCC CAG TAC CAT TAG CTT TGT AGC TTG ATG 3′ (the letters in italics signify the BamHI site—SEQ ID NO: 53)) and sub-cloned in the plasmid pBluKS+/TRP1 (FIG. 53) to obtain the plasmid pBlu/5′PRA1-TRP1 (FIG. 54).

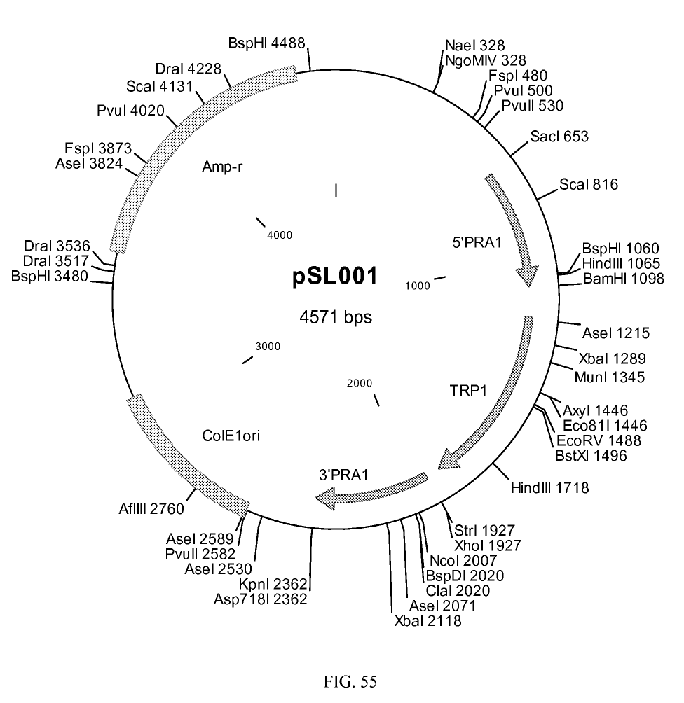

In the second step, an XhoI-KpnI fragment of the 3′ end of the PRA41 gene (446 bp, SEQ ID No 54) was isolated by PCR (using 5′ PCR primer: 5′-CCG CTCGAG GTT CGT CGT AAG GCT TAC TGG GAA GTC AAG TTT G-3′ (the letters in italics signify the XhoI site—SEQ ID No 55) & 3′ PCR primer: 5′-GG GGTACC TCA AAT TGC TTT GGC CAA ACC AAC CGC ATT GTT GCC C-3′ (the letters in italics signify the KpnI site—SEQ ID No 56)) and sub-cloned in the plasmid pBlu/5′PRA1-TRP1 (FIG. 54) to obtain the gene disruption plasmid pSLOO1 (FIG. 55) that would allow disruption of the PRA1 gene with a functional TRP1.

### Example 6.2

### Construction of the Plasmid that would Allow HRD1 Deletion in a Yeast Strain Through Homologous Recombination

In order to delete the HRD1 gene (SEQ ID No 57) from its chromosomal locus, a disruption plasmid was constructed in 2 steps.

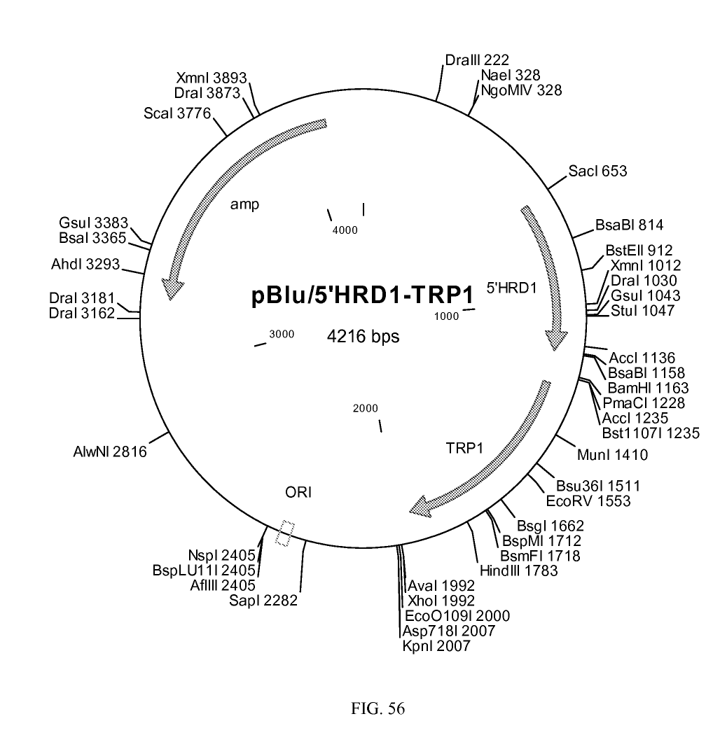

First, a SacI-BamHI fragment of the 5′ end of the HRD1 gene (520 bp, SEQ ID No 58) was isolated by PCR (using 5′ PCR primer: 5′-GC GAGCTC ATG GYG CCA GAA AAT AGA AGG AAA CAG TTG GC-3′ (the letters in italics signify the SacI site—SEQ ID No 59) & 3′ PCR primer: 5′-CG GGATCC GCA TCG TGT TAT TAT CTG GTA GTC TAC AAC CGC C-3′ (the letters in italics signify the BamHI site —SEQ ID No 60)) and sub-cloned in the plasmid pBluKS+/TRP1 (FIG. 53) to obtain the plasmid pBlu/5′HRD1-TRP1 (FIG. 56).

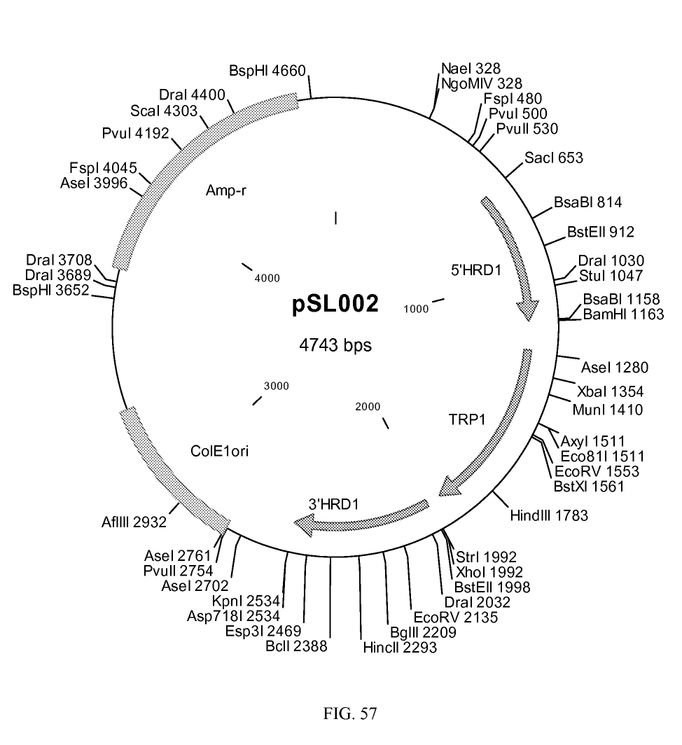

In the second step, anXhoI-KpnI fragment of the 3′ end of the HRD1 gene (553 bp, SEQ ID No 61) was isolated by PCR (using 5′ PCR primer: 5′-CCG CTCGAG GGT TAC CTT GTG GCC ACA TAC TTC ATT TGT CG-3′ (the letters in italics signify the XhoI site—SEQ ID No 62) & 3′ PCR primer: 5′-GG GGTACC CTA GAT ATG CTG GAT AAA TTT ATC TGG TAT GAC-3′ (the letters in italics signify the KpnI site—SEQ ID No 63)) and sub-cloned in the plasmid pBlu/5′HRD1-TRP1 (FIG. 54) to obtain the gene disruption plasmid pSLOO2 (FIG. 57) that would allow disruption of the HRD1 gene with a functional TRP1.

### Example 6.4

### Construction of the Plasmid that would Allow HRD2 Deletion in a Yeast Strain Through Homologous Recombination

In order to delete the HRD2 gene (SEQ ID No 64) from its chromosomal locus, a disruption plasmid was constructed in 2 steps.

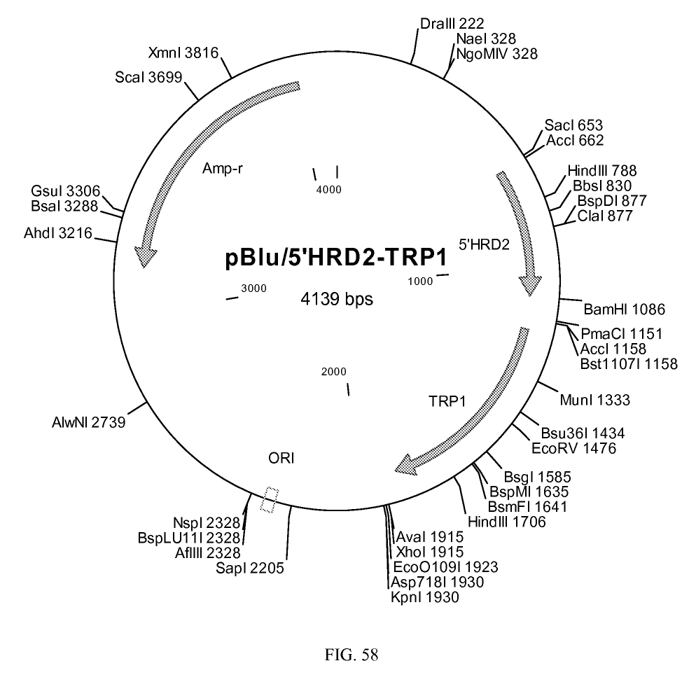

First, a SacI-BamHI fragment of the 5′ end of the HRD2 gene (443 bp, SEQ ID No 65) was isolated by PCR (using 5′ PCR primer: 5′-GC GAGCTC ATG GTA GAC GAA AGT GAT AAG AAA CAA CAG AC-3′ (the letters in italics signify the SacI site—SEQ ID No 66) & 3′ PCR primer: 5′-CG GGATCC CGT CGG AGA GTA ATC TAT ATC TCA ATG AAT CGT G-3′ (the letters in italics signify the BamHI site —SEQ ID No 67)) and sub-cloned in the plasmid pBluKS+/TRP1 (FIG. 53) to obtain the plasmid pBlu/5′HRD2-TRP1 (FIG. 58).

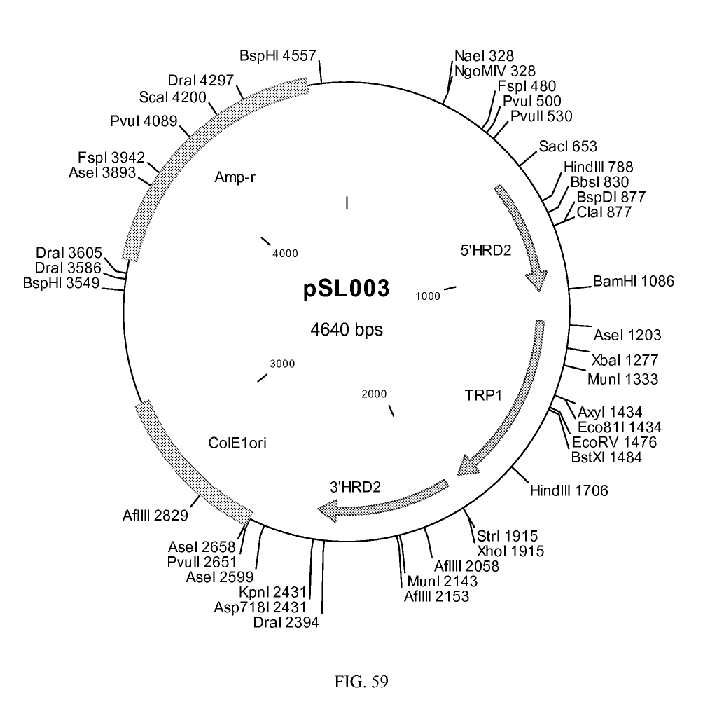

In the second step, an XhoI-KpnI fragment of the 3′ end of the HRD2 gene (526 bp, SEQ ID No 68) was isolated by PCR (using 5′ PCR primer: 5′-CCG CTCGAG GCA AGG TTA GCT CAA CTA TTA AGA CAG TTG GC-3′ (the letters in italics signify the XhoI site—SEQ ID No 69) & 3′ PCR primer: 5′-GG GGTACC TTA CTC CTC TTC ACG ATA GTC AGG GTT CTT C-3′ (the letters in italics signify the KpnI site—SEQ ID No 70)) and sub-cloned in the plasmid pBlu/5′HRD2-TRP1 (FIG. 54) to obtain the gene disruption plasmid pSLOO3 (FIG. 59) that would allow disruption of the HRD2 gene with a functional TRP1.

### Example 6.5

### Construction of the Plasmid that would Allow UBC7 Deletion in a Yeast Strain Through Homologous Recombination

In order to delete the UBC7 gene (SEQ ID No 71) from its chromosomal locus, a disruption plasmid was constructed in 2 steps.

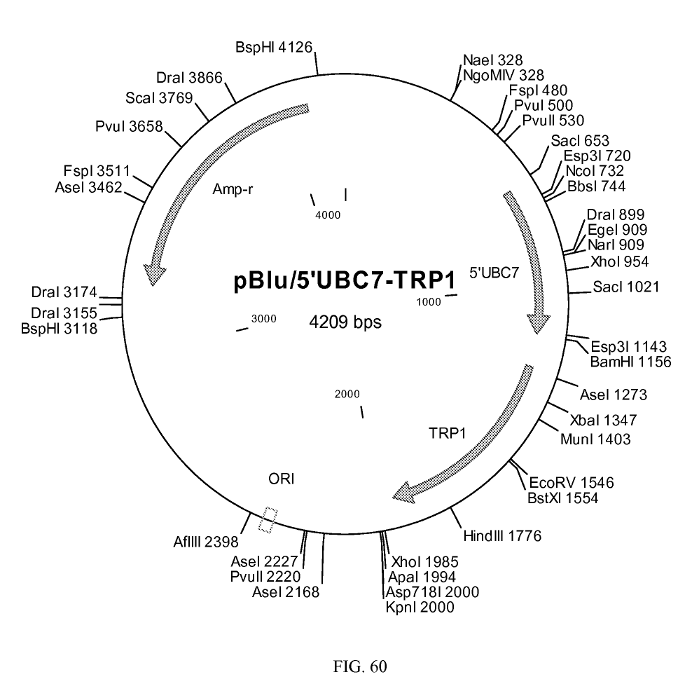

First, a SacI-BamHI fragment of the 5′ end of the UBC7 gene (520 bp, SEQ ID No 72) was isolated by PCR (using 5′ PCR primer: 5′-GC GA GCTC CCT TCA ATT TGT GCA CCA TTT TCG TAT TCT G-3′ (the letters in italics signify the SacI site—SEQ ID NO: 73) & 3′ PCR primer: 5′-CG GGATCC CCT TGA GGA GAC GTT TCT GAG CGG TTT TCG ACA T-3′ (the letters in italics signify the BamHI site—SEQ ID No 74)) and sub-cloned in the plasmid pBluKS+/TRP1 (FIG. 53) to obtain the plasmid pBlu/5′UBC7-TRP1 (FIG. 60).

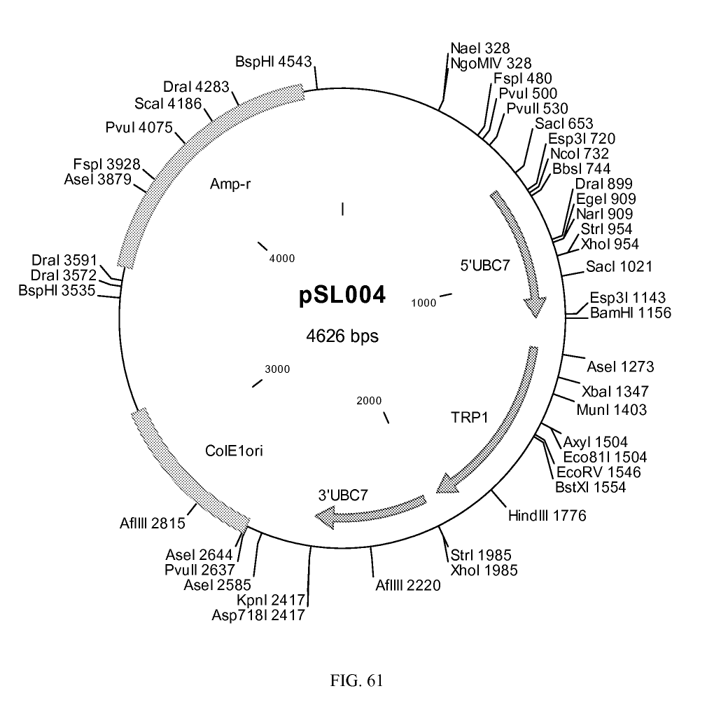

In the second step, an XhoI-KpnI fragment of the 3′ end of the UBC7 gene (553 bp, SEQ ID No 75) was isolated by PCR (using 5′ PCR primer: 5′-CCG CTCGAG GTG GCT GGT CCC AAA TCG GAG AAT AACATA TTC-3′ (the letters in italics signify the XhoI site—SEQ ID NO: 76) & 3′ PCR primer: 5′-GG GGTACC TCA GAA TCC TAA TGA TTT CAA AAT GGA TAA CTT TAC CTG TCT CTC-3′ (the letters in italics signify the KpnI site—SEQ ID No 77)) and sub-cloned in the plasmid pBlu/5′UBC7-TRP1 (FIG. 60) to obtain the gene disruption plasmid pSL004 (FIG. 61) that would allow disruption of the UBC7 gene with a functional TRP1.

### Example 6.6

### Construction of Yeast Strains Deficient in the PEP4, HRDJ, HRD2 and UBC7 Genes by Gene Disruption

The plasmids pSL001, pSL002, pSL003 and pSL004 (see Examples 6.2 to 6.5) were used for disruption of PEP4, HRDJ, HRD2 and UBC7 genes employing the one-step gene disruption method.

Purified fragments of Jpral/TRP1, Jhrd1/TRP1, Jhrd2/TRP1 and Jubc7/TRP1 (using a QIAGEN kit) were isolated from pSL001, pSL002, pSL003 and pSL004 by digesting the plasmids with SacI and KpnI restriction enzymes. The fragments were integrated into the yeast chromosomes through homologous recombination using a high efficiency yeast transformation method. The integrants were selected on minimal medium SD plates that contain requisite nutrients but lack tryptophan.

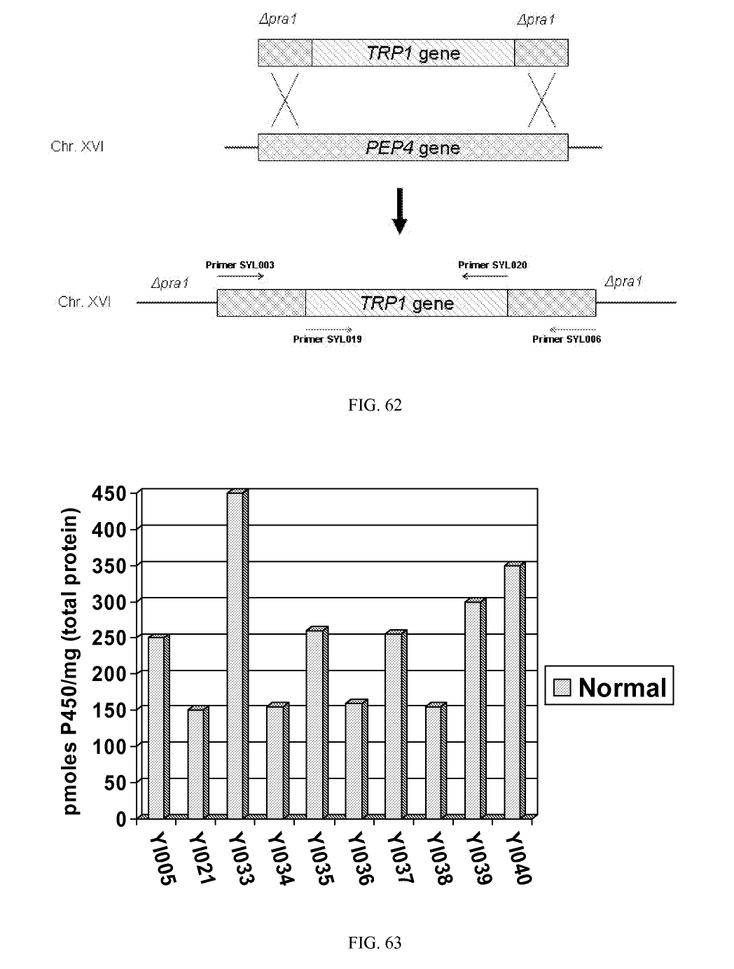

FIG. 62 depicts the general strategy used for gene disruption, using protocol used for disruption of the PEP4 gene as an example. The target gene (PEP4) has been interrupted by a DNA fragment containing the selectable marker gene (TRP1) and the DNA (Δpra1) flanking each side of the selectable marker. Recombination between the ends of this DNA fragment replaces the target gene with the disrupted gene sequence. A successful gene disruption is verified by PCR using a 5′-primer used to amplify a 5′-end fragment of any of the four genes and the 3′-TRP1 primer (SEQ ID No 49), or alternatively a 5′-TRP1 primer (SEQ ID No 48) and a 3′-primer used to amplify a 3′-end fragment of any of the four genes.

The Trains

(1) YI001 (bearing a copy of ΔN24hRDStop at the LEU2 locus), and
(2) YI016 (bearing 2 copies of ΔN24hRDStop at the LEU2 locus) were used for disruption of PEP4, HRDJ, HRD2 and UBC7 genes. The resultant strains were named:
(i) YI025 (YI101, pra1::TRP1),
(ii) YI026 (YI16, pra1::TRP1),
(iii) YI027 (YI101, hrd1::TRP1),
(iv) YI028 (YI16, hrd1::TRP1),
(v) YI029 (YI101, hrd2::TRP1),
(vi) YI030 (YI16, hrd2::TRP1),
(vii) YI031 (YI101, ubc7::TRP1),
(viii) YI032 (YI16, ubc7::TRP1).

### Example 6.7

### Transformation of pSYE225 in the Protease Deficient Strains Described in Example 6.6

The yeast strains, constructed in Example 6.6, were transformed with the 2-micron yeast expression plasmid pSYE225 (Example 14), that bears the CYP1B1 gene, to obtain the resultant strains:

(1) YI033 (YI025:: pSYE225),
(2) YI034 (YI026:: pSYE225),
(3) YI035 (YI027:: pSYE225),
(4) YI036 (YI028:: pSYE225),
(5) YI037 (YI029:: pSYE225),
(6) YI038 (YI030:: pSYE225),
(7) YI039 (YI031:: pSYE225),
(8) YI040 (YI032:: pSYE225).

### Example 6.8

### Comparison of P450 Amounts in Strains, Described in Example 6.5, Via CO-Difference Spectroscopy

P450 amounts in the microsomes obtained from the strains YI005 (YI001::pSYE225), YI021 (YI016::pSYE225), YI033, YI034, YI035, YI036, YI037, YI038, YI039 and YI040 were measured using the protocol “Determination of P450 amounts via CO-difference spectroscopy), as described in Example 1.15.

### Results

The values depicted in FIG. 63 are an average of at least 3 individual experiments. The cells were grown by the “normal” method used for “Growing yeast cultures for microsome preparation” as in Example 1.15 and the microsomes were also prepared by the same method as in Example 1.15.

### Conclusions

(1) There is ˜80% increase in CYP1B1 amounts in the strain YI033 that contains a copy of ΔN24hRDStop but lacks the PRA1 gene, as compared with the strain YI005 that contains a copy of ΔN24hRDStop but also has the PRA1 gene present.
(2) There is a 2-fold increase in CYP1B1 amounts in the strain YI040 that contains two copies of ΔN24hRDStop but lacks the UBC7 gene, as compared with the strain YI021 that contains two copies of ΔN24hRDStop but also has the UBC7 gene present. CYP1B1 produced in YI021 is about twice as active than the CYP1B1 produced in YI005, presumably because 2 copies of ΔN24hRD provides more reductase activity than a single copy of ΔN24hRD. However, YI021 produces much less P450 protein than YI005. These protease deletion strains are therefore very useful in the production of highly active P450 isozymes from the strains that co-express ΔN24hRD-cmyc fusion which is much more potent in reductase activity than ΔN24hRD (see Example 5).
(3) The protease deletion strains could be also very useful in strains that co-express the yeast P450 reductase (yRD) and P450 isozymes. The yRD enzyme is very much more potent than hRD and its variants. However, the P450 amounts produced in these strains is minimal (although very highly active). This leads us to believe that the protease deficient strains would again be very useful during co-expression of yRD and P450 isozymes.

### Example 7

### Construction of Yeast Strains that Bear yRD at Different Chromosomal Loci: Comparative Analysis of Yrd Activities and Relationship with CYP Activities

### Example 7.1

### Cloning of the Endogenous Yeast P450 Reductase (yRD) Gene from S. cerevisiae Genomic Library in the Basic Plasmid pSP73

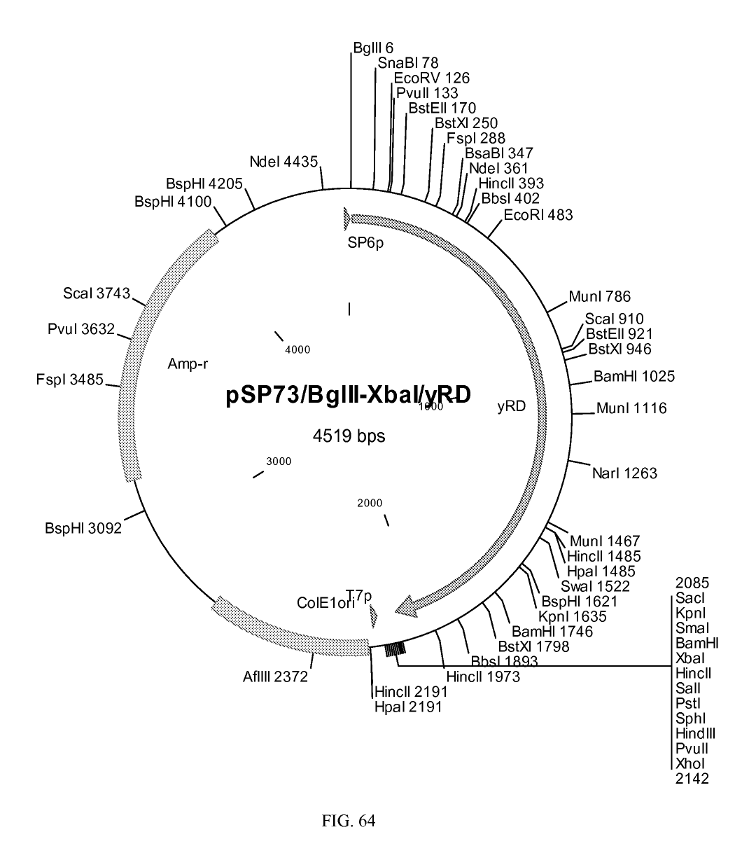

The 2076 bp yeast P450 reductase (hRD) gene (SEQ ID No. 78), that encodes the full-length protein, was amplified as a BglII-XbaI fragment. 100 pmoles each of the PCR primers (5′ PCR primer: 5′-ATagatctATGCCGTTTGGAATAGACAACACCG-3′ (letters in lower casing represent the BglII site—SEQ ID NO: 79) & 3′ PCR primer: 5′-ATtctagaTTACCAGACATCTTCTTGGTATCTACCTGAAG-3′ (letters in lower casing represent the XbaI site—SEQ ID NO: 80)) were used with DNA (500 ng) from a yeast genomic library, derived from the strain S288C, as template. The yRD gene was subcloned in the basic plasmid pSP73 (purchased from Promega) to obtain the plasmid pSP73/BglII-XbaI/yRD (FIG. 64). The sequence of the insert was confirmed by DNA sequencing.

### Example 7.2

### Deletion of the yRD Gene in a Yeast Strain

### Step 1

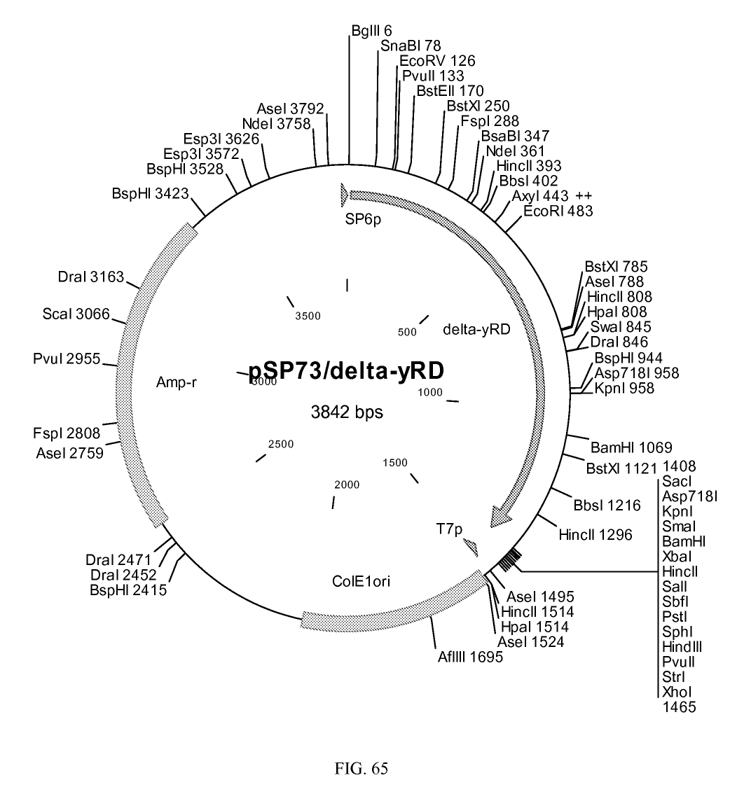

The plasmid pSP73/BglII-XbaI/yRD (FIG. 64) was digested with the MunI restriction enzyme. This deletes 682 bp of the coding sequence of yRD. The 5′ and 3′-ends were flushed with Klenow polymerase and the plasmid was relegated. The resultant plasmid is named pSP73/delta-yRD (FIG. 65).

### Step 2: Construction of the yRD Gene Disruption Plasmid

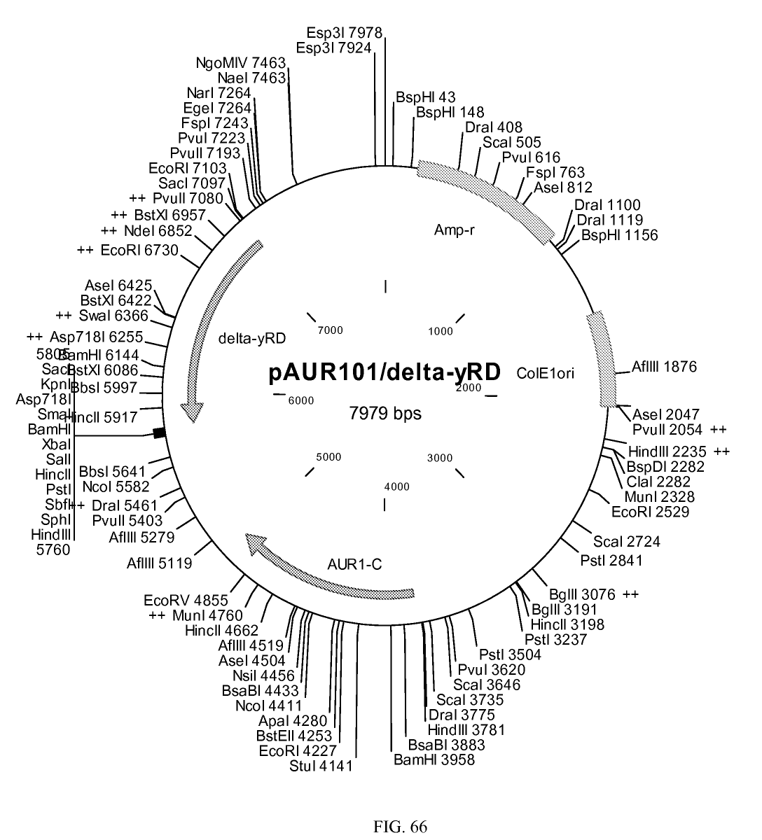

An EcoRV-SalI fragment from ΔyRD was isolated from pSP73/delta-yRD (FIG. 65) and was inserted into pAUR10 (purchased from TakaRa) digested with SmaI and SalI restriction enzymes to obtain a new plasmid pAUR101/delta-yRD (FIG. 66).

Step 3: Integration of Linearised pAUR/delta-yRD for yRD Gene Disruption

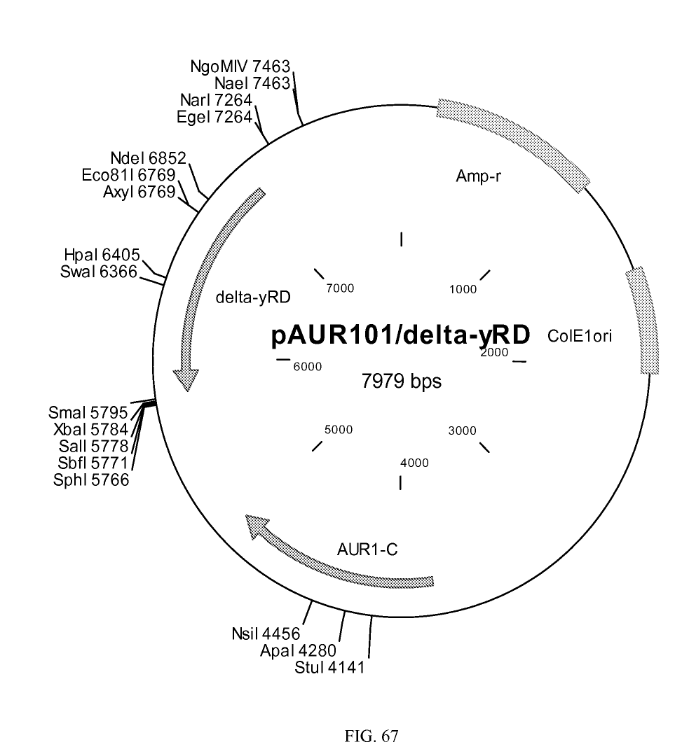

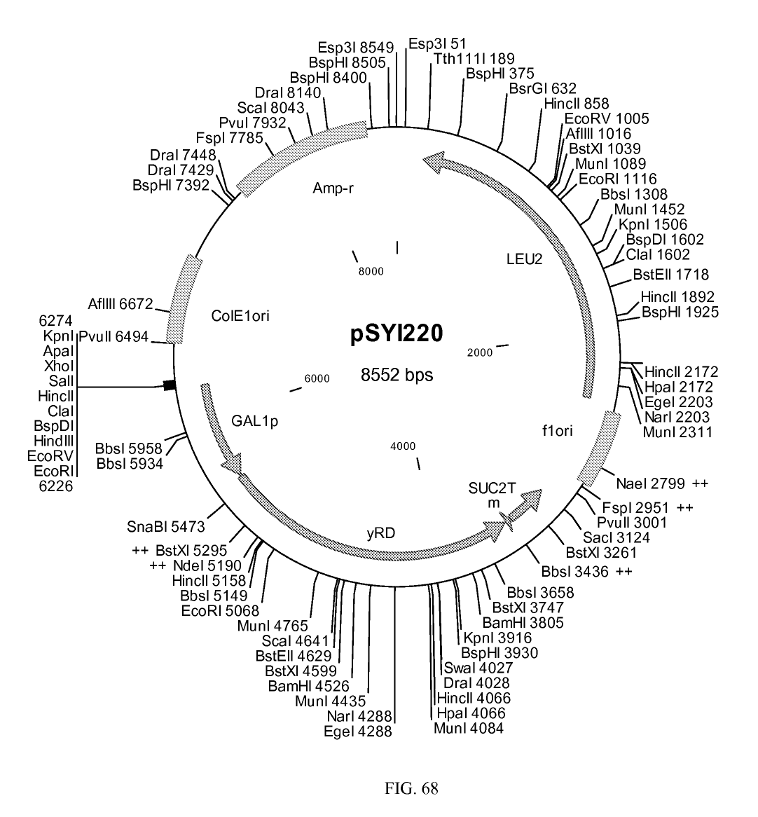

The plasmid pAUR101/delta-yRD (FIG. 66) was digested with SwaI, a restriction site that occurs roughly in the middle of the delta-yRD fragment. SwaI is also a unique restriction site in the plasmid pAUR101/delta-yRD (FIG. 67). SwaI digested DNA linearises pAUR101/delta-yRD and allows homologous recombination in the yeast strain BC300 at the yRD chromosomal locus, resulting in the disruption of the endogenous yRD gene. The cells are selected via screening of aureobasidine resistance. Yeast is extremely sensitive to this antibiotic.

The delta-yRD gene has the sequence of SEQ ID No. 81.

The integrants were selected on SD plates containing 0.5 μg/μl of the antibiotic aureobasidine. To confirm the disruption of the yRD gene, PCR analysis was performed using primers (SEQ ID Nos. 82 & 83) designed to amplify the delta-yRD gene. The DNA amplified from the wild type strain BC300, with intactyRD, has a larger size (+650 bp) than the yRD obtained from the strain where yRD has been disrupted using the disruption plasmid, pAUR101/ΔyRD (FIG. 67). The observed 1.3 kb band was consistent with the expected size of delta-yRD. The resultant strain was named YI041 (BC300, yrd::Aur⁺).

### Step 4: Measurement of P450 Reductase Activity Using the MTT Assay

Reductase activities in the yeast microsomes (Example 1.15) obtained the strain YI041 was measured using the protocol described in Example 2.13. Within the experimental error, the reductase activity was zero.

### Example 7.3

### Construction of Plasmids that would Allow Integration of yRD Expression Cassettes at Different Chromosomal Loci of YI041, a Yeast Strain that Totally Lacks yRD Activity

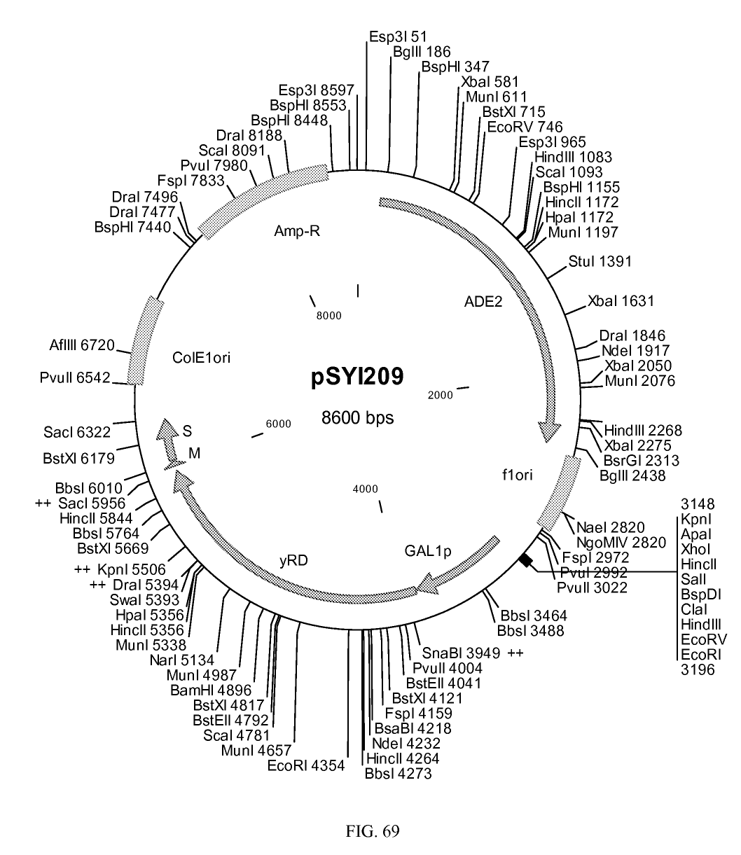

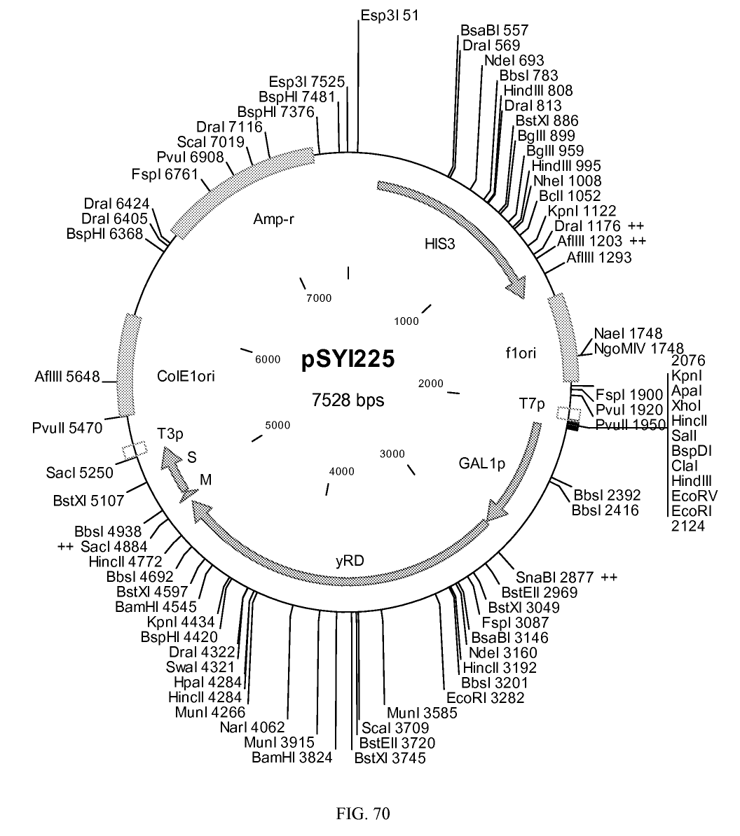

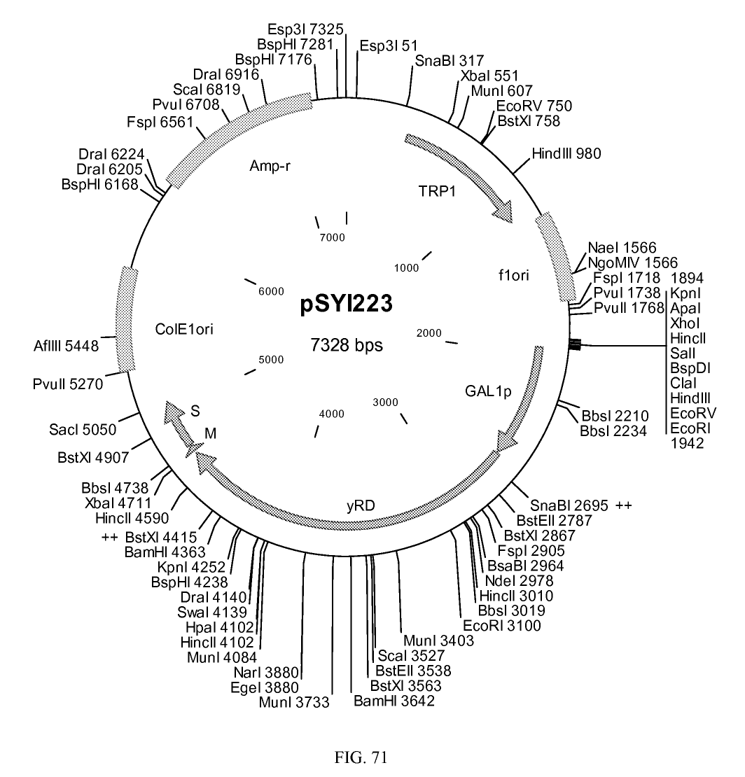

BamHI-XbaI digested plasmids YILeuGAL1MS (FIG. 23), YIAdeGAL1MS (FIG. 24), YIHisGAL1MS (FIG. 25) and YITrpGAL1MS (FIG. 26) were used for cloning the BglII-XbaI fragment of the yeast P450 reductase gene, yRD, obtained from pSP73/BglII-XbaI/yRD (FIG. 64). The resultant plasmids were named pSYI220 (FIG. 68), pSYI209 (FIG. 69), pSYI225 (FIG. 70) and pSYI223 (FIG. 71).

### Example 7.4

### Integration of yRD Expression Cassettes at Different Chromosomal Loci in the Yeast Strain YI041

The plasmids pSYI220 (FIG. 68), pSYI209 (FIG. 69), pSYI225 (FIG. 70) and pSYI223 (FIG. 71) were linearised with the following restriction enzymes AflII (for pSYI220), StuI (for pSYI209), NheI (for pSYI225) and NarI (for pSYI223) in order to integrate into the yeast strain YI041 that lacks all endogenous yRD activity. Linearised pSYI220 integrates at the LEU2 locus, pSYI209 at the ADE2 locus and pSYI225 at the HIS3 locus. The plasmid pSYI223 is used to integrate at the yRD locus of a functional yRD gene driven by our GAL1-675 promoter.

(1) YI042 (pSYI041, yRD⁻, GAL1p675-YRD::LEU2⁺),
(2) YI043 (pSYI041, yRD⁻, GAL1p675-YRD::ADE2⁺),
(3) YI044 (pSYI041, yRD⁻, GAL1p675-YRD::HIS3⁺),
(4) YI045 (pSYI041, yRD⁻, GAL1p675-YRD⁺, TRP1⁺).

### Example 7.5

### Comparison of P450 Reductase Activities in Yeast Strains Containing GALJp-675 Promoter yRD

Microsomes were prepared from the strains YI042, YI043, YI044, YI044 and the control strains YI016 (bearing 2 copies of ΔN24hRDStop at the LEU2 locus) and YI017 (bearing 2 copies of ΔN24hRD-cmyc at the LEU2 locus) as in Example 1.15.

Reductase activities in the microsomes were measured using the protocol described in Example 2.13.

### Results

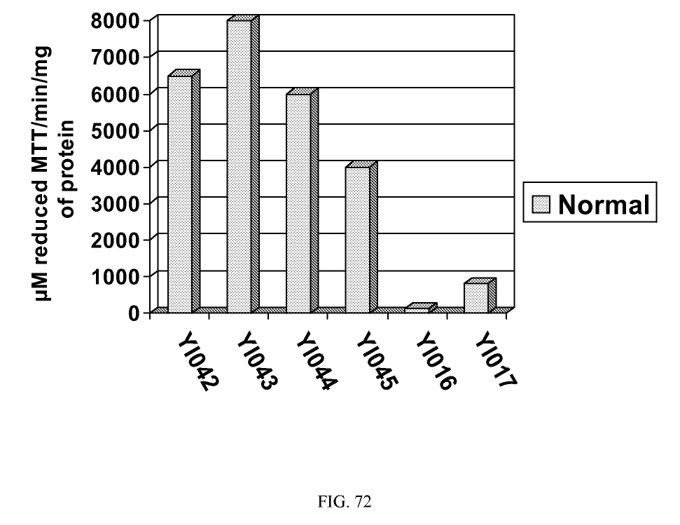

The reductase activity values depicted in FIG. 72 are an average of at least 3 individual experiments.

### Conclusion

(1) The “yeast” P450 reductase (yRD) activity expressed at different chromosomal loci is extraordinarily high compared to the strain YI016 and YI017 which bear the two “human” P450 reductase (hRD) variants, ΔN24hRD and the ΔN24hRD-cmyc fusion protein.
(2) GAL1p-675 promoter driven yRD activity differs depending on the locus. Recombinant yRD expressed at the yeast strain's yRD locus gives the least activity.
(3) There is therefore the possibility of providing a variety of P450 expression systems which have an array of reductase activities.

### Example 7.6

### Transformation of pSYE225, a 2-Micron Plasmid that Encodes the Human CYP1B1 Gene, into Yeast Strains Described in Example 7.4

The resultant strains are:

(1) YI046 (pSYI042::pSYE225),
(2) YI047 (pSYI043::pSYE225),
(3) YI048 (pSYI044::pSYE225),
(4) YI049 (pSYI045::pSYE225).

### Example 7.6

### Comparison of P450 Amounts and Activities Obtained from Strains in Example 7.6

P450 amounts in the microsomes from the strains YI046, YI047, YI048, YI049 were measured using the protocol “Determination of P450 amounts via CO-difference spectroscopy), as described in Example 1.15.

CYP1B1 P450 activities in the microsomes (prepared under normal growth conditions, as described in Example 1.15) obtained from the strains YI046, YI047, YI048, YI049 were measured using the EROD assay, as described in Example 3.4.

### Results

Interestingly, there was no measurable P450 protein in any of these strains but the activity measured by the EROD assay was very high compared to the other strains where P450 amounts could be measured.

### Conclusion

The results can only imply that very high reductase activities prevent measurable P450-s to be formed. Although CO-difference spectroscopy may not be sensitive enough to measure minute amounts of P450 produced, these small amounts have extraordinary high activity due to very high reductase activities in the cell.

The protease deficient strains (as described in Example 6) can be used in order to circumvent this conundrum.

### Example 8

### Construction of a Yeast 2-Micron Plasmid that Contains the LEU2 Auxotrophic Marker and the Gal1p-675 Promoter

### Rationale

To allow co-expression of two P450 genes from the same yeast cell, one of them being borne by a URA3-based 2-micron yeast shuttle vector (i.e. pSYE225) and the other by a LEU2-based 2-micron yeast shuttle vector.

### Example 8.1

### Replacement of the URA3 Gene in pYES2 Plasmid (Invitrogen) with the S. cerevisiae LEU2 Gene

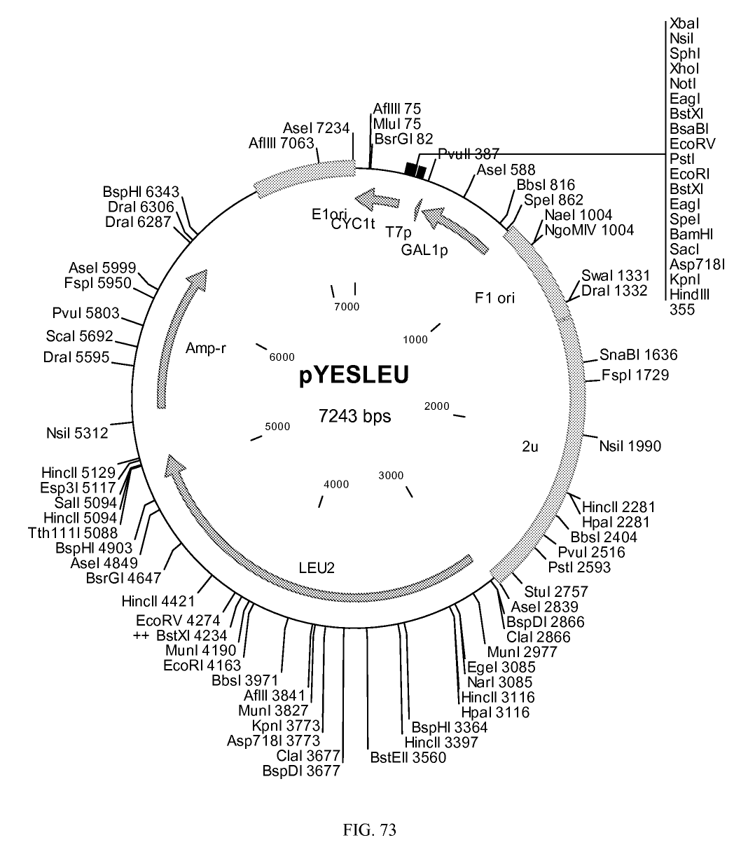

The plasmid pYES2 (purchased from Invitrogen) was digested with Esp31 and NheI, the ends were flushed with Klenow polymerase to obtain blunt ends. The large vector fragment was isolated. The yeast shuttle vector Yep13 (purchased from ATCC) was digested with XhoI, flushed with Klenow polymerase to obtain a blunt end, followed by digestion with ScaI (which produces a blunt end). The 2220 bp LEU2 was ligated to the large vector fragment obtained from pYES2. A plasmid was selected via diagnostic restriction enzyme digests that had the LEU2 gene in the same orientation as the beta-lactamase gene (Amp-r; used for selection in E. coli). This plasmid was named pYESLEU (FIG. 73).

### Example 8.2

### Replacement of the 450 bp GAL1 Promoter Present in the pYES2 Plasmid (Invitrogen) by 675 bp GAL1 Promoter

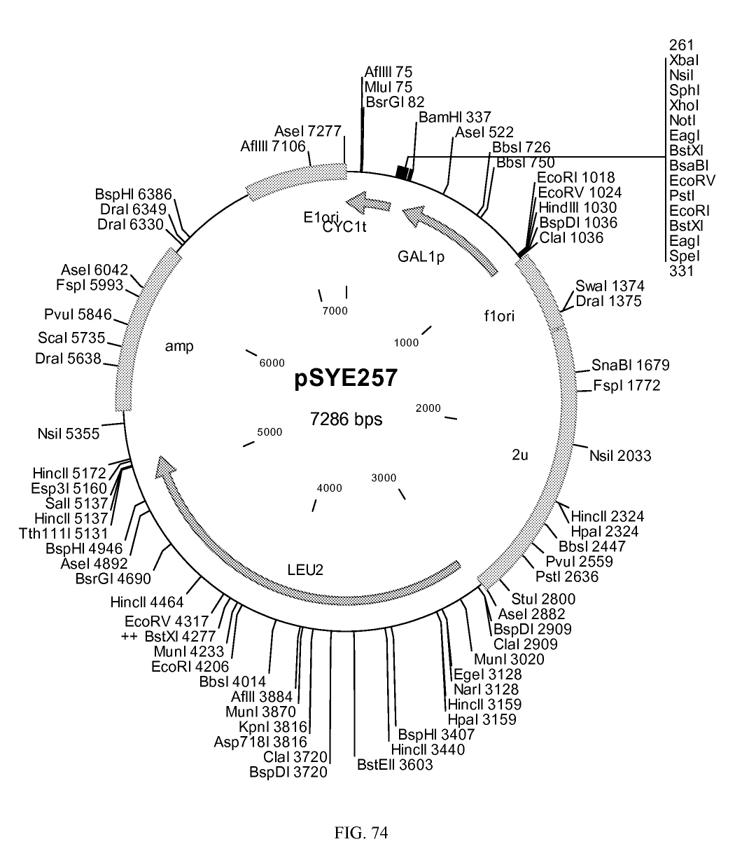

pYESLEU was digested NgoMIV, the end was flushed, followed by digestion with BamHI. The large vector fragment was isolated. The ˜675 bp HincII-BamHI fragment of the GAL1 promoter fragment was isolated from the plasmid pBluSK/Ngo-Bam/Gal1p-675 (FIG. 3; Example 1.4) after digestion with HincII (creates blunt end) and BamHI. The vector and the promoter fragment were ligated to result in the plasmid pSYE257 (FIG. 74) that is a 2-micron, LEU2-based yeast shuttle vector. The vector contains the BamHI, SpeI, XbaI restriction sites that lie downstream of the promoter and upstream of the terminator.

### Example 9

### Expression of the b5 Protein Using the GAL1p-675 Promoter

Like the P450 reductase, the b5 protein is another co-factor that contributes to P450 activity.

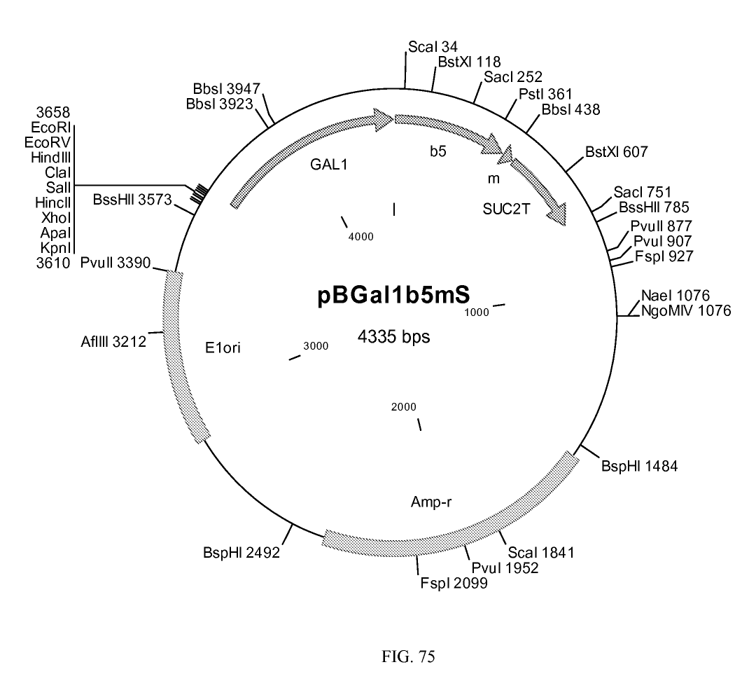

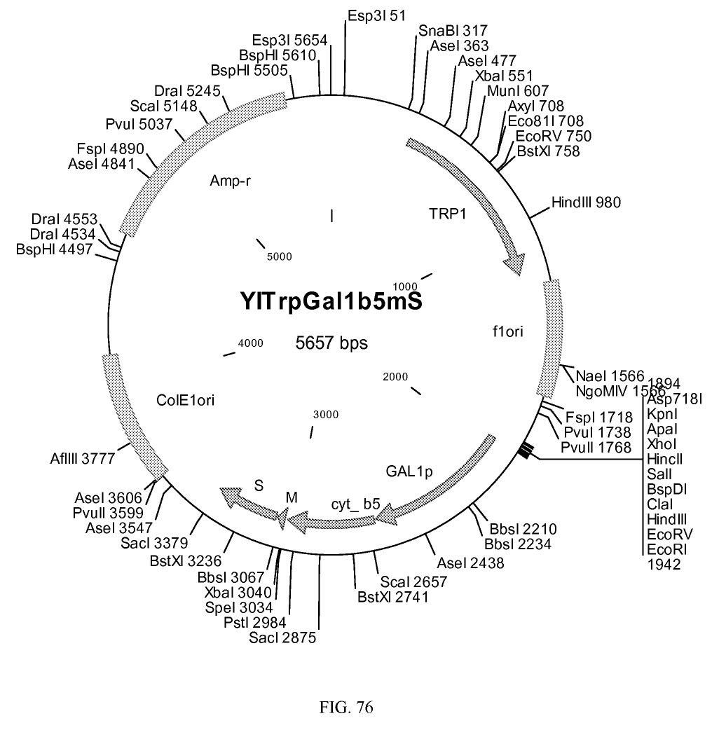

The human cytochrome b5 was cloned as a BglII-SpeI fragment (SEQ ID No. 86) using two PCR primers (5′ PCR primer: 5′-GCagatactAT GGCAGAGCAG TCGGACGAGG CCGTG-3′ (letters in lower casing represent the BglII site—SEQ ID No 87) & 3′ PCR primer: 5′-CGactagtTC AGTCCTCTGC CATGTATAGG CGATACATC-3′ (letters in lower casing represent the SpeI site—SEQ ID No 88)) into the vectors pBluKS(+)/Gal1mS (FIG. 22) and YITrpGal1mS (FIG. 26) to obtain the plasmids pBGal1b5 mS (FIG. 75) and YITrpGal1b5 mS (FIG. 76).

The plasmid pBGal1b5 mS (FIG. 75) was further used to construct b5 expression plasmids (see below) whereas YITrpGal1b5 mS was directly used for integration into yeast at the TRP1 locus.

Three other plasmids were constructed for expression of human b5.

Plasmid 1: A 2-Micron Plasmid that could be Used for Expressing Human B5 and Any P450 of Choice (Between the Bam HI-Xba/Spe Sites)

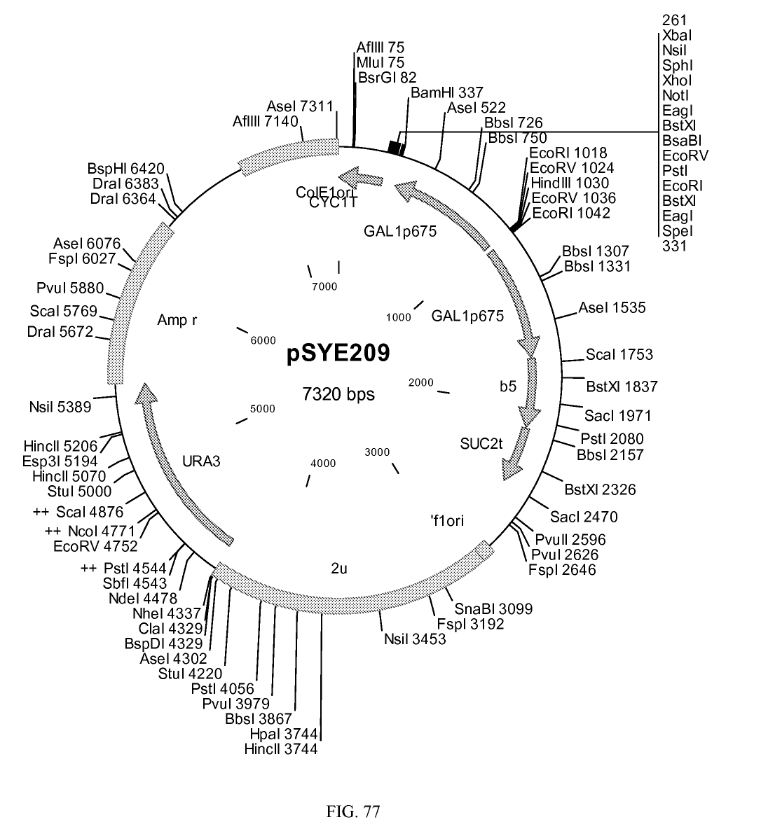

A 5553 bp HindIII-SwaI fragment from pSYE224 (FIG. 4) was ligated to a 1767 bp fragment of Gal1pb5 mS (FIG. 75) to obtain the plasmid pSYE209 (FIG. 77).

Plasmid 2: An integrating Plasmid Based on pAUR101 Which Would Allow Selection in Aureobasidin (Antibiotic)

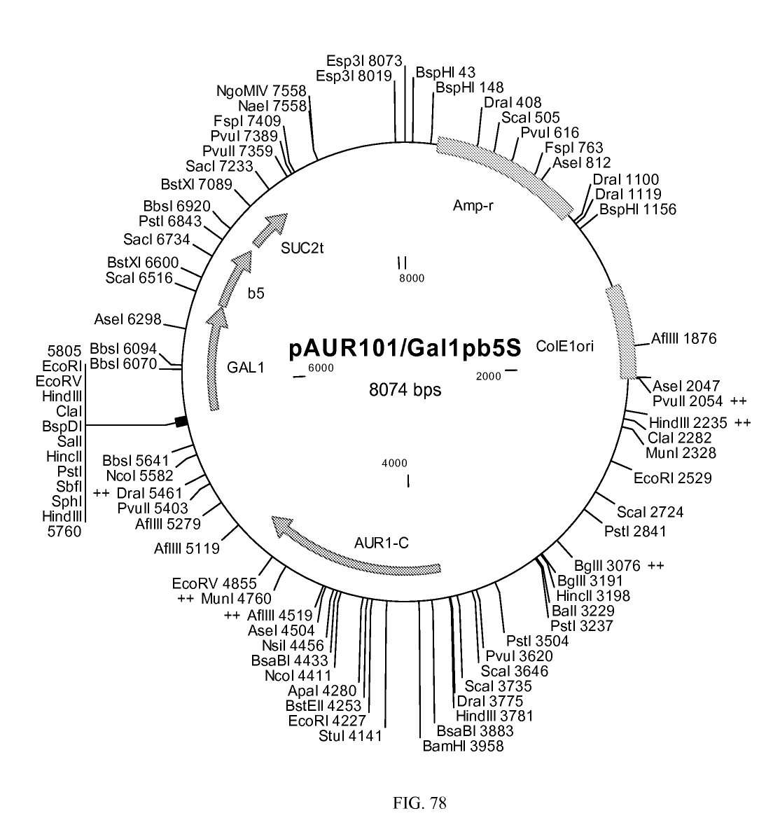

A 6292 bp SaII-NaeI fragment from pAUR101 (TakaRa) was ligated to a 1782 bp SaII-NaeI fragment from pBGal1pb5 mS (FIG. 75) to obtain the plasmid pAUR101/Gal1pb5S (FIG. 78).

Plasmid 3: An Integrating Plasmid Based on pAUR135 which would Allow Initial selection in aureobasidin (antibiotic) but later the selection marker can be removed through intra-plasmid homologous recombination so that yeast cells could be further used to select on aureobasidin medium

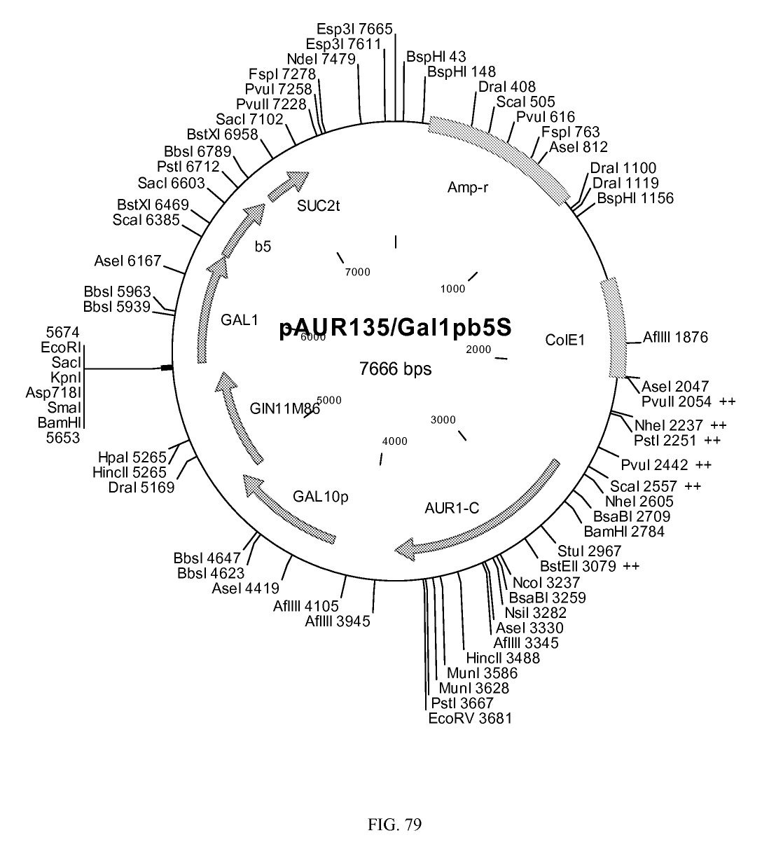

A 5911 bp EcoRI-EgeI fragment from pAUR135 (TakaRa) was ligated to a 1755 bp EcoRI-NaeI fragment from pBGal1pb5 mS (FIG. 75) to obtain the plasmid pAUR135/Gal1pb5S (FIG. 79).

### Example 10

### Combining Different Cells that Individually Produce a CYP, a Reductase or the b5 Protein Before Microsome Preparation for Production of CYP-s with High Levels of Specific Activity

The P450 reductases are deleterious to any eukaryotic cell (yeast, human or insect cell). Human P450 reductase (hRD) variants have been created in order to circumvent the problem of latent toxicity of hRD. We find that, as a general rule, specific activity of any cytochrome P450 (CYP) is inversely proportional to the “amounts” of CYP made in the yeast cell which co-expresses (i.e. co-produces) a CYP and a reductase.

The inventors have tried to solve this conundrum by expressing in distinct cell cultures a CYP of choice, a reductase or the b5 protein. The two or three types of cells that contain CYP, reductase or the b5 protein are combined during cell harvest and then microsomes are prepared from the cell mixture. One can conjecture that during microsome preparation, there would be some likelihood of membrane fusion and these fused membranes may produce different levels of activity. The inventors have surprisingly observed that microsomes produced by this method consistently produce high levels of CYP activity.

### Experimental Outline

In order to find out if the final microsomal specific activity of a CYP expressed in a yeast strain of interest would be augmented by reductase, the cells are mixed (blended) with another strain containing either a hRD variant or the yeast P450 reductase (yRD). This can be done through various permutations/combinations as shown in the examples below:

1. CYP blended with ΔN24hRD plus low endogenous levels of yRD (endogenous=native yRD present in any yeast strain)
2. CYP blended with ΔN24hRD-cmyc plus low endogenous levels of yRD
3. CYP blended with high levels of yRD
4. CYP blended with ΔN24hRD
5. CYP blended with ΔN24hRD-cmyc

Steps 1-5 can be also carried out by blending cells which separately contain b5 to achieve further increase in CYP activity in the resultant microsomes.

### The Experimental Protocol

The optical density (OD) at 600 nm is measured and diluted 1/10. Once the culture reaches the required length of time for expression (i.e. the appropriate OD), the cells are harvested.

It is at this stage that the CYP containing cells (harvested from different yeast cell cultures) and the cells containing reductase or b5 (harvested separately from different sets of yeast cell cultures) are blended together in various ratios to achieve a maximal increase in microsomal CYP activity.

For example, 100 ml of yeast cells containing the expressed CYP are mixed with 100 ml of yeast cells containing the yeast reductase are blended together to produce a homogeneous cell mixture. Weights of empty dry sterile centrifuge buckets are recorded before the transferring of the yeast cell culture mix. The buckets are balanced, if required, with sterile media. The cultures are centrifuged at 2831 g (3622 rpm in the Sorvall) for 12 min at 4° C. The supernatants are poured away and the combined cells are resuspended gently in 100 ml of Wash Solution A (0.65M sorbitol, 10 mM Tris-HCl pH 7.5, 0.1 mM EDTA pH 8.0). The resuspended cells are centrifuged and washed twice with Wash Solution A after pelleting (via centrifugation) the washed cells each time. The weights of the buckets are finally recorded to indicate the final pellet weight. The pellet is then frozen at minus 80° C. immediately or the pellets are processed immediately without freezing.

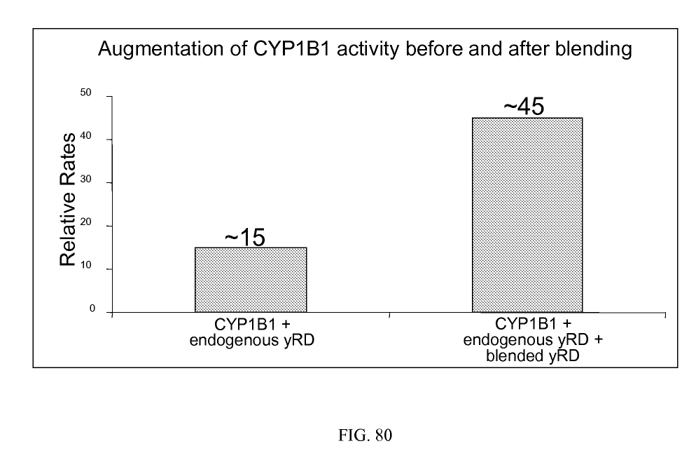

FIG. 80 is a representative example of the increase of CYP activity that can be seen through a typical blending experiment. It shows a dramatic increase in CYP1B1 activity (i.e. 7-ethoxyresorufin activity) when microsomes are prepared from two sets of cultures (one producing CYP1B1 alone and the other producing yRD alone) which have been blended via the protocol outlined above.

### Example 11

### Whole Cell Live Assay

This assay is a rapid and relatively inexpensive means (compared to the analysis of microsome activities) of determining the specific activities of various CYP-s in a kinetic live assay. The live assays will allow screening for inhibitors of CYP-s without the necessity of making microsomes. The activity in whole cells is 2 to 3-fold higher than in microsomes.

Cells are taken at various time points during the growth of yeast cells and the metabolism of the fluorescence substrate is analysed to determine CYP specific activities. The time for which cells are grown and the quantity of cells required for the assay may vary depending on the CYP being expressed, the strain of yeast being utilised and the substrate used for the live assay.

Cells are taken during exponential growth approximately 12-13 hours after addition of galactose, a sugar used for induction of expression of CYP-s from the GAL1 promoter.

150-250 μl of cells (approximately 2×10 E+07 to 2×10 E+09 cells) are aliquoted into 1.5 ml eppendorf tubes and centrifuged in a bench top centrifuge for thirty seconds at 13000 rpm.

The supernatant is removed by careful pipetting so as not to dislodge the cell pellet. The cell pellet is then resuspended in 450 μl of TE buffer (50 mM Tris-HCl pH. 7.4, 1 mM EDTA).

The resuspended cells are centrifuged again at 13000 rpm for 30 seconds. The supernatant is removed by careful pipetting and resuspended in 450 μl TE buffer. The suspension is centrifuged for 30 seconds at 13000 rpm. The supernatant is removed by careful pipetting and the cell pellet is finally resuspended in 50 μl of TE buffer.

The resulting cell suspension is then transferred to a black clear flat bottom 96-well plate ready for the addition of the relevant fluorescent substrate for the particular cytochrome P450 (CYP) that is to be analysed.

The relevant substrate (see Table 3 below) is diluted in 50 μl TE buffer which is added to the resuspended cells in the 96-well plate.

### Assay Parameters

The measurements are made during a time period of 30-40 minutes using the appropriate extinction/emission filters (see Table 3) and appropriate gain sensitivity setting to obtain the best kinetic output from the Synergy HT BioTek plate reader. The plate reader is set up to shake the plate for 5 seconds at an intensity of 4 between each reading. Kinetic analysis is carried out at 30° C.

Table 3 below contains some of the substrates which are used to assay CYP activity.

TABLE 3
The CYP substrates and the fluorescent products that are measured at specific
excitation/emission wavelengths to determine specific activity of CYP-s.

| | | | Excitation | Emission |
|---|---|---|---|---|
| | | | (Bandwidth of | (Bandwidth of |
| Enzyme | Substrate | Product | filter) | filter) |
| CYP1A1 | BzRes | Resorufin | 530 nm | 590 nm |
| CYP1B1 | BzRes | Resorufin | 530 nm | 590 nm |
| | CEC | CHC | 409 nm (20 nm) | 460 nm (40 nm) |
| CYP1A2 | CEC | CHC | 409 nm (20 nm) | 460 nm (40 nm) |
| CYP2C8 | DBF | Fluorescein | 485 nm (20 nm) | 538 nm (25 nm) |
| CYP2C9 | 7-MFC | HFC | 409 nm (20 nm) | 530 nm (25 nm) |
| | DBF | Fluorescein | 485 nm (20 nm) | 538 nm (25 nm) |
| | Vivid BOMCC | Blue Standard | 405 nm (20 nm) | 460 nm (40 nm) |
| | Vivid BOMF | Green Standard | 485 nm (20 nm) | 530 nm (25 nm) |
| CYP2C19 | CEC | CHC | 409 nm (20 nm) | 460 nm (40 nm) |
| | DBF | Fluorescein | 485 nm (20 nm) | 538 nm (25 nm) |
| | Vivid EOMCC | Blue Standard | 405 nm (20 nm) | 460 nm (40 nm) |
| CYP2D6 | AMMC | AHMC | 390 nm (20 nm) | 460 nm (40 nm) |
| | CEC | CHC | 409 nm (20 nm) | 460 nm (40 nm) |
| | Vivid EOMCC | Blue Standard | 405 nm (20 nm) | 460 nm (40 nm) |
| CYP2D6 | Vivid MOBFC | Cyan Standard | 405 nm (40 nm) | 490 nm (40 nm) |
| CYP2E1 | 7-MFC | HFC | 409 nm (20 nm) | 530 nm (25 nm) |
| | Vivid EOMCC | Blue Standard | 405 nm (20 nm) | 460 nm (40 nm) |
| CYP3A4 | 7-BQ | Quinolinol | 409 nm (20 nm) | 530 nm (25 nm) |
| | BFC | HFC | 409 nm (20 nm) | 530 nm (25 nm) |
| | BzRes | Resorufin | 530 nm (25 nm) | 590 nm (35 nm) |
| | DBF | Fluorescein | 485 nm (20 nm) | 538 nm (25 nm) |
| | Vivid BOMCC | Blue Standard | 405 nm (20 nm) | 460 nm (40 nm) |

### Example 12

### The Yeast ADH2 Promoter, Inducible by Ethanol, Allows Higher Expression of CYP2D6, CYP1A2, and Other CYP-s

ADH2 promoter driven expression of (a) CYP2D6 and (b) CYP1A2 was compared with the expression of these enzymes from the PGK1 (constitutive), GAPDH (constitutive) and the GAL1 (inducible) promoters. In order to do so, first (A) the CYP2D6 and CYP1A2 genes were cloned downstream of

(1) ADH2 promoter,

(2) PGK1 promoter,

(3) GAPDH promoter, or

(4) GAL1 promoter

in yeast 2-micron plasmids, and
(B) the delN24hRD-cmyc (ΔN24hRD-cmyc) gene was cloned downstream of

(5) ADH2 promoter,

(6) PGK1 promoter,

(7) GAPDH promoter, or

(8) GAL1 promoter

in yeast LEU2 integrating plasmids.

Then, the yeast LEU2 integrating plasmids bearing the ΔN24hRD-cmyc gene, driven by different promoters, were integrated into the yeast strain W303B (as used previously) to obtain four different strains. Each of these four strains was transformed with two 2-micron plasmids, one bearing the CYP2D6 and the other the CYP1A2 gene.

Amounts and enzyme activities of P450 obtained from the CYP2D6-producing and the CYP1A2-producing strains were compared. Results indicate that the ADH2 promoter driven CYP expression is superior than expression from the other promoters.

### Example 12.1

### Construction of the Plasmids that Allow Expression of Functional Enzymes CYP2D6 and CYP1A2 Using the Yeast ADH2 Promoter are Set Out Below

### Example 12.1.1

### Cloning of the 573 bp Yeast ADH2 Promoter as a SaI(NgoMIV)-(HindIII)BamHI Fragment

The cloning of a SalI(NgoMIV)-(HindIII)BamHI ADH2 promoter fragment (SEQ ID No. 89) in pBlueScriptII SK(+) was performed as in Example 1.2, using ADH2 promoter sequence specific primers (5′ PCR primer: 5′-CCGGTCGACG CCGGCGGCAA AACGTAGGGG CAAACAAACG G-3′ (the first six letters in italics signify the SalI site and the next six letters represent the NgoMIV site—SEQ ID No. 90) & 3′ PCR primer: 5′-CGGGATCCAA GCTTTGTGTA TTACGATATA GTTAATAG-3′ (the first six letters in italics signify the BamHI site and the next six letters represent the HindIII site—SEQ ID No. 91)). The amplified fragment, digested with SalI-BamHI, was cloned in pBlueScriptII KS(+) digested with SalI-BamHI.

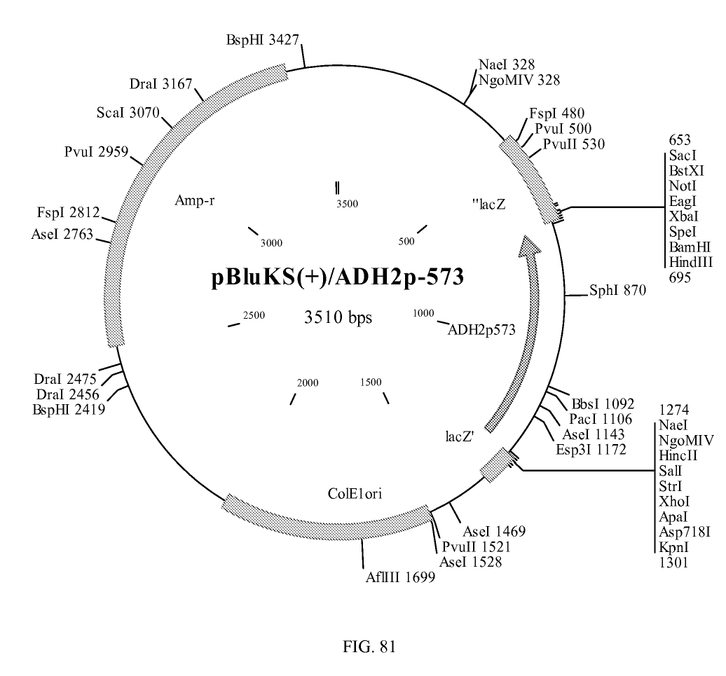

One correct clone obtained after ligation and transformation in DH5alpha bacterial cells was named pBluKS(+)/ADH2p-573 (FIG. 81) and was used for further cloning in a 2-micron and an integrating yeast expression vector. The veracity of the clone was confirmed by restriction enzyme analysis and corroborated by DNA sequencing.

### Example 12.1.2

### Cloning of the ADH2 Promoter in a Yeast 2-Micron Vector

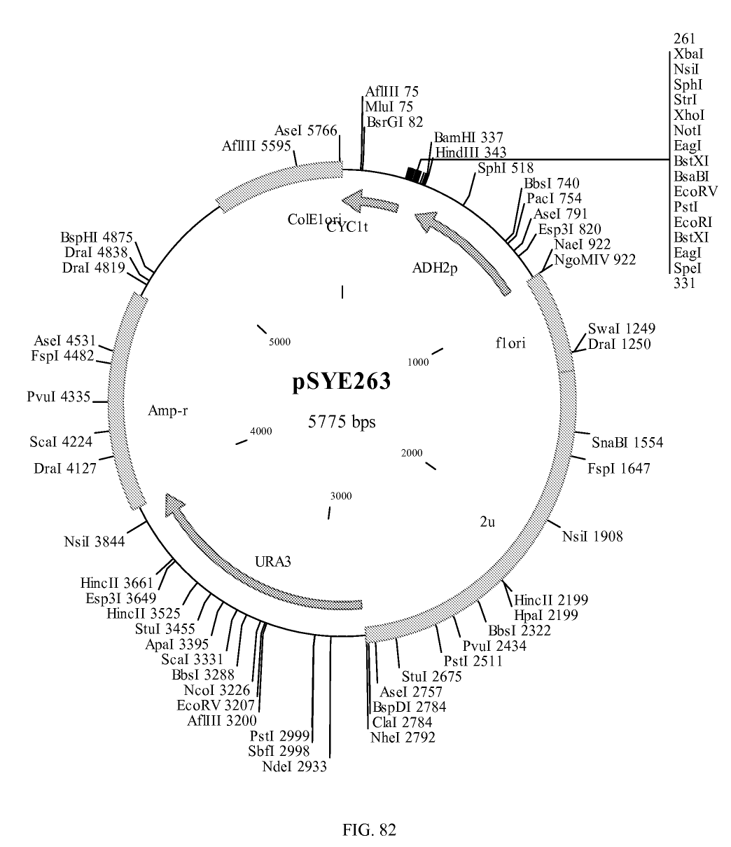

A 585 bp NgoMIV-BamHI ADH2 promoter fragment was isolated from pBluKS(+)/ADH2p-573 (FIG. 81) and cloned in pYES2 (a yeast 2-micron vector obtained from Invitrogen) digested with NgoMIV and BamHI to obtain the plasmid pSYE263 (FIG. 82).

### Example 12.1.3

### Cloning of the Human CYP2D6 Gene in pSYE263

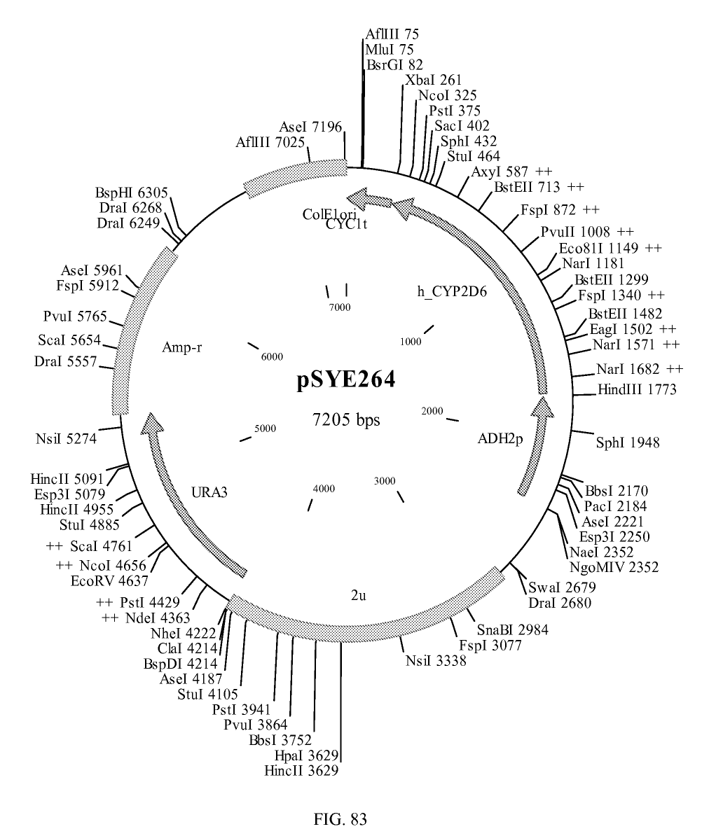

A 1506 bp BamHI-XbaI fragment containing the human CYP2D6 gene (SEQ ID No. 92) is cloned in pSYE263 (FIG. 82), digested with BamHI and XbaI, to obtain the plasmid pSYE264 (FIG. 83).

### Example 12.1.4

### Cloning of the Human CYP1A2 Gene in pSYE263

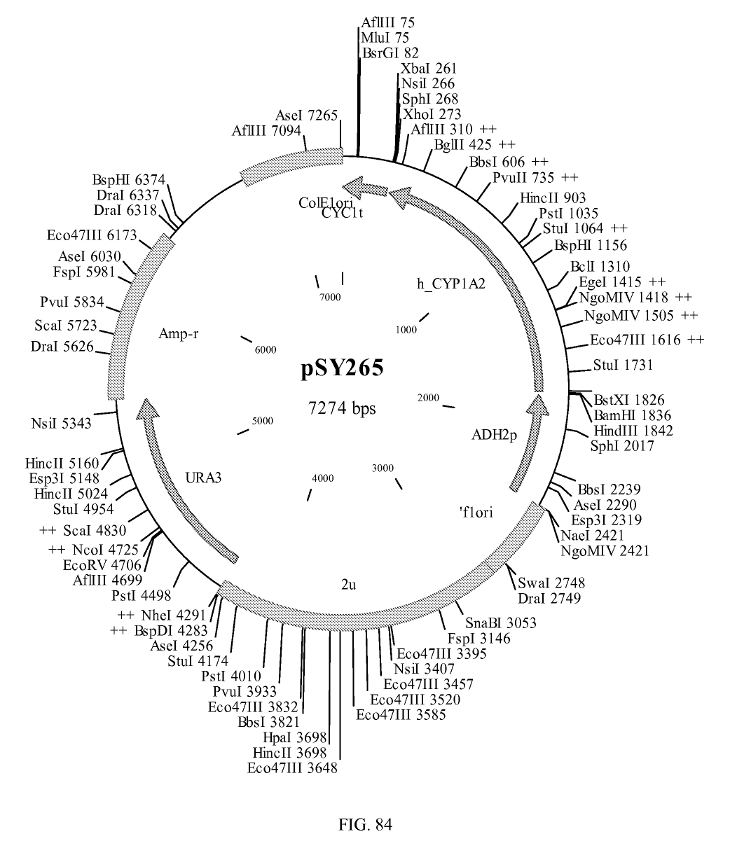

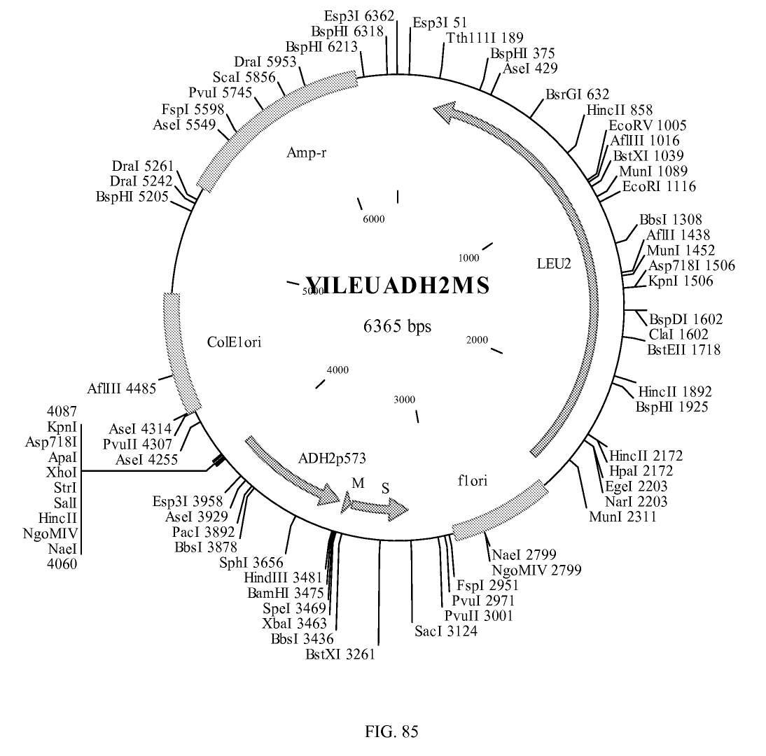

A 1563 bp BamHI-XhoI fragment containing the human CYP1A2 gene (SEQ ID No. 93) is cloned in pSYE263 (FIG. 82), digested with BamHI and XhoI, to obtain the plasmid pSYE265 (FIG. 84).

### Example 12.1.5

### Cloning of the ADH2 Promoter in a Yeast Integrating Vector

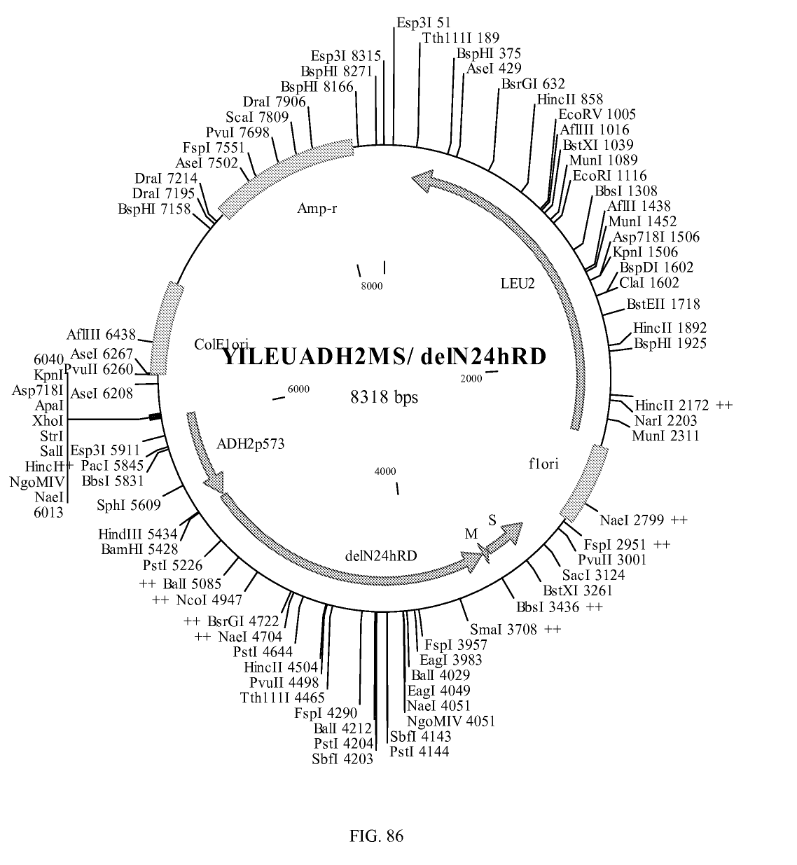

A 591 bp bp SalI-BamHI ADH2 promoter fragment was isolated from pBluKS(+)/ADH2p-573 (FIG. 81) and cloned in YILEUGAL1MS (FIG. 23; a yeast LEU2-integrating vector) digested with SalI and BamHI to obtain the plasmid YILEUADH2MS (FIG. 85).

### Example 12.1.6

### Cloning of the delN24hRD (ΔN24hRD) gene in YILEUADH2MS

The BamHI-Xba11965 bp fragment of the delN24hRD (ΔN24hRD) gene (SEQ ID No 37) was cloned in the yeast integrating vector YILEUADH2MS (FIG. 85), digested with BamHI and XbaI, to obtain the plasmid YILEUADH2MS/delN24hRD (FIG. 86). The plasmid encodes the ΔN24hRD-cmyc gene.

### Example 12.2

### Construction of Plasmids that Allow Expression of Functional Enzymes CYP2D6 and CYP1A2 Using the Yeast PGK1 Promoter

### Example 12.2.1

### Cloning of the 650 bp Yeast PGK1 Promoter as a SaI-BamHI Fragment

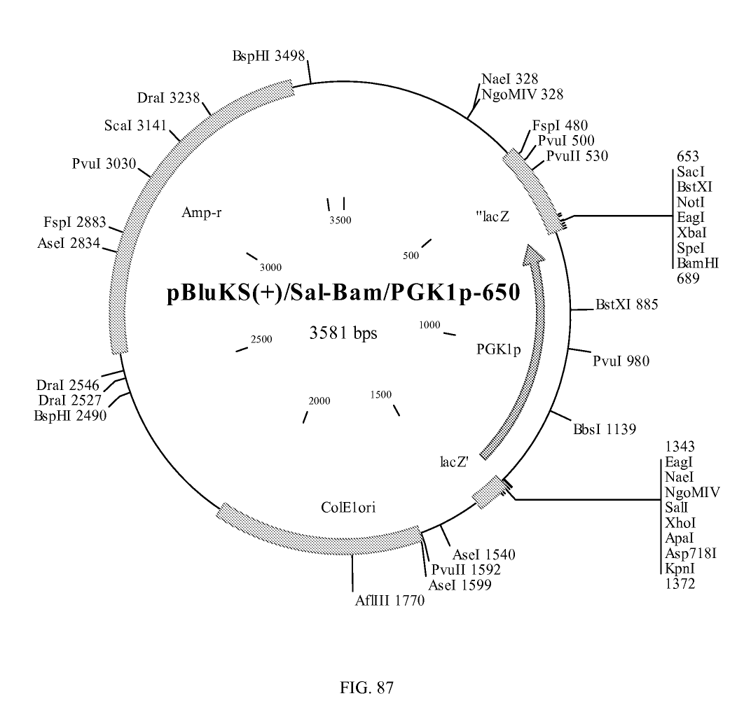

The cloning of a SalI(NgoMIV)-BamHI PGK1 promoter fragment (SEQ ID No. 94) in pBluKS(+) was performed as in Example 1.2, using PGK1 promoter sequence specific primers (5′ PCR primer: 5′-ATGTCGACGC CGGCCGATTT GGGCGCGAAT CCTTTATTTT GGC-3′ (the first six letters in italics signify the SalI site and the next six letters represent the NgoMIV site—SEQ ID No. 95) & 3′ PCR primer: 5′-TAGGATCCTG TTTTATATTT GTTGTAAAAA GTAGATAATT AC-3′ (the letters in italics signify the BamHI site—SEQ ID No. 96)). The amplified fragment, digested with SalI-BamHI, was cloned in pBlueScriptII KS(+) digested with SalI-BamHI. One correct clone obtained after ligation and transformation in DH5alpha bacterial cells was named pBluKS(+)/Sal-Bam/PGK1p-650 (FIG. 87) and was used for further cloning in a 2-micron and an integrating yeast expression vector. The veracity of the clone was confirmed by restriction enzyme analysis and corroborated by DNA sequencing.

### Example 12.2.2

### Cloning of the PGK1 Promoter in a Yeast 2-Micron Vector

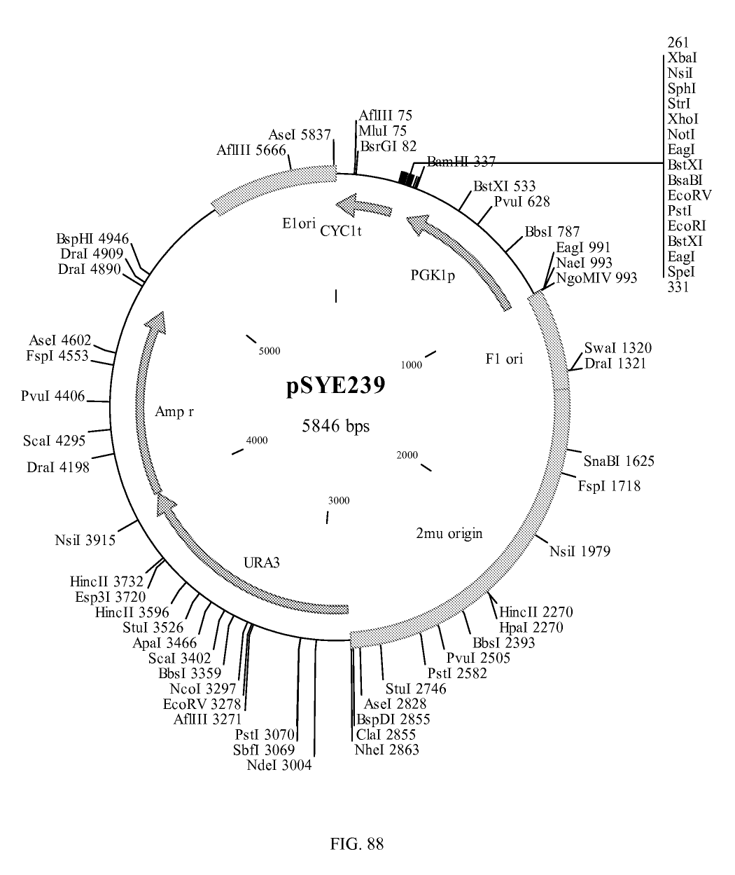

A 656 bp NgoMIV-BamHI PGK1 promoter fragment was isolated from pBluKS(+)/Sal-Bam/PGK1p-650 (FIG. 87) and cloned in pYES2 (a yeast 2-micron vector obtained from Invitrogen) digested with NgoMIV and BamHI to obtain the plasmid pSYE239 (FIG. 88).

### Example 12.2.3

### Cloning of the Human CYP2D6 Gene in pSYE239

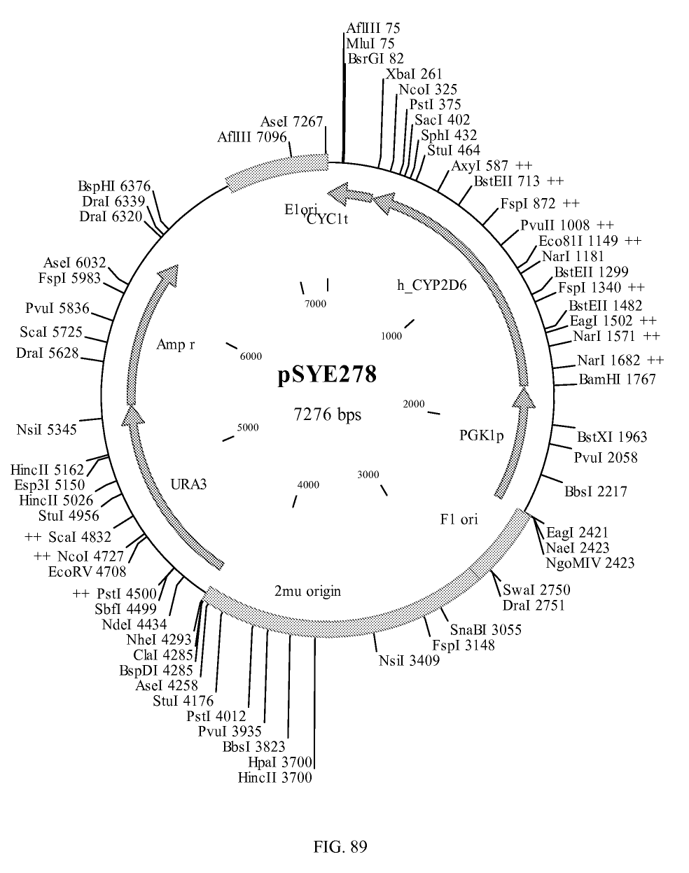

A 1506 bp BamHI-XbaI fragment containing the human CYP2D6 gene (SEQ ID No. 92) is cloned in pSYE239 (FIG. 88), digested with BamHI and XbaI, to obtain the plasmid pSYE278 (FIG. 89).

### Example 12.2.4

### Cloning of the Human CYP1A2 Gene in pSYE239

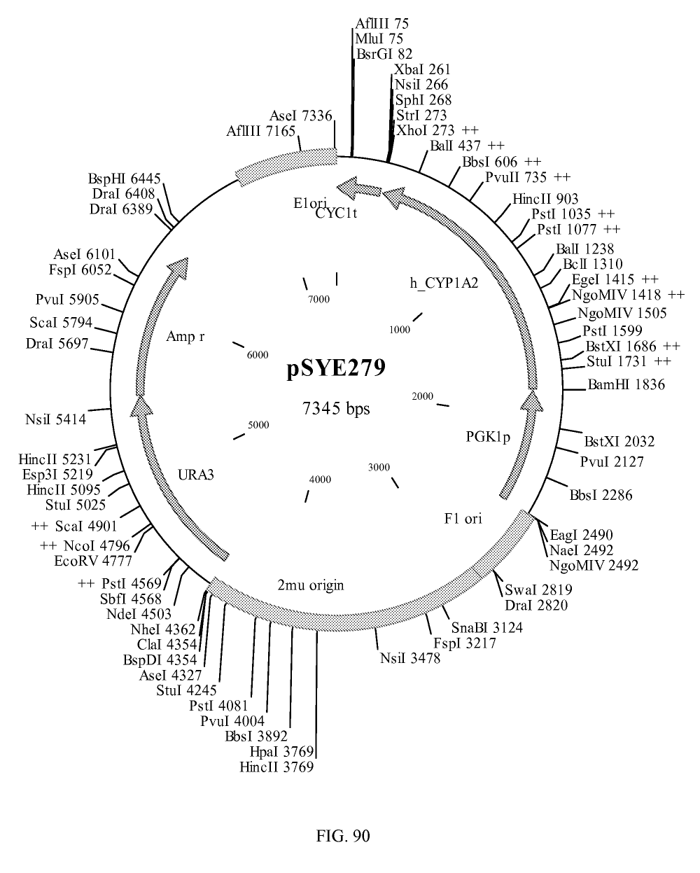

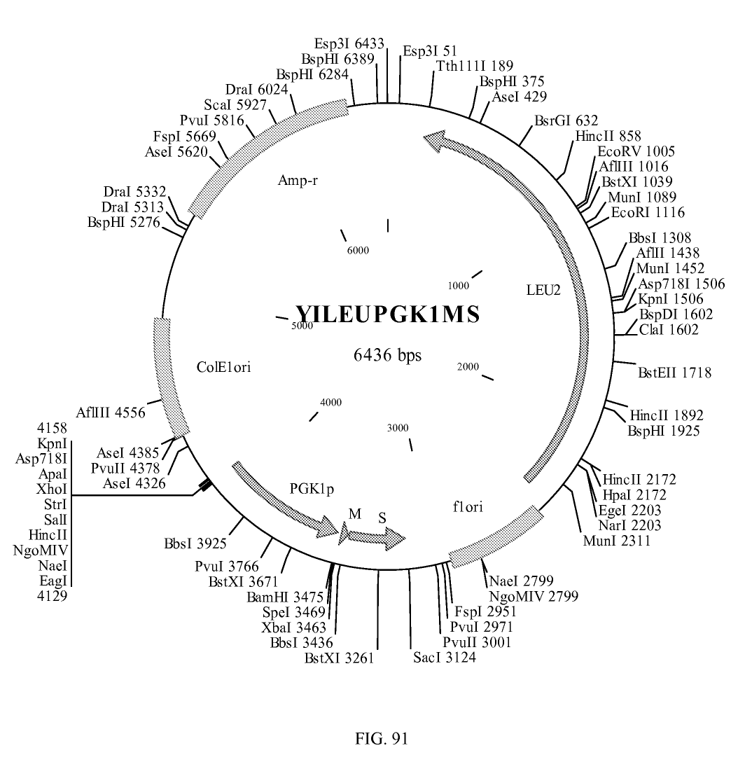

A 1563 bp BamHI-XhoI fragment containing the human CYP1A2 gene (SEQ ID No. 93) is cloned in pSYE239 (FIG. 88), digested with BamHI and XhoI, to obtain the plasmid pSYE279 (FIG. 90).

### Example 12.2.5

### Cloning of the PGK1 Promoter in a Yeast Integrating Vector

A 662 bp SalI-BamHI ADH2 promoter fragment was isolated from pBluKS(+)/Sal-Bam/PGK1p-650 (FIG. 87) and cloned in YILEUGAL1MS (FIG. 23; a yeast LEU2-integrating vector) digested with SalI and BamHI to obtain the plasmid YILEUPGKIMS (FIG. 91).

### Example 12.2.6

### Cloning of the delN24hRD (ΔN24hRD) Gene in YILEUPGK1MS

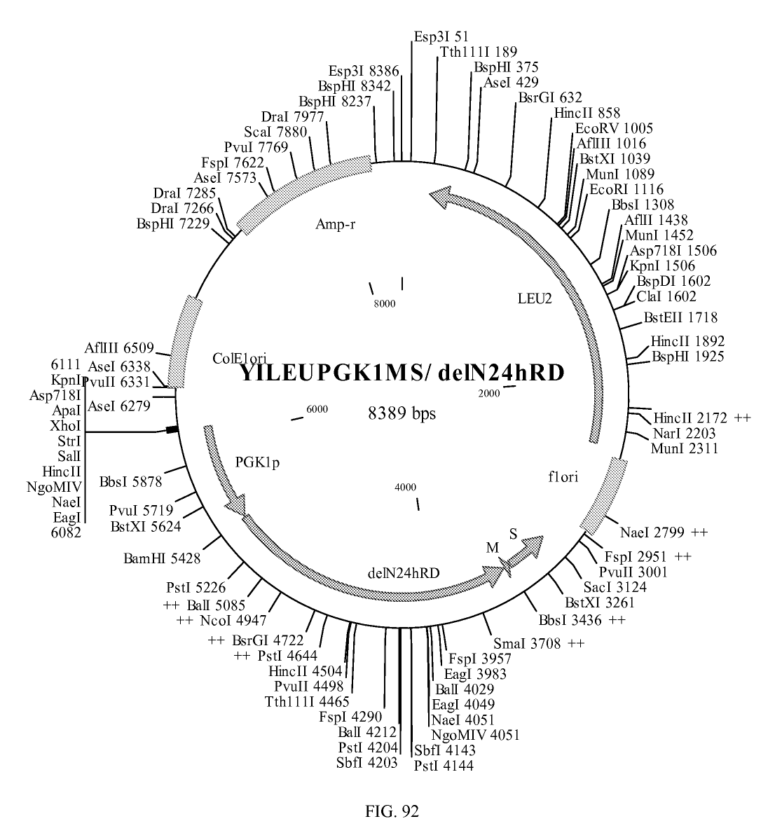

The BamHI-Xba11965 bp fragment of the delN24hRD (ΔN24hRD) gene (SEQ ID No 37) was cloned in the yeast integrating vector YILEUPGK1MS (FIG. 91), digested with BamHI and XbaI, to obtain the plasmid YTLEUPGK1MS/delN24hRD (FIG. 92). The plasmid encodes the ΔN24hRD-cmyc gene.

### Example 12.3

### Construction of Plasmids that would Allow Expression of Functional Enzymes CYP2D6 and CYP1A2 Using the Yeast GAPDH Promoter

### Example 12.3.1

### Cloning of the 696 bp Yeast GAPDH Promoter, Linked Downstream of a 275 bp pBR322 Fragment, as a SalI-BamHI Fragment

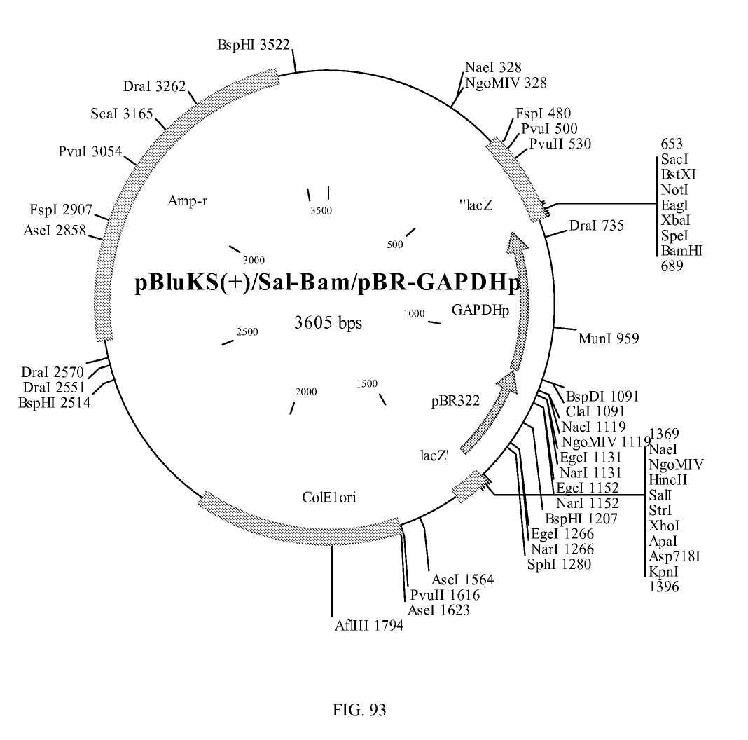

The cloning of a SalI(NgoMIV)-BamHI pBR-GAPDH promoter fragment (SEQ ID No. 97) in pBluKS(+) was performed as in Example 1.2, using pBR-GAPDH promoter sequence specific primers (5′ PCR primer: 5′-ATGTCGACGC CGGCGCTCTC CCTTATGCGA CTCCTGCATT AGG-3′ (the first six letters in italics signify the SalI site and the next six letters represent the NgoMIV site—SEQ ID No. 98)& 3′ PCR primer: 5′-TAGGATCCTT TGTTTATGTG TGTTTATTCG AAACTAAGTT CTTGG-3′ (the letters in italics signify the BamHI site—SEQ ID No. 102)). The amplified fragment, digested with SalI-BamHI, was cloned in pBlueScriptII KS(+) digested with SalI-BamHI. One correct clone obtained after ligation and transformation in DH5alpha bacterial cells was named pBluKS(+)/Sal-Bam/pBR-GAPDHp (FIG. 93) and was used for further cloning in a 2-micron and an integrating yeast expression vector. The veracity of the clone was confirmed by restriction enzyme analysis and corroborated by DNA sequencing.

### Example 12.3.2

### Cloning of the pBR-GAPDH Promoter in a Yeast 2-Micron Vector

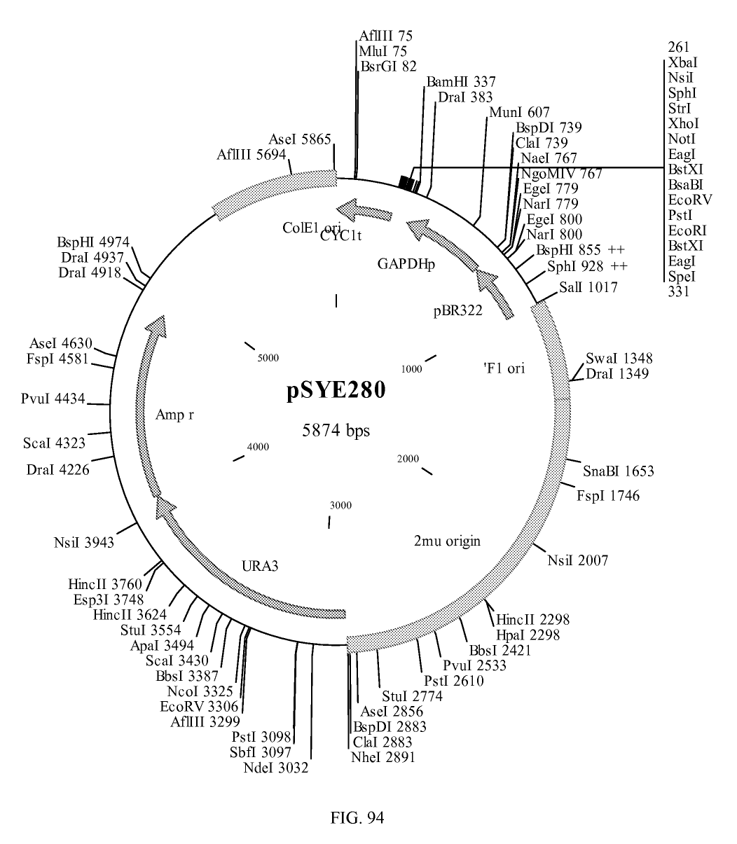

A 680 bp NgoMIV-BamHI pBR-GAPDH promoter fragment (from a partial digest) was isolated from pBluKS(+)/Sal-Bam/pBR-GAPDHp (FIG. 93) and cloned in pYES2 (a yeast 2-micron vector obtained from Invitrogen) digested with NgoMIV and BamHI to obtain the plasmid pSYE280 (FIG. 94).

### Example 12.3.3

### Cloning of the Human CYP2D6 Gene in pSYE280

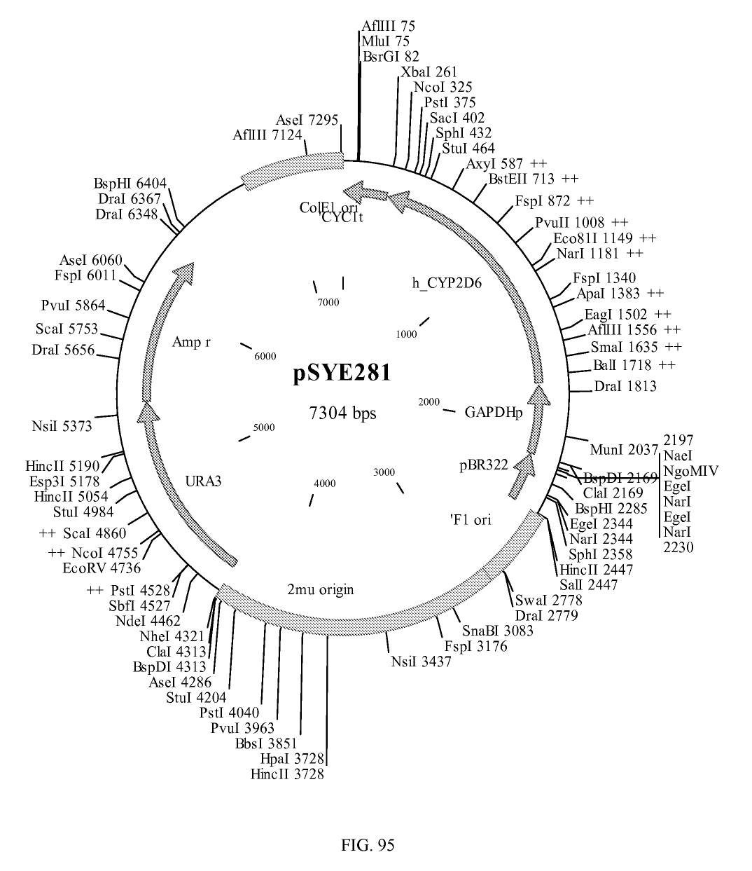

A 1506 bp BamHI-XbaI fragment containing the human CYP2D6 gene (SEQ ID No. 92) is cloned in pSYE280 (FIG. 88), digested with BamHI and XbaI, to obtain the plasmid pSYE281 (FIG. 95).

### Example 12.3.4

### Cloning of the Human CYP1A2 Gene in pSYE280

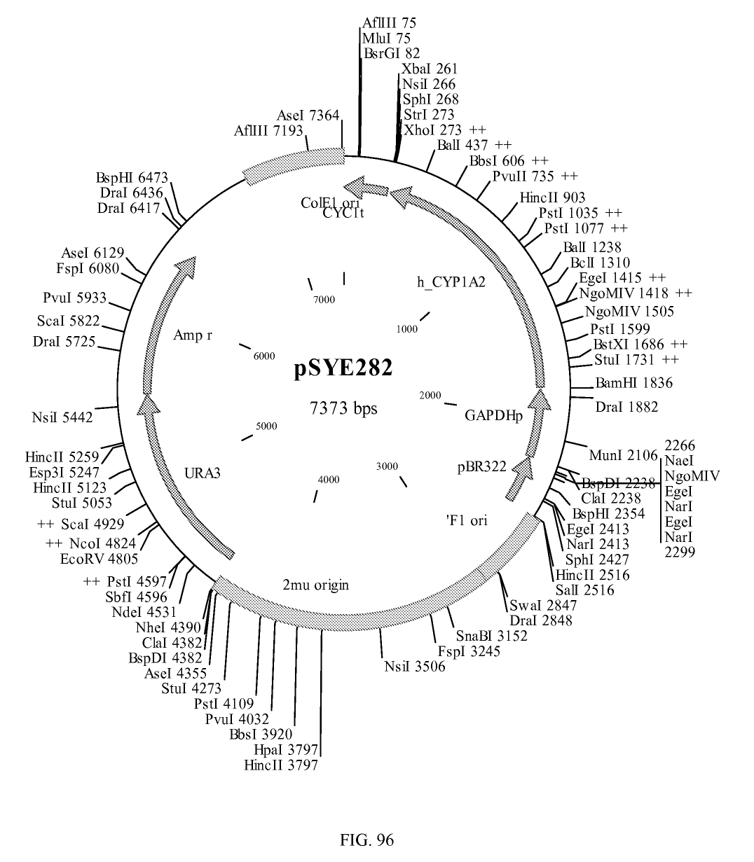

A 1563 bp BamHI-XhoI fragment containing the human CYP1A2 gene (SEQ ID No. 93) is cloned in pSYE280 (FIG. 88), digested with BamHI and XhoI, to obtain the plasmid pSYE282 (FIG. 96).

### Example 12.3.5

### Cloning of the PGK1 Promoter in a Yeast Integrating Vector

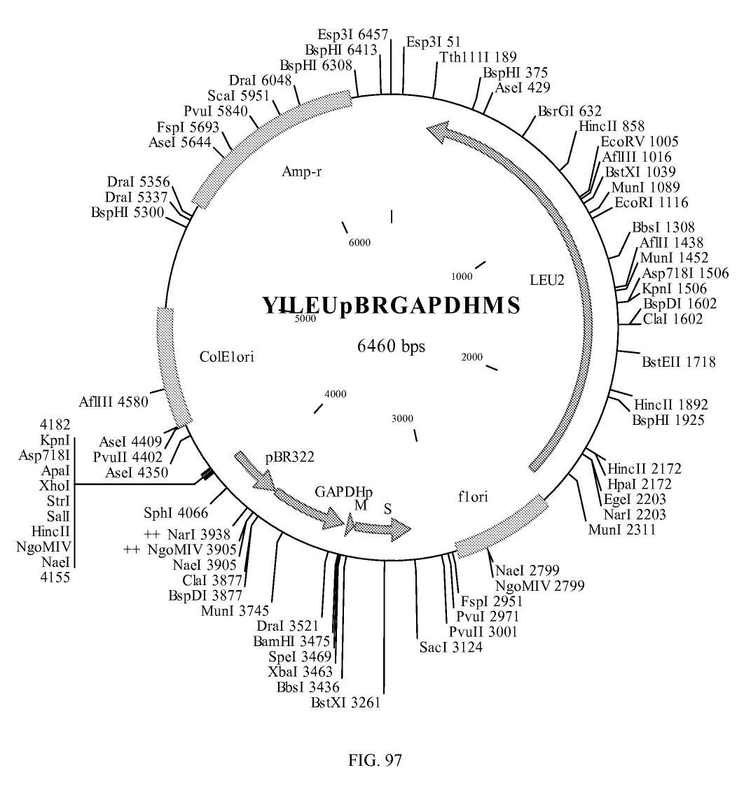

A 686 bp SalI-BamHI pBR-GAPDH promoter fragment (from a partial digest) was isolated from pBluKS(+)/Sal-Bam/pBR-GAPDHp (FIG. 93) and cloned in YILEUGAL1MS (FIG. 23; a yeast LEU2-integrating vector) digested with SalI and BamHI to obtain the plasmid YILEUpBRGAPDHMS (FIG. 97).

### Example 12.3.6

### Cloning of the delN24hRD (ΔN24hRD) Gene in YILEUpBRGAPDHMS

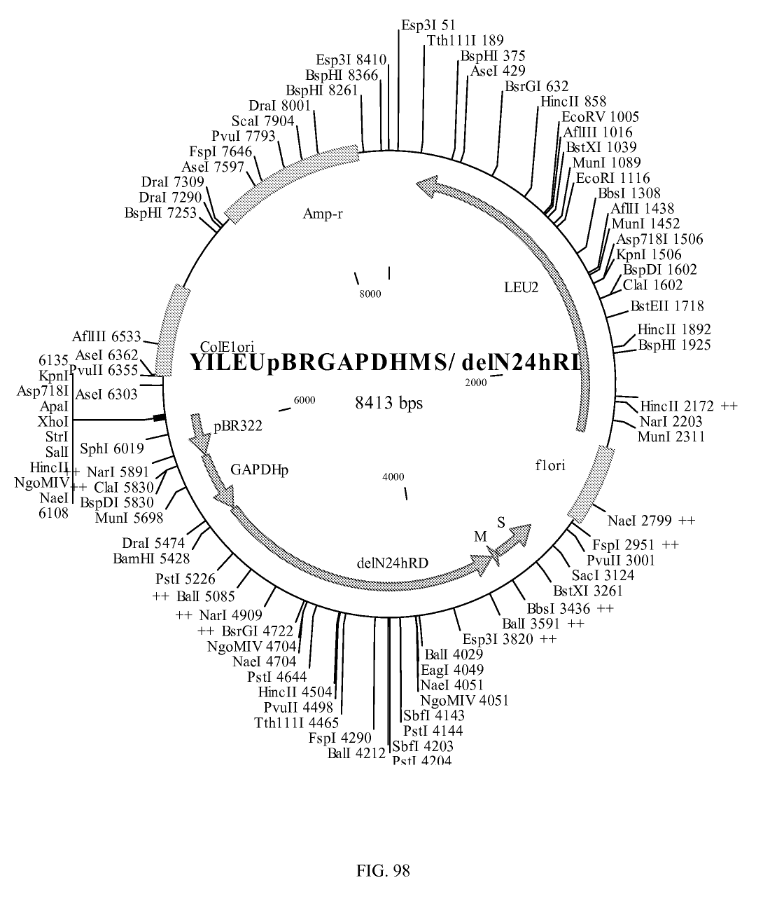

The BamHI-Xba11965 bp fragment of the delN24hRD (ΔN24hRD) gene (SEQ ID No 37) was cloned in the yeast integrating vector YILEUpBRGAPDHMS (FIG. 97), digested with BamHI and XbaI, to obtain the plasmid YILEUpBRGAPDHMS/delN24hRD (FIG. 98). The plasmid encodes the ΔN24hRD-cmyc gene.

### Example 12.4

### Construction of Plasmids that Allow Expression of Functional Enzymes

CYP2D6 and CYP1A2 Using the Yeast GAL1 Promoter

### Example 12.4.1

### Cloning of the Human CYP2D6 Gene in pSYE224

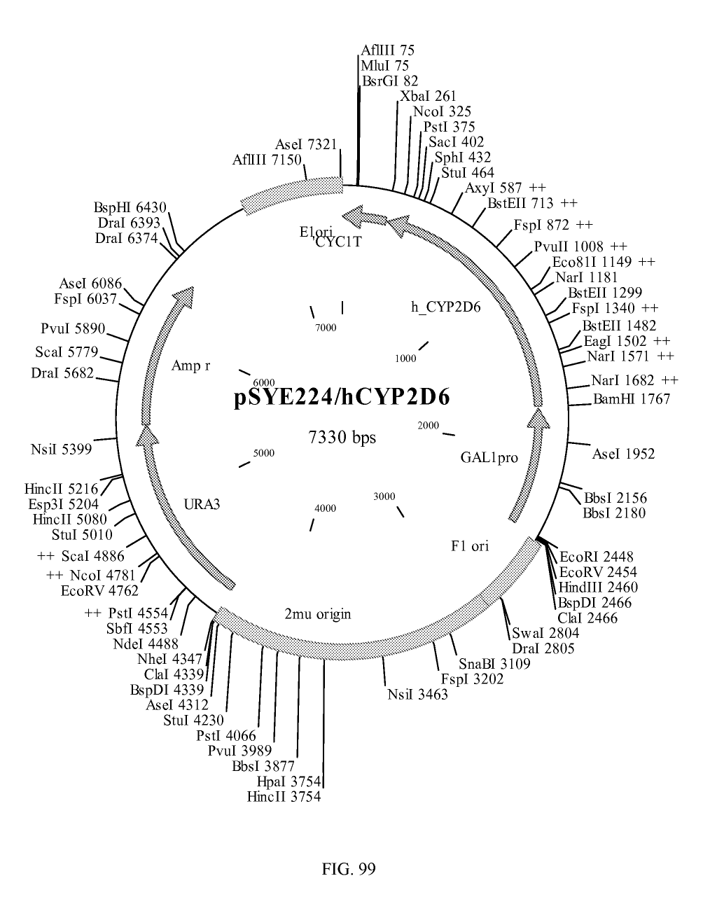

A 1506 bp BamHI-XbaI fragment containing the human CYP2D6 gene (SEQ ID No. 92) is cloned in pSYE224 (FIG. 4), digested with BamHI and XbaI, to obtain the plasmid pSYE224/hCYP2D6 (FIG. 99).

### Example 12.4.2

### Cloning of the Human CYP1A2 Gene in pSYE224

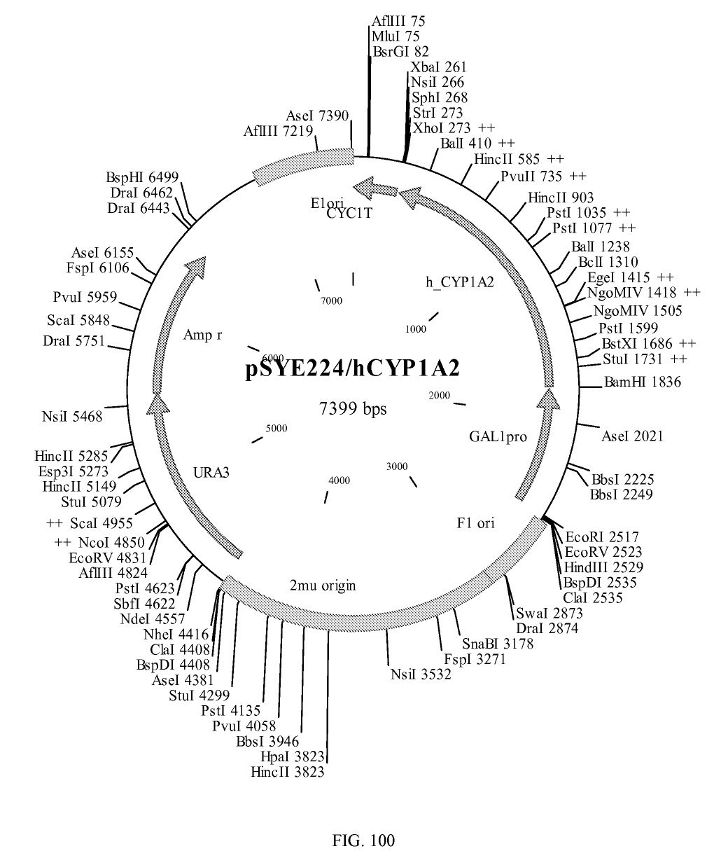

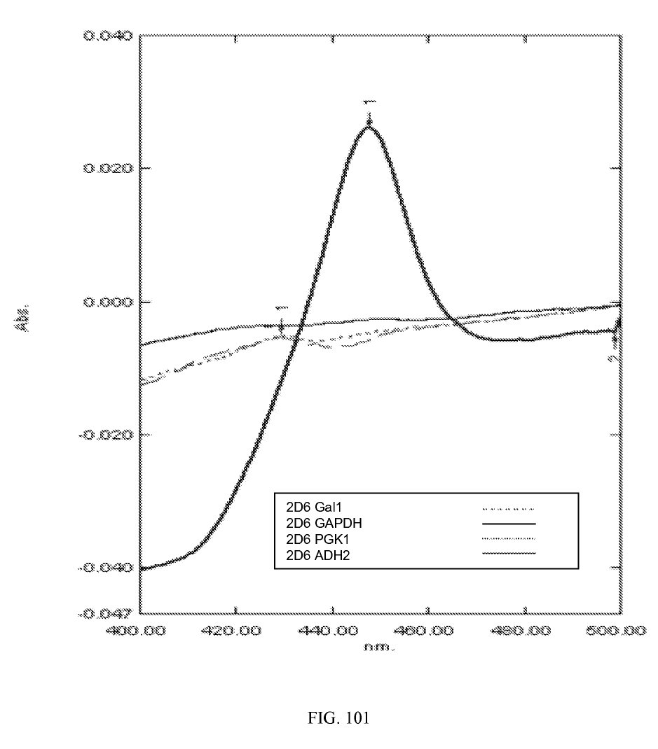

A 1563 bp BamHI-XhoI fragment containing the human CYP1A2 gene (SEQ ID No. 93) is cloned in pSYE224 (FIG. 4), digested with BamHI and XhoI, to obtain the plasmid pSYE224/hCYP1A2 (FIG. 100).

### Example 12.5

### Integration of the ΔN24hRD-c-myc Gene, a Human NADPH P450 Reductase Variant, which are Driven by Different Promoters into the Yeast Strain W303B at the LEU2 Locus

The integrations were performed with the plasmids YILEUADH2MS/delN24hRD (FIG. 86), YILEUPGK1MS/delN24hRD (FIG. 92), YILEUpBRGAPDHMS/delN24hRD (FIG. 97) and pSYI201 (FIG. 31), as in Example 2.12. All plasmids were linearised with BstEII before integration. The resultant strains were named

(1) YI050 (W303B:: YILEUADH2MS/delN24hRD),

(2) YI051 (W303B:: YILEUPGK1MS/delN24hRD),

(3) YI052 (W303B:: YILEUpBRGAPDHMS/delN24hRD) and

(4) YI002 (W303B:: pSYI201).

All integrations were confirmed using PCR with primers specific for the ΔN24hRD-cmyc gene.

### Example 12.6

### Yeast Transformation

The strains (1) to (4), as elaborated in Example 12.4,

(1) YI050 (W303B:: YILEUADH2MS/delN24hRD),

(2) YI051 (W303B:: YILEUPGK1MS/delN24hRD),

(3) YI052 (W303B:: YILEUpBRGAPDHMS/delN24hRD) and

(4) YI002 (W303B:: pSYI201)

were transformed with the plasmids (Strain #1 with plasmids A & B, strain #2 with plasmids C &D, strain #3 with plasmids E & F, and strain #4 with plasmids pSYE224/hCYP2D6 and pSYE224/hCYP1A2)

(A) pSYE264 (ADH2p-CYP2D6),

(B) pSYE265 (ADH2p-CYP1A2),

(C) pSYE278 (PGK1p-CYP2D6),

(D) pSYE279 (PGK1p-CYP1A2),

(E) pSYE281 (pBR-GAPDHp-CYP2D6),

(F) pSYE282 (pBR-GAPDHp-CYP1A2),

(G) pSYE224/hCYP2D6, and

(H) pSYE224/hCYP1A2).

The following strains were obtained using the DMSO method of yeast transformation (as described in Example 1.15):

(i) YI053 (YI050::pSYE264),

(ii) YI054 (YI050::pSYE265),

(iii) YI055 (YI051::pSYE278),

(iv) YI056 (YI051::pSYE279),

(v) YI057 (YI052::pSYE281),

(vi) YI058 (YI052::pSYE281),

(vii) YI059 (YI002::pSYE224/hCYP2D6),

(viii) YI059 (YI002:: pSYE224/hCYP1A2).

### Example 12.7

### Growth of Yeast Cultures for Preparation of Yeast Microsomes

The yeast cells from the strains YI053 to YI059 were grown by the method used for “Growing yeast cultures for microsome preparation” as in Example 1.15. For PGK1 and the pBR-GAPDH promoters, cell were grown in glucose for 40 h. For the ADH2 promoter, ethanol was added together after an overnight in glucose. Microsomes were prepared as in Example 1.15.

### Example 12.8

### P450 Measurement and Activities

P450 amounts were measured via CO-difference spectroscopy as described in Example 1.15. CYP2D6 and CYP1A2 activities were measured fluorimetrically as described in the protocols in the Gentest website.

### Example 12.9

### Results

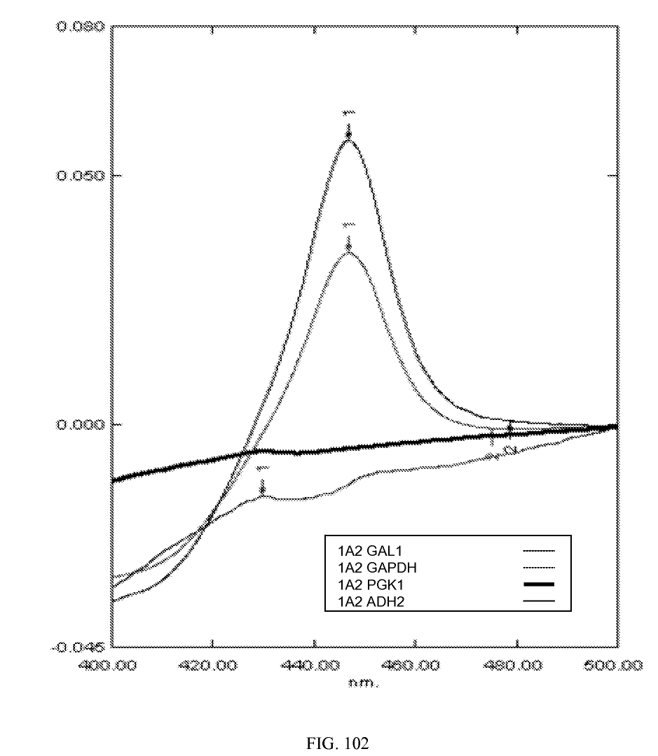

The results for the effects for the different promoters on amount of CYP2D6 and CYP1A1 are shown in FIGS. 100 and 102, respectively. A comparison of the amount and activity of P450 is shown in Table 4 below.

TABLE 4

| | Protein | P450 pmol/ | |
|---|---|---|---|
| Clone | mg/ml | mg protein | P450 activity |
| 2D6 GAL1 | 15-25 | 0 | −ve |
| 2D6 GAPDH | 15-20 | 0 | −ve |
| 2D6 PGK1 | 10-20 | 0 | −ve |
| 2D6 ADH2 | 15-25 | 200-300 | +++ |
| 1A2 GAL1 | 15-20 | 0 | −ve |
| 1A2 GAPDH | 10-20 | 100-300 | ++ |
| 1A2 PGK1 | 15-20 | 0 | −ve |
| 1A2 ADH2 | 10-20 | 150-400 | ++++ |

It can be seen that, for expression of cytochrome P450s, ADH2 can be an extremely useful promoter.

While this invention has been particularly shown and described with references to preferred embodiments thereof, it will be understood by those skilled in the art that various changes in form and details may be made therein without departing from the scope of the invention encompassed by the appended claims.

1-206. (canceled)

207. An isolated or recombinant nucleic acid molecule comprising a nucleotide sequence encoding a P450 reductase lacking N-terminal amino acids, wherein the P450 reductase, when co-expressed with a cytochrome P450, increases the activity and/or expression of the cytochrome P450 compared to the activity and/or expression of the cytochrome P450 when co-expressed with a wild type P450 reductase, the P450 reductase comprising a c-myc tepitope tag at the C-terminal end thereof.

208. The nucleic acid molecule of claim 207, wherein the nucleic acid molecule encodes a human P450 reductase.

209. The nucleic acid molecule of claim 208, wherein the P450 reductase lacks at least the 24 N-terminal amino acids.

210. The nucleic acid molecule of claim 209, wherein the human P450 reductase comprises the amino acid sequence of SEQ ID NO: 26.

211. The nucleic acid molecule of claim 207 comprising or consisting of:
a) a nucleotide sequence of SEQ ID NO: 37;
b) a nucleotide sequence having at least 80% identity to the sequence of a) and encoding a P450 reductase which, when co-expressed with a cytochrome P450, increases the activity and/or expression of the cytochrome P450 compared to the activity and/or expression of the cytochrome P450 when co-expressed with a wild type P450 reductase;
c) a nucleotide sequence which is complementary to the sequence of a) or b); or
d) a nucleotide sequence which codes for the same polypeptide as the sequence of a), b) or c).

212. The nucleic acid molecule of claim 207, wherein the nucleic acid molecule comprises or consists of the sequence of SEQ ID NO: 42.

213. The nucleic acid molecule of claim 207, wherein the nucleic acid molecule further comprises a promoter which controls expression of the nucleotide sequence.

214. The nucleic acid molecule of claim 213, wherein the promoter comprises a truncated GAL promoter.

215. An isolated or recombinant nucleic acid molecule comprising a truncated GAL promoter for controlling the expression of a nucleotide sequence.

216. The nucleic acid molecule of claim 215, wherein the truncated GAL promoter is a truncated GAL1 promoter.

217. The nucleic acid molecule of claim 216, wherein the truncated GAL1 promoter is a GAL1 promoter truncated at nucleotide 202.

218. The nucleic acid molecule of claim 217, wherein the truncated GAL1 promoter comprises or consists of the sequence of SEQ ID NO: 2.

219. A polypeptide encoded by the nucleic acid molecule of claim 207.

220. The polypeptide of claim 219, which comprises or consists of the amino acid sequence of SEQ ID NO: 27.

221. A vector comprising a nucleic acid molecule of claim 207.

222. The vector of claim 221, wherein the vector comprises two copies of the nucleic acid molecule, each copy under the control of a respective promoter.

223. A cell transformed with the nucleic acid molecule of claim 207.

224. A method of expressing a nucleic acid molecule in a cell, comprising transforming the cell with a nucleic acid molecule of claim 207.

225. A protein expression system comprising:
i) a cell of claim 223; and
ii) a vector comprising a nucleotide sequence encoding a target protein, said sequence under the control of a promoter which causes expression of the nucleotide sequence.

226. A protein expression system of claim 225, wherein the nucleotide sequence encodes a heterologous cytochrome P450 or a cytochrome b5 protein.
